SEQUENCE LISTING (42 sequences)

SEQ ID NO: 1 - protein, 133 aa, Artificial Sequence [sequence omitted]

SEQ ID NO: 2 - protein, 103 aa, Artificial Sequence
  Cholera toxin B subunit
TPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 3 - protein, 310 aa, Artificial Sequence
  Neisseria PorB
DVTLYGTIKAGVETSRSVEHNGGQVVSVETGTGIVDLGSKIGFKGQEDLGNGLKAIWQVEQKASIAGTDSGWGNRQSFIGLKGGFGKLRVGRLNSVLKDTGDINPWDSKSDYLGVNKIAEPEARLISVRYDSPEFAGLSGSVQYALNDNAGRHNSESYHAGFNYKNGGFFVQYGGAYKRHQDVDDVKIEKYQIHRLVSGYDNDALYASVAVQQQDAKLVEDNSHNSQTEVAATLAYRFGNVTPRVSYAHGFKGSVDDAKRDNTYDQVVVGAEYDFSKRTSALVSAGWLQEGKGENKFVATAGGVGLRHKF

SEQ ID NO: 4 - protein, 130 aa, Artificial Sequence
  50s ribosomal protein L7/L12
MAKMSTDDLLDAFKEMTLLELSDFVKKFEETFEVTAAAPVAVAAAGPAAGGAPAEAAEEQSEFDVILESAGDKKIGVIKVVREIVSGLGLKEAKDLVDGAPKPLLEKVAKEAADDAKAKLEAAGATVTVK

SEQ ID NO: 5 - protein, 47 aa, Artificial Sequence
  Human beta-defensin 1
GNFLTGLGHRSDHYNCVSSGGQCLYSACPIFTKIQGTCYRGKAKCCK

SEQ ID NO: 6 - protein, 41 aa, Artificial Sequence
  Human beta-defensin 2
GIGDPVTCLKSGAICHPVFCPRRYKQIGTCGLPGTKCCKKP

SEQ ID NO: 7 - protein, 45 aa, Artificial Sequence
  Human beta-defensin 3
GIINTLQKYYCRVRGGRCAVLSCLPKEEQIGKCSTRGRKCCRRKK

SEQ ID NO: 8 - protein, 50 aa, Artificial Sequence
  Human beta-defensin 4
EFELDRICGYGTARCRKKCRSQEYRIGRCPNTYACCLRKWDESLLNRTKP

SEQ ID NO: 9 - protein, 5 aa, Artificial Sequence
  Linker
EAAAK

SEQ ID NO: 10 - protein, 7 aa, Artificial Sequence
  Linker
DPRVPSS

SEQ ID NO: 11 - protein, 1273 aa, Artificial Sequence
  Spike glycoprotein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 222 aa, Artificial Sequence
  Membrane glycoprotein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 13 - protein, 75 aa, Artificial Sequence
  Envelope protein
MYSFVSEETGTLIVNSVLLFLAFVVFLLVTLAILTALRLCAYCCNIVNVSLVKPSFYVYSRVKNLNSSRVPDLLV

SEQ ID NO: 14 - protein, 419 aa, Artificial Sequence
  Nucleocapsid protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 15 - protein, 183 aa, Artificial Sequence
  SARS-CoV2 antigen
GTTLPKKKFFGMSRIGMEVTPSGTWKKLLPAADGPGPGAALALLLLDRLNQLEGPGPGGTWLTYTGAIKLDDKGPGPGFPRGQGVPIAAYFPRGQGVPIAAYFPRGQGVPIAAYLSPRWYFYYAAYLLLDRLNQLAAYKSAAEASKKAAYKPRQKRTATAAYGMSRIGMEVAAYKTFPPTEPK

SEQ ID NO: 16 - protein, 123 aa, Artificial Sequence
  SARS-CoV2 antigen
GTTLPKKKFFGMSRIGMEVTPSGTWKKLLPAADGPGPGAALALLLLDRLNQLEGPGPGGTWLTYTGAIKLDDKGPGPGFPRGQGVPIAAYFPRGQGVPIAAYFPRGQGVPIAAYLSPRWYFYY

SEQ ID NO: 17 - protein, 85 aa, Artificial Sequence
  SARS-CoV2 antigen
AALALLLLDRLNQLEGPGPGGTWLTYTGAIKLDDKGPGPGFPRGQGVPIAAYFPRGQGVPIAAYFPRGQGVPIAAYLSPRWYFYY

SEQ ID NO: 18 - protein, 145 aa, Artificial Sequence
  SARS-CoV2 antigen
AALALLLLDRLNQLEGPGPGGTWLTYTGAIKLDDKGPGPGFPRGQGVPIAAYFPRGQGVPIAAYFPRGQGVPIAAYLSPRWYFYYAAYLLLDRLNQLAAYKSAAEASKKAAYKPRQKRTATAAYGMSRIGMEVAAYKTFPPTEPK

SEQ ID NO: 19 - protein, 60 aa, Artificial Sequence
  HylA secretion signal peptide
LAYGSQGDLNPLINEISKIISAAGSFDVKEERTAASLLQLSGNASDFSYGRNSITLTTSA

SEQ ID NO: 20 - protein, 21 aa, Artificial Sequence
  PhoA signal peptide
MKQSTIALALLPLLFTPVTKA

SEQ ID NO: 21 - protein, 21 aa, Artificial Sequence
  OmpA signal peptide
MKKTAIAIAVALAGFATVAQA

SEQ ID NO: 22 - protein, 23 aa, Artificial Sequence
  BLA signal peptide
MSIQHFRVALIPFFAAFCLPVFA

SEQ ID NO: 23 - protein, 34 aa, Artificial Sequence
  BLA signal peptide
MSIQHFRVALIPFFAAFCLPVFAHPETLVKVKDA

SEQ ID NO: 24 - protein, 21 aa, Artificial Sequence
  C-terminal sequence of BLA
ATMDERNRQIAEIGASLIKHW

SEQ ID NO: 25 - protein, 32 aa, Artificial Sequence
  N-terminal sequence of HylA
MPTITTAQIKSTLQSAKQSAANKLHSAGQSTK

SEQ ID NO: 26 - protein, 9 aa, Artificial Sequence
  HA-Tag
YPYDVPDYA

SEQ ID NO: 27 - protein, 5 aa, Artificial Sequence
  Cleavable linker
DDDDK

SEQ ID NO: 28 - protein, 6 aa, Artificial Sequence
  Cleavable linker
LVPRGS

SEQ ID NO: 29 - protein, 5 aa, Artificial Sequence
  Linker
GPGPG

SEQ ID NO: 30 - protein, 446 aa, Artificial Sequence
  HlyAN-linker- CtxB- linker-RBD (S-Protein)-FlagTag-Linker-HlyAS-CDS
MPTITTAQIKSTLQSAKQSAANKLHSAGQSTKDASEAAAKTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANEAAAKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFDYKDDDDKEAAAKHALAYGSQGDLNPLINEISKIISAAGSFDVKEERTAASLLQLSGNASDFSYGRNSITLTTSA

SEQ ID NO: 31 - DNA, 1062 bp, Artificial Sequence
  Fusion protein DNA
atgcatcagaagcggcggcgaaaaccccgcagaacatcaccgacctgtgcgcggaataccacaacacccagatccacaccctgaacgacaaaatcttctcctacaccgaatccctggcgggcaaacgtgaaatggcgatcatcaccttcaaaaacggcgcgaccttccaggttgaagttccgggctcccagcacatcgactcccagaaaaaagcgatcgaacgtatgaaagacaccctgcgtatcgcgtacctgaccgaagcgaaagttgaaaaactgtgcgtttggaacaacaaaaccccgcacgcgatcgcggcgatctccatggcgaacgaagcggcggcgaaacgtgttcagccgaccgaatccatagttaggttcccgaacatcactaacctgtgtccgtttggcgaagtgttcaacgcgacccgttttgcgtccgtctacgcctggaaccgtaaacgtatctccaactgcgttgcggactactccgttctgtacaactccgcgtccttctccaccttcaaatgctacggcgtttccccgaccaaactgaacgacctgtgcttcaccaacgtttacgcggactccttcgttatccgtggcgacgaagttcgtcagatcgcgccgggccagaccggcaaaatcgcggactacaactacaaactgccggacgacttcaccggctgcgttatcgcgtggaactccaacaacctggactccaaagttggcggcaactacaactacctgtaccgtctgttccgtaaatccaacctgaaaccgttcgaacgtgacatctccaccgaaatctaccaggcgggctccaccccgtgcaacggcgttgaaggcttcaactgctacttcccgctgcagtcctacggcttccagccgaccaacggcgttggctaccagccgtaccgtgttgttgttctgtccttcgaactgctgcacgcgccggcgaccgtttgcggcccgaaaaaatccaccaacctggttaaaaacaaatgcgttaacttcgactacaaagacgacgacgacaaagaagcggcggcgaaacatgcat

SEQ ID NO: 32 - DNA, 1341 bp, Artificial Sequence
  Fusion protein DNA
atgccaacaataaccactgcacaaattaaaagcacactgcagtctgcaaagcaatccgctgcaaataaattgcactcagcaggacaaagcacgaaagatgcatcagaagcggcggcgaaaaccccgcagaacatcaccgacctgtgcgcggaataccacaacacccagatccacaccctgaacgacaaaatcttctcctacaccgaatccctggcgggcaaacgtgaaatggcgatcatcaccttcaaaaacggcgcgaccttccaggttgaagttccgggctcccagcacatcgactcccagaaaaaagcgatcgaacgtatgaaagacaccctgcgtatcgcgtacctgaccgaagcgaaagttgaaaaactgtgcgtttggaacaacaaaaccccgcacgcgatcgcggcgatctccatggcgaacgaagcggcggcgaaacgtgttcagccgaccgaatccatagttaggttcccgaacatcactaacctgtgtccgtttggcgaagtgttcaacgcgacccgttttgcgtccgtctacgcctggaaccgtaaacgtatctccaactgcgttgcggactactccgttctgtacaactccgcgtccttctccaccttcaaatgctacggcgtttccccgaccaaactgaacgacctgtgcttcaccaacgtttacgcggactccttcgttatccgtggcgacgaagttcgtcagatcgcgccgggccagaccggcaaaatcgcggactacaactacaaactgccggacgacttcaccggctgcgttatcgcgtggaactccaacaacctggactccaaagttggcggcaactacaactacctgtaccgtctgttccgtaaatccaacctgaaaccgttcgaacgtgacatctccaccgaaatctaccaggcgggctccaccccgtgcaacggcgttgaaggcttcaactgctacttcccgctgcagtcctacggcttccagccgaccaacggcgttggctaccagccgtaccgtgttgttgttctgtccttcgaactgctgcacgcgccggcgaccgtttgcggcccgaaaaaatccaccaacctggttaaaaacaaatgcgttaacttcgactacaaagacgacgacgacaaagaagcggcggcgaaacatgcattagcctatggaagtcagggtgatcttaatccattaattaatgaaatcagcaaaatcatttcagctgcaggtagcttcgatgttaaagaggaaagaactgcagcttctttattgcagttgtccggtaatgccagtgatttttcatatggacggaactcaataaccctgaccacatcagcataa

SEQ ID NO: 33 - DNA, 78 bp, Artificial Sequence
  lacIEC
gacaccatcgaatggcgcaaaacctttcgcggtatggcatgatagcgcccggaagagagtcaattcagggtggtgaat

SEQ ID NO: 34 - DNA, 103 bp, Artificial Sequence
  lacIEC-like
gctagcgacaccatcgaatggcgcaaacctttcgcggtatggcatgatagcgcccgaagtcgtgtaccggcaaaggtgagtcgttatatacatggagattttg

SEQ ID NO: 35 - DNA, 313 bp, Artificial Sequence
  tyrS of E. coli
gtaaattcctggagctgaagcagaagtttcaacagggcgaagtgccattgccgagcttttggggcggttttcgcgtcagccttgaacagattgagttctggcagggtggtgagcatcgcctgcatgaccgctttttgtaccagcgtgaaaatgatgcgtggaagattgatcgtcttgcaccctgaaaagatgcaaaaatcttgctttaatcgctggtactcctgattctggcactttattctatgtctctttcgcatctggcgaaaagtcgtgtaccggcaaaggtgcagtcgttatatacatggagattttg

SEQ ID NO: 36 - DNA, 195 bp, Artificial Sequence
  tyrS of E. coli
cctgcatgaccgctttttgtaccagcgtgaaaatgatgcgtggaagattgatcgtcttgcaccctgaaaagatgcaaaaatcttgctttaatcgctggtactcctgattctggcactttattctatgtctctttcgcatctggcgaaaagtcgtgtaccggcaaaggtgcagtcgttatatacatggagattttg

SEQ ID NO: 37 - DNA, 95 bp, Artificial Sequence
  tyrS of E. coli
ctcctgattctggcactttattctatgtctctttcgcatctggcgaaaagtcgtgtaccggcaaaggtgcagtcgttatatacatggagattttg

SEQ ID NO: 38 - DNA, 42 bp, Artificial Sequence
  Terminator region of TyrS-HisTag EPC
taatccacggccgccagtttgggctggcggcattttggtacc

SEQ ID NO: 39 - DNA, 48 bp, Artificial Sequence
  lacIEC E. coli
taatgagcgcaacgcaattaatgtgagttagctcactcattaggcacc

SEQ ID NO: 40 - DNA, 58 bp, Artificial Sequence
  tyrSEC E. coli
tgcattaagtggaaagggggagtgagaaatcactccccctggtttttatacagggaac

SEQ ID NO: 41 - protein, 270 aa, Artificial Sequence
  PhoA-human -defensin 1-N-Multiepitope unit Variant 1-T7-tag
MKQSTIALALLPLLFTPVTKAGNFLTGLGHRSDHYNCVSSGGQCLYSACPIFTKIQGTCYRGKAKCCKEAAAKGTTLPKKKFFGMSRIGMEVTPSGTWKKLLPAADGPGPGAALALLLLDRLNQLEGPGPGGTWLTYTGAIKLDDKGPGPGFPRGQGVPIAAYFPRGQGVPIAAYFPRGQGVPIAAYLSPRWYFYYAAYLLLDRLNQLAAYKSAAEASKKAAYKPRQKRTATAAYGMSRIGMEVAAYKTFPPTEPKAAYMASMTGGQQMG

SEQ ID NO: 42 - DNA, 13175 bp, Artificial Sequence
  pSalVac plasmid
gaattccaagcgaagtccatccccctccctcttgattacaagggtgataattattattcgcatttgtgtggtaatgggatagaaaggaatggatagaaaaagaacaaaattagtatagcaatagatatgcccactgcattgaatacttacagggcattattttattatgtttaaattgaagtggtctctggtttgatttatttgttattcaagggggctgtttggagatcggaaaattctgtacgttaagtgtattatttaaccagtttcgatgcgtaacagattgattttgcgtcagcggttatcgcttttaagttgttgctcttgcgctatcgcgtttaggttatccgattaaagtcaaatttcctgaaaatgctgtatagcgcgggagtgcaccttatagctgtaggtaagtatgttcaaaaaatagtcttgccgtacaataattttccatatccaaactcactccttcaagattctggtcccggtttacgggtagtttccggaagggcggtagcatgctgattcaaactgcaagatgaaacattgtcggagttggatggaattaagtcatggctatagcatttgggcgtgcataacaaaattggtcctcatattttagagtatgattgcatattcactaatatttttactttctgatgcgtggtggcatcatgctttatgagataaacaatcctggtagactagccccctgaatctccagacaaccaatatcacttatttaagtgatagtcttaatactagtgctagcgacaccatcgaatggcgcaaacctttcgcggtatggcatgatagcgcccgaagtcgtgtaccggcaaaggtgcagtcgttatatacatggagattttgatggcaagcagtaacttgattaaacaattgcaagagcgggggctggtagcccaggtgacggacgaggaagcgttagcagagcgactggcgcaaggcccgatcgcgctctattgcggcttcgatcctaccgctgacagcttgcatttggggcatcttgttccattgttatgcctgaaacgcttccagcaggcgggccacaagccggttgcgctggtaggcggcgcgacgggtctgattggcgacccgagcttcaaagctgccgagcgtaagctgaacaccgaagaaactgttcaggagtgggtggacaaaatccgtaagcaggttgccccgttcctcgatttcgactgtggagaaaactctgctatcgcggcgaacaactatgactggttcggcaatatgaatgtgctgaccttcctgcgcgatattggcaaacacttctccgttaaccagatgatcaacaaagaagcggttaagcagcgtctcaaccgtgaagatcaggggatttcgttcactgagttttcctacaacctgttgcagggttatgacttcgcctgtctgaacaaacagtacggtgtggtgctgcaaattggtggttctgaccagtggggtaacatcacttctggtatcgacctgacccgtcgtctgcatcagaatcaggtgtttggcctgaccgttccgctgatcactaaagcagatggcaccaaatttggtaaaactgaaggcggcgcagtctggttggatccgaagaaaaccagcccgtacaaattctaccagttctggatcaacactgcggatgccgacgtttaccgcttcctgaagttcttcacctttatgagcattgaagagatcaacgccctggaagaagaagataaaaacagcggtaaagcaccgcgcgcccagtatgtactggcggagcaggtgactcgtctggttcacggtgaagaaggtttacaggcggcaaaacgtattaccgaatgcctgttcagcggttctttgagtgcgctgagtgaagcggacttcgaacagctggcgcaggacggcgtaccgatggttgagatggaaaagggcgcagacctgatgcaggcactggtcgattctgaactgcaaccttcccgtggtcaggcacgtaaaactatcgcctccaatgccatcaccattaacggtgaaaaacagtccgatcctgaatacttctttaaagaagaagatcgtctgtttggtcgttttaccttactgcgtcgcggtaaaaagaattactgtctgatttgctggaaacatcaccatcaccatcactaatccacggccgccagtttgggctggcggcattttggtaccactagtgataatggttcatgctaccgggcgaatgaaacacgtcagttcgccaggatgttgggacttgaaccgaagaacacggcagtgcggagtccggagagtaacggaataacagagagcttcgtgaaaacgataaagcgtgattacataagtatcatgcccaaaccagacgggttaacggcagcaaagaaccttgcagaggcgttcgagcattataacgaatggcatccgcatagtgcgctgggttatcgctcgccacgggaatatctgcggcagcgggccagtaatgggttaagtgataacaggtatctggaaatataggggcaaatccacctggtcattatctggaatttgacgaagtgtgataactggtatagccagattaatctaaacctttgtctgacaaaatcagataaagaagagtagttcaaaagacaactcgtggactctcattcagagagataggcgttaccaaaatttgtttggaactgaacaagaaaattgtatttgtgtaactataatcttaatgtaaaataaaagacaccagttctgtagaatatgcttattgaagagagtgtaataataattttatatagatgttgtacaaagaacaggaatgagtaattatttatgcttgatgttttttgactcttgctttttatagttattatttttaagttagtcagcgcaataaaaacttgcttttaatattaatgcgagttatgacattaaacggaagaaacataaaggcatatttttgccacaatatttaatcatataatttaagttgtagtgagtttattatgaatataaacaaaccattagagattcttgggcatgtatcctggctatgggccagttctccactacacagaaactggccagtatctttgtttgcaataaatgtattacccgcaatacaggctaaccaatatgttttattaacccgggatgattaccctgtcgcgtattgtagttgggctaatttaagtttagaaaatgaaattaaatatcttaatgatgttacctcattagttgcagaagactggacttcaggtgatcgtaaatggttcattgactggattgctcctttcggggataacggtgccctgtacaaatatatgcgaaaaaaattccctgatgaactattcagagccatcagggtggatcccaaaactcatgttggtaaagtatcagaatttcatggaggtaaaattgataaacagttagcgaataaaatttttaaacaatatcaccacgagttaataactgaagtaaaaagaaagtcagattttaatttttcattaactggttaagaggtaattaaatgccaacaataaccactgcacaaattaaaagcacactgcagtctgcaaagcaatccgctgcaaataaattgcactcagcaggacaaagcacgaaagatgcattagcctatggaagtcagggtgatcttaatccattaattaatgaaatcagcaaaatcatttcagctgcaggtagcttcgatgttaaagaggaaagaactgcagcttctttattgcagttgtccggtaatgccagtgatttttcatatggacggaactcaataaccctgaccacatcagcataatatattaatttaaatgatagcaatcttactgggctgtgccacataagattgctattttttttggagtcataatggattcttgtcataaaattgattatgggttatacgccctggagattttagcccaataccataacgtctctgttaacccggaagaaattaaacatagatttgatacagacgggacaggtctgggattaacgtcatggttgcttgctgcgaaatctttagaactaaaggtaaaacaggtaaaaaaaacaattgatcgattaaactttatttttctgcccgcattagtctggagagaggatggacgtcattttattctgactaaaatcagcaaagaagtaaacagatatcttatttttgatttggagcagcgaaatccccgtgttctcgaacagtctgagtttgaggcgttatatcaggggcatattattcttattacttcccgttcttctgttaccgggaaactggcaaaatttgactttacctggtttattcctgccattataaaatacaggagaatatttattgaaacccttgttgtatctgtttttttacaattatttgcattaataacccccctttttttccaggtggttatggacaaagtattagtgcacagggggttttcaacccttaatgttattactgttgcattatctgttgtagtggtgtttgagattatactcagcggtttaagaacttacatttttgcacatagtacaagtcggattgatgttgagttgggtgccaaactcttccggcatttactggcgctaccgatctcttattttgagagtcgtcgtgttggtgatactgttgcgagggtaagagaattagaccagatccgtaattttctgacaggacaggcattaacatctgttttggacttattattttcactcatattttttgcggtaatgtggtattacagcccaaagcttactctggtgatcttattttcgctgccttgttatgctgcatggtctgtttttattagccccattttgcgacgtcgccttgatgataagttttcacggaatgcggataatcaatctttcctggtggaatcagtaacggcgattaacactataaaagctatggcagtctcacctcagatgacgaacatatgggacaaacaattggcaggatatgttgctgcaggctttaaagtgacagtattagcaaccattggtcaacaaggaatacagttaatacaaaagactgttatgatcatcaacctatggttgggagcacacctggttatttccggggatttaagtattggtcagttaattgcttttaatatgcttgctggtcagattgttgcaccggttattcgccttgcacaaatctggcaggatttccagcaggttggtatatcagttacccgccttggtgatgtgcttaactctccaactgaaagttatcatgggaaactgacattgccggaaattaatggtgatatcacttttcgtaatatccggtttcgctataaacctgattctccggttattttggacaatatcaatcttagtattaagcagggggaggttattggtattgtcggacgttctggttcaggaaaaagcacattaactaaattaattcaacgtttttatattcctgaaaatggccaggtattaattgatggacatgatcttgcgttggctgatcctaactggttacgtcgtcaggtgggggttgtgttgcaggacaatgtgctgcttaatcgcagtattattgataatatttcactggctaatcctggcatgtccgtcgaaaaagttatttatgcagcgaaattagcaggcgctcatgattttatttctgatttgcgtgaggggtataacaccattgtcggggaacagggggcaggattatccggaggtcaacgtcaacgcatcgcaattgcaagggcgctggtgaacaaccctaaaatactcatttttgatgaagcaaccagtgctctggattatgagtcggagcatgtcatcatgcgcaatatgcacaaaatatgtaagggcagaacggttataatcattgctcatcgtctgtctacagtaaaaaatgcagaccgcattattgtcatggaaaaagggaaaattgttgaacagggtaaacataaggagctgctttctgaaccggaaagtttatacagttacttatatcagttacagtcagactaacagaaagaacagaagaatatgaaaacatggttaatggggttcagcgagttcctgttgcgctataaacttgtctggagtgaaacatggaaaatccggaagcaattagatactccggtacgtgaaaaggacgaaaatgaattcttacccgctcatctggaattaattgaaacgccagtatccagacggccgcgtctggttgcttattttattatggggtttctggttattgcttttattttatctgttttaggccaagtggaaattgttgccactgcaaatgggaaattaacacacagtgggcgtagtaaagaaattaaacctattgaaaactcaatagttaaagaaattatcgtaaaagaaggagagtcagtccggaaaggggatgtgttattaaagcttacagcactgggagctgaagctgatacgttaaaaacacagtcatcactgttacaggccaggctggaacaaactcggtatcaaattctgagcaggtcaattgaattaaataaactacctgaactaaagcttcctgatgagccttattttcagaatgtatctgaagaggaagtactgcgtttaacttctttgataaaagaacagttttccacatggcaaaatcagaagtatcaaaaagaactgaatttggataagaaaagagcagagcgattaacagtacttgcccgtataaaccgttatgaaaatttatcaagggttgaaaaaagccgtctggatgatttcagtagtttattgcataaacaggcaattgcaaaacatgctgtacttgagcaggagaataaatatgtcgaagcagtaaatgaattacgagtttataaatcacaactggagcaaattgagagtgagatattgtctgcaaaagaagaatatcagcttgttacgcagctttttaaaaatgaaattttagataagctaagacaaacaacagacaacattgggttattaactctggaattagcgaaaaatgaagagcgtcaacaggcttcagtaatcagggccccagtttcgggaaaagttcagcaactgaaggttcatactgaaggtggggttgttacaacagcggaaacactgatggtcatcgttccggaagatgacacgctggaggttactgctctggtacaaaataaagatattggttttattaacgtcgggcagaatgccatcattaaagtggaggcatttccttatacacgatatggttatctggtgggtaaggtgaaaaatataaatttagatgcaatagaagaccagagactgggacttgtttttaatgttattatttctattgaagagaattgtttgtcaaccgggaataaaaacattccattaagctcgggtatggcagtcactgcagaaataaagacaggtatgcgaagtgtaatcagttatcttcttagtcctttagaagagtcagtaacagaaagtttacgtgagcgttaagtttcagaagtccagtatttgctgctatacgtgctgcgtggcacttgccgtctgaacggcattgatccggaagccaagtcaaacaacagcgtgatgagcgtcagggcaaaacaccaaggctctctcgatgacaccagaacaaattgaaatacgtgagctgaggaaaaagctaccgagttcttgatgttggactccctgaacagttctctgtaatcgggaaactcaggacgcgttatcctgtggtcacactctgccatgtgtttagggttcatcacagcagctacagatactggtaaaaccgtcctgaaaaaccagacggcagacgggctgtattacgtagtcaggtacttgagctacatggcatcagtcacggtttggccggagcaagacgtatcaccacaatggcaacccggagaggtgtcagcgccagtgatataagacggttaacggttaaaaatcgtggcgttgacaacatcccagtggactgaggtcacacaggcctggcagcattcctcttccggccggatgacccggatttcacggggaaagtacgccgataacagtttacgggctgaagattggcgtagggaggatagcagacgttttgccgcccccattgtctggagttgggtgagaaggcatcatttcaccaacaccaacatttcacagttacaccccacagctacatgaagcgcttccatgaattatcgctttgatttatcatgttaaaatagctctacacggttggttcaggattgcgcaccgaaaccctctaaaatccactgacgcgcctgcgaattatccagcaccgcgcctttcgagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccatatgaatatcctccttagttcctattccgaagttcctattctctagaaagtataggaacttcagagcgcttttgaagctggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgggggatcttgaagttcctattccgaagttcctattctctagaaagtataggaacttcgaagcagctccagcctacaccaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgatggacattatttttgtggagccggaggaaacagaccagacggttcagatgaggcgcttaccaccagaaccgctgttgtcccaccattctggcgattcccaaacgctatttggataaaaagtagccttaacgtggtttattttcc

(19) United States
(12) Patent Application Publication
(10) Pub. No.: US 2022/0047697 A1
(43) Pub. Date: Feb. 17, 2022

(54) SALMONELLA VACCINE FOR THE TREATMENT OF CORONAVIRUS

(71) Applicant: JULIUS-MAXIMILIANS-UNIVERSITÄT WÜRZBURG, Würzburg (DE)

(72) Inventors: Thomas Rudel, Würzburg (DE); Birgit Bergmann, Würzburg (DE)

(21) Appl. No.: 17/402,014

(22) Filed: Aug. 13, 2021

(30) Foreign Application Priority Data
Aug. 14, 2020 (EP) 20 191 142.7

(51) Int. Cl. A61K 39/215; C07K 14/005; C07K 14/705; C07K 14/55; C07K 14/28; C07K 14/22; C07K 14/47; A61P 31/14; A61K 39/39

(52) U.S. Cl. CPC A61K 39/215; C07K 14/005; C07K 14/705; C07K 14/55; C07K 14/28; A61K 2039/522; C07K 14/47; A61P 31/14; A61K 39/39; C07K 2319/00; C07K 2319/55; C07K 14/22

(57) ABSTRACT

The present invention provides live-attenuated bacterium of the genus Salmonella comprising a recombinant plasmid encoding a fusion protein, wherein the fusion protein comprises a coronavirus antigen and an adjuvant peptide.

### CROSS REFERENCE TO RELATED APPLICATIONS

This application claims the benefit of European Patent Application No. 20 191 142.7, filed Aug. 14, 2020, the entire contents of each of which are fully incorporated herein by reference.

### INCORPORATION BY REFERENCE OF MATERIAL SUBMITTED ELECTRONICALLY

A Sequence Listing, which is a part of the present disclosure, is submitted concurrently with the specification as a text file. The name of the text file containing the Sequence Listing is “56989_Seqlisting.txt.” The Sequence Listing was created on Jul. 30, 2021, and is 64,132 bytes in size. The subject matter of the Sequence Listing is incorporated by reference herein in its entirety.

### TECHNICAL FIELD

The present invention aims to provide a novel vaccine for the treatment and/or prevention of coronavirus diseases. Thus, the present invention is within the field of coronavirus vaccines.

### TECHNICAL BACKGROUND

Severe acute respiratory syndrome coronavirus 2 (SARS-CoV-2) is the strain of coronavirus that causes coronavirus disease 2019 (COVID-19), the respiratory illness responsible for the COVID-19 pandemic. SARS-CoV-2 has wreaked havoc around the world crippling healthcare systems and devastating economies. More particularly, SARS-CoV-2 is an emerging virus that is highly pathogenic and caused the recent global pandemic, officially known as coronavirus disease (COVID-19). It belongs to the family of Coronaviruses (CoVs), which can cause mild to lethal respiratory tract infections in mammals and birds. Members causing more lethal infections in humans include SARS-CoV, Middle East respiratory syndrome (MERS) and SARS-CoV-2. These are cytoplasmic replicating, single-stranded RNA viruses with four structural proteins: The Spike (S) glycoprotein, the envelope protein (E), the membrane protein (M), and the nucleocapsid protein (N) (Chen et al., 2020). The S protein plays a critical role in triggering the immune response in the disease process (To et al., 2020). SARS-CoV-2 enters host cells via the receptor angiotensin converting enzyme 2 (ACE2) and the S protein is required for cell entry (Hoffmann et al., 2020, Ou et al., 2020, Zhou et al., 2020). The trimeric S protein contains two subunits, S1 and S2, which mediate receptor binding and membrane fusion, respectively. The S1 subunit contains a fragment called the receptor-binding domain (RBD) that is capable of binding ACE2 (Letko et al., 2020, Wan et al., 2020). Binding of the S protein to the ACE2 receptor triggers complex conformational changes that move the S protein from a prefusion conformation to a postfusion conformation. In view of previous studies and the experience of previously approved SARS-CoV-2 vaccines, the inventors considered that the S protein elicits potent cellular and humoral immune responses. The S protein of SARS-CoV-2, particularly the RBD, is capable of inducing neutralizing antibody and T cell immune responses (Suthar et al., 2020).

In addition to the S protein, the nucleocapsid protein (N protein) may function as promising antigen in vaccines. For the CoV N protein it has been demonstrated to induce protective specific cytotoxic T lymphocytes (Gao et al., 2003, Kim et al., 2004).

Live attenuated S. enterica serovar Typhi (S. typhi) are candidates for the engineering of live recombinant mucosal vaccines. One strategy to develop new vaccines is the use of live attenuated bacteria as carriers for the presentation of heterologous antigens (Cheminay et al., 2008). Salmonella strains are useful since these strains can be administered orally, i.e. by the natural route of infection, and may induce mucosal as well as systemic immune responses. Both humoral and cellular immune responses can be primed by this form of application. Furthermore, convenient methods for the genetic manipulation of Salmonella are available, and one can express single or multiple heterologous antigens from other bacteria or from viruses or parasites, allowing to create a single recombinant vaccine for simultaneous protection against S. typhi and other pathogens. More than 20 years of experience with a licensed live attenuated Salmonella vaccine, S. typhi Ty21a (Typhoral® L) (Xu et al., 2013) are available and indicate that this strain is safe in mass vaccination against typhoid fever.

To produce foreign antigens in S. typhi, plasmids carrying genetic cassettes for the expression and delivery of cargo proteins have been generated. Therefore, plasmid stability is the most critical parameter for the successful delivery of cargo proteins (antigens) in vaccinated humans. Plasmid stability in general has been achieved by integrating genes conferring antibiotics resistance into the plasmid. However, the use of antibiotic resistance genes as a selective determinant for plasmid maintenance is impractical in vivo. This problem was first addressed by the construction of a balanced-lethal system in which the asd gene of St. mutans was introduced in a plasmid that complements an asd mutation in the chromosome of an diaminopimelic acid auxotrophic Salmonella strain (Galan et al., 1990).

Recently, the inventors developed a balanced-lethal-system (BLS) for the antibiotic-free stabilization of plasmids in S. typhi Ty21a which is independent of any auxotrophy. The system depends on the complementation of an essential gene and therefore does not require cost-intensive defined media for selection. The BLS the inventors designed is made up of the chromosomal knockout of the putative essential gene tyrS encoding for the tyrosyl-tRNA-synthetase and the in trans complementation of this gene on the respective antigen-delivery-plasmid (Diessner, 2009, Gesser, 2010). For the construction of the chromosomal tyrS-knockout the inventors modified the method of “one-step inactivation of chromosomal genes using PCR products” which was described by Datsenko and Wanner (2000) (Datsenko et al., 2000). As tyrS is an essential gene, the approach described by Datsenko and Wanner (2000) has to be adapted since the knockout without genetic compensation would be lethal. For this reason, tyrS was replaced by a knock-in fragment encoding for the antibiotic resistance and also for a gene encoding E. coli tyrS flanked by two flippase recognition targets (FRT) for the conditional deletion in complemented strains resulting in the newly generated (FRT-tyrS Cm FRT)-knock-in-strain (->S.t. Typhi Ty21a (ΔtyrS (tyrS Cm)⁺) (Diessner, 2009). Based on this intermediate strain, the balanced lethal stabilized vaccine strains can be constructed.

Antigens expressed by the Salmonella carriers can be secreted as hemolysin fusion proteins via the hemolysin (HlyA) secretion system of Escherichia coli, which allows efficient protein secretion (Gentschev et al., 1996). The secretion of antigens from the carrier strain has been used for anti-infective vaccination and for cancer vaccines (Hess et al., 1996, Gomez-Duarte et al., 2001, Fensterle et al., 2008). Protein antigens can be fused to cholera toxin subunit B (CtxB) (Arakawa et al., 1998, Yuki et al., 2001, Sadeghi et al., 2002), one of the most effective experimental mucosal adjuvants (Holmgren et al., 2005, Lycke, 2005). U.S. Pat. No. 10,973,908 B1 (date of patent: Apr. 13, 2021) relates to the expression of Sars-Cov-2 spike protein receptor binding domain in attenuated salmonella as a vaccine.

In summary, there is currently a dire need for a vaccine that can prevent SARS-CoV-2 infections. In particular, there is still an urgent need for a SARS-CoV-2 vaccine that can be used globally and with less stringent handling requirements, i.e. provided at moderate costs, stored without a need for ultra-low temperature freezers or other high-tech equipment, and administered without the need for medical equipment or trained medical personnel.

### FIGURES

FIG. 1: Map of plasmid pSalVac 001 A0_B0 KanR for expressing one or more fusion proteins of the present invention. Basic cloning vector for integration of NsiI- and SalI-fragments into A- (->NsiI-), respectively B-(->SalI-) Site (SEQ ID NO: 42)

FIG. 2: Map of plasmid pSalVac 101 A1_B0 KanR of the present invention. NsiI-fragment No. 1 (improved DNA) (SEQ ID NO: 31) has been inserted into the NsiI site of pSalVac 001 A0_B0 KanR resulting in pSalVac 101 A1_B0 KanR with CDS of fusion protein A1 (SEQ ID NO: 30).

FIG. 3: Features of the nucleic acids that can be inserted at the A) NsiI site and B) SalI site.

FIG. 4: Antigenic plot for SEQ ID NO: 30.

FIG. 5: Antigenic plot for SEQ ID NO: 41.

FIG. 6: Flowchart for the generation of vaccine strains.

FIG. 7: Codon-optimized sequence (SEQ ID NO: 177) of the CtxB adjuvant for expression in Salmonella typhi (strain ATCC 700931/Ty2) using JCat http://www.jcat.de (Grote et al., 2005). A total of 79 codons of CtxB coding sequence (CDS CtxB mature protein: 103 codons, AAC34728.1 (SEQ ID NO: 176) were modified for optimal codon utilization (A), which resulted in no change in the amino acid sequence (SEQ ID NO: 2) of the encoded protein (B). The sequence alignments were performed by SnapGene software using global alignment (Needleman-Wunsch).

FIG. 8:

A) Codon-optimized sequence (SEQ ID NO: 119) of CDS RBD (Receptor-binding domain) of S-Protein in fusion protein A1. CodonUsage adapted to Salmonella typhi (strain ATCC 700931/Ty2) using JCat http://www.jcat.de. A total of 76 codons of RBD coding sequence (CDS RBD: 223 codons, S-Protein Wuhan Hu-1, GeneID 43740568—NC_045512.2, (SEQ ID NO: 179)) were modified for optimal codon utilization, which resulted in no change in the amino acid sequence of the encoded protein. The sequence alignments were performed using the SnapGene software using global alignment (Needleman-Wunsch).

B) Codon usage optimization of the Dimerization Region (DR) of N-Protein (SEQ ID NO: 169). CodonUsage adapted to Salmonella typhi (strain ATCC 700931/Ty2) using JCat: http://www.jcat.de. A total of 65 codons of DR coding sequence (CDS DR: 104 codons, (SEQ ID NO: 182) CDS N-Protein NC_045512.2, GeneID: 43740575) were modified for optimal codon utilization, which resulted in no change in the amino acid sequence of the encoded protein. The sequence alignments were performed by SnapGene software using global alignment (Needleman-Wunsch)

FIG. 9: Plasmid maps of pSalVac 101 A1_B3f ΔKanR (A), pSalVac 101 A1_B10f KanR (B), pSalVac 101 A1_B10f ΔKanR (C)

FIG. 10: Demonstration of the deletion of chromosomal tyrS in one of the JMU-SalVac-100 strains (exemplary JMU-SalVac-104) harboring a BLS-stabilized plasmid of the pSalVac 101 Ax_By series.

A. Shown is the sequence of the ΔtyrS locus of the BLS strains. (TAA in bold: Stop codon of ΔtyrS upstream-gene pdxH; ATG in bold: Start codon of ΔtyrS downstream-gene pdxY; FRT-Site (minimal): underlined). SEQ ID NO: 184

B. Validation of the tyrS deletion in the indicated strains by PCR amplification. (Primer sequences (17/18; SEQ ID NO: 47/48)) correspond to regions flanking tyrS gene on chromosome.)

FIG. 11:

A: Expression and secretion of fusion proteins A1 (49.1 kDa) and A3 (45.8 kDa) detected in the lysate of bacteria (pellet) and the supernatant using anti-CtxB and anti-S-protein antisera. Proteins precipitated from supernatant (S) of bacterial culture or pellets of whole cell lysate (P) were loaded. The immunoblots were developed with anti-CtxB antibody and anti-RBD-Antibody. Arrow: 55 kDa.

B: Expression of fusion proteins B3 (27.6 kDa), B5 (20.7 kDa) and B7 (23.0 kDa). Whole cell lysate of mid-log cultures were analyzed by Western blot. The immunoblots were developed with anti-hBD1 antibody (abeam). Black arrow indicates the mol. mass of 35 kDa

FIG. 12: Expression of RNAs of the SalVac plasmids. cDNA was made from the indicated strains as described in chapter 2.10. A: mRNA made from the A site amplified with primers 4 and 5 (table 8 and table 12). B: mRNA made from the B site amplified with primers 57 and 58 (table 12). C: mRNA made from the plasmid encoded hlyB gene amplified with primers 62 and 63 (table 12). D: mRNA made from the plasmid encoded hlyD gene amplified with primers 64 and 65 (table 12).

FIG. 13: Growth curves of JMU-SalVac 100 strains and S. typhi Ty21a Growth of the indicated strains was measured as described in chapter 2.9.

FIG. 14: Stability of plasmids with and without BLS Stability of plasmids was determined as described in chapter 2.11. A: Data of the experiment explained in Example 3, chapter 3.7.11. B: Chromosomal tyrS was amplified with the primers 17 and 18 (Table 8) and the gene insert in the A site with the primers 68 and 69 (Table 8) to determine stability of the plasmid in the BLS strains. Numbers refer to: 1: size marker; 2: No template, control (water); 3: S. typhi Ty21a, control; 4: JMU-SalVac-101, control; 5: JMU-SalVac-104, control; 6-8: samples JMU-SalVac-101; 9-11: samples JMU-SalVac-104; 12: 1 kb Marker; 13: No template, control (water); 14: Ty21a; 15: JMU-SalVac-101, control; 16: JMU-SalVac-104, control; 17-19: samples JMU-SalVac-101; 20-22: samples JMU-SalVac-104. C: Data shown in (A) depicted as bar diagram. D: Plasmid stability testing example. Day 4: Low stability of pMKhly1 w/o BLS stabilization. Example shows colonies of S. typhi 21a with pMKhly1 grown for 4 days under the conditions as explained in Example 3, chapter 3.7.11. Left plate TS agar, right plate TS agar+25 g/mL Kanamycin. Only few colonies retain the plasmid and are therefore antibiotic resistant. E: Copy number determination of BLS strains. Plasmid copy number was determined on day 1 and day 5 as described in chapter 2.11.

FIG. 15: Expression of proteins in strains prepared for immunization Expression and Secretion of fusion protein A1 in JMU-SalVac-100-strains. Whole cell lysate and proteins precipitated from supernatant of mid-log (A) JMU-SalVac-100 vaccine strains and of late-log cultures (B) of S. typhimurium SL7207 vaccine strains were analyzed by Western blot. The immunoblots were developed with anti-ctxB antibody (Zytomed) (black arrow: 55 kDa)

FIG. 16: Tolerability study Tolerability of JMU-SalVac-100 (A) and S. typhimurium SL7207 (B) vaccine strains were tested over a period of 10 days as described in chapter 2.12.2.

### SUMMARY OF THE INVENTION

The present invention provides a live-attenuated bacterium of the genus Salmonella comprising a recombinant plasmid encoding a fusion protein, wherein the fusion protein comprises a coronavirus antigen, and an adjuvant peptide.

The present invention also provides a combination product comprising the bacterium of the present invention and at least one of the one or more fusion proteins encoded by the plasmid of said bacterium.

Further, the present invention provides a vaccine comprising the bacterium of the present invention or the combination product of the present invention.

The bacterium, combination product or vaccine may be used as a medicament. In particular, they may be used in a method of treating a disease or disorder caused by a member of the coronavirus family.

The present invention also provides a kit comprising a live-attenuated bacterium of the genus Salmonella, and a recombinant plasmid encoding a fusion protein, wherein the fusion protein comprises a coronavirus antigen and an adjuvant peptide.

### DETAILED DESCRIPTION OF THE INVENTION

### Definitions

Any term not defined in the present application should be given the normal meaning in the art.

As used herein, the term “adjuvant” refers to a substance used in combination with a specific antigen that produces a more robust immune response than the antigen alone.

The term “combination product” can refer to (i) a product comprised of two or more regulated components that are physically, chemically, or otherwise combined or mixed and produced as a single entity; (ii) two or more separate products packaged together in a single package or as a unit and comprised of drug and device products, device and biological products, or biological and drug products; (iii) a drug, device, or biological product packaged separately that according to its investigational plan or proposed labeling is intended for use only with an approved individually specified drug, device, or biological product where both are required to achieve the intended use, indication, or effect and where upon approval of the proposed product the labeling of the approved product would need to be changed, e.g., to reflect a change in intended use, dosage form, strength, route of administration, or significant change in dose; or (iv) any investigational drug, device, or biological product packaged separately that according to its proposed labeling is for use only with another individually specified investigational drug, device, or biological product where both are required to achieve the intended use, indication, or effect. This definition is in accordance with 21 CFR 3.2(e) (see US Code of Federal Regulations).

As used herein, the term “coronavirus antigen” refers to a peptide encoded by the genome of a member of the coronavirus family that can elicit an adaptive immune system response in a subject. An exemplary member of the coronavirus family is SARS-CoV-2.

As used herein, the term “effective amount” is that amount sufficient to effect beneficial or desired results, for example, clinical results, and, as such, an “effective amount” depends upon the context in which it is being applied. The term “effective amount” can be used interchangeably with “effective dose”, “therapeutically effective amount”, or “therapeutically effective dose”.

The terms “identical” or “percent identity”, in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. For example, preferred algorithms suitable for determining percent sequence identity and sequence similarity are the BLAST and BLAST 2.0 algorithms, which are described in Altschul et al., 1977. Nucleic Acids Res. 25:3389 and Altschul et al., 1990. J Mol Biol. 215:403, respectively.

The terms “individual”, “patient” or “subject” are used interchangeably in the present application and refer to any multicellular eukaryotic heterotroph which can be infected by a coronavirus. The subject is preferably a mammal. Mammals which would be infected by a coronavirus include humans, cats, dogs, pigs, ferrets, rabbits, gerbils, hamsters, guinea pigs, horses, rats, mice, cows, sheep, goats, alpacas, camels, donkeys, llamas, yaks, giraffes, elephants, meerkats, lemurs, lions, tigers, kangaroos, koalas, bats, monkeys, chimpanzees, gorillas, bears, dugongs, manatees, seals and rhinoceroses. Most preferably, the subject is human.

As used herein, the expression “live-attenuated bacterium” refers to a prokaryote that has been rendered less virulent through modification and/or selection so that it can no longer cause a systemic infection in an immunocompetent subject.

As used herein, “pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington: The Science and Practice of Pharmacy 22^{nd} edition, Pharmaceutical press (2012), ISBN-13: 9780857110626 may also be included.

As used herein, the term “plasmid” refers to a genetic structure in a cell that can replicate independently of the cell's chromosome or it can also refer to a genetic structure that can be integrated into the chromosome of the cell (e.g., using a FLP/FRT recombination system or a Cre-Lox recombination system). A plasmid used in accordance with the invention is preferably a plasmid which can replicate independently of the chromosome of the bacterium and does not require antibiotic selection to ensure its maintenance in the bacterium. This has the advantage that no antibiotic resistance genes are administered when administering the vaccine of the invention, resulting in improved safety of the vaccine.

The term “protein” is used interchangeably with the term “peptide” in the present application. Both terms, as used in the present application, refer to molecules comprising one or more chains of amino acid residues. A “fusion protein”, as used in the present application, refers to a protein created through the joining of two or more genes that originally coded for separate proteins via recombinant DNA techniques.

As used herein, the term “recombinant” refers to any material that is derived from or contains a nucleic acid molecule that was made through the combination or insertion of one or more nucleic acid molecules that would not normally occur together.

The terms “treatment” and “therapy”, as used in the present application, refer to a set of hygienic, pharmacological, surgical and/or physical means used with the intent to cure and/or alleviate a disease and/or symptom with the goal of remediating the health problem. The terms “treatment” and “therapy” include preventive and curative methods, since both are directed to the maintenance and/or reestablishment of the health of an individual or animal. Regardless of the origin of the symptoms, disease and disability, the administration of a suitable medicament to alleviate and/or cure a health problem should be interpreted as a form of treatment or therapy within the context of this application.

### Bacterium

The present invention provides a live-attenuated bacterium of the genus Salmonella comprising a recombinant plasmid encoding a fusion protein, wherein the fusion protein comprises a coronavirus antigen, and an adjuvant peptide.

Methods for generating live-attenuated bacteria of the genus Salmonella are known in the art (Tennant & Levine, 2015. Vaccine. 33(0 3):C36-41, doi: 10.1016/j.vaccine.2015.04.029).

In some embodiments, the bacterium is of the species Salmonella enterica. In some embodiments, the bacterium is a Salmonella enterica serovar Typhi strain, Salmonella enterica serovar Paratyphi A strain, Salmonella enterica serovar Paratyphi B strain, Salmonella enterica serovar Typhimurium strain, Salmonella enterica serovar Enteritidis strain or Salmonella enterica serovar Choleraesuis strain. In some embodiments, the bacterium is a Salmonella enterica serovar Typhi strain.

In some embodiments, the bacterium has one of the genotypes disclosed in Table 1 of Tennant & Levine, 2015. Vaccine. 33(0 3):C36-41 which is incorporated herein in its entirety by reference. In some embodiments, the bacterium is galE negative and Vi-capsule negative (see Germanier & Füer, 1975. J Infect Dis. 131(5):553-8).

In some embodiments, the bacterium is the Salmonella enterica serovar Typhi Ty21a strain (Germanier & Füer, 1975. J Infect Dis. 131(5):553-8). The genotype of the Ty21a strain is provided in Table 1 of Dharmasena et al., 2016. PLoS One. 11(9): e0163511. Ty21a is available for purchase from the American Type Culture Collection (ATCC 33459).

In some embodiments, the plasmid encodes one fusion protein comprising a coronavirus antigen and an adjuvant peptide. In some embodiments, the adjuvant promotes a Th1 or Th2-mediate response.

In some embodiments, the adjuvant is a mucosal adjuvant (see Aoshi, 2017. Viral Immunol. 30(6): 463-470). Exemplary mucosal adjuvants include interleukin-2 (IL-2) and cholera toxin B subunit.

IL-2 (SEQ ID NO: 1; UniProtKB - P60568)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT
Cholera toxin B subunit
(SEQ ID NO: 2; UniProtKB - Q57193)
TPQNITDLCAEYHNTQIHTLNDKIFSYTESLAGKREMAIITFKNGATFQV
EVPGSQHIDSQKKAIERMKDTLRIAYLTEAKVEKLCVWNNKTPHAIAAIS
MAN

In some embodiments, the adjuvant is SEQ ID NO: 1 or a peptide that has at least 95% sequence identity with SEQ ID NO: 1. In some embodiments, the adjuvant is SEQ ID NO: 1 or a peptide that has at least 98% sequence identity with SEQ ID NO: 1. In some embodiments, the adjuvant is SEQ ID NO: 1 or a peptide that has at least 99% sequence identity with SEQ ID NO: 1.

In some embodiments, the adjuvant is SEQ ID NO: 2 or a peptide that has at least 95% sequence identity with SEQ ID NO: 2. In some embodiments, the adjuvant is SEQ ID NO: 2 or a peptide that has at least 98% sequence identity with SEQ ID NO: 2. In some embodiments, the adjuvant is SEQ ID NO: 2 or a peptide that has at least 99% sequence identity with SEQ ID NO: 2.

In some embodiments, the adjuvant is a toll-like receptor agonist. Exemplary toll-like receptor agonists include Neisseria PorB and 50 s ribosomal protein L7/L12.

Neisseria PorB (SEQ ID NO: 3; UniProtKB - X5EGH0)
DVTLYGTIKAGVETSRSVEHNGGQVVSVETGTGIVDLGSKIGFKGQEDLG
NGLKAIWQVEQKASIAGTDSGWGNRQSFIGLKGGFGKLRVGRLNSVLKDT
GDINPWDSKSDYLGVNKIAEPEARLISVRYDSPEFAGLSGSVQYALNDNA
GRHNSESYHAGFNYKNGGFFVQYGGAYKRHQDVDDVKIEKYQIHRLVSGY
DNDALYASVAVQQQDAKLVEDNSHNSQTEVAATLAYRFGNVTPRVSYAHG
FKGSVDDAKRDNTYDQVVVGAEYDFSKRTSALVSAGWLQEGKGENKFVAT
AGGVGLRHKF
50s ribosomal protein L7/L12
(SEQ ID NO: 4; UniProtKB - Q735E8)
MAKMSTDDLLDAFKEMTLLELSDFVKKFEETFEVTAAAPVAVAAAGPAAG
GAPAEAAEEQSEFDVILESAGDKKIGVIKVVREIVSGLGLKEAKDLVDGA
PKPLLEKVAKEAADDAKAKLEAAGATVTVK

In some embodiments, the adjuvant is SEQ ID NO: 3 or a peptide that has at least 95% sequence identity with SEQ ID NO: 3. In some embodiments, the adjuvant is SEQ ID NO: 3 or a peptide that has at least 98% sequence identity with SEQ ID NO: 3. In some embodiments, the adjuvant is SEQ ID NO: 3 or a peptide that has at least 99% sequence identity with SEQ ID NO: 3.

In some embodiments, the adjuvant is SEQ ID NO: 4 or a peptide that has at least 95% sequence identity with SEQ ID NO: 4. In some embodiments, the adjuvant is SEQ ID NO: 4 or a peptide that has at least 98% sequence identity with SEQ ID NO: 4. In some embodiments, the adjuvant is SEQ ID NO: 4 or a peptide that has at least 99% sequence identity with SEQ ID NO: 4.

In some embodiments, the adjuvant is a β-defensin. Exemplary β-defensins include human β-defensin 1, human β-defensin 2, human β-defensin 3 and human β-defensin 4. In some embodiments, the adjuvant is human β-defensin 1.

Human β-defensin 1
(SEQ ID NO: 5; UniProtKB - P60022)
GNFLTGLGHRSDHYNCVSSGGQCLYSACPIFTKIQGTCYRGKAKCCK
Human β-defensin 2
(SEQ ID NO: 6; UniProtKB - O15263)
GIGDPVTCLKSGAICHPVFCPRRYKQIGTCGLPGTKCCKKP
Human β-defensin 3
(SEQ ID NO: 7; UniProtKB - P81534)
GIINTLQKYYCRVRGGRCAVLSCLPKEEQIGKCSTRGRKCCRRKK
Human β-defensin 4
(SEQ ID NO: 8; UniProtKB - Q8WTQ1)
EFELDRICGYGTARCRKKCRSQEYRIGRCPNTYACCLRKWDESLLNRTKP

In some embodiments, the adjuvant is SEQ ID NO: 5 or a peptide that has at least 90% sequence identity with SEQ ID NO: 5. In some embodiments, the adjuvant is SEQ ID NO: 5 or a peptide that has at least 95% sequence identity with SEQ ID NO: 5.

In some embodiments, the adjuvant is SEQ ID NO: 6 or a peptide that has at least 90% sequence identity with SEQ ID NO: 6. In some embodiments, the adjuvant is SEQ ID NO: 6 or a peptide that has at least 95% sequence identity with SEQ ID NO: 6.

In some embodiments, the adjuvant is SEQ ID NO: 7 or a peptide that has at least 90% sequence identity with SEQ ID NO: 7. In some embodiments, the adjuvant is SEQ ID NO: 7 or a peptide that has at least 95% sequence identity with SEQ ID NO: 7.

In some embodiments, the adjuvant is SEQ ID NO: 8 or a peptide that has at least 90% sequence identity with SEQ ID NO: 8. In some embodiments, the adjuvant is SEQ ID NO: 8 or a peptide that has at least 95% sequence identity with SEQ ID NO: 8.

In some embodiments, the fusion protein comprises the following structure:

Av-L-Ag (from N-terminus to C-terminus),

wherein Av is an adjuvant peptide, L is a linker and Ag is a coronavirus antigen.

The linker may be any genetically encodable linker known in the art (see Chen et al., 2013. Adv Drug Deliv Rev. 65(10):1357-1369). In some embodiments, the linker is EAAAK (SEQ ID NO: 9) or DPRVPSS (SEQ ID NO: 10).

In some embodiments, the plasmid encodes a first fusion protein and a second fusion protein, wherein each fusion protein comprises a coronavirus antigen and an adjuvant peptide.

An advantage of the present invention is that it allows for the combination of multiple antigens wherein one fusion protein may, for example, preferentially induce an antibody response whereas the second fusion protein may, for example, preferentially induce a T-cell response. The combination of an antibody response and T-cell response would be particularly advantageous for the treatment of a coronavirus infection.

In some embodiments, the first fusion protein comprises an adjuvant that promotes a Th1-mediated response and the second fusion protein comprises an adjuvant that promotes a Th2-mediated response.

In some embodiments, the first fusion protein comprises a mucosal adjuvant and the second fusion protein comprises an adjuvant that is a toll-like receptor agonist. In some embodiments, the first fusion protein comprises a mucosal adjuvant and the second fusion protein comprises an adjuvant that is a β-defensin.

In some embodiments, the first fusion protein comprises SEQ ID NO: 2 or a peptide that has at least 95, 98 or 99% sequence identity with SEQ ID NO: 2 and the second fusion protein comprises an adjuvant that is a toll-like receptor agonist. In some embodiments, the first fusion protein comprises SEQ ID NO: 2 or a peptide that has at least 95, 98 or 99% sequence identity with SEQ ID NO: 2 and the second fusion protein comprises an adjuvant that is a β-defensin.

In some embodiments, the coronavirus antigen is a SARS-CoV-2 antigen.

In some embodiments, the SARS-CoV-2 antigen is the spike glycoprotein or an antigenic fragment thereof, the membrane glycoprotein or an antigenic fragment thereof, the envelope protein, or the nucleocapsid protein or an antigenic fragment thereof.

Spike glycoprotein
(SEQ ID NO: 11; UniProtKB - P0DTC2)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT
Membrane glycoprotein
(SEQ ID NO: 12; UniProtKB - P0DTC5)
MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDHSSSSDNIALLVQ
Envelope protein
(SEQ ID NO: 13; UniProtKB - P0DTC4)
MYSFVSEETGTLIVNSVLLFLAFVVFLLVTLAILTALRLCAYCCNIVNVS
LVKPSFYVYSRVKNLNSSRVPDLLV
Nucleocapsid protein
(SEQ ID NO: 14; UniProtKB - P0DTC9)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA

In some embodiments, the coronavirus antigen comprises SEQ ID NO: 11 or a sequence that has at least 99% sequence identity with SEQ ID NO: 11. In some embodiments, the coronavirus antigen comprises residues 2-1273 of SEQ ID NO: 11 or a sequence that has at least 99% sequence identity with residues 2-1273 of SEQ ID NO: 11. In some embodiments, the coronavirus antigen comprises residues 13-303 of SEQ ID NO: 11 or a sequence that has at least 99% sequence identity with residues 13-303 of SEQ ID NO: 11. In some embodiments, the coronavirus antigen comprises residues 319-541 of SEQ ID NO: 11 or a sequence that has at least 99% sequence identity with residues 319-541 of SEQ ID NO: 11. In some embodiments, the coronavirus antigen comprises residues 334-527 of SEQ ID NO: 11 or a sequence that has at least 99% sequence identity with residues 334-527 of SEQ ID NO: 11. In some embodiments, the coronavirus antigen comprises residues 437-508 of SEQ ID NO: 11 or a sequence that has at least 98% sequence identity with residues 437-508 of SEQ ID NO: 11. In some embodiments, the coronavirus antigen comprises residues 788-806 of SEQ ID NO: 11 or a sequence that has at least 94% sequence identity with residues 788-806 of SEQ ID NO: 11. In some embodiments, the coronavirus antigen comprises residues 920-970 of SEQ ID NO: 11 or a sequence that has at least 98% sequence identity with residues 920-970 of SEQ ID NO: 11. In some embodiments, the coronavirus antigen comprises residues 1163-1202 of SEQ ID NO: 11 or a sequence that has at least 97% sequence identity with residues 1163-1202 of SEQ ID NO: 11. In some embodiments, the coronavirus antigen comprises residues 1235-1273 of SEQ ID NO: 11 or a sequence that has at least 97% sequence identity with residues 1235-1273 of SEQ ID NO: 11.

In some embodiments, the coronavirus antigen comprises SEQ ID NO: 12 or a sequence that has at least 99% sequence identity with SEQ ID NO: 12. In some embodiments, the coronavirus antigen comprises residues 2-222 of SEQ ID NO: 12 or a sequence that has at least 99% sequence identity with residues 2-222 of SEQ ID NO: 12. In some embodiments, the coronavirus antigen comprises residues 2-100 of SEQ ID NO: 12 or a sequence that has at least 99% sequence identity with residues 2-100 of SEQ ID NO: 12.

In some embodiments, the coronavirus antigen comprises SEQ ID NO: 13 or a sequence that has at least 98% sequence identity with SEQ ID NO: 13.

In some embodiments, the coronavirus antigen comprises SEQ ID NO: 14 or a sequence that has at least 99% sequence identity with SEQ ID NO: 14. In some embodiments, the coronavirus antigen comprises residues 2-419 of SEQ ID NO: 14 or a sequence that has at least 99% sequence identity with residues 2-419 of SEQ ID NO: 14. In some embodiments, the coronavirus antigen comprises residues 41-186 of SEQ ID NO: 14 or a sequence that has at least 99% sequence identity with residues 41-186 of SEQ ID NO: 14. In some embodiments, the coronavirus antigen comprises residues 258-361 of SEQ ID NO: 14 or a sequence that has at least 99% sequence identity with residues 258-361 of SEQ ID NO: 14.

Other SARS-CoV-2 antigens include SEQ ID NOs: 15-18 provided below.

SEQ ID NO: 15
GTTLPKKKFFGMSRIGMEVTPSGTWKKLLPAADGPGPGAALALLLLDRLN
QLEGPGPGGTWLTYTGAIKLDDKGPGPGFPRGQGVPIAAYFPRGQGVPIA
AYFPRGQGVPIAAYLSPRWYFYYAAYLLLDRLNQLAAYKSAAEASKKAAY
KPRQKRTATAAYGMSRIGMEVAAYKTFPPTEPK
SEQ ID NO: 16
GTTLPKKKFFGMSRIGMEVTPSGTWKKLLPAADGPGPGAALALLLLDRLN
QLEGPGPGGTWLTYTGAIKLDDKGPGPGFPRGQGVPIAAYFPRGQGVPIA
AYFPRGQGVPIAAYLSPRWYFYY
SEQ ID NO: 17
AALALLLLDRLNQLEGPGPGGTWLTYTGAIKLDDKGPGPGFPRGQGVPIA
AYFPRGQGVPIAAYFPRGQGVPIAAYLSPRWYFYY
SEQ ID NO: 18
AALALLLLDRLNQLEGPGPGGTWLTYTGAIKLDDKGPGPGFPRGQGVPIA
AYFPRGQGVPIAAYFPRGQGVPIAAYLSPRWYFYYAAYLLLDRLNQLAAY
KSAAEASKKAAYKPRQKRTATAAYGMSRIGMEVAAYKTFPPTEPK

In some embodiments, the coronavirus antigen comprises SEQ ID NO: 15 or a sequence that has at least 99% sequence identity with SEQ ID NO: 15. In some embodiments, the coronavirus antigen comprises SEQ ID NO: 16 or a sequence that has at least 99% sequence identity with SEQ ID NO: 16. In some embodiments, the coronavirus antigen comprises SEQ ID NO: 17 or a sequence that has at least 98% sequence identity with SEQ ID NO: 17. In some embodiments, the coronavirus antigen comprises SEQ ID NO: 18 or a sequence that has at least 99% sequence identity with SEQ ID NO: 18.

In some embodiments, the coronavirus antigen comprises any one of SEQ ID NOs: 11-18 or an antigenic fragment thereof. In some embodiments, the coronavirus antigen is selected from any one of SEQ ID NOs: 11-18 or is an antigenic fragment of any one of SEQ ID NOs: 11-18.

In some embodiments, the fusion protein comprises:

(i) residues 319-541 of SEQ ID NO: 11 or a sequence that has at least 99% sequence identity with residues 319-541 of SEQ ID NO: 11; and

(ii) SEQ ID NO: 2 or a peptide that has at least 95% sequence identity with SEQ ID NO: 2.

In some embodiments, the fusion protein comprises the following structure:

Av-L-Ag (from N-terminus to C-terminus),

wherein Av is SEQ ID NO: 2 or a peptide that has at least 95% sequence identity with SEQ ID NO: 2, L is EAAAK; and

Ag is residues 319-541 of SEQ ID NO: 11 or a sequence that has at least 99% sequence identity with residues 319-541 of SEQ ID NO: 11.

In some embodiments, the fusion protein comprises:

(i) SEQ ID NO: 15 or a sequence that has at least 99% sequence identity with SEQ ID NO: 15; and

(ii) SEQ ID NO: 5 or a peptide that has at least 95% sequence identity with SEQ ID NO: 5.

In some embodiments, the fusion protein comprises the following structure:

Av-L-Ag (from N-terminus to C-terminus),

wherein Av is SEQ ID NO: 5 or a peptide that has at least 95% sequence identity with SEQ ID NO: 5, L is EAAAK; and

Ag is SEQ ID NO: 15 or a sequence that has at least 99% sequence identity with SEQ ID NO: 15.

In some embodiments, the plasmid comprises a nucleic acid encoding a first fusion protein and a nucleic acid encoding a second fusion protein,

wherein the first fusion protein comprises:

(i) residues 319-541 of SEQ ID NO: 11 or a sequence that has at least 99% sequence identity with residues 319-541 of SEQ ID NO: 11; and

(ii) SEQ ID NO: 2 or a peptide that has at least 95% sequence identity with SEQ ID NO: 2; and the second fusion protein comprises:

(i) SEQ ID NO: 15 or a sequence that has at least 99% sequence identity with SEQ ID NO: 15; and

(ii) SEQ ID NO: 5 or a peptide that has at least 95% sequence identity with SEQ ID NO: 5.

In some embodiments, the one or more fusion proteins further comprise a secretion signal peptide. The secretion signal peptide may be a hemolysin A secretion signal peptide, a PhoA signal peptide, an OmpA signal peptide, or a BLA signal peptide.

An example of a hemolysin A (HlyA) secretion signal peptide is SEQ ID NO: 19:

LAYGSQGDLNPLINEISKIISAAGSFDVKEERTAASLLQLSGNASDFSYG
RNSITLTTSA

An example of a PhoA signal peptide is SEQ ID NO: 20:

MKQSTIALALLPLLFTPVTKA

An example of an OmpA signal peptide is SEQ ID NO: 21:

MKKTAIAIAVALAGFATVAQA

An example of a BLA signal peptide is SEQ ID NO: 22:

MSIQHFRVALIPFFAAFCLPVFA

In some embodiments, the fusion protein comprises the BLA signal peptide according to SEQ ID NO: 23 and the C-terminal sequence of BLA according to SEQ ID NO: 24 (Xin et al., 2008. Infect Immun. 76(7):3241-3254).

SEQ ID NO: 23
MSIQHFRVALIPFFAAFCLPVFAHPETLVKVKDA
SEQ ID NO: 24
ATMDERNRQIAEIGASLIKHW

In embodiments wherein the fusion protein comprises the C-terminal signal peptide of HlyA (e.g., SEQ ID NO: 19), it may be advantageous to include the N-terminal sequence of HlyA (e.g., SEQ ID NO: 25).

SEQ ID NO: 25
MPTITTAQIKSTLQSAKQSAANKLHSAGQSTK

Thus, in some embodiments the fusion protein comprises the following structure:

HlyA_{N}-L-Av-L-Ag-L-HlyA_{S} (from N-terminus to C-terminus),

wherein HlyA_{N} is the N-terminal sequence of HlyA (e.g., SEQ ID NO: 25),

Av is an adjuvant peptide,

L is a linker,

Ag is a coronavirus antigen, and

HlyA_{S} is the signal peptide of HlyA (e.g., SEQ ID NO: 19).

In embodiments where the fusion protein comprises the HlyA secretion signal peptide, the plasmid may further encode HlyB and HlyD. Alternatively, a further nucleic acid encoding HlyB and HlyD is inserted into the bacterium. The plasmid may also further encode HlyC and/or HlyR or a further nucleic acid encoding HlyC and/or HlyR could be used.

In some embodiments, the bacterium and/or the plasmid does not comprise an antibiotic marker. In some embodiments, the bacterium is a ΔtyrS (i.e., the gene encoding tyrosyl-tRNA-synthetase has been removed or inactivated) strain and the plasmid further encodes tyrS. This provides a balanced lethal system which allows for the maintenance of the plasmid in the bacterium without the need of an antibiotic resistance cassette.

In some embodiments, the plasmid is integrated into the chromosome of the bacterium or replicates independently of the chromosome of the bacterium. Preferably, the plasmid replicates independently of the chromosome of the bacterium.

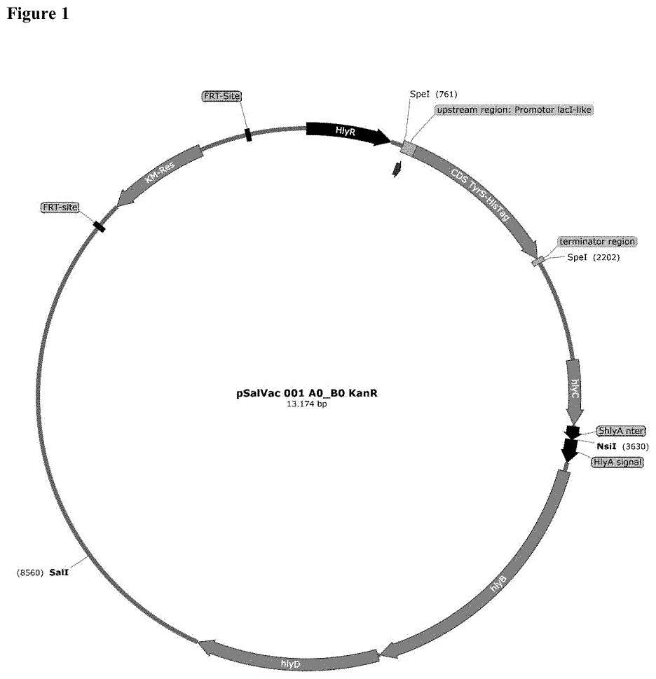

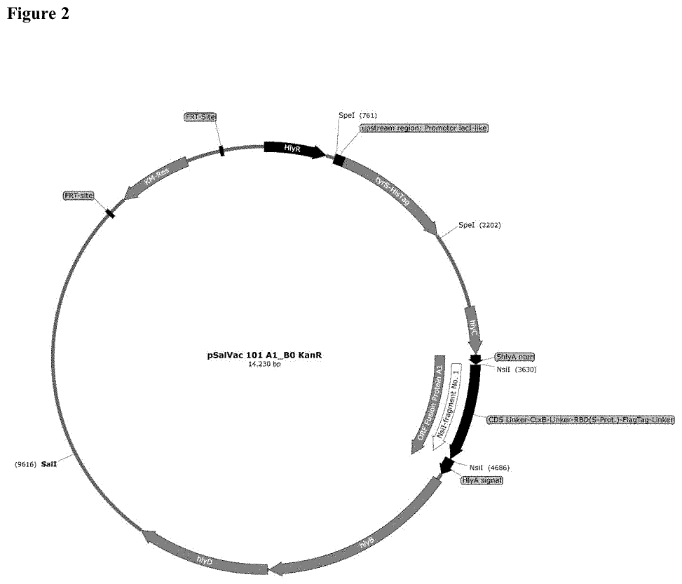

FIG. 1 depicts Map of plasmid pSalVac 001 A0_B0 KanR, the first generation of basic cloning vectors of the present invention. The plasmid has the capacity for inserting fragments encoding fusion proteins at two sites. The first site, depicted as A-Site, is the NsiI cleavage site which results in the secretion of a fusion protein via the HlyA secretion system (see FIG. 2). The second site, depicted as B-site is the SalI site which allows for more flexibility (e.g., can use different promoter regions and signal peptides). Furthermore, the plasmid harbours a kanamycin resistance gene flanked by two FRT-sites (Fensterle et al., 2008). This feature allows the excision of the kanamycin gene by the site-specific enzyme FLP recombinase, which acts on the directly repeated FRT (FLP recognition/recombination target). All genes of the hemolysin secretion system gene cluster (including the hlyA˜-fused hybrid gene) are transcribed from the promoter PhlyI in front of hlyC (Vogel et al., 1988, Gentschev et al., 1996). The enhancing sequence hlyR is separated from this promoter by more than 1.5 kb including an IS2 element (Vogel et al., 1988). As Vogel et al. (1988) could have shown that the IS2-like sequence is not directly involved in the enhancement mechanism of hlyR, we decided to delete this region creating a single SpeI-site which represents an integration-site for subsequent alternate tyrS-complementing expression cassettes. In pSalVac 001 A0_B0 KanR the tyrS expression cassette is under control of the lacI-like promotor (Promotor region PR 2, SEQ ID NO: 34).

Thus, in some embodiments, the first fusion protein comprises a HlyA secretion signal peptide and the second fusion protein comprises a HlyA secretion signal peptide, a PhoA signal peptide, an OmpA signal peptide, or a BLA signal peptide.

In some embodiments, the fusion protein further comprises a purification tag. Different purification tags and purification systems are known to the skilled person. The purification tag may be any one of those disclosed in Table 9.9.1 of Kimple et al., 2013. Curr Protoc Protein Sci. 73(1): 9.9.1-9.9.23 which is incorporated by reference in its entirety. In some embodiments, the purification tag is a polyhistidine tag, FLAG-tag or HA-tag. The HA-tag may consist of YPYDVPDYA (SEQ ID NO: 26).

In some embodiments, the purification tag may be attached to the fusion protein via a cleavable linker. Cleavable linkers are known in the art (see Chen et al., 2013. Adv Drug Deliv Rev. 65(10):1357-1369). In some embodiments, the cleavable linker consists of DDDDK (SEQ ID NO: 27) or LVPRGS (SEQ ID NO: 28).

In a preferred embodiment of the invention, the fusion protein selected from any one of the constructs of Table 4 or Table 5.

In another preferred embodiment of the invention, the fusion protein selected from any one of the constructs of Table 13 or Table 15.

In another preferred embodiment of the invention, the fusion protein is a protein consisting of an amino acid sequence of any one of SEQ ID NO: 30, 92, 94, 96, 98, 100, 102, 106, 108, 110, 112, 114, 116, 118, 146, 148, 150, 152, 154, 156, 162, 164, or 166, or a protein consisting of an amino acid sequence at least 99% identical to the amino acid sequence of any one of SEQ ID NO: 30, 92, 94, 96, 98, 100, 102, 106, 108, 110, 112, 114, 116, 118, 146, 148, 150, 152, 154, 156, 162, 164, or 166.

In another preferred embodiment of the invention, the fusion protein is encoded by any one of the coding sequences (CDS) of Tables 13 or 15.

In a very preferred embodiment of the invention, the first fusion protein is selected from any one of the constructs of Table 4, and the second fusion protein is selected from any one of the constructs of Table 5.

In a very preferred embodiment of the invention, the first fusion protein is selected from any one of the constructs of Table 13, and the second fusion protein is selected from any one of the constructs of Table 15.

In some embodiments, the plasmid comprises a nucleic acid encoding the following components:

Tg-L-Av-L-Ag; or

Av-L-Ag-L-Tg,

wherein Av is an adjuvant peptide, L is a linker, Ag is a coronavirus antigen and Tg is a purification tag.

In some embodiments, the plasmid comprises the following components:

HlyA_{N}-X-L₁-Av-L₂-Ag-L₃-X-HlyA_{S};

HlyA_{N}-X-L₁-Av-L₂-Ag-L₄-Tg-L₃-X-HlyA_{S}; or

HlyA_{N}-X-Tg-L₁-Av-L₂-Ag-L₃-X-HlyA_{S},

wherein HlyA_{N} encodes the N-terminal sequence of HlyA (e.g., SEQ ID NO: 25),

X is a restriction recognition site,

Tg encodes a purification tag,

L₁ encodes SEQ ID NO: 9 or SEQ ID NO: 10,

Av encodes an adjuvant peptide (preferably a mucosal adjuvant),

L₂ encodes SEQ ID NO: 9 or SEQ ID NO: 10,

Ag encodes a coronavirus antigen,

L₃ encodes SEQ ID NO: 9,

L₄ encodes AAY, GPGPG (SEQ ID NO: 29), or KK, and

HlyA_{S} encodes the signal peptide of HlyA (e.g., SEQ ID NO: 19). In some embodiments, the restriction recognition site is the NsiI recognition site (i.e., ATGCAT).

In some embodiments, the plasmid comprises a sequence that encodes SEQ ID NO: 30 or a sequence that has at least 95% identity with SEQ ID NO: 30. In some embodiments, the plasmid comprises a sequence that encodes SEQ ID NO: 30 or a sequence that has at least 98% identity with SEQ ID NO: 30. In some embodiments, the plasmid comprises a sequence that encodes SEQ ID NO: 30 or a sequence that has at least 99% identity with SEQ ID NO: 30.

HlyA_{N}-linker-CtxB-linker-RBD (S-Protein)-FlagTag-
Linker-HlyA_{S}-CDS
(SEQ ID NO: 30)
MPTITTAQIKSTLQSAKQSAANKLHSAGQSTKDASEAAAKTPQNITDLC
AEYHNTQIHTLNDKIFSYTESLAGKREMAIITFKNGATFQVEVPGSQHI
DSQKKAIERMKDTLRIAYLTEAKVEKLCVWNNKTPHAIAAISMANEAAA
KRVQPTESIVRFPNITNLCPFGEVFNATRFASVYAWNRKRISNCVADYS
VLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPF
ERDISTEIYQAGSTPCNGVEGFNCYFPLQSYGFQPTNGVGYQPYRVVVL
SFELLHAPATVCGPKKSTNLVKNKCVNFDYKDDDDKEAAAKHALAYGSQ
GDLNPLINEISKIISAAGSFDVKEERTAASLLQLSGNASDFSYGRNSIT
LTTSA

In some embodiments, the fusion proteins have been codon optimized for optimal expression in the bacterium.

In some embodiments, the plasmid comprises SEQ ID NO: 31 or a sequence that has 75, 80, 85, 90, 95, 98 or 99% identity with SEQ ID NO: 31.

SEQ ID NO: 31
Atgcatcagaagcggcggcgaaaaccccgcagaacatcaccgacctgtg
cgcggaataccacaacacccagatccacaccctgaacgacaaaatcttc
tcctacaccgaatccctggcgggcaaacgtgaaatggcgatcatcacct
tcaaaaacggcgcgaccttccaggttgaagttccgggctcccagcacat
cgactcccagaaaaaagcgatcgaacgtatgaaagacaccctgcgtatc
gcgtacctgaccgaagcgaaagttgaaaaactgtgcgtttggaacaaca
aaaccccgcacgcgatcgcggcgatctccatggcgaacgaagcggcggc
gaaacgtgttcagccgaccgaatccatagttaggttcccgaacatcact
aacctgtgtccgtttggcgaagtgttcaacgcgacccgttttgcgtccg
tctacgcctggaaccgtaaacgtatctccaactgcgttgcggactactc
cgttctgtacaactccgcgtccttctccaccttcaaatgctacggcgtt
tccccgaccaaactgaacgacctgtgcttcaccaacgtttacgcggact
ccttcgttatccgtggcgacgaagttcgtcagatcgcgccgggccagac
cggcaaaatcgcggactacaactacaaactgccggacgacttcaccggc
tgcgttatcgcgtggaactccaacaacctggactccaaagttggcggca
actacaactacctgtaccgtctgttccgtaaatccaacctgaaaccgtt
cgaacgtgacatctccaccgaaatctaccaggcgggctccaccccgtgc
aacggcgttgaaggcttcaactgctacttcccgctgcagtcctacggct
tccagccgaccaacggcgttggctaccagccgtaccgtgttgttgttct
gtccttcgaactgctgcacgcgccggcgaccgtttgcggcccgaaaaaa
tccaccaacctggttaaaaacaaatgcgttaacttcgactacaaagacg
acgacgacaaagaagcggcggcgaaacatgcat

In some embodiments, the plasmid comprises SEQ ID NO: 32 or a sequence that has 75, 80, 85, 90, 95, 98 or 99% sequence identity with SEQ ID NO: 32.

SEQ ID NO: 32
atgccaacaataaccactgcacaaattaaaagcacactgcagtctgcaa
agcaatccgctgcaaataaattgcactcagcaggacaaagcacgaaaga
tgcatcagaagcggcggcgaaaaccccgcagaacatcaccgacctgtgc
gcggaataccacaacacccagatccacaccctgaacgacaaaatcttct
cctacaccgaatccctggcgggcaaacgtgaaatggcgatcatcacctt
caaaaacggcgcgaccttccaggttgaagttccgggctcccagcacatc
gactcccagaaaaaagcgatcgaacgtatgaaagacaccctgcgtatcg
cgtacctgaccgaagcgaaagttgaaaaactgtgcgtttggaacaacaa
aaccccgcacgcgatcgcggcgatctccatggcgaacgaagcggcggcg
aaacgtgttcagccgaccgaatccatagttaggttcccgaacatcacta
acctgtgtccgtttggcgaagtgttcaacgcgacccgttttgcgtccgt
ctacgcctggaaccgtaaacgtatctccaactgcgttgcggactactcc
gttctgtacaactccgcgtcctctccaccttcaaatgctacggcgtttc
cccgaccaaactgaacgacctgtgcttcaccaacgtttacgcggactcc
ttcgttatccgtggcgacgaagttcgtcagatcgcgccgggccagaccg
gcaaaatcgcggactacaactacaaactgccggacgacttcaccggctg
cgttatcgcgtggaactccaacaacctggactccaaagttggcggcaac
tacaactacctgtaccgtctgttccgtaaatccaacctgaaaccgttcg
aacgtgacatctccaccgaaatctaccaggcgggctccaccccgtgcaa
cggcgttgaaggcttcaactgctacttcccgctgcagtcctacggcttc
cagccgaccaacggcgttggctaccagccgtaccgtgttgttgttctgt
ccttcgaactgctgcacgcgccggcgaccgtttgcggcccgaaaaaatc
caccaacctggttaaaaacaaatgcgttaacttcgactacaaagacgac
gacgacaaagaagcggcggcgaaacatgcattagcctatggaagtcagg
gtgatcttaatccattaattaatgaaatcagcaaaatcatttcagctgc
aggtagcttcgatgttaaagaggaaagaactgcagcttctttattgcag
ttgtccggtaatgccagtgatttttcatatggacggaactcaataaccc
tgaccacatcagcataa

In some embodiments, the plasmid comprises the following components:

X-Pr-Av-L₁-Ag-Tr-X;

X-Pr-Sp-Av-L₁-Ag-Tr-X;

X-Pr-Av-L₁-Ag-L₂-Tg-Tr-X;

X-Pr-Sp-Av-L₁-Ag-Tg-Tr-X; or

X-Pr-Sp-Av-L₁-Ag-L₂-Tg-Tr-X, wherein

X is a restriction recognition site,

Pr is a Promoter region,

Tr is a Terminator region,

Sp encodes a secretion signal peptide,

Tg encodes a purification tag,

Av encodes an adjuvant peptide (preferably a toll-like receptor agonist or β-defensin),

L₁ encodes SEQ ID NO: 9, and

L₂ encodes SEQ ID NO: 9, AAY, SEQ ID NO: 29 or KK, and

Ag encodes a coronavirus antigen. In some embodiments, L₂ is optional. In some embodiments, the restriction recognition site is the SalI recognition site (i.e., GTCGAC). In some embodiments, Sp encodes a PhoA signal peptide, an OmpA signal peptide or a BLA signal peptide.

Exemplary promoter regions include:

lacI_{EC}
(SEQ ID NO: 33)
GACACCATCGAATGGCGCAAAACCTTTCGCGGTATGGCATGATAGCGCC
CGGAAGAGAGTCAATTCAGGGTGGTGAAT
lacI_{EC}-like
(SEQ ID NO: 34)
GCTAGCGACACCATCGAATGGCGCAAACCTTTCGCGGTATGGCATGATA
GCGCCCGAAGTCGTGTACCGGCAAAGGTGAGTCGTTATATACATGGAGA
TTTTG
tyrS of E. coli
(SEQ ID NO: 35)
GTAAATTCCTGGAGCTGAAGCAGAAGTTTCAACAGGGCGAAGTGCCATT
GCCGAGCTTTTGGGGCGGTTTTCGCGTCAGCCTTGAACAGATTGAGTTC
TGGCAGGGTGGTGAGCATCGCCTGCATGACCGCTTTTTGTACCAGCGTG
AAAATGATGCGTGGAAGATTGATCGTCTTGCACCCTGAAAAGATGCAAA
AATCTTGCTTTAATCGCTGGTACTCCTGATTCTGGCACTTTATTCTATG
TCTCTTTCGCATCTGGCGAAAAGTCGTGTACCGGCAAAGGTGCAGTCGT
TATATACATGGAGATTTTG
tyrS of E. coli
(SEQ ID NO: 36)
CCTGCATGACCGCTTTTTGTACCAGCGTGAAAATGATGCGTGGAAGATT
GATCGTCTTGCACCCTGAAAAGATGCAAAAATCTTGCTTTAATCGCTGG
TACTCCTGATTCTGGCACTTTATTCTATGTCTCTTTCGCATCTGGCGAA
AAGTCGTGTACCGGCAAAGGTGCAGTCGTTATATACATGGAGATTTTG
and
tyrS of E. coli
(SEQ ID NO: 37)
CTCCTGATTCTGGCACTTTATTCTATGTCTCTTTCGCATCTGGCGAAAA
GTCGTGTACCGGCAAAGGTGCAGTCGTTATATACATGGAGATTTTG.

Exemplary terminator regions include

Terminator region of TyrS-HisTag EPC
(SEQ ID NO: 38)
TAATCCACGGCCGCCAGTTTGGGCTGGCGGCATTTTGGTACC
lacI_{EC} E. coli
(SEQ ID NO: 39)
TAATGAGCGCAACGCAATTAATGTGAGTTAGCTCACTCATTAGGCACC
tyrS_{EC} E. coli
(SEQ ID NO: 40)
TGCATTAAGTGGAAAGGGGGAGTGAGAAATCACTCCCCCTGGTTTTTAT
ACAGGGAAC
Terminator Region TR 2
(SEQ ID NO: 43)
TGACGCAACGCAATTAATGTGAGTTAGCTCACTCATTAGGCACC
and
Terminator region T0: BBA_K864600 T0-TERMINATOR
(SEQ ID NO: 44)
TTGTTCAGAACGCTCGGTCTTGCACACCGGGCGTTTTTTCTTTGTGAGT
CCA

In some embodiments, the plasmid comprises a sequence that encodes SEQ ID NO: 41 or a sequence that has at least 95% identity with SEQ ID NO: 41. In some embodiments, the plasmid comprises a sequence that encodes SEQ ID NO: 41 or a sequence that has at least 98% identity with SEQ ID NO: 41. In some embodiments, the plasmid comprises a sequence that encodes SEQ ID NO: 41 or a sequence that has at least 99% identity with SEQ ID NO: 41.

PhoA-human β-defensin 1-N-Multiepitope unit
Variant 1-T7-tag
(SEQ ID NO: 41)
MKQSTIALALLPLLFTPVTKAGNFLTGLGHRSDHYNCVSSGGQCLYSAC
PIFTKIQGTCYRGKAKCCKEAAAKGTTLPKKKFFGMSRIGMEVTPSGTW
KKLLPAADGPGPGAALALLLLDRLNQLEGPGPGGTWLTYTGAIKLDDKG
PGPGFPRGQGVPIAAYFPRGQGVPIAAYFPRGQGVPIAAYLSPRWYFYY
AAYLLLDRLNQLAAYKSAAEASKKAAYKPRQKRTATAAYGMSRIGMEVA
AYKTFPPTEPKAAYMASMTGGQQMG

In some embodiments, the plasmid comprises:

(i) a sequence that encodes SEQ ID NO: 41 or a sequence that has at least 95% identity with SEQ ID NO: 41; and

(ii) a sequence that encodes SEQ ID NO: 30 or a sequence that has at least 95% identity with SEQ ID NO: 30.

In some embodiments, the plasmid comprises:

(i) a sequence that encodes SEQ ID NO: 41 or a sequence that has at least 98% identity with SEQ ID NO: 41; and

(ii) a sequence that encodes SEQ ID NO: 30 or a sequence that has at least 98% identity with SEQ ID NO: 30.

In some embodiments, the plasmid comprises:

(i) a sequence that encodes SEQ ID NO: 41 or a sequence that has at least 99% identity with SEQ ID NO: 41; and

(ii) a sequence that encodes SEQ ID NO: 30 or a sequence that has at least 99% identity with SEQ ID NO: 30.

In some embodiments, the plasmid comprises:

(i) a sequence that encodes SEQ ID NO: 41; and

(ii) a sequence that encodes SEQ ID NO: 30.

In a preferred embodiment of the invention, the coronavirus antigen is selected from any one of the viral antigen units of Table 4 or Table 5.

In another preferred embodiment of the invention, the coronavirus antigen is selected from any one of the viral antigen units of Table 14 or Table 16.

In another preferred embodiment of the invention, the coronavirus antigen consists of an amino acid sequence of any one of SEQ ID NOs: 11-18, 120, 122, 124, 126, 128, 130, 132, 134, 136, 138, 140, 142, 144, 168, or 170, or consists of an amino acid sequence at least 99% identical to the amino acid sequence of any one of SEQ ID NOs: 11-18, 120, 122, 124, 126, 128, 130, 132, 134, 136, 138, 140, 142, 144, 168, or 170.

In another preferred embodiment of the invention, the coronavirus antigen is encoded by any one of the coding sequences (CDS) of Table 14 or Table 16 or by the coding sequences (CDS) of any one of SEQ ID Nos 178-183.

### Combination Product

The inclusion of a purification tag allows one to express and purify the one or more fusion proteins encoded by the plasmid comprised in the bacterium. After cleavage of the purification tags and removal of LPS, the fusion protein can be used in prime-boost vaccines (e.g. oral, nasal) or can be added to the live vaccine as an adjuvant-antigen-fusion protein to increase amount of the antigenic fusion protein and/or to deliver an additional set of adjuvant-antigen-combinations.

Thus, in another aspect the present invention provides a combination product comprising (i) the live-attenuated bacterium of the present invention and (ii) the one or more fusion proteins encoded by the recombinant plasmid found within the bacterium of the present invention.

### Vaccine and Pharmaceutical Compositions

In another aspect, the present invention provides a vaccine comprising the bacterium of the present invention or the combination product of the present invention. In some embodiments, the vaccine further comprises a pharmaceutically acceptable carrier or diluent.

The vaccine may also be referred to as a “pharmaceutical composition”.

A pharmaceutical composition as described herein may also contain other substances. These substances include, but are not limited to, cryoprotectants, lyoprotectants, surfactants, bulking agents, anti-oxidants, and stabilizing agents. In some embodiments, the pharmaceutical composition may be lyophilized.

The term “cryoprotectant” as used herein, includes agents which provide stability to the active ingredient against freezing-induced stresses, by being preferentially excluded from the active ingredient's surface. Cryoprotectants may also offer protection during primary and secondary drying and long-term product storage. Non-limiting examples of cryoprotectants include sugars, such as sucrose, glucose, trehalose, mannitol, mannose, and lactose; polymers, such as dextran, hydroxyethyl starch and polyethylene glycol; surfactants, such as polysorbates (e.g., PS-20 or PS-80); and amino acids, such as glycine, arginine, leucine, and serine. A cryoprotectant exhibiting low toxicity in biological systems is generally used.

In one embodiment, a lyoprotectant is added to a pharmaceutical composition described herein. The term “lyoprotectant” as used herein, includes agents that provide stability to the active ingredient during the freeze-drying or dehydration process (primary and secondary freeze-drying cycles), by providing an amorphous glassy matrix and by binding with the a's surface through hydrogen bonding, replacing the water molecules that are removed during the drying process. This helps to minimize product degradation during the lyophilization cycle and improve the long-term product stability. Non-limiting examples of lyoprotectants include sugars, such as sucrose or trehalose; an amino acid, such as monosodium glutamate, non-crystalline glycine or histidine; a metHlyAmine, such as betaine; a lyotropic salt, such as magnesium sulfate; a polyol, such as trihydric or higher sugar alcohols, e.g., glycerin, erythritol, glycerol, arabitol, xylitol, sorbitol, and mannitol; propylene glycol; polyethylene glycol; pluronics; and combinations thereof. The amount of lyoprotectant added to a pharmaceutical composition is generally an amount that does not lead to an unacceptable amount of degradation of the strain when the pharmaceutical composition is lyophilized.

In some embodiments, a bulking agent is included in the pharmaceutical composition. The term “bulking agent” as used herein, includes agents that provide the structure of the freeze-dried product without interacting directly with the pharmaceutical product. In addition to providing a pharmaceutically elegant cake, bulking agents may also impart useful qualities in regard to modifying the collapse temperature, providing freeze-thaw protection, and enhancing the strain stability over long-term storage. Non-limiting examples of bulking agents include mannitol, glycine, lactose, and sucrose. Bulking agents may be crystalline (such as glycine, mannitol, or sodium chloride) or amorphous (such as dextran, hydroxyethyl starch) and are generally used in formulations in an amount from 0.5% to 10%.

Other pharmaceutically acceptable carriers, excipients, or stabilizers, such as those described in Remington: The Science and Practice of Pharmacy 22nd edition, Pharmaceutical press (2012), ISBN-13: 9780857110626 may also be included in a pharmaceutical composition described herein, provided that they do not adversely affect the desired characteristics of the pharmaceutical composition. As used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone.

In some embodiments, the pharmaceutical composition may be suitable for oral, buccal, nasal, intravenous, intramuscular, conjunctival, transdermal, intraperitoneal and/or subcutaneous administration, preferably oral, nasal, intravenous and/or intramuscular administration.

The pharmaceutical composition may further comprise common excipients and carriers which are known in the state of the art. For solution for injection, the pharmaceutical composition may further comprise cryoprotectants, lyoprotectants, surfactants, bulking agents, anti-oxidants, stabilizing agents and pharmaceutically acceptable carriers.

### Medical Uses

In another aspect, the present invention provides the bacterium of the present invention, the combination product of the present invention or the vaccine of the present invention for use as a medicament.

In another aspect, the present invention provides the bacterium of the present invention, the combination product of the present invention or the vaccine of the present invention for use in a method of treating a disease or disorder caused by a member of the coronavirus family. In some embodiments, the method comprises administering a therapeutically effective amount of the bacterium, combination product or vaccine to a subject.

In some embodiments, the disease or disorder is COVID-19. In some embodiments, the coronavirus is SARS-CoV-2.

In some embodiments, the bacterium, combination product or vaccine is administered orally, buccally, intranasally, intravenously, intramuscularly, transdermally, intraperitoneally or subcutaneously. In some embodiments, administration is performed orally, intranasally, intravenously or intramuscularly.

### Kit

In another aspect, the present invention provides a kit comprising a live-attenuated bacterium of the genus Salmonella and a recombinant plasmid encoding a fusion protein, wherein the fusion protein comprises a coronavirus antigen and an adjuvant peptide.

The bacterium, plasmid and fusion protein may be in accordance with any aspect and/or embodiment disclosed throughout this application.

For the avoidance of any doubt, any instance wherein the term “comprising” is used throughout the entirety of the present application may optionally be replaced by the expression “consisting of”.

### Items

The present invention also provides the following items which may be combined with any aspect or embodiment described throughout the entirety of the present application.

[1] A live-attenuated bacterium of the genus Salmonella comprising a recombinant plasmid encoding a fusion protein, wherein the fusion protein comprises:

(i) a coronavirus antigen; and

(ii) an adjuvant peptide.

[2] The bacterium of [1], wherein the bacterium is of the species Salmonella enterica.

[3] The bacterium of [1] or [2], wherein the bacterium is a Salmonella enterica serovar Typhi strain.

[4] The bacterium of [3], wherein the bacterium is the Ty21a strain.

[5] The bacterium of any one of [1]-[4], wherein the adjuvant is a (i) mucosal adjuvant, or (ii) a toll-like receptor agonist or β-defensin.

[6] The bacterium of any one of [1]-[5], wherein the plasmid encodes a first fusion protein and a second fusion protein, wherein each fusion protein comprises:

(i) a coronavirus antigen; and

(ii) an adjuvant peptide.

[7] The bacterium of [6], wherein the first fusion protein comprises:

(i) a coronavirus antigen; and

(ii) a mucosal adjuvant peptide.

[8] The bacterium of [7], wherein the second fusion protein comprises:

(i) a coronavirus antigen; and

(ii) a toll-like receptor agonist or β-defensin.

[9] The bacterium of [5] or [7], wherein the mucosal adjuvant is an interleukin-2 or a cholera toxin B subunit, wherein, optionally, the mucosal adjuvant is a cholera toxin B subunit.

[10] The bacterium of [5] or [8], wherein the toll-like receptor agonist is a Neisseria PorB or 50 s ribosomal protein L7/L12.

[11] The bacterium of [5], [8] or [10], wherein the β-defensin is human β-defensin 1, human β-defensin 2, human β-defensin 3 or human β-defensin 4, wherein, optionally the β-defensin is human β-defensin 1.

[12] The bacterium of any one of [1]-[11], wherein the coronavirus antigen is a SARS-CoV-2 antigen.

[13] The bacterium of any one of [1]-[12], wherein the coronavirus antigen is selected from any one of SEQ ID NOs: 11-18, 120, 122, 124, 126, 128, 130, 132, 134, 136, 138, 140, 142, 144, 168, or 170 or is an antigenic fragment of any one of SEQ ID NOs: 11-18, 120, 122, 124, 126, 128, 130, 132, 134, 136, 138, 140, 142, 144, 168, or 170.

[14] The bacterium of any one of [1]-[13], wherein the coronavirus antigen is SEQ ID NO: 11 or an antigenic fragment thereof.

[15] The bacterium of any one of [1]-[13], wherein the coronavirus antigen is SEQ ID NO: 12 or an antigenic fragment thereof.

[16] The bacterium of any one of [1]-[13], wherein the coronavirus antigen is SEQ ID NO: 13 or an antigenic fragment thereof.

[17] The bacterium of any one of [1]-[13], wherein the coronavirus antigen is SEQ ID NO: 14 or an antigenic fragment thereof.

[18] The bacterium of any one of [1]-[13], wherein the coronavirus antigen is SEQ ID NO: 15 or an antigenic fragment thereof.

[19] The bacterium of any one of [1]-[13], wherein the coronavirus antigen is SEQ ID NO: 16 or an antigenic fragment thereof.

[20] The bacterium of any one of [1]-[13], wherein the coronavirus antigen is SEQ ID NO: 17 or an antigenic fragment thereof.

[21] The bacterium of any one of [1]-[13], wherein the coronavirus antigen is SEQ ID NO: 18 or an antigenic fragment thereof.

[22] The bacterium of any one of [1]-[21], wherein the one or more fusion proteins further comprise a secretion signal peptide.

[23] The bacterium of [22], wherein the secretion signal peptide is the hemolysin A secretion signal peptide, and the plasmid further encodes HlyB and HlyD.

[24] The bacterium of [23], wherein the plasmid further encodes HlyC and/or HlyR.

[25] The bacterium of any one of [1]-[24], wherein the bacterium and/or the plasmid does not comprise an antibiotic marker.

[26] The bacterium of any one of [1]-[25], wherein the bacterium is a ΔtyrS strain and the plasmid further encodes tyrS.

[27] The bacterium of any one of [1]-[26], wherein the plasmid is integrated into the chromosome of the bacterium or replicates independently of the chromosome of the bacterium.

[28] A combination product comprising:

(a) the bacterium of any one of [1]-[27]; and

(b) at least one of the one or more fusion proteins encoded by the plasmid of said bacterium.

[29] A vaccine comprising the bacterium of any one of [1]-[27] or the combination product of [28].

[30] The bacterium of any one of [1]-[27], the combination product of [28] or the vaccine of [29] for use as a medicament.

[31] The bacterium of any one of [1]-[27], the combination product of [28] or the vaccine of [29] for use in a method of treating a disease or disorder caused by a member of the coronavirus family.

[32] The bacterium, combination product or vaccine for use of [31], wherein the disease or disorder is COVID-19.

[33] A kit comprising:

(a) a live-attenuated bacterium of the genus Salmonella; and

(b) a recombinant plasmid encoding a fusion protein, wherein the fusion protein comprises:
(i) a coronavirus antigen; and
(ii) an adjuvant peptide.

[34] The kit of [33], wherein the live-attenuated bacterium and the recombinant plasmid are according to any one of [1]-[126].

Exemplary materials which can be used in accordance with the invention are shown in the following tables. These materials may be combined with any aspect or embodiment described throughout the entirety of the present application.

TABLE 1
Bacterial strains

| Bacterial strains | Relevant characteristics/Plasmids | Source or reference |
|---|---|---|
| E. coli DH5α | F⁻, ø80dlacZ M15, (lacZYA- | Invitrogen |
| | argF)U169 deoR, recA1, endA1, | |
| | hsdR17(rk⁻, mk⁺), phoA, supE44, λ⁻, | |
| | thi-1, gyrA96, relA1 | |
| E. coli CC118 (λpir) | Δ(ara-leu), araD, ΔlacX74, galE, galK, | Herrero et al., (1990) |
| | phoA20, thi-1, rpsE, rpoB, argE(Am), | |
| | recA, λpir phage lysogen | |
| S. enterica serovar Typhi Ty21a | S. Typhi Ty2, galE, rpoS, yiaB | (Germanier et al., 1975), |
| | | Berna Biotech Ltd. |
| | | GenBank accession number |
| | | CP002099; (Xu et al., 2013) |
| S. enterica serovar Typhimurium | hisG46, DEL407 [aroA544::Tn10 | (Hoiseth et al., 1981) |
| ΔaroA SL7207 | (Tc^{s})] | |
| S. enterica serovar Typhi Ty21a ΔtyrS | Ty21a derivat, tyrS gene | (Diessner, 2009) |
| (tyrS Cm)⁺, clone 120 | replacement by a (FRT tyrS Cm | |
| | FRT)⁺-knock-in-Fragment | |

TABLE 2
In silico design - antigen selection of antigens in accordance with the invention

| | | | | Antigenic unit in |
|---|---|---|---|---|
| | UniProt; | Average antigenic | | fusion protein of the |
| | SEQ ID | propensity for this | Length | A-Site, respectively |
| | NO | sequence | (aa) | B-Site |
| Protein sequences of SARS- | | | | |
| CoV-2 | | | | |
| Structural proteins | | | | |
| S - spike glycoprotein (Wuhan | P0DTC2; | | | |
| Hu-1 isolate) | SEQ ID NO: | | | |
| | 11 | | | |
| Region 2-1273 | | 1.0417 | 1272 | |
| >sp \| P0DTC2 \| 1-1273 | | | | |
| BetaCoV S1-NTD | | 1.0364 | 291 | |
| >sp \| P0DTC2 \| 13-303 | | | | |
| Receptor binding domain | | 1.0432 | 223 | A1 |
| >sp \| P0DTC2 \| 319-541 | | | | |
| BetaCoV S1-CTD | | 1.0446 | 194 | A3 |
| >sp \| P0DTC2 \| 334-527 | | | | |
| RBM Receptor binding | | 1.0164 | 72 | |
| motif | | | | |
| >sp \| P0DTC2 \| 437-508 | | | | |
| Fusion peptide | | 1.0239 | 19 | |
| >sp \| P0DTC2 \| 788-806 | | | | |
| Heptad repeat 1 | | 1.0350 | 51 | |
| >sp \| P0DTC2 \| 920-970 | | | | |
| Heptad repeat 2 | | 1.0208 | 40 | |
| >sp \| P0DTC2 \| 1163-1202 | | | | |
| Cytoplasmic domain | | 1.1129 | 39 | |
| >sp \| P0DTC2 \| 1235-1273 | | | | |
| M - Membrane glycoprotein | P0DTC5; | | | |
| | SEQ ID NO: | | | |
| | 12 | | | |
| Region 2-222 | | 1.0542 | 221 | |
| >sp \| P0DTC5 \| 2-222 | | | | |
| Region 2-100 | | 1.0756 | 99 | |
| >sp \| P0DTC5\| 2-100 | | | | |
| E - Envelope-Protein | P0DTC4; | | | |
| | SEQ ID NO: | | | |
| | 13 | | | |
| Region 1-75 | | 1.1202 | 75 | |
| N - Nucleocapsid protein | P0DTC9; | | | |
| | SEQ ID NO: | | | |
| | 14 | | | |
| Region: 2-419: | | 0.9874 | 418 | |
| >sp \| P0DTC9 \| 2-419 | | | | |
| Region: 41-186: RNA- | | 0.9912 | 146 | |
| binding | | | | |
| >sp \| P0DTC9 \| 41-186 | | | | |
| Region: 258-361: | | 0.9975 | 104 | B5, B7, B9, B10, B11, |
| Dimerization | | | | B12, B14 |
| >sp \| P0DTC9 \| 258-361 | | | | A22 |
| Multi-epitope unit, | SEQ ID NO: | 1.0157 | 255 | B3, B15, B16 |
| Variant 6: | 167 | | | |
| aa 217-231, L, aa 249-371, | | | | |
| L, aa 361-371, L , aa 361- | | | | |
| 371 | | | | |
| Region aa | | | | A23 |

(aa = amino acid; L = Linker sequence)

TABLE 3
In silico design - adjuvant selection for use in the invention

| | | Average | | |
|---|---|---|---|---|
| | | antigenic | | |
| | UniProt; | propensity | | adjuvant unit in fusion |
| | SEQ ID | for this | Length | protein of the A-Site, |
| | NO: | sequence | (aa) | respectively B-Site |
| Protein sequences of Adjuvants | | | | |
| Mucosal adjuvants | | | | |
| Cholera enterotoxin B- | Q57193; | 1.0146 | 103 | A1, A3 |
| subunit | SEQ ID | | | A11, A12, A13, A14, A15, |
| >tr \| Q57193 \| 22-124 | NO: 2 | | | A17, A18, A19, A20, |
| | | | | A21, A22, A23 |
| | | | | B13, B14, B16 |
| IL2, (IL2_HUMAN) | P60568; | 1.0307 | 133 | |
| >sp \| P60568 \| 21-153 | SEQ ID | | | |
| | NO: 1 | | | |
| human β-defensin group | | | | |
| BD1 | P60022; | 1.0592 | 47 | B3, B5, B7 |
| >sp \| P60022 \| 22-68 | SEQ ID | | | |
| | NO: 5 | | | |
| BD2 | 015263; | 1.0779 | 41 | B9, B11, B12 |
| >sp \| O15263 \| 24-64 | SEQ ID | | | |
| | NO: 6 | | | |
| BD3 | P81534; | 1.0512 | 45 | |
| >sp \| P81534 \| 23-67 | SEQ ID | | | |
| | NO: 7 | | | |
| BD4 | Q8WTQ1; | 1.0256 | 50 | |
| >sp \| Q8WTQ1 \| 23-72) | SEQ ID | | | |
| | NO: 8 | | | |
| Bacterial adjuvants | | | | |
| 50S ribosomal protein L7/L12 | Q735E8; | 1.0319 | 130 | |
| (Rv0652) | SEQ ID | | | |
| Full length | NO: 4 | | | |
| Neisseria porB | X5EGHO; | 1.0185 | 310 | |
| PorB sequence is 310 | SEQ ID | | | |
| residues long | NO: 3 | | | |
| >tr \| X5EGH0 \| 20-329 | | | | |

(aa = amino acid; L = Linker sequence)

TABLE 4
Fusion protein design of the A-site in accordance with the invention (see Table 13 for
the amino acid sequences of the fusion protein constructs)

| | Fusion proteins of A-Site | | | | | | | | |
|---|---|---|---|---|---|---|---|---|---|
| | | | | | | Viral antigen | | | |
| | | | | | | unit, S-Protein, | | | |
| Construct | HlyA- | Nsil- | | | | VOC VOI VOM | | Nsil- | |
| # | Nter. | Site | Linker | Adjuvant | Linker | (SEQ ID NO) | Linker | Site | HlyAₛ |
| A1 | HlyA- | Nsil | EAAAK | CtxB | EAAAK | RBD Wuhan-Hu-1 | EAAAK | Nsil | HlyAs |
| | Nter. | | | | | Isolate | | | |
| A3 | HlyA- | Nsil | EAAAK | CtxB | EAAAK | BetaCoV S1-CTD | EAAAK | Nsil | HlyAs |
| | Nter. | | | | | Wuhan-Hu-1 | | | |
| | | | | | | Isolate | | | |
| A11 | HlyA- | Nsil | EAAAK | CtxB | EAAAK | RBD variant | EAAAK | Nsil | HlyAs |
| | Nter. | | | | | B.1.1.7, Alpha | | | |
| A12 | HlyA- | Nsil | EAAAK | CtxB | EAAAK | RBD variant | EAAAK | Nsil | HlyAs |
| | Nter. | | | | | B.1.1.7 plus | | | |
| | | | | | | E484K | | | |
| A13 | HlyA- | Nsil | EAAAK | CtxB | EAAAK | RBD variant | EAAAK | Nsil | HlyAs |
| | Nter. | | | | | B.1.351, Beta | | | |
| A14 | HlyA- | Nsil | EAAAK | CtxB | EAAAK | RBD variant | EAAAK | Nsil | HlyAs |
| | Nter. | | | | | B.1.351 plus RBD | | | |
| | | | | | | variant B.1.1.7 | | | |
| A15 | HlyA- | Nsil | EAAAK | CtxB | EAAAK | RBD variant P.1 | EAAAK | Nsil | HlyAs |
| | Nter. | | | | | (501Y.V.3), | | | |
| | | | | | | Gamma | | | |
| A16 | HlyA- | Nsil | EAAAK | CtxB | EAAAK | — | EAAAK | Nsil | HlyAs |
| | Nter. | | | | | | | | |
| A17 | HlyA- | Nsil | EAAAK | — | EAAAK | RBD Wuhan-Hu-1 | EAAAK | Nsil | HlyAs |
| | Nter. | | | | | Isolate | | | |
| A18 | HlyA- | Nsil | EAAAK | CtxB | EAAAK | RBD variant | EAAAK | Nsil | HlyAs |
| | Nter. | | | | | B.1.617.1, Kappa, | | | |
| | | | | | | B.1.617.3 | | | |
| A19 | HlyA- | Nsil | EAAAK | CtxB | EAAAK | RBD variant | EAAAK | Nsil | HlyAs |
| | Nter. | | | | | B.1.617-2, Delta | | | |
| A20 | HlyA- | Nsil | EAAAK | CtxB | EAAAK | RBD variant | EAAAK | Nsil | HlyAs |
| | Nter. | | | | | B.1.617-2.1 | | | |
| | | | | | | (Delta plus | | | |
| | | | | | | K417N ) | | | |
| A21 | HlyA- | Nsil | EAAAK | CtxB | EAAAK | RBD variant C.37 | EAAAK | Nsil | HlyAs |
| | Nter. | | | | | (Lambda) | | | |

Schematic structure of selected fusion proteins of the A-Site (aa = amino acid; L = Linker sequence) (VOC: variants of concern, VOI: variants of interest, VOM: variant under monitoring, HlyA-Nter (also referred to herein as “HlyAN”) is the N-terminal sequence of HlyA (SEQ ID NO: 25); HlyAs is the signal peptide of HlyA (SEQ ID NO: 19).

TABLE 5
Fusion protein design of the B-site in accordance with the invention (see
Table 15 for the amino acid sequences of the fusion protein constructs)

| | Fusion proteins of B-Site | | | | | | | | |
|---|---|---|---|---|---|---|---|---|---|
| Construct | Sall- | | Signal | | | Viral antigen | | | Sall- |
| # | Site | PR | peptide | Adjuvant | Linker | unit, N-Protein | Tag | TR | Site |
| B3 | Sall | PR4 | OmpA | hBD1 | EAAAK | aa 217-231, L, | T7 | TR2 | Sall |
| | | | | | | aa 249-371, L, | | | |
| | | | | | | aa 361-371, L , | | | |
| | | | | | | aa 361-371 | | | |
| B5 | Sall | PR4 | OmpA | hBD1 | EAAAK | aa 258-361 | T7 | TR2 | Sall |
| | | | | | | (dimerization | | | |
| | | | | | | region) | | | |
| B7 | Sall | PR4 | Bla | hBD1 | EAAAK | aa 258-361 | T7 | TR2 | Sall |
| | | | | | | (dimerization | | | |
| | | | | | | region) | | | |
| B9 | Sall | PR4 | Bla | hBD2 | EAAAK | aa 258-361 | T7 | TR2 | Sall |
| | | | | | | (dimerization | | | |
| | | | | | | region) | | | |
| B10 | Sall | PR3 | OmpA | — | EAAAK | aa 258-361 | His | T0 | Sall |
| | | | | | | (dimerization | | | |
| | | | | | | region) | | | |
| B11 | Sall | PR3 | OmpA | hBD2 | EAAAK | aa 258-361 | His | T0 | Sall |
| | | | | | | (dimerization | | | |
| | | | | | | region) | | | |
| B12 | Sall | PR3 | OmpA | hBD2 | EAAAK | — | His | T0 | Sall |
| B13 | Sall | PR3 | OmpA | CtxB | EAAAK | — | His | T0 | Sall |
| B14 | Sall | PR3 | OmpA | CtxB | EAAAK | aa 258-361 | His | T0 | Sall |
| | | | | | | (dimerization | | | |
| | | | | | | region) | | | |
| B15 | Sall | PR3 | OmpA | — | EAAAK | aa 217-231, L, | His | T0 | Sall |
| | | | | | | aa 249-371, L, | | | |
| | | | | | | aa 361-371, L, | | | |
| | | | | | | aa 361-371 | | | |
| B16 | Sall | PR3 | OmpA | CtxB | EAAAK | aa 217-231, L, | His | T0 | Sall |
| | | | | | | aa 249-371, L, | | | |
| | | | | | | aa 361-371, L , | | | |
| | | | | | | aa 361-371 | | | |

Schematic structure of selected fusion proteins of the A-Site (aa = amino acid; L = Linker sequence, VOC: variants of concern, VOI: variants of interest, VOM: variant under monitoring, PR: Promotor region; PR4: SEQ ID NO: 36; PR3: SEQ ID NO: 35; TR: Terminator region; TR 2 (SEQ ID NO: 43): TR T0: BBA_K864600 T0-TERMINATOR (SEQ ID NO: 44).

TABLE 6
Plasmids with codon optimized synthetic antigen fragments in accordance with the invention

| Plasmids | Relevant characteristics | Source/Manufacturer |
|---|---|---|
| Plasmids with | | |
| synthetic Nsil- | | |
| fragments for | | |
| cloning into A-site | | |
| of our vaccine | | |
| plasmids | | |
| Nsil 1 in pMK-RQ | Standard delivery vector Geneart, | Thermo Fisher Scientific GENEART |
| | KanR | GmbH |
| | carrying Nsil-Fragment Nsil 1 (->A1) | |
| Nsil 2 in pMK-RQ | Standard delivery vector Geneart, | Thermo Fisher Scientific GENEART |
| | KanR | GmbH |
| | carrying Nsil-Fragment Nsil 2 (->A3) | |
| A11 in pMK-RQ | Standard delivery vector Geneart, | Thermo Fisher Scientific GENEART |
| | KanR | GmbH |
| | carrying Nsil-Fragment A11 (->A11) | |
| A12 in pMK-RQ | Standard delivery vector Geneart, | Thermo Fisher Scientific GENEART |
| | KanR | GmbH |
| | carrying Nsil-Fragment A12 (->A12) | |
| A13 in pMK-RQ | Standard delivery vector Geneart, | Thermo Fisher Scientific GENEART |
| | KanR | GmbH |
| | carrying Nsil-Fragment A13 (->A13) | |
| A14 in pMK-RQ | Standard delivery vector Geneart, | Thermo Fisher Scientific GENEART |
| | KanR | GmbH |
| | carrying Nsil-Fragment A14 (->A14) | |
| A15 in pMK-RQ | Standard delivery vector Geneart, | Thermo Fisher Scientific GENEART |
| | KanR | GmbH |
| | carrying Nsil-Fragment A15 (->A15) | |
| A16 in pMA-RQ | Standard delivery vector Geneart, | Thermo Fisher Scientific GENEART |
| | AmpR | GmbH |
| | carrying Nsil-Fragment A16 (->A16) | |
| A17 in in pMK-RQ | Standard delivery vector Geneart, | Thermo Fisher Scientific GENEART |
| | KanR | GmbH |
| | carrying Nsil-Fragment A17 (->A17) | |
| Nsil_18 In pMA-RQ | Standard delivery vector Geneart, | Thermo Fisher Scientific GENEART |
| | AmpR | GmbH |
| | carrying Nsil-Fragment Nsil_18 (-> | |
| | A18) | |
| Nsil_19 In pMA-RQ | Standard delivery vector Geneart, | Thermo Fisher Scientific GENEART |
| | AmpR | GmbH |
| | carrying Nsil-Fragment Nsil_19 (-> | |
| | A19) | |
| Plasmids with | | |
| synthetic Sall- | | |
| fragments for | | |
| cloning into B-site | | |
| of our vaccine | | |
| plasmids | | |
| Sall3 in pMK-RQ | Standard delivery vector Geneart, | Thermo Fisher Scientific GENEART |
| | KanR | GmbH |
| | carrying Sall-Fragment Sall3 (->B3) | |
| Sall5 in pMK-RQ | Standard delivery vector Geneart, | Thermo Fisher Scientific GENEART |
| | KanR | GmbH |
| | carrying Sall-Fragment Sall5 (->B5) | |
| Sall7 in pMA-RQ | Standard delivery vector Geneart, | Thermo Fisher Scientific GENEART |
| | AmpR | GmbH |
| | carrying Sall-Fragment Sall7 (->B7) | |
| Sall-9 in pMK-RQ | Standard delivery vector Geneart, | Thermo Fisher Scientific GENEART |
| | KanR | GmbH |
| | carrying Sall-Fragment Sall-9 (->B9) | |
| Sall-Nr_B10 in | Standard delivery vector Geneart, | Thermo Fisher Scientific GENEART |
| pMK-RQ | KanR | GmbH |
| | carrying Sall-Fragment Sall-Nr_B10 | |
| | (->B10) | |
| Sall-Nr_B11 in | Standard delivery vector Geneart, | Thermo Fisher Scientific GENEART |
| pMK-RQ | KanR | GmbH |
| | carrying Sall-Fragment Sall-Nr_B11 | |
| | (->B11) | |
| Sall-Nr_B12 in | Standard delivery vector Geneart, | Thermo Fisher Scientific GENEART |
| pMK-RQ | KanR | GmbH |
| | carrying Sall-Fragment Sall-Nr_B12 | |
| | (->B12) | |
| Sall-Nr_B13 in | Standard delivery vector Geneart, | Thermo Fisher Scientific GENEART |
| pMK-RQ | KanR | GmbH |
| | carrying Sall-Fragment Sall-Nr_B13 | |
| | (->B13) | |
| Sall-Nr_614 in | Standard delivery vector Geneart, | Thermo Fisher Scientific GENEART |
| pMK-RQ | KanR | GmbH |
| | carrying Sall-Fragment Sall-Nr_B14 | |
| | (->B14) | |
| B15_PR3_Linker in | Standard delivery vector Geneart, | Thermo Fisher Scientific GENEART |
| pMA-RQ | AmpR | GmbH |
| | carrying Sall-Fragment | |
| | B15_PR3_Linker (->B15) | |
| B16_PR3_Linker in | Standard delivery vector Geneart, | Thermo Fisher Scientific GENEART |
| pMA-RQ | AmpR | GmbH |
| | carrying Sall-Fragment | |
| | B16_PR3_Linker (->B16) | |
| Plasmid with | | |
| synthetic Spel- | | |
| fragment for | | |
| cloning into Spel- | | |
| Site of our vaccine | | |
| plasmids | | |
| Spel-Nr_1 in pMA- | Standard delivery vector Geneart, | Thermo Fisher Scientific GENEART |
| RQ | AmpR | GmbH |
| | carrying Spel-Fragment Spel-Nr_1 | |
| | with | |
| | P_{lacltyrS}-His_{Tag}-T0-Expression cassette | |
| | (improved DNA) | |

TABLE 7A
Plasmids

| Plasmids | Relevant characteristics | Source/Reference |
|---|---|---|
| pCP20 | helper plasmid, AmpR, CmR bla cat | (Cherepanov et al, 1995) |
| | cl857 lPR flp pSC101 oriTS | |
| pKD46 | Helper plasmid, AmpR, encoding the | Datsenko and Wanner |
| | Red recombinase Expresses g, b and | (2000) |
| | exo from the arabinose-inducible | |
| | ParaB promoter | |
| pKD3 | helper plasmid, bla FRT cat FRT PS1 | Datsenko and Wanner |
| | PS2 oriR6K | (2000) |
| pKD3-SpeI | helper plasmid, bla FRT BcuI-site cat | Diessner (2009) |
| | FRT PSI PS2 oriR6K | |
| pKD3-SpeI tyrS HisTag-s | helper plasmid, bla FRT PWT | Diessner (2009) |
| | tyrSx6His, cat FRT PS1 PS2 oriR6K | |
| pMKhly1 | FRT Kan^{R} FRT, hlyR, hlyC, hlyAs | Fensterle et al. (2008) |
| | (encoding the hemolysin secretion | |
| | signal), hlyB, hlyD | |
| pMKhly-CtxB | FRT Kan^{R} FRT, derivate of pMKhly- | Fensterle et al. (2008) |
| | CtxB, encoding CtxB-hlyAs-fusion | |
| pMKhly-CtxB-PSA | FRT Kan^{R} FRT, derivate of pMKhly- | Fensterle et al. (2008) |
| | CtxB, encoding a CtxB-PSA-HlyAs | |
| | fusion | |
| pMKhlyΔIS2-CtxB-PSA | derivate of pMKhly-CtxB-PSA: | (Diessner, 2009) |
| | deletion of IS2-like fragment and | |
| | creation of single Spel-site | |
| pMKhlyΔIS2P_{lacl-like}tyrS | derivate of pMKhlyΔIS2-CtxB-PSA: | (Gesser, 2010) |
| CtxB-PSA | integration of an P_{lacl-like} tyrSx6His | |
| | expression cassette into single Spel- | |
| | site | |

TABLE 7B
Primers for the construction of S. enterica serovar Typhi Ty21a ΔtyrS (tyrS Cm)
(Diessner, 2009) and pMKhlyΔIS2 P_{lacI-like}tyrS CtxB-PSA (Gesser 2010)

| Name | SEQ ID NO: | Sequence (5'->3') | Note |
|---|---|---|---|
| Mut-pKD3-SpeI- | 185 | GTG ATC TTC CGT CAC TAG TAG | BcuI-site |
| forward | | GCG CGC CGA AG | |
| Mut-pKD3-SpeI- | 186 | CTT CGG CG GCC TAC TAG TGA | BcuI-site |
| reverse | | CGG AAG ATC AC | |
| SpeI-tyrS-EPK- | 187 | AAA AAA ACT AGT GTT CCC TGT | BcuI-site |
| forward | | ATA AAA ACC AGG GGG | |
| tyrS-EPK-SpeI- | 188 | TTT TTT ACT AGT GTA AAT TCC TGG | BcuI-site |
| reverse | | AGC TGA AGC AGA AG | |
| Ter-HisTag-1- | 189 | CCC CCT TTC CAC TTA ATG CAT TAG | x6HisTag |
| forward | | TGA TGG TGA TGG TGA TGT TTC | |
| | | CAG CAA ATC AGA CAG TAA TTC | |
| SpeI-Ter-HisTag-2- | 190 | AAA AAA ACT AGT GTT CCC TGT | BcuI-site |
| forward | | ATA AAA ACC AGG GGG AGT GAT | |
| | | TTC TCA CTC CCC CTT TCC ACT TAA | |
| | | TGC ATT AG | |
| tyrS-HisTag-reverse | 191 | CAT CAC CAT CAC CAT CAC GCA | x6HisTag |
| | | AGC AGT AAC TTG ATT AAA | |
| knockout-forward | 192 | GTG TAC CGG CAA AGG TGC AGT | |
| | | CGT TTT ATA CAT GGA GAT TTT | |
| | | GAT GGC AGT GTA GGC TGG AGC | |
| | | TGC TTC | |
| knockout-reverse | 193 | GAT AGT GAC AGC GTT GGA GGC | |
| | | GAT AGT CTT ACG CGC CTG ACC | |
| | | ACG TGA CGG ATG GG A ATT AGC | |
| | | CAT GGT CC | |
| SpeI-IS2-Deletion- | 194 | AAA AAC TAG TGA TAA TGG TTC | BcuI-site |
| forward | | ATG CTA CCG GGC GAA TG | |
| IS2-Deletion- | 195 | GTT TTG GGA TCC ACC CTG ATG | BamHI-site |
| BamHI-reverse | | GCT CTG | |
| LacI-Prom.for | 196 | AAA AGT CGA CTA GTG CTA GCG | SalI/SpeI- |
| | | ACA CCA TCG AAT GGC GCA AAC | sites |
| | | CTT TCG CGG TAT GGC ATG ATA | |
| | | GCG CCC GAA GTC GTG TAC CGG | |
| | | CAA AGG TGA GTC G | |
| LacI-Ter-rev | 197 | AAA AAA GTC GAC TAG TGG TAC | SalI/SpeI- |
| | | CAA AAT GCC GCC AGC CCA AAC | sites |
| | | TGG CGG CCG TGG ATT AGT GAT | |
| | | GGT GAT GGT GAT GTT TCC AGC | |
| pMO-tyrS-screen- | 198 | CCC TGA ATC TCC AGA CAA CCA | screening |
| forward | | ATA TCA | |
| pMO-tyrS-screen- | 199 | CCC GTA CAA ATT CTA CCA GTT | screening |
| forward | | CTG GA | |

TABLE 8
Primers for screening and sequencing

| Primer | | Sequence (5′→3′) | |
|---|---|---|---|
| No. | Name | (SEQ ID NO) | Used in PCR-Analysis of |
| 4 | 5 HlyA N-ter_screen | GCCAACAATAACCACTGC | A-Site |
| | forward 1 | (SEQ ID NO: 45) | |
| 6 | HlyA signal_screen | GCTACCTGCAGCTGAAATG | A-Site |
| | reverse 1 | (SEQ ID NO: 46) | |
| 17 | pdxH-forward | GAAGTGCCGTTACCCAGCTTCT | Chromosomal tyrS-region |
| | | G (SEQ ID NO: 47) | |
| 18 | pdxY reverse | GGGACTGGATAGCGAGGATAT | Chromosomal tyrS-region |
| | | TC (SEQ ID NO: 48) | |
| 21 | SalI-Site forward | CTCAACGGCCTCAACCTACTAC | B-Site |
| | | (SEQ ID NO: 49) | |
| 22 | SalI-Site reverse | GTCATAAGTGCGGCGACGATA | B-Site |
| | | G (SEQ ID NO: 50) | |
| 23 | RBD-S-P_screen | CGCGTGGAACTCCAACAAC | A-Site |
| | forward 1 | (SEQ ID NO: 51) | |
| 34 | TR-SalI-reverse | CGACGGTGCCTAATGAGTGAG | B-Site |
| | | CTAACTCAC (SEQ ID NO: 52) | |
| 37 | 37_FRT-Kan-for | CCAATGCTTAATCAGTGAGGCA | Kanamycin resistance |
| | | CC (SEQ ID NO: 53) | region |
| 38 | 38_FRT-Kan-rev | CCGCTCATGAGACAATAACCCT | Kanamycin resistance |
| | | G (SEQ ID NO: 54) | region |
| 39 | 39_SalI-Site for 2 | CATCTCCTTGCATGCACCATTCC | B-Site |
| | | TTG (SEQ ID NO: 55) | |
| 40 | 40_SalI-Site rev 2 | CATAAGTGCGGCGACGATAGTC | B-Site |
| | | ATGC (SEQ ID NO: 56) | |
| 45 | 45_CtxB_SalVac_rev | GCTTTTTTCTGGGAGTCGATG | A-Site |
| | | (SEQ ID NO: 57) | |
| 59 | 59_SalI-site for 3 | CTTGTTTCGGCGTGGGTATGGT | B-Site |
| | | GG (SEQ ID NO: 58) | |
| 68 | 68_5 HlyA N- | GCCAACAATAACCACTGCAC | A-Site |
| | ter_screen forward 2 | (SEQ ID NO: 59) | |
| 69 | 69_HlyA | GAAGCTACCTGCAGCTGAAATG | A-Site |
| | signal_screen reverse | (SEQ ID NO: 60) | |
| | 2 | | |
| 7 | DhF | GCTTAATGTCCAAGATGCCTAC | Multiplex-PCR-Primer for |
| | | (SEQ ID NO: 61) | Strain identification |
| 8 | DhR | GAGCAACGCCAGTACCATCTG | (Kumar et al., 2006) |
| | | (SEQ ID NO: 62) | |
| 9 | InvAF | CGAGCAGCCGCTTAGTATTGAG | |
| | | (SEQ ID NO: 63) | |
| 10 | InvAR | CCATCAAATTAGCGGAGGCTTC | |
| | | (SEQ ID NO: 64) | |
| 11 | PrtF | CGTTTGGGTTCCTTGGATCACG | |
| | | (SEQ ID NO: 65) | |
| 12 | PrtR | CTATAATGGCGGCGGCGAGTTC | |
| | | (SEQ ID NO: 66) | |
| 13 | ViaBF | CACGCACCATCATTTCACCG | |
| | | (SEQ ID NO: 67) | |
| 14 | ViaBR | AACAGGCTGTAGCGATTT | |
| | | AGG (SEQ ID NO: 68) | |

TABLE 9
Plasmids of the JMU-SalVac-100 series used in the invention

| Plasmids | Relevant characteristics | Features/notes |
|---|---|---|
| pSalVac 001 A0_B0 | pMKhlyΔIS2 PlacI-like tyrS, | First basic plasmid of the |
| KanR | hlyR, hlyC, hlyAs (encoding the hemolysin | JMU-SalVac-100 series |
| | secretion signal) hlyB, hlyD, FRT KanR | cloning vector |
| | FRT | Negative control plasmid |
| | contains two separate expressions sites: | |
| | single NsiII-site, located within the hly | |
| | gene cluster | |
| | ->A-Site | |
| | and single Sall site located outside the hly | |
| | gene cluster: | |
| | ->B-Site | |
| pSalVac001 A0_B0 | pSalVac 001 A0_B0 KanR-Derivat | |
| ΔKanR | BLS-stabilized in JMU-SalVac-101 | |
| Vaccine plasmids | | |
| of the JMU-SalVac | | |
| 100-Series | | |
| pSalVac 101 Ax_By | pMKhlyΔIS2 PlacI-like tyrS HisTag, | Schematic structure of |
| KanR | hlyR, hlyC, hlyB, hlyD, FRT KanR FRT | plasmids of the |
| | A-Site encodes fusion protein Ax-hlyAs | JMU-SalVac- |
| | B-Site contains promotor region, CDS of B- | 100 series |
| | Site fusion protein and terminator region, | |
| pSalVac 101 Ax_By | pMKhlyΔIS2 PlacI-like tyrS HisTag, | Schematic structure of |
| ΔKanR | hlyR, hlyC, hlyB, hlyD FRT, | plasmids |
| | A-Site encodes fusion protein Ax-hlyAs | JMU-SalVac-100 |
| | B-Site contains promotor region, CDS of B- | series after final |
| | Site fusion protein and terminator region | elimination |
| | | of antibiotic |
| | | resistance gene |
| pSalVac 101 A1_B0 | pSalVac 001 A0_B0 KanR-Derivat | First set of Plasmid |
| KanR | Fragment NsiI 1 in NsiI-Site of A-Site, | constructs |
| | contains CDS of fusion protein A1 | SARS-Cov-2 |
| | KanR | Wuhan-Hu-1 |
| | | Isolate |
| pSalVac 101 A1_B0 | pSalVac 001 A0_B0 KanR-Derivat | |
| ΔKanR | Fragment NsiI 1 in NsiI-Site of A-Site, | |
| | contains CDS of fusion protein A1 | |
| | BLS-stabilized in JMU-SalVac-102 | |
| pSalVac 101 A3_B0 | pSalVac 001 A0_B0 KanR-Derivat | |
| KanR | Fragment NsiI 2 in NsiI-Site of A-Site, | |
| | contains CDS of fusion protein A3 | |
| | KanR | |
| pSalVac 101 A3_B0 | pSalVac 001 A0_B0 KanR-Derivat | |
| ΔKanR | Fragment NsiI 2 in NsiI-Site of A-Site, | |
| | contains CDS of fusion protein A3 | |
| | BLS-stabilized in JMU-SalVac-103 | |
| pSalVac 101 | pSalVac 001 A0_B0 KanR-Derivat | |
| A1_B3f KanR | Fragment NsiI 1 in NsiI-Site of A-Site, | |
| | Fragment Sall3 in SalI-Site of B-Site, | |
| | forward. | |
| | contains CDS of fusion proteins A1 and B3 | |
| | KanR | |
| pSalVac 101 | pSalVac 001 A0_B0 KanR-Derivat | |
| A1_B3f ΔKanR | Fragment NsiI 1 in NsiI-Site of A-Site, | |
| | Fragment Sall3 in SalI-Site of B-Site, | |
| | forward, | |
| | contains CDS of fusion proteins A1 and B3, | |
| | BLS-stabilized in JMU-SalVac-104 | |
| pSalVac 101 | pSalVac 001 A0_B0 KanR-Derivat | |
| A3_B3f KanR | Fragment NsiI 2 in NsiI-Site of A-Site, | |
| | Fragment Sall3 in SalI-Site of B-Site, | |
| | forward, | |
| | contains CDS of fusion proteins A3 and B3, | |
| | KanR | |
| pSalVac 101 | pSalVac 001 A0_B0 KanR-Derivat | |
| A3_B3f ΔKanR | Fragment NsiI 2 in NsiI-Site of A-Site, | |
| | Fragment Sall3 in SalI-Site of B-Site, | |
| | forward, | |
| | contains CDS of fusion proteins A3 and B3, | |
| | BLS-stabilized in JMU-SalVac-105 | |
| pSalVac 101 | pSalVac 001 A0_B0 KanR-Derivat | |
| A1_B5f KanR | Fragment NsiI 1 in NsiI-Site of A-Site, | |
| | Fragment Sall5 in SalI-Site of B-Site, | |
| | forward, | |
| | contains CDS of fusion proteins A1 and B5, | |
| | KanR | |
| pSalVac 101 | pSalVac 001 A0_B0 KanR-Derivat | |
| A1_B5f 6,KanR | Fragment NsiI 1 in NsiI-Site of A-Site, | |
| | Fragment Sall5 in SalI-Site of B-Site, | |
| | forward, | |
| | contains CDS of fusion proteins A1 and B5, | |
| | BLS-stabilized in JMU-SalVac-106 | |
| pSalVac 101 | pSalVac 001 A0_B0 KanR-Derivat | |
| A1_B7r KanR | Fragment NsiI 1 in NsiI-Site of A-Site, | |
| | Fragment Sall7 in SalI-Site of B-Site, | |
| | reverse, | |
| | contains CDS of fusion proteins A1 and B7, | |
| | KanR | |
| pSalVac 101 | pSalVac 001 A0_B0 KanR-Derivat | |
| A1_B7r ΔKanR | Fragment NsiI 1 in NsiI-Site of A-Site, | |
| | Fragment Sall7 in SalI-Site of B-Site, | |
| | reverse, | |
| | contains CDS of fusion proteins A1 and B7, | |
| | BLS-stabilized in JMU-SalVac-107 | |
| pSalVac 101 | pSalVac 001 A0_B0 KanR-Derivat | |
| A1_B9f KanR | Fragment NsiI 1 in NsiI-Site of A-Site, | |
| | Fragment Sall9 in SalI-Site of B-Site, | |
| | forward, | |
| | contains CDS of fusion proteins A1 and B9, | |
| | KanR | |
| pSalVac 101 | pSalVac 001 A0_B0 KanR-Derivat | |
| A1_B9f ΔKanR | Fragment NsiI 1 in NsiI-Site of A-Site, | |
| | Fragment Sall9 in SalI-Site of B-Site, | |
| | forward, | |
| | contains CDS of fusion proteins A1 and B5, | |
| | BLS-stabilized in JMU-SalVac-108 | |
| pSalVac 101 | pSalVac 001 A0_B0 KanR-Derivat | |
| A1_B10f KanR | Fragment NsiI 1 in NsiI-Site of A-Site, | |
| | Fragment Sall10 in SalI-Site of B-Site, | |
| | forward, | |
| | contains CDS of fusion proteins A1 and | |
| | B10, | |
| | KanR | |
| pSalVac 101 | pSalVac 001 A0_B0 KanR-Derivat | |
| A1_B10f ΔKanR | Fragment NsiI 1 in NsiI-Site of A-Site, | |
| | Fragment Sall10 in SalI-Site of B-Site, | |
| | forward, | |
| | contains CDS of fusion proteins A1 and | |
| | B10, | |
| | BLS-stabilized in JMU-SalVac-109 | |
| pSalVac 101 | pSalVac 001 A0_B0 KanR-Derivat | Wuhan-Hu-1 Isolate |
| A0_B3f KanR | Fragment Sall3 in SalI-Site of B-Site, | |
| | forward. | |
| | contains CDS of fusion proteins B3 | |
| | KanR | |
| pSalVac 101 | pSalVac 001 A0_B0 KanR-Derivat | |
| A0_B3f ΔKanR | Fragment Sall3 in SalI-Site of B-Site, | |
| | forward. | |
| | contains CDS of fusion proteins B3 | |
| | BLS-stabilized | |
| pSalVac 101 | pSalVac 001 A0_B0 KanR-Derivat | Wuhan-Hu-1 Isolate |
| A0_B9f KanR | Fragment Sall9 in SalI-Site of B-Site, | |
| | forward. | |
| | contains CDS of fusion proteins B9 | |
| | KanR | |
| pSalVac 101 | pSalVac 001 A0_B0 KanR-Derivat | |
| A0_B9f ΔKanR | Fragment Sall9 in SalI-Site of B-Site, | |
| | forward. | |
| | contains CDS of fusion proteins B9 | |
| | BLS-stabilized | |
| pSalVac 101 | pSalVac 001 A0_B0 KanR-Derivat | Wuhan-Hu-1 Isolate |
| A0_B5f KanR | Fragment Sall5 in SalI-Site of B-Site, | |
| | forward. | |
| | contains CDS of fusion proteins B5 | |
| | KanR | |
| pSalVac 101 | pSalVac 001 A0_B0 KanR-Derivat | |
| A0_B5f ΔKanR | Fragment Sall5 in SalI-Site of B-Site, | |
| | forward. | |
| | contains CDS of fusion proteins B5 | |
| | BLS-stabilized | |
| pSalVac 101 | pSalVac 001 A0_B0 KanR-Derivat | Wuhan-Hu-1 Isolate |
| A1_B14f KanR | Fragment NsiI 1 in NsiI-Site of A-Site, | |
| | Fragment SalI-Nr_B14 in SalI-Site of B- | |
| | Site, forward, | |
| | contains CDS of fusion proteins A1 and | |
| | B14, | |
| | KanR | |
| pSalVac 101 | pSalVac 001 A0_B0 KanR-Derivat | |
| A1_B14f ΔKanR | Fragment NsiI 1 in NsiI-Site of A-Site, | |
| | Fragment SalI-Nr_B14 in SalI-Site of B- | |
| | Site, forward, | |
| | contains CDS of fusion proteins A1 and | |
| | B14, | |
| | BLS-stabilized | |
| pSalVac 101 | pSalVac 001 A0_B0 KanR-Derivat | Wuhan-Hu-1 Isolate |
| A1_B15f KanR | Fragment NsiI 1 in NsiI-Site of A-Site, | |
| | Fragment B15_PR3_Linker in SalI-Site of | |
| | B-Site, forward, | |
| | contains CDS of fusion proteins A1 and | |
| | B15, KanR | |
| pSalVac 101 | pSalVac 001 A0_B0 KanR-Derivat | |
| A1_B15f ΔKanR | Fragment NsiI 1 in NsiI-Site of A-Site, | |
| | Fragment B15_PR3_Linker in SalI-Site of | |
| | B-Site, forward, | |
| | contains CDS of fusion proteins A1 and | |
| | B15, | |
| | BLS-stabilized | |
| pSalVac 101 | pSalVac 001 A0_B0 KanR-Derivat | Wuhan-Hu-1 Isolate |
| A1_B16f KanR | Fragment NsiI 1 in NsiI-Site of A-Site, | |
| | Fragment B16_PR3_Linker in SalI-Site of | |
| | B-Site, forward, | |
| | contains CDS of fusion proteins A1 and | |
| | B16, | |
| | KanR | |
| pSalVac 101 | pSalVac 001 A0_B0 KanR-Derivat | |
| A1_B16f ΔKanR | Fragment NsiI 1 in NsiI-Site of A-Site, | |
| | Fragment B16_PR3_Linker in SalI-Site of | |
| | B-Site, forward, | |
| | contains CDS of fusion proteins A1 and | |
| | B16, | |
| | BLS-stabilized | |
| pSalVac 101 | pSalVac 001 A0_B0 KanR-Derivat | RBD S-Protein |
| A11_B3f KanR | Fragment A11 in NsiI-Site of A-Site, | variant B.1.1.7, |
| | Fragment Sall3 in SalI-Site of B-Site, | Alpha |
| | forward. | |
| | contains CDS of fusion proteins A11 and | |
| | B3, | |
| | KanR | |
| pSalVac 101 | pSalVac 001 A0_B0 KanR-Derivat | |
| A11_B3 ΔKanR | Fragment A11 in NsiI-Site of A-Site, | |
| | Fragment Sall3 in SalI-Site of B-Site, | |
| | forward. | |
| | contains CDS of fusion proteins A12 and | |
| | B3, | |
| | BLS-stabilized in JMU-SalVac-110 | |
| pSalVac 101 | pSalVac 001 A0_B0 KanR-Derivat | RBD S-Protein, |
| A12_B3f KanR | Fragment A12 in NsiI-Site of A-Site, | variant B.1.1.7 |
| | Fragment Sall3 in SalI-Site of B-Site, | plus E484K |
| | forward. | |
| | contains CDS of fusion proteins A12 and | |
| | B3, | |
| | KanR | |
| pSalVac 101 | pSalVac 001 A0_B0 KanR-Derivat | |
| A12_B3f ΔKanR | Fragment A12 in NsiI-Site of A-Site, | |
| | Fragment Sall3 in SalI-Site of B-Site, | |
| | forward. | |
| | contains CDS of fusion proteins A12 and | |
| | B3, | |
| | BLS-stabilized in JMU-SalVac-111 | |
| pSalVac 101 | pSalVac 001 A0_B0 KanR-Derivat | RBD S-Protein, |
| A13_B3f KanR | Fragment A13 in NsiI-Site of A-Site, | variant |
| | Fragment Sall3 in SalI-Site of B-Site, | B.1.351, Beta |
| | forward. | |
| | contains CDS of fusion proteins A13 and | |
| | B3, | |
| | KanR | |
| pSalVac 101 | pSalVac 001 A0_B0 KanR-Derivat | |
| A13_B3f ΔKanR | Fragment A13 in NsiI-Site of A-Site, | |
| | Fragment Sall3 in SalI-Site of B-Site, | |
| | forward. | |
| | contains CDS of fusion proteins A13 and | |
| | B3, | |
| | BLS-stabilized in JMU-SalVac-112 | |
| pSalVac 101 | pSalVac 001 A0_B0 KanR-Derivat | RBD S-Protein, |
| A15_B3f KanR | Fragment A15 in NsiI-Site of A-Site, | variant P.1, |
| | Fragment Sall3 in SalI-Site of B-Site, | Gamma |
| | forward. | |
| | contains CDS of fusion proteins A13 and | |
| | B3, | |
| | KanR | |
| pSalVac 101 | pSalVac 001 A0_B0 KanR-Derivat | |
| A15_B3f ΔKanR | Fragment A15 in NsiI-Site of A-Site, | |
| | Fragment Sall3 in SalI-Site of B-Site, | |
| | forward. | |
| | contains CDS of fusion proteins A15 and | |
| | B3, | |
| | BLS-stabilized in JMU-SalVac-113 | |
| pSalVac 101 | pSalVac 001 A0_B0 KanR-Derivat | RBD S-Protein |
| A19_B3f KanR | Fragment A19 in NsiI-Site of A-Site, | variant |
| | Fragment Sall3 in SalI-Site of B-Site, | B.1.617.2, Delta |
| | forward. | |
| | contains CDS of fusion proteins A15 and | |
| | B3, | |
| | KanR | |
| pSalVac 101 | pSalVac 001 A0_B0 KanR-Derivat | |
| A19_B3f ΔKanR | Fragment A19 in NsiI-Site of A-Site, | |
| | Fragment Sall3 in SalI-Site of B-Site, | |
| | forward. | |
| | contains CDS of fusion proteins A19 and | |
| | B3, | |
| | BLS-stabilized in JMU-SalVac-114 | |
| pSalVac 101 | pSalVac 001 A0_B0 KanR-Derivat | RBD S-Protein |
| A19_B10f KanR | Fragment A19 in NsiI-Site of A-Site, | variant |
| | Fragment Sall10 in SalI-Site of B-Site, | B.1.617.2, Delta |
| | forward, | |
| | contains CDS of fusion proteins A19 and | |
| | B10, | |
| | KanR | |
| pSalVac 101 | pSalVac 001 A0_B0 KanR-Derivat | |
| A19_B10f ΔKanR | Fragment A19 in NsiI-Site of A-Site, | |
| | Fragment Sall10 in SalI-Site of B-Site, | |
| | forward, | |
| | contains CDS of fusion proteins A19 and | |
| | B10, | |
| | BLS-stabilized in JMU-SalVac-115 | |
| pSalVac 101 | pSalVac 001 A0_B0 KanR-Derivat | RBD S-Protein |
| A19_B14f KanR | Fragment A19 in NsiI-Site of A-Site, | variant |
| | Fragment SalI-Nr_B14 in SalI-Site of B- | B.1.617.2, Delta |
| | Site, forward, | |
| | contains CDS of fusion proteins A19 and | |
| | B14, | |
| | KanR | |
| pSalVac 101 | pSalVac 001 A0_B0 KanR-Derivat | |
| A19_B14f ΔKanR | Fragment A19 in NsiI-Site of A-Site, | |
| | Fragment SalI-Nr_B14 in SalI-Site of B- | |
| | Site, forward, | |
| | contains CDS of fusion proteins A19 and | |
| | B14, | |
| | BLS-stabilized in JMU-SalVac-116 | |
| pSalVac 101 | pSalVac 001 A0_B0 KanR-Derivat | RBD S-Protein |
| A19_B15f KanR | Fragment A19 in NsiI-Site of A-Site, | variant |
| | Fragment B15_PR3_Linker in SalI-Site of | B.1.617.2, Delta |
| | B-Site, forward, | |
| | contains CDS of fusion proteins A19 and | |
| | B15, KanR | |
| pSalVac 101 | pSalVac 001 A0_B0 KanR-Derivat | |
| A19_B15f ΔKanR | Fragment A19 inNsiI-Site of A-site, | |
| | Fragment B15_PR3_Linker in SalI-Site of | |
| | B-Site, forward, | |
| | contains CDS of fusion proteins A19 and | |
| | B15, | |
| | BLS-stabilized BLS-stabilized in JMIU- | |
| | SalVac-117 | |
| pSalVac 101 | pSalVac 001 A0_B0 KanR-Derivat | RBD S-Protein |
| A19_B16f KanR | Fragment A19 in NsiI-Site of A-Site, | variant |
| | Fragment B16_PR3_Linker in SalI-Site of | B.1.617.2, Delta |
| | B-Site, forward, | |
| | contains CDS of fusion proteins A1 and | |
| | B16, | |
| | KanR | |
| pSalVac 101 | pSalVac 001 A0_B0 KanR-Derivat | |
| A19_B16f ΔKanR | Fragment A19 in NsiI-Site of A-Site, | |
| | Fragment B16_PR3_Linker in SalI-Site of | |
| | B-Site, forward, | |
| | contains CDS of fusion proteins A1 and | |
| | B10, | |
| | BLS-stabilized BLS-stabilized in JMU- | |
| | SalVac-118 | |

TABLE 10
BLS intermediate strains
BLS-relevant bacterial intermediate strains in this study

| Strain | Plasmid(s) | Feature(s) |
|---|---|---|
| S. enterica serovar Typhi | pCP20 | BLS-(R) recipient |
| Ty21a ΔtyrS | | strain, CmR, AmpR |
| (tyrS Cm)⁺, | | |
| clone 1 | | |
| S. enterica serovar Typhi | — | BLS-(R) recipient strain |
| Ty21a @deltatyrS | | Depletion of pCP20 by |
| (tyrS Cm)⁺, | | incubation at 37° C. |
| clone 1 | | overnight in liquid LB, |
| | | vegetal (Roth) (-> BLS- |
| | | R ΔpCP20) |
| S. enterica serovar Typhi | pCP20, pSalVac | Schematic structure of BLS- |
| Ty21a ΔtyrS | Ax_By Kan^{R} | intermediate strains |
| (tyrS Cm)⁺, | | CmR, AmpR, KanR |
| clone 1 | | |

TABLE 11
BLS vaccine strains used in the invention
BLS stabilized final vaccine strains and control strain:

| Strain | Plasmid(s) | Feature(s) |
|---|---|---|
| S. enterica serovar Typhi | pSalVac 101 Ax_By | Schematic structure of JMU-SalVac-100 |
| Ty21a ΔtyrS | ΔKanR | Vaccine Strains |
| JMU-SalVac-101 | pSalVac 001 A0_B0 | Control strain |
| | ΔKanR | |
| JMU-SalVac-102 | pSalVac 101 A1_B0 | SARS-Cov-2 Wuhan-Hu-1 Isolate |
| | ΔKanR | |
| JMU-SalVac-103 | pSalVac 101 A3_B0 | SARS-Cov-2 Wuhan-Hu-1 Isolate |
| | ΔKanR | |
| JMU-SalVac-104 | pSalVac 101 A1_B3f | SARS-Cov-2 Wuhan-Hu-1 Isolate |
| | ΔKanR | |
| JMU-SalVac-105 | pSalVac 101 A3_B3f | SARS-Cov-2 Wuhan-Hu-1 Isolate |
| | ΔKanR | |
| JMU-SalVac-106 | pSalVac 101 A1_B5f | SARS-Cov-Wuhan-Hu-1 Isolate |
| | ΔKanR | |
| JMU-SalVac-107 | pSalVac 101 A1_B7r | SARS-Cov-2 Wuhan-Hu-1 Isolate |
| | ΔKanR | |
| JMU-SalVac-108 | pSalVac 101 A1_B9f | SARS-Cov-2 Wuhan-Hu-1 Isolate |
| | ΔKanR | |
| JMU-SalVac-109 | pSalVac 101 A1_B10 | SARS-Cov-2 Wuhan-Hu-1 Isolate |
| | ΔKanR | |
| JMU-SalVac-110 | pSalVac 101 A11_Bf3 | RBD S-Protein ,variant B.1.1.7 Alpha |
| | ΔKanR | |
| JMU-SalVac-111 | pSalVac 101 A12_B3f | RBD S-Protein, variant B.1.1.7 plus E484K |
| | ΔKanR | |
| JMU-SalVac-112 | pSalVac 101 A13_B3f | RBD S-Protein, variant B.1.351 Beta |
| | ΔKanR | |
| JMU-SalVac-113 | pSalVac 101 A15_B3f | RBD S-Protein, variant P.1 Gamma |
| | ΔKanR | |
| JMU-SalVac-114 | pSalVac 101 A19_B3f | RBD S-Protein variant B.1.617.2, Delta |
| | ΔKanR | |
| JMU-SalVac-115 | pSalVac 101 A19_B10f | RBD S-Protein variant B.1.617.2, Delta |
| | ΔKanR | |
| JMU-SalVac-116 | pSalVac 101 A19_B14f | RBD S-Protein variant B.1.617.2, Delta |
| | ΔKanR | |
| JMU-SalVac-117 | pSalVac 101 A19_B15f | RBD S-Protein variant B.1.617.2, Delta |
| | ΔKanR | |
| JMU-SalVac-118 | pSalVac 101 A19_B16f | RBD S-Protein variant B.1.617.2, Delta |
| | ΔKanR | |

TABLE 12
primers for qPCR-Analysis

| Primer | | Sequence (5′→3′) | |
|---|---|---|---|
| No. | Name | (SEQ ID NO) | qPCR-Analysis |
| | | | For detection of mRNA |
| 4 | 5 HlyA N-ter_screen | GCCAACAATAA | With 4 or 68: 278 bp-hlyA-Fragment |
| | forward 1 | CCACTGC (SEQ | templates: pSalVac A0_B0 or |
| | | ID NO: 69) | pMKhly1 |
| 43 | 43_HlyAsignal_ | CTGATGTGGTC | Detection of mRNA of HlyA Nter-HlyA |
| | reverse | AGGGTTATTG | signal-fusion |
| | | (SEQ ID NO: 70) | |
| 44 | 44_CtxB_AEZS120_ | GTTGACTACCT | With 4 or 68: 269 bp-fragment template: |
| | rev | GGTACTTCTAC | pMKhly1-CtxB-PSA |
| | | (SEQ ID NO: 71) | Detection of mRNA CtxB-PSA-HlyAs |
| | | | fusion |
| 45 | 45_CtxB_SalVac_ | GCTTTTTTCTGG | with 4 or 68: 309 bp-ctxB-Fragment |
| | rev | GAGTCGATG | templates: pSalVac Ax_By with CtxB as |
| | | (SEQ ID NO: 72) | adjuvant unit |
| | | | Detection of mRNA of A-Site fusion |
| | | | protein |
| 51 | 51_16S-for | GAGCCCGGGGA | housekeeping gene, control |
| | | TTTCACATC | |
| | | (SEQ ID NO: 73) | |
| 52 | 52_16S-rev | CGGGGAGGAAG | housekeeping gene, control |
| | | GTGTTGTG (SEQ | With 51: 178 bp 16S-fragment |
| | | ID NO: 74) | |
| 53 | 53_165_rev2 | CAGACTCCTAC | housekeeping gene, control |
| | | GGGAGGCAG | With 51: 286 bp 16S-fragment |
| | | (SEQ ID NO: 75) | |
| 57 | 57_Dimer_for | CGGAAGCGTCC | 303 bp, detection of mRNA of B-Site |
| | | AAAAAACCGC | fusion protein (Binding dimerization region |
| | | (SEQ ID NO: 76) | N-Protein) |
| 58 | 58_Dimer_rev | GCAGGATAACC | |
| | | TGGTCTTTGAA | |
| | | G (SEQ ID NO: 77) | |
| 62 | 62_HlyB for | CCATAACGTCT | 301 bp-Fragment, detection of mRNA of |
| | | CTGTTAACCCG | HlyB |
| | | GAAG (SEQ ID | |
| | | NO: 78) | |
| 63 | 63_HlyB rev | CCCCTGATATA | |
| | | ACGCCTCAAAC | |
| | | TCAG (SEQ ID | |
| | | NO: 79) | |
| 64 | 64_HlyD for | GAATTCTTACCC | 321 bp-Fragment, detection of mRNA of |
| | | GCTCATCTGG | HlyD |
| | | (SEQ ID NO: 80) | |
| 65 | 65_HlyD rev | GGCCTGTAACA | |
| | | GTGATGACTGT | |
| | | G (SEQ ID NO: 81) | |
| 66 | 66_tyrS for | CCATTGTTATGC | 310 bp-Fragment, detection of mRNA of |
| | | CTGAAACGCTT | TyrS |
| | | CCAGC (SEQ ID | |
| | | NO: 82) | |
| 67 | 67_tyrS rev | CCGCTTCTTTGT | |
| | | TGATCATCTGGT | |
| | | TAACGG (SEQ ID | |
| | | NO: 83) | |
| | | | For determination of plasmid Copy |
| | | | number |
| 73 | 73_SlyB-for | GGTTTTATTCAT | with 74 or 75 detection SlyB (control) |
| | | TGCGCTCTGGA | |
| | | CGC | |
| | | (SEQ ID NO: 84) | |
| 74 | 74_SlyB-rev 113 | GATTCCTCGGC | with 73: 113 bp-fragment |
| | | AACACTATCGG | |
| | | (SEQ ID NO: 85) | |
| 75 | 75_SlyB-rev 302 | CACTGATGGGG | with 73: 302 bp-fragment |
| | | TTATCCTTAGCT | |
| | | GGG | |
| | | (SEQ ID NO: 86) | |
| 62 | 62_HlyB for | CCATAACGTCT | 104 bp-Fragment |
| | | CTGTTAACCCG | |
| | | GAAG (SEQ ID | |
| | | NO: 87) | |
| 76 | 76_HlyB rev 104 | GTTCTAAAGAT | |
| | | TTCGCAGCAAG | |
| | | CAAC (SEQ ID | |
| | | NO: 88) | |
| 62 | 62_HlyB for | CCATAACGTCT | 301 bp-Fragment |
| | | CTGTTAACCCG | |
| | | GAAG (SEQ ID | |
| | | NO: 89) | |
| 63 | 63_HlyB rev | CCCCTGATATA | |
| | | ACGCCTCAAAC | |
| | | TCAG (SEQ ID | |
| | | NO: 90) | |

TABLE 13
optimized CDS and amino acid (aa) sequences of fusion proteins of A-site in
accordance with the invention

| | | DNA-sequence: 5′→3 |
|---|---|---|
| | | NsiI-Sites: ATGCAT |
| | | DNA with optimized codon usage: underlined |
| | | CDS of RBD, respectively BetaCoV S1-CTD and fusions of RBD plus |
| | | regions of N-Protein (A22, A23) in bold |
| | | Amino acid-sequence: Start→end |
| | | Amino acids (aa) with optimized codon usage: underlined |
| Fusion | SEQ | RBD, respectively BetaCoV S1-CTD and fusions of RBD plus regions of N- |
| Protein | ID | Protein (A22, A23) in bold |
| A1 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 32 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAAAATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCTGTACCGTCTGTTCC |
| | | GTAAATCCAACCTGAAACCGTTCGAACGTGACATCTCCACCGAAATCTACCAGGCGG |
| | | GCTCCACCCCGTGCAACGGCGTTGAAGGCTTCAACTGCTACTTCCCGCTGCAGTCCTA |
| | | CGGCTTCCAGCCGACCAACGGCGTTGGCTACCAGCCGTACCGTGTTGTTGTTCTGTCC |
| | | TTCGAACTGCTGCACGCGCCGGCGACCGTTTGCGGCCCGAAAAAATCCACCAACCTG |
| | | GTTAAAAACAAATGCGTTAACTTCGACTACAAAGACGACGACGACAAAGAAGCGGC |
| | | GGCGAAACATGCATTAGCCTATGGAAGTCAGGGTGATCTTAATCCATTAATTAATGAA |
| | | ATCAGCAAAATCATTTCAGCTGCAGGTAGCTTCGATGTTAAAGAGGAAAGAACTGCA |
| | | GCTTCTTTATTGCAGTTGTCCGGTAATGCCAGTGATTTTTCATATGGACGGAACTCAAT |
| | | AACCCTGACCACATCAGCA |
| A1 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 30 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGKIADYNYKLPDDFTGCVIAWNSNNLDSKVGGNYNYLYRLFRKSNLKPFERDISTEI |
| | | YQAGSTPCNGVEGFNCYFPLQSYGFQPTNGVGYQPYRVVVLSFELLHAPATVCGPKKS |
| | | TNLVKNKCVNFDYKDDDDKEAAAKHALAYGSQGDLNPLINEISKIISAAGSFDVKEERTAA |
| | | SLLQLSGNASDFSYGRNSITLTTSA |
| A3 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 91 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAAAACCTGTGTCCGTT |
| | | TGGCGAAGTGTTCAACGCGACCCGTTTTGCGTCCGTCTACGCCTGGAACCGTAAACG |
| | | TATCTCCAACTGCGTTGCGGACTACTCCGTTCTGTACAACTCCGCGTCCTTCTCCACCT |
| | | TCAAATGCTACGGCGTTTCCCCGACCAAACTGAACGACCTGTGCTTCACCAACGTTTA |
| | | CGCGGACTCCTTCGTTATCCGTGGCGACGAAGTTCGTCAGATCGCGCCGGGCCAGAC |
| | | CGGCAAAATCGCGGACTACAACTACAAACTGCCGGACGACTTCACCGGCTGCGTTAT |
| | | CGCGTGGAACTCCAACAACCTGGACTCCAAAGTTGGCGGCAACTACAACTACCTGTA |
| | | CCGTCTGTTCCGTAAATCCAACCTGAAACCGTTCGAACGTGACATCTCCACCGAAATC |
| | | TACCAGGCGGGCTCCACCCCGTGCAACGGCGTTGAAGGCTTCAACTGCTACTTCCCG |
| | | CTGCAGTCCTACGGCTTCCAGCCGACCAACGGCGTTGGCTACCAGCCGTACCGTGTT |
| | | GTTGTTCTGTCCTTCGAACTGCTGCACGCGCCGGCGACCGTTTGCGGCCCGGACTACA |
| | | AAGACGACGACGACAAAGAAGCGGCGGCGAAACATGCATTAGCCTATGGAAGTCAG |
| | | GGTGATCTTAATCCATTAATTAATGAAATCAGCAAAATCATTTCAGCTGCAGGTAGCTT |
| | | CGATGTTAAAGAGGAAAGAACTGCAGCTTCTTTATTGCAGTTGTCCGGTAATGCCAGT |
| | | GATTTTTCATATGGACGGAACTCAATAACCCTGACCACATCAGCATAA |
| A3 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 92 | KLCVWNNKTPHAIAAISMANEAAAKNLCPFGEVFNATRFASVYAWNRKRISNCVADYS |
| | | VLYNSASFSTFKCYGVSPTKLNDLCFTNVYADSFVIRGDEVRQIAPGQTGKIADYNYKLP |
| | | DDFTGCVIAWNSNNLDSKVGGNYNYLYRLFRKSNLKPFERDISTEIYQAGSTPCNGVEG |
| | | FNCYFPLQSYGFQPTNGVGYQPYRVVVLSFELLHAPATVCGPDYKDDDDKEAAAKHAL |
| | | AYGSQGDLNPLINEISKIISAAGSFDVKEERTAASLLQLSGNASDFSYGRNSITLTTSA* |
| A11 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 93 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAAAATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCTGTACCGTCTGTTCC |
| | | GTAAATCCAACCTGAAACCGTTCGAACGTGACATCTCCACCGAAATCTACCAGGCGG |
| | | GCTCCACCCCGTGCAACGGCGTTGAAGGCTTCAACTGCTACTTCCCGCTGCAGTCCTA |
| | | CGGCTTCCAGCCGACCTACGGCGTTGGCTACCAGCCGTACCGTGTTGTTGTTCTGTCC |
| | | TTCGAACTGCTGCACGCGCCGGCGACCGTTTGCGGCCCGAAAAAATCCACCAACCTG |
| | | GTTAAAAACAAATGCGTTAACTTCGACTACAAAGACGACGACGACAAAGAAGCGGC |
| | | GGCGAAACATGCATTAGCCTATGGAAGTCAGGGTGATCTTAATCCATTAATTAATGAA |
| | | ATCAGCAAAATCATTTCAGCTGCAGGTAGCTTCGATGTTAAAGAGGAAAGAACTGCA |
| | | GCTTCTTTATTGCAGTTGTCCGGTAATGCCAGTGATTTTTCATATGGACGGAACTCAAT |
| | | AACCCTGACCACATCAGCATAA |
| A11 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 94 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGKIADYNYKLPDDFTGCVIAWNSNNLDSKVGGNYNYLYRLFRKSNLKPFERDISTEI |
| | | YQAGSTPCNGVEGFNCYFPLQSYGFQPTYGVGYQPYRVVVLSFELLHAPATVCGPKKST |
| | | NLVKNKCVNFDYKDDDDKEAAAKHALAYGSQGDLNPLINEISKIISAAGSFDVKEERTAAS |
| | | LLQLSGNASDFSYGRNSITLTTSA* |
| A12 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 95 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAAAATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCTGTACCGTCTGTTCC |
| | | GTAAATCCAACCTGAAACCGTTCGAACGTGACATCTCCACCGAAATCTACCAGGCGG |
| | | GCTCCACCCCGTGCAACGGCGTTAAAGGCTTCAACTGCTACTTCCCGCTGCAGTCCTA |
| | | CGGCTTCCAGCCGACCTACGGCGTTGGCTACCAGCCGTACCGTGTTGTTGTTCTGTCC |
| | | TTCGAACTGCTGCACGCGCCGGCGACCGTTTGCGGCCCGAAAAAATCCACCAACCTG |
| | | GTTAAAAACAAATGCGTTAACTTCGACTACAAAGACGACGACGACAAAGAAGCGGC |
| | | GGCGAAACATGCATTAGCCTATGGAAGTCAGGGTGATCTTAATCCATTAATTAATGAA |
| | | ATCAGCAAAATCATTTCAGCTGCAGGTAGCTTCGATGTTAAAGAGGAAAGAACTGCA |
| | | GCTTCTTTATTGCAGTTGTCCGGTAATGCCAGTGATTTTTCATATGGACGGAACTCAAT |
| | | AACCCTGACCACATCAGCATAA |
| A12 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 96 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGKIADYNYKLPDDFTGCVIAWNSNNLDSKVGGNYNYLYRLFRKSNLKPFERDISTEI |
| | | YQAGSTPCNGVKGFNCYFPLQSYGFQPTYGVGYQPYRVVVLSFELLHAPATVCGPKKST |
| | | NLVKNKCVNFDYKDDDDKEAAAKHALAYGSQGDLNPLINEISKIISAAGSFDVKEERTAAS |
| | | LLQLSGNASDFSYGRNSITLTTSA* |
| A13 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 97 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAACATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCTGTACCGTCTGTTCC |
| | | GTAAATCCAACCTGAAACCGTTCGAACGTGACATCTCCACCGAAATCTACCAGGCGG |
| | | GCTCCACCCCGTGCAACGGCGTTAAAGGCTTCAACTGCTACTTCCCGCTGCAGTCCTA |
| | | CGGCTTCCAGCCGACCTACGGCGTTGGCTACCAGCCGTACCGTGTTGTTGTTCTGTCC |
| | | TTCGAACTGCTGCACGCGCCGGCGACCGTTTGCGGCCCGAAAAAATCCACCAACCTG |
| | | GTTAAAAACAAATGCGTTAACTTCGACTACAAAGACGACGACGACAAAGAAGCGGC |
| | | GGCGAAACATGCATTAGCCTATGGAAGTCAGGGTGATCTTAATCCATTAATTAATGAA |
| | | ATCAGCAAAATCATTTCAGCTGCAGGTAGCTTCGATGTTAAAGAGGAAAGAACTGCA |
| | | GCTTCTTTATTGCAGTTGTCCGGTAATGCCAGTGATTTTTCATATGGACGGAACTCAAT |
| | | AACCCTGACCACATCAGCATAA |
| A13 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 98 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGNIADYNYKLPDDFTGCVIAWNSNNLDSKVGGNYNYLYRLFRKSNLKPFERDISTE |
| | | IYQAGSTPCNGVKGFNCYFPLQSYGFQPTYGVGYQPYRVVVLSFELLHAPATVCGPKKS |
| | | TNLVKNKCVNFDYKDDDDKEAAAKHALAYGSQGDLNPLINEISKIISAAGSFDVKEERTAA |
| | | SLLQLSGNASDFSYGRNSITLTTSA* |
| A14 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 99 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAACATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCTGTACCGTCTGTTCC |
| | | GTAAATCCAACCTGAAACCGTTCGAACGTGACATCTCCACCGAAATCTACCAGGCGG |
| | | GCTCCACCCCGTGCAACGGCGTTAAAGGCTTCAACTGCTACTTCCCGCTGCAGTCCTA |
| | | CGGCTTCCAGCCGACCTACGGCGTTGGCTACCAGCCGTACCGTGTTGTTGTTCTGTCC |
| | | TTCGAACTGCTGCACGCGCCGGCGACCGTTTGCGGCCCGAAAAAATCCACCAACCTG |
| | | GTTAAAAACAAATGCGTTAACTTCCGTGTTCAGCCGACCGAATCCATAGTTAGGTTC |
| | | CCGAACATCACTAACCTGTGTCCGTTTGGCGAAGTGTTCAACGCGACCCGTTTTGCGT |
| | | CCGTCTACGCCTGGAACCGTAAACGTATCTCCAACTGCGTTGCGGACTACTCCGTTCT |
| | | GTACAACTCCGCGTCCTTCTCCACCTTCAAATGCTACGGCGTTTCCCCGACCAAACTG |
| | | AACGACCTGTGCTTCACCAACGTTTACGCGGACTCCTTCGTTATCCGTGGCGACGAA |
| | | GTTCGTCAGATCGCGCCGGGCCAGACCGGCAAAATCGCGGACTACAACTACAAACT |
| | | GCCGGACGACTTCACCGGCTGCGTTATCGCGTGGAACTCCAACAACCTGGACTCCAA |
| | | AGTTGGCGGCAACTACAACTACCTGTACCGTCTGTTCCGTAAATCCAACCTGAAACC |
| | | GTTCGAACGTGACATCTCCACCGAAATCTACCAGGCGGGCTCCACCCCGTGCAACGG |
| | | CGTTGAAGGCTTCAACTGCTACTTCCCGCTGCAGTCCTACGGCTTCCAGCCGACCTAC |
| | | GGCGTTGGCTACCAGCCGTACCGTGTTGTTGTTCTGTCCTTCGAACTGCTGCACGCGC |
| | | CGGCGACCGTTTGCGGCCCGAAAAAATCCACCAACCTGGTTAAAAACAAATGCGTT |
| | | AACTTCGACTACAAAGACGACGACGACAAAGAAGCGGCGGCGAAACATGCATTAGC |
| | | CTATGGAAGTCAGGGTGATCTTAATCCATTAATTAATGAAATCAGCAAAATCATTTCA |
| | | GCTGCAGGTAGCTTCGATGTTAAAGAGGAAAGAACTGCAGCTTCTTTATTGCAGTTGT |
| | | CCGGTAATGCCAGTGATTTTTCATATGGACGGAACTCAATAACCCTGACCACATCAGC |
| | | ATAA |
| A14 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 100 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGNIADYNYKLPDDFTGCVIAWNSNNLDSKVGGNYNYLYRLFRKSNLKPFERDISTE |
| | | IYQAGSTPCNGVKGFNCYFPLQSYGFQPTYGVGYQPYRVVVLSFELLHAPATVCGPKKS |
| | | TNLVKNKCVNFRVQPTESIVRFPNITNLCPFGEVFNATRFASVYAWNRKRISNCVADYS |
| | | VLYNSASFSTFKCYGVSPTKLNDLCFTNVYADSFVIRGDEVRQIAPGQTGKIADYNYKLP |
| | | DDFTGCVIAWNSNNLDSKVGGNYNYLYRLFRKSNLKPFERDISTEIYQAGSTPCNGVEG |
| | | FNCYFPLQSYGFQPTYGVGYQPYRVVVLSFELLHAPATVCGPKKSTNLVKNKCVNFDYK |
| | | DDDDKEAAAKHALAYGSQGDLNPLINEISKIISAAGSFDVKEERTAASLLQLSGNASDFSYG |
| | | RNSITLTTSA* |
| A15 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 101 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCACCATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCTGTACCGTCTGTTCC |
| | | GTAAATCCAACCTGAAACCGTTCGAACGTGACATCTCCACCGAAATCTACCAGGCGG |
| | | GCTCCACCCCGTGCAACGGCGTTAAAGGCTTCAACTGCTACTTCCCGCTGCAGTCCTA |
| | | CGGCTTCCAGCCGACCTACGGCGTTGGCTACCAGCCGTACCGTGTTGTTGTTCTGTCC |
| | | TTCGAACTGCTGCACGCGCCGGCGACCGTTTGCGGCCCGAAAAAATCCACCAACCTG |
| | | GTTAAAAACAAATGCGTTAACTTCGACTACAAAGACGACGACGACAAAGAAGCGGC |
| | | GGCGAAACATGCATTAGCCTATGGAAGTCAGGGTGATCTTAATCCATTAATTAATGAA |
| | | ATCAGCAAAATCATTTCAGCTGCAGGTAGCTTCGATGTTAAAGAGGAAAGAACTGCA |
| | | GCTTCTTTATTGCAGTTGTCCGGTAATGCCAGTGATTTTTCATATGGACGGAACTCAAT |
| | | AACCCTGACCACATCAGCATAA |
| A15 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 102 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGTIADYNYKLPDDFTGCVIAWNSNNLDSKVGGNYNYLYRLFRKSNLKPFERDISTEI |
| | | YQAGSTPCNGVKGFNCYFPLQSYGFQPTYGVGYQPYRVVVLSFELLHAPATVCGPKKST |
| | | NLVKNKCVNFDYKDDDDKEAAAKHALAYGSQGDLNPLINEISKIISAAGSFDVKEERTAAS |
| | | LLQLSGNASDFSYGRNSITLTTSA* |
| A16 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 103 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAAGACTACAAAGACG |
| | | ACGACGACAAAGAAGCGGCGGCGAAACATGCATTAGCCTATGGAAGTCAGGGTGAT |
| | | CTTAATCCATTAATTAATGAAATCAGCAAAATCATTTCAGCTGCAGGTAGCTTCGATGT |
| | | TAAAGAGGAAAGAACTGCAGCTTCTTTATTGCAGTTGTCCGGTAATGCCAGTGATTTT |
| | | TCATATGGACGGAACTCAATAACCCTGACCACATCAGCATAA |
| A16 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 104 | KLCVWNNKTPHAIAAISMANEAAAKDYKDDDDKEAAAKHALAYGSQGDLNPLINEISKIIS |
| | | AAGSFDVKEERTAASLLQLSGNASDFSYGRNSITLTTSA* |
| A17 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 105 | GCGAAACGTGTTCAGCCGACCGAATCCATAGTTAGGTTCCCGAACATCACTAACCTG |
| | | TGTCCGTTTGGCGAAGTGTTCAACGCGACCCGTTTTGCGTCCGTCTACGCCTGGAACC |
| | | GTAAACGTATCTCCAACTGCGTTGCGGACTACTCCGTTCTGTACAACTCCGCGTCCTT |
| | | CTCCACCTTCAAATGCTACGGCGTTTCCCCGACCAAACTGAACGACCTGTGCTTCACC |
| | | AACGTTTACGCGGACTCCTTCGTTATCCGTGGCGACGAAGTTCGTCAGATCGCGCCG |
| | | GGCCAGACCGGCAAAATCGCGGACTACAACTACAAACTGCCGGACGACTTCACCGG |
| | | CTGCGTTATCGCGTGGAACTCCAACAACCTGGACTCCAAAGTTGGCGGCAACTACAA |
| | | CTACCTGTACCGTCTGTTCCGTAAATCCAACCTGAAACCGTTCGAACGTGACATCTCC |
| | | ACCGAAATCTACCAGGCGGGCTCCACCCCGTGCAACGGCGTTGAAGGCTTCAACTGC |
| | | TACTTCCCGCTGCAGTCCTACGGCTTCCAGCCGACCAACGGCGTTGGCTACCAGCCGT |
| | | ACCGTGTTGTTGTTCTGTCCTTCGAACTGCTGCACGCGCCGGCGACCGTTTGCGGCCC |
| | | GAAAAAATCCACCAACCTGGTTAAAAACAAATGCGTTAACTTCGACTACAAAGACGA |
| | | CGACGACAAAGAAGCGGCGGCGAAACATGCATTAGCCTATGGAAGTCAGGGTGATC |
| | | TTAATCCATTAATTAATGAAATCAGCAAAATCATTTCAGCTGCAGGTAGCTTCGATGTT |
| | | AAAGAGGAAAGAACTGCAGCTTCTTTATTGCAGTTGTCCGGTAATGCCAGTGATTTTT |
| | | CATATGGACGGAACTCAATAACCCTGACCACATCAGCATAA |
| A17 | SEQ ID | MPTITTAQIKSTLQSAKQSAANKLHSAGQSTKDASEAAAKRVQPTESIVRFPNITNLCPFG |
| aa | NO: | EVFNATRFASVYAWNRKRISNCVADYSVLYNSASFSTFKCYGVSPTKLNDLCFTNVYAD |
| | 106 | SFVIRGDEVRQIAPGQTGKIADYNYKLPDDFTGCVIAWNSNNLDSKVGGNYNYLYRLFR |
| | | KSNLKPFERDISTEIYQAGSTPCNGVEGFNCYFPLQSYGFQPTNGVGYQPYRVVVLSFEL |
| | | LHAPATVCGPKKSTNLVKNKCVNFDYKDDDDKEAAAKHALAYGSQGDLNPLINEISKIISA |
| | | AGSFDVKEERTAASLLQLSGNASDFSYGRNSITLTTSA* |
| A18 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 107 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAAAATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCGTTACCGTCTGTTCC |
| | | GTAAATCCAACCTGAAACCGTTCGAACGTGACATCTCCACCGAAATCTACCAGGCGG |
| | | GCTCCACCCCGTGCAACGGCGTTCAGGGCTTCAACTGCTACTTCCCGCTGCAGTCCTA |
| | | CGGCTTCCAGCCGACCAACGGCGTTGGCTACCAGCCGTACCGTGTTGTTGTTCTGTCC |
| | | TTCGAACTGCTGCACGCGCCGGCGACCGTTTGCGGCCCGAAAAAATCCACCAACCTG |
| | | GTTAAAAACAAATGCGTTAACTTCGACTACAAAGACGACGACGACAAAGAAGCGGC |
| | | GGCGAAACATGCATTAGCCTATGGAAGTCAGGGTGATCTTAATCCATTAATTAATGAA |
| | | ATCAGCAAAATCATTTCAGCTGCAGGTAGCTTCGATGTTAAAGAGGAAAGAACTGCA |
| | | GCTTCTTTATTGCAGTTGTCCGGTAATGCCAGTGATTTTTCATATGGACGGAACTCAAT |
| | | AACCCTGACCACATCAGCATAA |
| A18 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 108 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGKIADYNYKLPDDFTGCVIAWNSNNLDSKVGGNYNYRYRLFRKSNLKPFERDISTE |
| | | IYQAGSTPCNGVQGFNCYFPLQSYGFQPTNGVGYQPYRVVVLSFELLHAPATVCGPKK |
| | | STNLVKNKCVNFDYKDDDDKEAAAKHALAYGSQGDLNPLINEISKIISAAGSFDVKEERTA |
| | | ASLLQLSGNASDFSYGRNSITLTTSA* |
| A19 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 109 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAAAATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCGTTACCGTCTGTTCC |
| | | GTAAATCCAACCTGAAACCGTTCGAACGTGACATCTCCACCGAAATCTACCAGGCGG |
| | | GCTCCAAACCGTGCAACGGCGTTGAAGGCTTCAACTGCTACTTCCCGCTGCAGTCCT |
| | | ACGGCTTCCAGCCGACCAACGGCGTTGGCTACCAGCCGTACCGTGTTGTTGTTCTGT |
| | | CCTTCGAACTGCTGCACGCGCCGGCGACCGTTTGCGGCCCGAAAAAATCCACCAACC |
| | | TGGTTAAAAACAAATGCGTTAACTTCGACTACAAAGACGACGACGACAAAGAAGCG |
| | | GCGGCGAAACATGCATTAGCCTATGGAAGTCAGGGTGATCTTAATCCATTAATTAATG |
| | | AAATCAGCAAAATCATTTCAGCTGCAGGTAGCTTCGATGTTAAAGAGGAAAGAACTG |
| | | CAGCTTCTTTATTGCAGTTGTCCGGTAATGCCAGTGATTTTTCATATGGACGGAACTCA |
| | | ATAACCCTGACCACATCAGCATAA |
| A19 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 110 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGKIADYNYKLPDDFTGCVIAWNSNNLDSKVGGNYNYRYRLFRKSNLKPFERDISTE |
| | | IYQAGSKPCNGVEGFNCYFPLQSYGFQPTNGVGYQPYRVVVLSFELLHAPATVCGPKKS |
| | | TNLVKNKCVNFDYKDDDDKEAAAKHALAYGSQGDLNPLINEISKIISAAGSFDVKEERTAA |
| | | SLLQLSGNASDFSYGRNSITLTTSA* |
| A20 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 111 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAACATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCGTTACCGTCTGTTCC |
| | | GTAAATCCAACCTGAAACCGTTCGAACGTGACATCTCCACCGAAATCTACCAGGCGG |
| | | GCTCCAAACCGTGCAACGGCGTTGAAGGCTTCAACTGCTACTTCCCGCTGCAGTCCT |
| | | ACGGCTTCCAGCCGACCAACGGCGTTGGCTACCAGCCGTACCGTGTTGTTGTTCTGT |
| | | CCTTCGAACTGCTGCACGCGCCGGCGACCGTTTGCGGCCCGAAAAAATCCACCAACC |
| | | TGGTTAAAAACAAATGCGTTAACTTCGACTACAAAGACGACGACGACAAAGAAGCG |
| | | GCGGCGAAACATGCATTAGCCTATGGAAGTCAGGGTGATCTTAATCCATTAATTAATG |
| | | AAATCAGCAAAATCATTTCAGCTGCAGGTAGCTTCGATGTTAAAGAGGAAAGAACTG |
| | | CAGCTTCTTTATTGCAGTTGTCCGGTAATGCCAGTGATTTTTCATATGGACGGAACTCA |
| | | ATAACCCTGACCACATCAGCATAA |
| A20 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 112 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGNIADYNYKLPDDFTGCVIAWNSNNLDSKVGGNYNYRYRLFRKSNLKPFERDISTE |
| | | IYQAGSKPCNGVEGFNCYFPLQSYGFQPTNGVGYQPYRVVVLSFELLHAPATVCGPKKS |
| | | TNLVKNKCVNFDYKDDDDKEAAAKHALAYGSQGDLNPLINEISKIISAAGSFDVKEERTAA |
| | | SLLQLSGNASDFSYGRNSITLTTSA* |
| A21 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 113 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAAAATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCAGTACCGTCTGTTCC |
| | | GTAAATCCAACCTGAAACCGTTCGAACGTGACATCTCCACCGAAATCTACCAGGCGG |
| | | GCTCCACCCCGTGCAACGGCGTTGAAGGCTTCAACTGCTACTCCCCGCTGCAGTCCTA |
| | | CGGCTTCCAGCCGACCAACGGCGTTGGCTACCAGCCGTACCGTGTTGTTGTTCTGTCC |
| | | TTCGAACTGCTGCACGCGCCGGCGACCGTTTGCGGCCCGAAAAAATCCACCAACCTG |
| | | GTTAAAAACAAATGCGTTAACTTCGACTACAAAGACGACGACGACAAAGAAGCGGC |
| | | GGCGAAACATGCATTAGCCTATGGAAGTCAGGGTGATCTTAATCCATTAATTAATGAA |
| | | ATCAGCAAAATCATTTCAGCTGCAGGTAGCTTCGATGTTAAAGAGGAAAGAACTGCA |
| | | GCTTCTTTATTGCAGTTGTCCGGTAATGCCAGTGATTTTTCATATGGACGGAACTCAAT |
| | | AACCCTGACCACATCAGCATAA |
| A21 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 114 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGKIADYNYKLPDDFTGCVIAWNSNNLDSKVGGNYNYQYRLFRKSNLKPFERDISTE |
| | | IYQAGSTPCNGVEGFNCYSPLQSYGFQPTNGVGYQPYRVVVLSFELLHAPATVCGPKKS |
| | | TNLVKNKCVNFDYKDDDDKEAAAKHALAYGSQGDLNPLINEISKIISAAGSFDVKEERTAA |
| | | SLLQLSGNASDFSYGRNSITLTTSA* |
| A22 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 115 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAAAATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCGTTACCGTCTGTTCC |
| | | GTAAATCCAACCTGAAACCGTTCGAACGTGACATCTCCACCGAAATCTACCAGGCGG |
| | | GCTCCAAACCGTGCAACGGCGTTGAAGGCTTCAACTGCTACTTCCCGCTGCAGTCCT |
| | | ACGGCTTCCAGCCGACCAACGGCGTTGGCTACCAGCCGTACCGTGTTGTTGTTCTGT |
| | | CCTTCGAACTGCTGCACGCGCCGGCGACCGTTTGCGGCCCGAAAAAATCCACCAACC |
| | | TGGTTAAAAACAAATGCGTTAACTTCCCGCGTCAGAAACGTACCGCGACCAAAGCG |
| | | TACAACGTTACCCAGGCGTTCGGCCGTCGTGGCCCGGAACAGACCCAGGGCAACTTC |
| | | GGCGACCAGGAACTGATCCGTCAGGGCACCGACTACAAACACTGGCCGCAGATCGC |
| | | GCAGTTCGCGCCGTCCGCGTCCGCGTTCTTCGGCATGTCCCGTATCGGCATGGAAGT |
| | | TACCCCGTCCGGCACCTGGCTGACCTACACCGGCGCGATCAAACTGGACGACAAAG |
| | | ACCCGAACTTCAAAGACCAGGTTATCCTGCTGAACAAACACATCGACGCGTACAAA |
| | | GACTACAAAGACGACGACGACAAAGAAGCGGCGGCGAAACATGCATTAGCCTATGG |
| | | AAGTCAGGGTGATCTTAATCCATTAATTAATGAAATCAGCAAAATCATTTCAGCTGCA |
| | | GGTAGCTTCGATGTTAAAGAGGAAAGAACTGCAGCTTCTTTATTGCAGTTGTCCGGTA |
| | | ATGCCAGTGATTTTTCATATGGACGGAACTCAATAACCCTGACCACATCAGCATAA |
| A22 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 116 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGKIADYNYKLPDDFTGCVIAWNSNNLDSKVGGNYNYRYRLFRKSNLKPFERDISTE |
| | | IYQAGSKPCNGVEGFNCYFPLQSYGFQPTNGVGYQPYRVVVLSFELLHAPATVCGPKKS |
| | | TNLVKNKCVNFPRQKRTATKAYNVTQAFGRRGPEQTQGNFGDQELIRQGTDYKHWP |
| | | QIAQFAPSASAFFGMSRIGMEVTPSGTWLTYTGAIKLDDKDPNFKDQVILLNKHIDAYK |
| | | DYKDDDDKEAAAKHALAYGSQGDLNPLINEISKIISAAGSFDVKEERTAASLLQLSGNASDF |
| | | SYGRNSITLTTSA* |
| A23 | SEQ ID | ATGCCAACAATAACCACTGCACAAATTAAAAGCACACTGCAGTCTGCAAAGCAATCCG |
| CDS | NO: | CTGCAAATAAATTGCACTCAGCAGGACAAAGCACGAAAGATGCATCAGAAGCGGCG |
| | 117 | GCGAAAACCCCGCAGAACATCACCGACCTGTGCGCGGAATACCACAACACCCAGATC |
| | | CACACCCTGAACGACAAAATCTTCTCCTACACCGAATCCCTGGCGGGCAAACGTGAAA |
| | | TGGCGATCATCACCTTCAAAAACGGCGCGACCTTCCAGGTTGAAGTTCCGGGCTCCCA |
| | | GCACATCGACTCCCAGAAAAAAGCGATCGAACGTATGAAAGACACCCTGCGTATCGC |
| | | GTACCTGACCGAAGCGAAAGTTGAAAAACTGTGCGTTTGGAACAACAAAACCCCGCA |
| | | CGCGATCGCGGCGATCTCCATGGCGAACGAAGCGGCGGCGAAACGTGTTCAGCCGA |
| | | CCGAATCCATAGTTAGGTTCCCGAACATCACTAACCTGTGTCCGTTTGGCGAAGTGTT |
| | | CAACGCGACCCGTTTTGCGTCCGTCTACGCCTGGAACCGTAAACGTATCTCCAACTGC |
| | | GTTGCGGACTACTCCGTTCTGTACAACTCCGCGTCCTTCTCCACCTTCAAATGCTACG |
| | | GCGTTTCCCCGACCAAACTGAACGACCTGTGCTTCACCAACGTTTACGCGGACTCCTT |
| | | CGTTATCCGTGGCGACGAAGTTCGTCAGATCGCGCCGGGCCAGACCGGCAAAATCG |
| | | CGGACTACAACTACAAACTGCCGGACGACTTCACCGGCTGCGTTATCGCGTGGAACT |
| | | CCAACAACCTGGACTCCAAAGTTGGCGGCAACTACAACTACCGTTACCGTCTGTTCC |
| | | GTAAATCCAACCTGAAACCGTTCGAACGTGACATCTCCACCGAAATCTACCAGGCGG |
| | | GCTCCAAACCGTGCAACGGCGTTGAAGGCTTCAACTGCTACTTCCCGCTGCAGTCCT |
| | | ACGGCTTCCAGCCGACCAACGGCGTTGGCTACCAGCCGTACCGTGTTGTTGTTCTGT |
| | | CCTTCGAACTGCTGCACGCGCCGGCGACCGTTTGCGGCCCGAAAAAATCCACCAACC |
| | | TGGTTAAAAACAAATGCGTTAACTTCGCGGCGCTGGCGCTGCTGCTGCTGGACCGTC |
| | | TGAACCAGCTGGAATCCAAAATGTCCGGCAAAGGCCAGCAGCAGCAGGGCCAGAC |
| | | CGTTACCAAAAAATCCGCGGCGGAAGCGTCCAAAAAACCGCGTCAGAAACGTACCG |
| | | CGACCAAAGCGTACAACGTTACCCAGGCGTTCGGCCGTCGTGGCCCGGAACAGACC |
| | | CAGGGCAACTTCGGCGACCAGGAACTGATCCGTCAGGGCACCGACTACAAACACTG |
| | | GCCGCAGATCGCGCAGTTCGCGCCGTCCGCGTCCGCGTTCTTCGGCATGTCCCGTATC |
| | | GGCATGGAAGTTACCCCGTCCGGCACCTGGCTGACCTACACCGGCGCGATCAAACTG |
| | | GACGACAAAGACCCGAACTTCAAAGACCAGGTTATCCTGCTGAACAAACACATCGA |
| | | CGCGTACAAAACCTTCCCGCCGACCGAACCGAAAGACTACAAAGACGACGACGACA |
| | | AAGAAGCGGCGGCGAAACATGCATTAGCCTATGGAAGTCAGGGTGATCTTAATCCAT |
| | | TAATTAATGAAATCAGCAAAATCATTTCAGCTGCAGGTAGCTTCGATGTTAAAGAGGA |
| | | AAGAACTGCAGCTTCTTTATTGCAGTTGTCCGGTAATGCCAGTGATTTTTCATATGGAC |
| | | GGAACTCAATAACCCTGACCACATCAGCATAA |
| A23 | SEQ ID | MPTITTAQIKSTLQSAKQSAANKLHSAGQSTKDASEAAAKTPQNITDLCAEYHNTQIHTLN |
| aa | NO: | DKIFSYTESLAGKREMAIITFKNGATFQVEVPGSQHIDSQKKAIERMKDTLRIAYLTEAKVE |
| | 118 | KLCVWNNKTPHAIAAISMANEAAAKRVQPTESIVRFPNITNLCPFGEVFNATRFASVYA |
| | | WNRKRISNCVADYSVLYNSASFSTFKCYGVSPTKLNDLCFTNVYADSFVIRGDEVRQIAP |
| | | GQTGKIADYNYKLPDDFTGCVIAWNSNNLDSKVGGNYNYRYRLFRKSNLKPFERDISTE |
| | | IYQAGSKPCNGVEGFNCYFPLQSYGFQPTNGVGYQPYRVVVLSFELLHAPATVCGPKKS |
| | | TNLVKNKCVNFAALALLLLDRLNQLESKMSGKGQQQQGQTVTKKSAAEASKKPRQKR |
| | | TATKAYNVTQAFGRRGPEQTQGNFGDQELIRQGTDYKHWPQIAQFAPSASAFFGMS |
| | | RIGMEVTPSGTWLTYTGAIKLDDKDPNFKDQVILLNKHIDAYKTFPPTEPKDYKDDDDK |
| | | EAAAKHALAYGSQGDLNPLINEISKIISAAGSFDVKEERTAASLLQLSGNASDFSYGRNSITL |
| | | TTSA* |

Note that the end of the translated sequence is denoted by an asterisk (*).

TABLE 14
optimized CDS and amino acid sequences (aa) of viral antigen units in fusion proteins
of A-site in accordance with the invention

| Viral | | |
|---|---|---|
| antigen | SEQ | DNA-sequence: 5′→3 |
| unit in | ID | Amino acid-sequence: Start→end |
| A1 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 119 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAAAATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCTGTACCGTCTGTTCCGTAAATCCAACC |
| | | TGAAACCGTTCGAACGTGACATCTCCACCGAAATCTACCAGGCGGGCTCCACC |
| | | CCGTGCAACGGCGTTGAAGGCTTCAACTGCTACTTCCCGCTGCAGTCCTACGG |
| | | CTTCCAGCCGACCAACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTC |
| A1 | SEQ | RVQPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGKIADYNYKLPDDFT |
| | NO: | GCVIAWNSNNLDSKVGGNYNYLYRLFRKSNLKPFERDISTEIYQAGSTPCNGVEGF |
| | 120 | NCYFPLQSYGFQPTNGVGYQPYRVVVLSFELLHAPATVCGPKKSTNLVKNKCVNF |
| A3 | SEQ ID | AACCTGTGTCCGTTTGGCGAAGTGTTCAACGCGACCCGTTTTGCGTCCGTCTAC |
| CDS | NO: | GCCTGGAACCGTAAACGTATCTCCAACTGCGTTGCGGACTACTCCGTTCTGTA |
| | 121 | CAACTCCGCGTCCTTCTCCACCTTCAAATGCTACGGCGTTTCCCCGACCAAACT |
| | | GAACGACCTGTGCTTCACCAACGTTTACGCGGACTCCTTCGTTATCCGTGGCG |
| | | ACGAAGTTCGTCAGATCGCGCCGGGCCAGACCGGCAAAATCGCGGACTACAA |
| | | CTACAAACTGCCGGACGACTTCACCGGCTGCGTTATCGCGTGGAACTCCAACA |
| | | ACCTGGACTCCAAAGTTGGCGGCAACTACAACTACCTGTACCGTCTGTTCCGT |
| | | AAATCCAACCTGAAACCGTTCGAACGTGACATCTCCACCGAAATCTACCAGGC |
| | | GGGCTCCACCCCGTGCAACGGCGTTGAAGGCTTCAACTGCTACTTCCCGCTGC |
| | | AGTCCTACGGCTTCCAGCCGACCAACGGCGTTGGCTACCAGCCGTACCGTGTT |
| | | GTTGTTCTGTCCTTCGAACTGCTGCACGCGCCGGCGACCGTTTGCGGCCCG |
| A3 | SEQ | NLCPFGEVFNATRFASVYAWNRKRISNCVADYSVLYNSASFSTFKCYGVSPTKLND |
| aa | ID | LCFTNVYADSFVIRGDEVRQIAPGQTGKIADYNYKLPDDFTGCVIAWNSNNLDSK |
| | NO: | VGGNYNYLYRLFRKSNLKPFERDISTEIYQAGSTPCNGVEGFNCYFPLQSYGFQPT |
| | 122 | NGVGYQPYRVVVLSFELLHAPATVCGP |
| A11 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 123 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAAAATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCTGTACCGTCTGTTCCGTAAATCCAACC |
| | | TGAAACCGTTCGAACGTGACATCTCCACCGAAATCTACCAGGCGGGCTCCACC |
| | | CCGTGCAACGGCGTTGAAGGCTTCAACTGCTACTTCCCGCTGCAGTCCTACGG |
| | | CTTCCAGCCGACCTACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTC |
| A11 | SEQ | RVQPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGKIADYNYKLPDDFT |
| | NO: | GCVIAWNSNNLDSKVGGNYNYLYRLFRKSNLKPFERDISTEIYQAGSTPCNGVEGF |
| | 124 | NCYFPLQSYGFQPTYGVGYQPYRVVVLSFELLHAPATVCGPKKSTNLVKNKCVNF |
| A12 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 125 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAAAATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCTGTACCGTCTGTTCCGTAAATCCAACC |
| | | TGAAACCGTTCGAACGTGACATCTCCACCGAAATCTACCAGGCGGGCTCCACC |
| | | CCGTGCAACGGCGTTAAAGGCTTCAACTGCTACTTCCCGCTGCAGTCCTACGG |
| | | CTTCCAGCCGACCTACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTC |
| A12 | SEQ | RVQPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGKIADYNYKLPDDFT |
| | NO: | GCVIAWNSNNLDSKVGGNYNYLYRLFRKSNLKPFERDISTEIYQAGSTPCNGVKGF |
| | 126 | NCYFPLQSYGFQPTYGVGYQPYRVVVLSFELLHAPATVCGPKKSTNLVKNKCVNF |
| A13 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 127 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAACATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCTGTACCGTCTGTTCCGTAAATCCAACC |
| | | TGAAACCGTTCGAACGTGACATCTCCACCGAAATCTACCAGGCGGGCTCCACC |
| | | CCGTGCAACGGCGTTAAAGGCTTCAACTGCTACTTCCCGCTGCAGTCCTACGG |
| | | CTTCCAGCCGACCTACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTC |
| A13 | SEQ | RVQPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGNIADYNYKLPDDFT |
| | NO: | GCVIAWNSNNLDSKVGGNYNYLYRLFRKSNLKPFERDISTEIYQAGSTPCNGVKGF |
| | 128 | NCYFPLQSYGFQPTYGVGYQPYRVVVLSFELLHAPATVCGPKKSTNLVKNKCVNF |
| A14 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 129 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAACATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCTGTACCGTCTGTTCCGTAAATCCAACC |
| | | TGAAACCGTTCGAACGTGACATCTCCACCGAAATCTACCAGGCGGGCTCCACC |
| | | CCGTGCAACGGCGTTAAAGGCTTCAACTGCTACTTCCCGCTGCAGTCCTACGG |
| | | CTTCCAGCCGACCTACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTCCGTGTTCAGCCGACCGAATCCATA |
| | | GTTAGGTTCCCGAACATCACTAACCTGTGTCCGTTTGGCGAAGTGTTCAACGC |
| | | GACCCGTTTTGCGTCCGTCTACGCCTGGAACCGTAAACGTATCTCCAACTGCGT |
| | | TGCGGACTACTCCGTTCTGTACAACTCCGCGTCCTTCTCCACCTTCAAATGCTA |
| | | CGGCGTTTCCCCGACCAAACTGAACGACCTGTGCTTCACCAACGTTTACGCGG |
| | | ACTCCTTCGTTATCCGTGGCGACGAAGTTCGTCAGATCGCGCCGGGCCAGACC |
| | | GGCAAAATCGCGGACTACAACTACAAACTGCCGGACGACTTCACCGGCTGCG |
| | | TTATCGCGTGGAACTCCAACAACCTGGACTCCAAAGTTGGCGGCAACTACAAC |
| | | TACCTGTACCGTCTGTTCCGTAAATCCAACCTGAAACCGTTCGAACGTGACATC |
| | | TCCACCGAAATCTACCAGGCGGGCTCCACCCCGTGCAACGGCGTTGAAGGCTT |
| | | CAACTGCTACTTCCCGCTGCAGTCCTACGGCTTCCAGCCGACCTACGGCGTTG |
| | | GCTACCAGCCGTACCGTGTTGTTGTTCTGTCCTTCGAACTGCTGCACGCGCCG |
| | | GCGACCGTTTGCGGCCCGAAAAAATCCACCAACCTGGTTAAAAACAAATGCGT |
| | | TAACTTC |
| A14 | SEQ | RVOPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGNIADYNYKLPDDFT |
| | NO: | GCVIAWNSNNLDSKVGGNYNYLYRLFRKSNLKPFERDISTEIYQAGSTPCNGVKGF |
| | 130 | NCYFPLQSYGFQPTYGVGYQPYRVVVLSFELLHAPATVCGPKKSTNLVKNKCVNF |
| | | RVQPTESIVRFPNITNLCPFGEVFNATRFASVYAWNRKRISNCVADYSVLYNSASF |
| | | STFKCYGVSPTKLNDLCFTNVYADSFVIRGDEVRQIAPGQTGKIADYNYKLPDDFT |
| | | GCVIAWNSNNLDSKVGGNYNYLYRLFRKSNLKPFERDISTEIYQAGSTPCNGVEGF |
| | | NCYFPLQSYGFQPTYGVGYQPYRVVVLSFELLHAPATVCGPKKSTNLVKNKCVNF |
| A15 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 131 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCACCATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCTGTACCGTCTGTTCCGTAAATCCAACC |
| | | TGAAACCGTTCGAACGTGACATCTCCACCGAAATCTACCAGGCGGGCTCCACC |
| | | CCGTGCAACGGCGTTAAAGGCTTCAACTGCTACTTCCCGCTGCAGTCCTACGG |
| | | CTTCCAGCCGACCTACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTC |
| A15 | SEQ | RVQPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGTIADYNYKLPDDFT |
| | NO: | GCVIAWNSNNLDSKVGGNYNYLYRLFRKSNLKPFERDISTEIYQAGSTPCNGVKGF |
| | 132 | NCYFPLQSYGFQPTYGVGYQPYRVVVLSFELLHAPATVCGPKKSTNLVKNKCVNF |
| A16 | none none | |
| CDS | | |
| A16 | none none | |
| aa | | |
| A17 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 119 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAAAATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCTGTACCGTCTGTTCCGTAAATCCAACC |
| | | TGAAACCGTTCGAACGTGACATCTCCACCGAAATCTACCAGGCGGGCTCCACC |
| | | CCGTGCAACGGCGTTGAAGGCTTCAACTGCTACTTCCCGCTGCAGTCCTACGG |
| | | CTTCCAGCCGACCAACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTC |
| A17 | SEQ | RVQPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGKIADYNYKLPDDFT |
| | NO: | GCVIAWNSNNLDSKVGGNYNYLYRLFRKSNLKPFERDISTEIYQAGSTPCNGVEGF |
| | 120 | NCYFPLQSYGFQPTNGVGYQPYRVVVLSFELLHAPATVCGPKKSTNLVKNKCVNF |
| A18 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 133 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAAAATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCGTTACCGTCTGTTCCGTAAATCCAACC |
| | | TGAAACCGTTCGAACGTGACATCTCCACCGAAATCTACCAGGCGGGCTCCACC |
| | | CCGTGCAACGGCGTTCAGGGCTTCAACTGCTACTTCCCGCTGCAGTCCTACGG |
| | | CTTCCAGCCGACCAACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTC |
| A18 | SEQ | RVQPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGKIADYNYKLPDDFT |
| | NO: | GCVIAWNSNNLDSKVGGNYNYRYRLFRKSNLKPFERDISTEIYQAGSTPCNGVQG |
| | 134 | FNCYFPLQSYGFQPTNGVGYQPYRVVVLSFELLHAPATVCGPKKSTNLVKNKCVN |
| | | F |
| A19 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 135 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAAAATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCGTTACCGTCTGTTCCGTAAATCCAACC |
| | | TGAAACCGTTCGAACGTGACATCTCCACCGAAATCTACCAGGCGGGCTCCAAA |
| | | CCGTGCAACGGCGTTGAAGGCTTCAACTGCTACTTCCCGCTGCAGTCCTACGG |
| | | CTTCCAGCCGACCAACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTC |
| A19 | SEQ ID | RVQPTESIVRFPNITNLCPFGEVFNATRFASVYAWNRKRISNCVADYSVLYNSASF |
| aa | NO: | STFKCYGVSPTKLNDLCFTNVYADSFVIRGDEVRQIAPGQTGKIADYNYKLPDDFT |
| | 136 | GCVIAWNSNNLDSKVGGNYNYRYRLFRKSNLKPFERDISTEIYQAGSKPCNGVEG |
| | | FNCYFPLQSYGFQPTNGVGYQPYRVVVLSFELLHAPATVCGPKKSTNLVKNKCVN |
| | | F |
| A20 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 137 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAACATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCGTTACCGTCTGTTCCGTAAATCCAACC |
| | | TGAAACCGTTCGAACGTGACATCTCCACCGAAATCTACCAGGCGGGCTCCAAA |
| | | CCGTGCAACGGCGTTGAAGGCTTCAACTGCTACTTCCCGCTGCAGTCCTACGG |
| | | CTTCCAGCCGACCAACGGCGTTGGCTACCAGCCGTACCGTGTTGTTGTTCTGTC |
| | | CTTCGAACTGCTGCACGCGCCGGCGACCGTTTGCGGCCCGAAAAAATCCACCA |
| | | ACCTGGTTAAAAACAAATGCGTTAACTTC |
| A20 | SEQ | RVQPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGNIADYNYKLPDDFT |
| | NO: | GCVIAWNSNNLDSKVGGNYNYRYRLFRKSNLKPFERDISTEIYQAGSKPCNGVEG |
| | 138 | FNCYFPLQSYGFQPTNGVGYQPYRVVVLSFELLHAPATVCGPKKSTNLVKNKCVN |
| | | F |
| A21 | SEQ ID | CGTGTTCAGCCGACCGAATCCATAGTTAGGTTCCCGAACATCACTAACCTGTG |
| CDS | NO: | TCCGTTTGGCGAAGTGTTCAACGCGACCCGTTTTGCGTCCGTCTACGCCTGGA |
| | 139 | ACCGTAAACGTATCTCCAACTGCGTTGCGGACTACTCCGTTCTGTACAACTCCG |
| | | CGTCCTTCTCCACCTTCAAATGCTACGGCGTTTCCCCGACCAAACTGAACGACC |
| | | TGTGCTTCACCAACGTTTACGCGGACTCCTTCGTTATCCGTGGCGACGAAGTTC |
| | | GTCAGATCGCGCCGGGCCAGACCGGCAAAATCGCGGACTACAACTACAAACT |
| | | GCCGGACGACTTCACCGGCTGCGTTATCGCGTGGAACTCCAACAACCTGGACT |
| | | CCAAAGTTGGCGGCAACTACAACTACCAGTACCGTCTGTTCCGTAAATCCAAC |
| | | CTGAAACCGTTCGAACGTGACATCTCCACCGAAATCTACCAGGCGGGCTCCAC |
| | | CCCGTGCAACGGCGTTGAAGGCTTCAACTGCTACTCCCCGCTGCAGTCCTACG |
| | | GCTTCCAGCCGACCAACGGCGTTGGCTACCAGCCGTACCGTGTTGTTGTTCTG |
| | | TCCTTCGAACTGCTGCACGCGCCGGCGACCGTTTGCGGCCCGAAAAAATCCAC |
| | | CAACCTGGTTAAAAACAAATGCGTTAACTTC |
| A21 | SEQ | RVQPTESIVRFPNITNLCPFGEVFNATRFASVYAWNRKRISNCVADYSVLYNSASF |
| aa | ID | STFKCYGVSPTKLNDLCFTNVYADSFVIRGDEVRQIAPGQTGKIADYNYKLPDDFT |
| | NO: | GCVIAWNSNNLDSKVGGNYNYQYRLFRKSNLKPFERDISTEIYQAGSTPCNGVEG |
| | 140 | FNCYSPLQSYGFQPTNGVGYQPYRVVVLSFELLHAPATVCGPKKSTNLVKNKCVN |
| | | F |
| A22 | SEQ ID | CGTGTTCAGCCGACCGAATCCATAGTTAGGTTCCCGAACATCACTAACCTGTGTCCGTT |
| CDS | NO: | TGGCGAAGTGTTCAACGCGACCCGTTTTGCGTCCGTCTACGCCTGGAACCGTAAACGT |
| | 141 | ATCTCCAACTGCGTTGCGGACTACTCCGTTCTGTACAACTCCGCGTCCTTCTCCACCTTC |
| | | AAATGCTACGGCGTTTCCCCGACCAAACTGAACGACCTGTGCTTCACCAACGTTTACG |
| | | CGGACTCCTTCGTTATCCGTGGCGACGAAGTTCGTCAGATCGCGCCGGGCCAGACCG |
| | | GCAAAATCGCGGACTACAACTACAAACTGCCGGACGACTTCACCGGCTGCGTTATCGC |
| | | GTGGAACTCCAACAACCTGGACTCCAAAGTTGGCGGCAACTACAACTACCGTTACCGT |
| | | CTGTTCCGTAAATCCAACCTGAAACCGTTCGAACGTGACATCTCCACCGAAATCTACCA |
| | | GGCGGGCTCCAAACCGTGCAACGGCGTTGAAGGCTTCAACTGCTACTTCCCGCTGCA |
| | | GTCCTACGGCTTCCAGCCGACCAACGGCGTTGGCTACCAGCCGTACCGTGTTGTTGTT |
| | | CTGTCCTTCGAACTGCTGCACGCGCCGGCGACCGTTTGCGGCCCGAAAAAATCCACCA |
| | | ACCTGGTTAAAAACAAATGCGTTAACTTCCCGCGTCAGAAACGTACCGCGACCAAAGC |
| | | GTACAACGTTACCCAGGCGTTCGGCCGTCGTGGCCCGGAACAGACCCAGGGCAACTT |
| | | CGGCGACCAGGAACTGATCCGTCAGGGCACCGACTACAAACACTGGCCGCAGATCGC |
| | | GCAGTTCGCGCCGTCCGCGTCCGCGTTCTTCGGCATGTCCCGTATCGGCATGGAAGTT |
| | | ACCCCGTCCGGCACCTGGCTGACCTACACCGGCGCGATCAAACTGGACGACAAAGAC |
| | | CCGAACTTCAAAGACCAGGTTATCCTGCTGAACAAACACATCGACGCGTACAAA |
| A22 | SEQ | RVQPTESIVRFPNITNLCPFGEVFNATRFASVYAWNRKRISNCVADYSVLYNSASFSTFKCY |
| aa | ID | GVSPTKLNDLCFTNVYADSFVIRGDEVRQIAPGQTGKIADYNYKLPDDFTGCVIAWNSNN |
| | NO: | LDSKVGGNYNYRYRLFRKSNLKPFERDISTEIYQAGSKPCNGVEGFNCYFPLQSYGFQPTN |
| | 142 | GVGYQPYRVVVLSFELLHAPATVCGPKKSTNLVKNKCVNFPRQKRTATKAYNVTQAFGRR |
| | | GPEQTQGNFGDQELIRQGTDYKHWPQIAQFAPSASAFFGMSRIGMEVTPSGTWLTYTG |
| | | AIKLDDKDPNFKDQVILLNKHIDAYK |
| A23 | SEQ ID | CGTGTTCAGCCGACCGAATCCATAGTTAGGTTCCCGAACATCACTAACCTGTGTCCGTT |
| CDS | NO: | TGGCGAAGTGTTCAACGCGACCCGTTTTGCGTCCGTCTACGCCTGGAACCGTAAACGT |
| | 143 | ATCTCCAACTGCGTTGCGGACTACTCCGTTCTGTACAACTCCGCGTCCTTCTCCACCTTC |
| | | AAATGCTACGGCGTTTCCCCGACCAAACTGAACGACCTGTGCTTCACCAACGTTTACG |
| | | CGGACTCCTTCGTTATCCGTGGCGACGAAGTTCGTCAGATCGCGCCGGGCCAGACCG |
| | | GCAAAATCGCGGACTACAACTACAAACTGCCGGACGACTTCACCGGCTGCGTTATCGC |
| | | GTGGAACTCCAACAACCTGGACTCCAAAGTTGGCGGCAACTACAACTACCGTTACCGT |
| | | CTGTTCCGTAAATCCAACCTGAAACCGTTCGAACGTGACATCTCCACCGAAATCTACCA |
| | | GGCGGGCTCCAAACCGTGCAACGGCGTTGAAGGCTTCAACTGCTACTTCCCGCTGCA |
| | | GTCCTACGGCTTCCAGCCGACCAACGGCGTTGGCTACCAGCCGTACCGTGTTGTTGTT |
| | | CTGTCCTTCGAACTGCTGCACGCGCCGGCGACCGTTTGCGGCCCGAAAAAATCCACCA |
| | | ACCTGGTTAAAAACAAATGCGTTAACTTCGCGGCGCTGGCGCTGCTGCTGCTGGACC |
| | | GTCTGAACCAGCTGGAATCCAAAATGTCCGGCAAAGGCCAGCAGCAGCAGGGCCAG |
| | | ACCGTTACCAAAAAATCCGCGGCGGAAGCGTCCAAAAAACCGCGTCAGAAACGTACC |
| | | GCGACCAAAGCGTACAACGTTACCCAGGCGTTCGGCCGTCGTGGCCCGGAACAGACC |
| | | CAGGGCAACTTCGGCGACCAGGAACTGATCCGTCAGGGCACCGACTACAAACACTGG |
| | | CCGCAGATCGCGCAGTTCGCGCCGTCCGCGTCCGCGTTCTTCGGCATGTCCCGTATCG |
| | | GCATGGAAGTTACCCCGTCCGGCACCTGGCTGACCTACACCGGCGCGATCAAACTGG |
| | | ACGACAAAGACCCGAACTTCAAAGACCAGGTTATCCTGCTGAACAAACACATCGACG |
| | | CGTACAAAACCTTCCCGCCGACCGAACCGAAA |
| A23 | SEQ | RVQPTESIVRFPNITNLCPFGEVFNATRFASVYAWNRKRISNCVADYSVLYNSASFSTFKCY |
| aa | ID | GVSPTKLNDLCFTNVYADSFVIRGDEVRQIAPGQTGKIADYNYKLPDDFTGCVIAWNSNN |
| | NO: | LDSKVGGNYNYRYRLFRKSNLKPFERDISTEIYQAGSKPCNGVEGFNCYFPLQSYGFQPTN |
| | 144 | GVGYQPYRVVVLSFELLHAPATVCGPKKSTNLVKNKCVNFAALALLLLDRLNQLESKMSG |
| | | KGQQQQGQTVTKKSAAEASKKPRQKRTATKAYNVTQAFGRRGPEQTQGNFGDQELIRQ |
| | | GTDYKHWPQIAQFAPSASAFFGMSRIGMEVTPSGTWLTYTGAIKLDDKDPNFKDQVILL |
| | | NKHIDAYKTFPPTEPK |

TABLE 15
Sequences of Sall-fragments, optimized CDS and amino acid sequences (aa) of fusion
proteins of B-site in accordance with the invention

| | | DNA-sequence: 5′→3 |
|---|---|---|
| | | Sal-Sites: GTCATG |
| | | Promotor and Terminator regions: not underlined |
| | | CDS with optimized codon usage: underlined |
| Sal- | | CDS of viral antigenic unit (inclusive internal Linker) in bold |
| fragment/ | | Amino acid-sequence: Start→end |
| fusion | SEQ | Amino acids (aa) with optimized codon usage: underlined |
| proteins | ID | CDS of viral antigenic unit (inclusive Linker) in bold |
| B3 | SEQ ID | GTCGACCCTGCATGACCGCTTTTTGTACCAGCGTGAAAATGATGCGTGGAAGA |
| CDS | NO: | TTGATCGTCTTGCACCCTGAAAAGATGCAAAAATCTTGCTTTAATCGCTGGTAC |
| | 145 | TCCTGATTCTGGCACTTTATTCTATGTCTCTTTCGCATCTGGCGAAAAGTCGTGT |
| | | ACCGGCAAAGGTGCAGTCGTTATATACATGGAGATTTTGATGAAAAAAACCG |
| | | CGATCGCGATCGCGGTTGCGCTGGCGGGCTTCGCGACCGTTGCGCAGGCGGG |
| | | CAACTTCCTGACCGGCCTGGGCCACCGTTCCGACCACTACAACTGCGTTTCCTC |
| | | CGGCGGCCAGTGCCTGTACTCCGCGTGCCCGATCTTCACCAAAATCCAGGGCA |
| | | CCTGCTACCGTGGCAAAGCGAAATGCTGCAAAGAAGCGGCGGCGAAAGCGG |
| | | CGCTGGCGCTGCTGCTGCTGGACCGTCTGAACCAGCTGGAAGGCCCGGGCCC |
| | | GGGCAAATCCGCGGCGGAAGCGTCCAAAAAACCGCGTCAGAAACGTACCGC |
| | | GACCAAAGCGTACAACGTTACCCAGGCGTTCGGCCGTCGTGGCCCGGAACA |
| | | GACCCAGGGCAACTTCGGCGACCAGGAACTGATCCGTCAGGGCACCGACTA |
| | | CAAACACTGGCCGCAGATCGCGCAGTTCGCGCCGTCCGCGTCCGCGTTCTTC |
| | | GGCATGTCCCGTATCGGCATGGAAGTTACCCCGTCCGGCACCTGGCTGACCT |
| | | ACACCGGCGCGATCAAACTGGACGACAAAGACCCGAACTTCAAAGACCAGG |
| | | TTATCCTGCTGAACAAACACATCGACGCGTACAAAACCTTCCCGCCGACCGA |
| | | ACCGAAAAAAGCGGCGTACAAAACCTTCCCGCCGACCGAACCGAAAAAAGC |
| | | GGCGTACAAAACCTTCCCGCCGACCGAACCGAAAAAAGCGGCGTACATGGC |
| | | GTCCATGACCGGCGGCCAGCAGATGGGCTAATGACGCAACGCAATTAATGTG |
| | | AGTTAGCTCACTCATTAGGCACCGTCGAC |
| B3 | SEQ | MKKTAIAIAVALAGFATVAQAGNFLTGLGHRSDHYNCVSSGGQCLYSACPIFTKIQ |
| aa | ID | GTCYRGKAKCCKEAAAKAALALLLLDRLNQLEGPGPGKSAAEASKKPRQKRTAT |
| | NO: | KAYNVTQAFGRRGPEQTQGNFGDQELIRQGTDYKHWPQIAQFAPSASAFFG |
| | 146 | MSRIGMEVTPSGTWLTYTGAIKLDDKDPNFKDQVILLNKHIDAYKTFPPTEPKK |
| | | AAYKTFPPTEPKKAAYKTFPPTEPKKAAYMASMTGGQQMG* |
| B5 | SEQ ID | GTCGACCCTGCATGACCGCTTTTTGTACCAGCGTGAAAATGATGCGTGGAAGA |
| CDS | NO: | TTGATCGTCTTGCACCCTGAAAAGATGCAAAAATCTTGCTTTAATCGCTGGTAC |
| | 147 | TCCTGATTCTGGCACTTTATTCTATGTCTCTTTCGCATCTGGCGAAAAGTCGTGT |
| | | ACCGGCAAAGGTGCAGTCGTTATATACATGGAGATTTTGATGAAAAAAACCG |
| | | CGATCGCGATCGCGGTTGCGCTGGCGGGCTTCGCGACCGTTGCGCAGGCGGG |
| | | CAACTTCCTGACCGGCCTGGGCCACCGTTCCGACCACTACAACTGCGTTTCCTC |
| | | CGGCGGCCAGTGCCTGTACTCCGCGTGCCCGATCTTCACCAAAATCCAGGGCA |
| | | CCTGCTACCGTGGCAAAGCGAAATGCTGCAAAGAAGCGGCGGCGAAACCGC |
| | | GTCAGAAACGTACCGCGACCAAAGCGTACAACGTTACCCAGGCGTTCGGCC |
| | | GTCGTGGCCCGGAACAGACCCAGGGCAACTTCGGCGACCAGGAACTGATCC |
| | | GTCAGGGCACCGACTACAAACACTGGCCGCAGATCGCGCAGTTCGCGCCGTC |
| | | CGCGTCCGCGTTCTTCGGCATGTCCCGTATCGGCATGGAAGTTACCCCGTCCG |
| | | GCACCTGGCTGACCTACACCGGCGCGATCAAACTGGACGACAAAGACCCGA |
| | | ACTTCAAAGACCAGGTTATCCTGCTGAACAAACACATCGACGCGTACAAAGC |
| | | GGCGTACATGGCGTCCATGACCGGCGGCCAGCAGATGGGCTAATGACGCAAC |
| | | GCAATTAATGTGAGTTAGCTCACTCATTAGGCACCGTCGAC |
| B5 | SEQ | MKKTAIAIAVALAGFATVAQAGNFLTGLGHRSDHYNCVSSGGQCLYSACPIFTKIQ |
| Aa | ID | GTCYRGKAKCCKEAAAKPRQKRTATKAYNVTQAFGRRGPEQTQGNFGDGELIR |
| | NO: | QGTDYKHWPQIAQFAPSASAFFGMSRIGMEVTPSGTWLTYTGAIKLDDKDPN |
| | 148 | FKDQVILLNKHIDAYKAAYMASMTGGQQMG* |
| B7 | SEQ ID | GTCGACCCTGCATGACCGCTTTTTGTACCAGCGTGAAAATGATGCGTGGAAGA |
| CDS | NO: | TTGATCGTCTTGCACCCTGAAAAGATGCAAAAATCTTGCTTTAATCGCTGGTAC |
| | 149 | TCCTGATTCTGGCACTTTATTCTATGTCTCTTTCGCATCTGGCGAAAAGTCGTGT |
| | | ACCGGCAAAGGTGCAGTCGTTATATACATGGAGATTTTGATGTCCATCCAGCA |
| | | CTTCCGTGTTGCGCTGATCCCGTTCTTCGCGGCGTTCTGTCTCCCGGTATTCGC |
| | | CCACCCGGAAACCCTGGTTAAAGTTAAAGACGCGGAAGCGGCGGCGAAAGG |
| | | CAACTTCCTGACCGGCCTGGGCCACCGTTCCGACCACTACAACTGCGTTTCCTC |
| | | CGGCGGCCAGTGCCTGTACTCCGCGTGCCCGATCTTCACCAAAATCCAGGGCA |
| | | CCTGCTACCGTGGCAAAGCGAAATGCTGCAAAGAAGCGGCGGCGAAACCGC |
| | | GTCAGAAACGTACCGCGACCAAAGCGTACAACGTTACCCAGGCGTTCGGCC |
| | | GTCGTGGCCCGGAACAGACCCAGGGCAACTTCGGCGACCAGGAACTGATCC |
| | | GTCAGGGCACCGACTACAAACACTGGCCGCAGATCGCGCAGTTCGCGCCGTC |
| | | CGCGTCCGCGTTCTTCGGCATGTCCCGTATCGGCATGGAAGTTACCCCGTCCG |
| | | GCACCTGGCTGACCTACACCGGCGCGATCAAACTGGACGACAAAGACCCGA |
| | | ACTTCAAAGACCAGGTTATCCTGCTGAACAAACACATCGACGCGTACAAAGC |
| | | GGCGTACATGGCGTCCATGACCGGCGGCCAGCAGATGGGCTAATGACGCAAC |
| | | GCAATTAATGTGAGTTAGCTCACTCATTAGGCACCGTCGAC |
| B7 | SEQ | MSIQHFRVALIPFFAAFCLPVFAHPETLVKVKDAEAAAKGNFLTGLGHRSDHYNC |
| aa | ID | VSSGGQCLYSACPIFTKIQGTCYRGKAKCCKEAAAKPRQKRTATKAYNVTQAFGR |
| | NO: | RGPEQTQGNFGDQELIRQGTDYKHWPQIAQFAPSASAFFGMSRIGMEVTPSG |
| | 150 | TWLTYTGAIKLDDKDPNFKDQVILLNKHIDAYKAAYMASMTGGQQMG* |
| B9 | SEQ ID | GTCGACCCTGCATGACCGCTTTTTGTACCAGCGTGAAAATGATGCGTGGAAGA |
| CDS | NO: | TTGATCGTCTTGCACCCTGAAAAGATGCAAAAATCTTGCTTTAATCGCTGGTAC |
| | 151 | TCCTGATTCTGGCACTTTATTCTATGTCTCTTTCGCATCTGGCGAAAAGTCGTGT |
| | | ACCGGCAAAGGTGCAGTCGTTATATACATGGAGATTTTGATGTCCATCCAGCA |
| | | CTTCCGTGTTGCGCTGATCCCGTTCTTCGCGGCGTTCTGTCTCCCGGTATTCGC |
| | | CCACCCGGAAACCCTGGTTAAAGTTAAAGACGCGGAAGCGGCGGCGAAAGG |
| | | CATCGGCGACCCGGTTACCTGCCTGAAATCCGGCGCGATCTGCCACCCGGTTT |
| | | TCTGCCCGCGTCGTTACAAACAGATCGGCACCTGCGGCCTGCCGGGCACCAAA |
| | | TGCTGCAAAAAACCGGAAGCGGCGGCGAAACCGCGTCAGAAACGTACCGCG |
| | | ACCAAAGCGTACAACGTTACCCAGGCGTTCGGCCGTCGTGGCCCGGAACAG |
| | | ACCCAGGGCAACTTCGGCGACCAGGAACTGATCCGTCAGGGCACCGACTAC |
| | | AAACACTGGCCGCAGATCGCGCAGTTCGCGCCGTCCGCGTCCGCGTTCTTCG |
| | | GCATGTCCCGTATCGGCATGGAAGTTACCCCGTCCGGCACCTGGCTGACCTA |
| | | CACCGGCGCGATCAAACTGGACGACAAAGACCCGAACTTCAAAGACCAGGT |
| | | TATCCTGCTGAACAAACACATCGACGCGTACAAAGCGGCGTACATGGCGTCC |
| | | ATGACCGGCGGCCAGCAGATGGGCTAATGACGCAACGCAATTAATGTGAGTT |
| | | AGCTCACTCATTAGGCACCGTCGAC |
| B9 | SEQ | MSIQHFRVALIPFFAAFCLPVFAHPETLVKVKDAEAAAKGIGDPVTCLKSGAICHPV |
| aa | ID | FCPRRYKQIGTCGLPGTKCCKKPEAAAKPRQKRTATKAYNVTQAFGRRGPEQTQ |
| | NO: | GNFGDQELIRQGTDYKHWPQIAQFAPSASAFFGMSRIGMEVTPSGTWLTYTG |
| | 152 | AIKLDDKDPNFKDQVILLNKHIDAYKAAYMASMTGGQQMG* |
| B10 | SEQ ID | GTCGACGTAAATTCCTGGAGCTGAAGCAGAAGTTTCAACAGGGCGAAGTGCC |
| CDS | NO: | ATTGCCGAGCTTTTGGGGCGGTTTTCGCGTCAGCCTTGAACAGATTGAGTTCT |
| | 153 | GGCAGGGTGGTGAGCATCGCCTGCATGACCGCTTTTTGTACCAGCGTGAAAA |
| | | TGATGCGTGGAAGATTGATCGTCTTGCACCCTGAAAAGATGCAAAAATCTTGC |
| | | TTTAATCGCTGGTACTCCTGATTCTGGCACTTTATTCTATGTCTCTTTCGCATCT |
| | | GGCGAAAAGTCGTGTACCGGCAAAGGTGCAGTCGTTATATACATGGAGATTT |
| | | TGATGAAAAAAACCGCGATCGCGATCGCGGTTGCGCTGGCGGGCTTCGCGAC |
| | | CGTTGCGCAGGCGCCGCGTCAGAAACGTACCGCGACCAAAGCGTACAACGT |
| | | TACCCAGGCGTTCGGCCGTCGTGGCCCGGAACAGACCCAGGGCAACTTCGGC |
| | | GACCAGGAACTGATCCGTCAGGGCACCGACTACAAACACTGGCCGCAGATC |
| | | GCGCAGTTCGCGCCGTCCGCGTCCGCGTTCTTCGGCATGTCCCGTATCGGCAT |
| | | GGAAGTTACCCCGTCCGGCACCTGGCTGACCTACACCGGCGCGATCAAACTG |
| | | GACGACAAAGACCCGAACTTCAAAGACCAGGTTATCCTGCTGAACAAACAC |
| | | ATCGACGCGTACAAACACCACCACCACCACCACTAATTGTTCAGAACGCTCGG |
| | | TCTTGCACACCGGGCGTTTTTTCTTTGTGAGTCCAGTCGAC |
| B10 | SEQ | MKKTAIAIAVALAGFATVAQAPRQKRTATKAYNVTQAFGRRGPEQTQGNFGD |
| aa | ID | QELIRQGTDYKHWPQIAQFAPSASAFFGMSRIGMEVTPSGTWLTYTGAIKLDD |
| | NO: | KDPNFKDQVILLNKHIDAYKHHHHHH* |
| | 154 | |
| B11 | SEQ ID | GTCGACGTAAATTCCTGGAGCTGAAGCAGAAGTTTCAACAGGGCGAAGTGCC |
| CDS | NO: | ATTGCCGAGCTTTTGGGGCGGTTTTCGCGTCAGCCTTGAACAGATTGAGTTCT |
| | 155 | GGCAGGGTGGTGAGCATCGCCTGCATGACCGCTTTTTGTACCAGCGTGAAAA |
| | | TGATGCGTGGAAGATTGATCGTCTTGCACCCTGAAAAGATGCAAAAATCTTGC |
| | | TTTAATCGCTGGTACTCCTGATTCTGGCACTTTATTCTATGTCTCTTTCGCATCT |
| | | GGCGAAAAGTCGTGTACCGGCAAAGGTGCAGTCGTTATATACATGGAGATTT |
| | | TGATGAAAAAAACCGCGATCGCGATCGCGGTTGCGCTGGCGGGCTTCGCGAC |
| | | CGTTGCGCAGGCGGGCATCGGCGACCCGGTTACCTGCCTGAAATCCGGCGCG |
| | | ATCTGCCACCCGGTTTTCTGCCCGCGTCGTTACAAACAGATCGGCACCTGCGG |
| | | CCTGCCGGGCACCAAATGCTGCAAAAAACCGGAAGCGGCGGCGAAACCGCGT |
| | | CAGAAACGTACCGCGACCAAAGCGTACAACGTTACCCAGGCGTTCGGCCGTC |
| | | GTGGCCCGGAACAGACCCAGGGCAACTTCGGCGACCAGGAACTGATCCGTC |
| | | AGGGCACCGACTACAAACACTGGCCGCAGATCGCGCAGTTCGCGCCGTCCGC |
| | | GTCCGCGTTCTTCGGCATGTCCCGTATCGGCATGGAAGTTACCCCGTCCGGCA |
| | | CCTGGCTGACCTACACCGGCGCGATCAAACTGGACGACAAAGACCCGAACTT |
| | | CAAAGACCAGGTTATCCTGCTGAACAAACACATCGACGCGTACAAACACCAC |
| | | CACCACCACCACTAATTGTTCAGAACGCTCGGTCTTGCACACCGGGCGTTTTTT |
| | | CTTTGTGAGTCCAGTCGAC |
| B11 | SEQ | MKKTAIAIAVALAGFATVAQAGIGDPVTCLKSGAICHPVFCPRRYKQIGTCGLPGT |
| aa | ID | KCCKKPEAAAKPRQKRTATKAYNVTGAFGRRGPEQTQGNFGDQELIRQGTDYK |
| | NO: | HINPQIAQFAPSASAFFGMSRIGMEVTPSGTWLTYTGAIKLDDKDPNFKDQVIL |
| | 156 | LNKHIDAYKHHHHHH* |
| B12 | SEQ ID | GTCGACGTAAATTCCTGGAGCTGAAGCAGAAGTTTCAACAGGGCGAAGTGCC |
| CDS | NO: | ATTGCCGAGCTTTTGGGGCGGTTTTCGCGTCAGCCTTGAACAGATTGAGTTCT |
| | 157 | GGCAGGGTGGTGAGCATCGCCTGCATGACCGCTTTTTGTACCAGCGTGAAAA |
| | | TGATGCGTGGAAGATTGATCGTCTTGCACCCTGAAAAGATGCAAAAATCTTGC |
| | | TTTAATCGCTGGTACTCCTGATTCTGGCACTTTATTCTATGTCTCTTTCGCATCT |
| | | GGCGAAAAGTCGTGTACCGGCAAAGGTGCAGTCGTTATATACATGGAGATTT |
| | | TGATGAAAAAAACCGCGATCGCGATCGCGGTTGCGCTGGCGGGCTTCGCGAC |
| | | CGTTGCGCAGGCGGGCATCGGCGACCCGGTTACCTGCCTGAAATCCGGCGCG |
| | | ATCTGCCACCCGGTTTTCTGCCCGCGTCGTTACAAACAGATCGGCACCTGCGG |
| | | CCTGCCGGGCACCAAATGCTGCAAAAAACCGGAAGCGGCGGCGAAACACCAC |
| | | CACCACCACCACTAATTGTTCAGAACGCTCGGTCTTGCACACCGGGCGTTTTTT |
| | | CTTTGTGAGTCCAGTCGAC |
| B12 | SEQ | MKKTAIAIAVALAGFATVAQAGIGDPVTCLKSGAICHPVFCPRRYKQIGTCGLPGT |
| aa | ID | KCCKKPEAAAKHHHHHH* |
| | NO: | |
| | 158 | |
| B13 | SEQ ID | GTCGACGTAAATTCCTGGAGCTGAAGCAGAAGTTTCAACAGGGCGAAGTGCC |
| CDS | NO: | ATTGCCGAGCTTTTGGGGCGGTTTTCGCGTCAGCCTTGAACAGATTGAGTTCT |
| | 159 | GGCAGGGTGGTGAGCATCGCCTGCATGACCGCTTTTTGTACCAGCGTGAAAA |
| | | TGATGCGTGGAAGATTGATCGTCTTGCACCCTGAAAAGATGCAAAAATCTTGC |
| | | TTTAATCGCTGGTACTCCTGATTCTGGCACTTTATTCTATGTCTCTTTCGCATCT |
| | | GGCGAAAAGTCGTGTACCGGCAAAGGTGCAGTCGTTATATACATGGAGATTT |
| | | TGATGAAAAAAACCGCGATCGCGATCGCGGTTGCGCTGGCGGGCTTCGCGAC |
| | | CGTTGCGCAGGCGACCCCGCAGAACATCACCGACCTGTGCGCGGAATACCAC |
| | | AACACCCAGATCCACACCCTGAACGACAAAATCTTCTCCTACACCGAATCCCTG |
| | | GCGGGCAAACGTGAAATGGCGATCATCACCTTCAAAAACGGCGCGACCTTCC |
| | | AGGTTGAAGTTCCGGGCTCCCAGCACATCGACTCCCAGAAAAAAGCGATCGA |
| | | ACGTATGAAAGACACCCTGCGTATCGCGTACCTGACCGAAGCGAAAGTTGAA |
| | | AAACTGTGCGTTTGGAACAACAAAACCCCGCACGCGATCGCGGCGATCTCCAT |
| | | GGCGAACGAAGCGGCGGCGAAACACCACCACCACCACCACTAATTGTTCAGA |
| | | ACGCTCGGTCTTGCACACCGGGCGTTTTTTCTTTGTGAGTCCAGTCGAC |
| B13 | SEQ | MKKTAIAIAVALAGFATVAQATPQNITDLCAEYHNTQIHTLNDKIFSYTESLAGKRE |
| aa | ID | MAIITFKNGATFQVEVPGSQHIDSQKKAIERMKDTLRIAYLTEAKVEKLCVWNNK |
| | NO: | TPHAIAAISMANEAAAKHHHHHH* |
| | 160 | |
| B14 | SEQ ID | GTCGACGTAAATTCCTGGAGCTGAAGCAGAAGTTTCAACAGGGCGAAGTGCC |
| CDS | NO: | ATTGCCGAGCTTTTGGGGCGGTTTTCGCGTCAGCCTTGAACAGATTGAGTTCT |
| | 161 | GGCAGGGTGGTGAGCATCGCCTGCATGACCGCTTTTTGTACCAGCGTGAAAA |
| | | TGATGCGTGGAAGATTGATCGTCTTGCACCCTGAAAAGATGCAAAAATCTTGC |
| | | TTTAATCGCTGGTACTCCTGATTCTGGCACTTTATTCTATGTCTCTTTCGCATCT |
| | | GGCGAAAAGTCGTGTACCGGCAAAGGTGCAGTCGTTATATACATGGAGATTT |
| | | TGATGAAAAAAACCGCGATCGCGATCGCGGTTGCGCTGGCGGGCTTCGCGAC |
| | | CGTTGCGCAGGCGACCCCGCAGAACATCACCGACCTGTGCGCGGAATACCAC |
| | | AACACCCAGATCCACACCCTGAACGACAAAATCTTCTCCTACACCGAATCCCTG |
| | | GCGGGCAAACGTGAAATGGCGATCATCACCTTCAAAAACGGCGCGACCTTCC |
| | | AGGTTGAAGTTCCGGGCTCCCAGCACATCGACTCCCAGAAAAAAGCGATCGA |
| | | ACGTATGAAAGACACCCTGCGTATCGCGTACCTGACCGAAGCGAAAGTTGAA |
| | | AAACTGTGCGTTTGGAACAACAAAACCCCGCACGCGATCGCGGCGATCTCCAT |
| | | GGCGAACGAAGCGGCGGCGAAACCGCGTCAGAAACGTACCGCGACCAAAGC |
| | | GTACAACGTTACCCAGGCGTTCGGCCGTCGTGGCCCGGAACAGACCCAGGG |
| | | CAACTTCGGCGACCAGGAACTGATCCGTCAGGGCACCGACTACAAACACTG |
| | | GCCGCAGATCGCGCAGTTCGCGCCGTCCGCGTCCGCGTTCTTCGGCATGTCCC |
| | | GTATCGGCATGGAAGTTACCCCGTCCGGCACCTGGCTGACCTACACCGGCGC |
| | | GATCAAACTGGACGACAAAGACCCGAACTTCAAAGACCAGGTTATCCTGCTG |
| | | AACAAACACATCGACGCGTACAAACACCACCACCACCACCACTAATTGTTCAG |
| | | AACGCTCGGTCTTGCACACCGGGCGTTTTTTCTTTGTGAGTCCAGTCGA |
| B14 | SEQ | MKKTAIAIAVALAGFATVAQATPQNITDLCAEYHNTQIHTLNDKIFSYTESLAGKRE |
| aa | ID | MAIITFKNGATFQVEVPGSQHIDSQKKAIERMKDTLRIAYLTEAKVEKLCVWNNK |
| | NO: | TPHAIAAISMANEAAAKPRQKRTATKAYNVTGAFGRRGPEQTQGNFGDQELIR |
| | 162 | QGTDYKHWPQIAQFAPSASAFFGMSRIGMEVTPSGTWLTYTGAIKLDDKDPN |
| | | FKDQVILLNKHIDAYKHHHHHH* |
| B15 | SEQ ID | GTCGACGTAAATTCCTGGAGCTGAAGCAGAAGTTTCAACAGGGCGAAGTGCC |
| CDS | NO: | ATTGCCGAGCTTTTGGGGCGGTTTTCGCGTCAGCCTTGAACAGATTGAGTTCT |
| | 163 | GGCAGGGTGGTGAGCATCGCCTGCATGACCGCTTTTTGTACCAGCGTGAAAA |
| | | TGATGCGTGGAAGATTGATCGTCTTGCACCCTGAAAAGATGCAAAAATCTTGC |
| | | TTTAATCGCTGGTACTCCTGATTCTGGCACTTTATTCTATGTCTCTTTCGCATCT |
| | | GGCGAAAAGTCGTGTACCGGCAAAGGTGCAGTCGTTATATACATGGAGATTT |
| | | TGATGAAAAAAACCGCGATCGCGATCGCGGTTGCGCTGGCGGGCTTCGCGAC |
| | | CGTTGCGCAGGCGGCGGCGCTGGCGCTGCTGCTGCTGGACCGTCTGAACCAG |
| | | CTGGAAGGCCCGGGCCCGGGCAAATCCGCGGCGGAAGCGTCCAAAAAACC |
| | | GCGTCAGAAACGTACCGCGACCAAAGCGTACAACGTTACCCAGGCGTTCGG |
| | | CCGTCGTGGCCCGGAACAGACCCAGGGCAACTTCGGCGACCAGGAACTGAT |
| | | CCGTCAGGGCACCGACTACAAACACTGGCCGCAGATCGCGCAGTTCGCGCCG |
| | | TCCGCGTCCGCGTTCTTCGGCATGTCCCGTATCGGCATGGAAGTTACCCCGTC |
| | | CGGCACCTGGCTGACCTACACCGGCGCGATCAAACTGGACGACAAAGACCC |
| | | GAACTTCAAAGACCAGGTTATCCTGCTGAACAAACACATCGACGCGTACAAA |
| | | ACCTTCCCGCCGACCGAACCGAAAAAAGCGGCGTACAAAACCTTCCCGCCGA |
| | | CCGAACCGAAAAAAGCGGCGTACAAAACCTTCCCGCCGACCGAACCGAAAA |
| | | AAGCGGCGTACCACCACCACCACCACCACTAATTGTTCAGAACGCTCGGTCTT |
| | | GCACACCGGGCGTTTTTTCTTTGTGAGTCCAGTCGAC |
| B15 | SEQ | MKKTAIAIAVALAGFATVAQAAALALLLLDRLNQLEGPGPGKSAAEASKKPRQK |
| aa | ID | RTATKAYNVTQAFGRRGPEQTQGNFGDGELIRQGTDYKHWPGIAQFAPSASA |
| | NO: | FFGMSRIGMEVTPSGTWLTYTGAIKLDDKDPNFKDQVILLNKHIDAYKTFPPTE |
| | 164 | PKKAAYKTFPPTEPKKAAYKTFPPTEPKKAAYHHHHHH* |
| B16 | SEQ ID | GTCGACGTAAATTCCTGGAGCTGAAGCAGAAGTTTCAACAGGGCGAAGTGCC |
| CDS | NO: | ATTGCCGAGCTTTTGGGGCGGTTTTCGCGTCAGCCTTGAACAGATTGAGTTCT |
| | 165 | GGCAGGGTGGTGAGCATCGCCTGCATGACCGCTTTTTGTACCAGCGTGAAAA |
| | | TGATGCGTGGAAGATTGATCGTCTTGCACCCTGAAAAGATGCAAAAATCTTGC |
| | | TTTAATCGCTGGTACTCCTGATTCTGGCACTTTATTCTATGTCTCTTTCGCATCT |
| | | GGCGAAAAGTCGTGTACCGGCAAAGGTGCAGTCGTTATATACATGGAGATTT |
| | | TGATGAAAAAAACCGCGATCGCGATCGCGGTTGCGCTGGCGGGCTTCGCGAC |
| | | CGTTGCGCAGGCGACCCCGCAGAACATCACCGACCTGTGCGCGGAATACCAC |
| | | AACACCCAGATCCACACCCTGAACGACAAAATCTTCTCCTACACCGAATCCCTG |
| | | GCGGGCAAACGTGAAATGGCGATCATCACCTTCAAAAACGGCGCGACCTTCC |
| | | AGGTTGAAGTTCCGGGCTCCCAGCACATCGACTCCCAGAAAAAAGCGATCGA |
| | | ACGTATGAAAGACACCCTGCGTATCGCGTACCTGACCGAAGCGAAAGTTGAA |
| | | AAACTGTGCGTTTGGAACAACAAAACCCCGCACGCGATCGCGGCGATCTCCAT |
| | | GGCGAACGAAGCGGCGGCGAAAGCGGCGCTGGCGCTGCTGCTGCTGGACCG |
| | | TCTGAACCAGCTGGAAGGCCCGGGCCCGGGCAAATCCGCGGCGGAAGCGTC |
| | | CAAAAAACCGCGTCAGAAACGTACCGCGACCAAAGCGTACAACGTTACCCA |
| | | GGCGTTCGGCCGTCGTGGCCCGGAACAGACCCAGGGCAACTTCGGCGACCA |
| | | GGAACTGATCCGTCAGGGCACCGACTACAAACACTGGCCGCAGATCGCGCA |
| | | GTTCGCGCCGTCCGCGTCCGCGTTCTTCGGCATGTCCCGTATCGGCATGGAA |
| | | GTTACCCCGTCCGGCACCTGGCTGACCTACACCGGCGCGATCAAACTGGACG |
| | | ACAAAGACCCGAACTTCAAAGACCAGGTTATCCTGCTGAACAAACACATCGA |
| | | CGCGTACAAAACCTTCCCGCCGACCGAACCGAAAAAAGCGGCGTACAAAAC |
| | | CTTCCCGCCGACCGAACCGAAAAAAGCGGCGTACAAAACCTTCCCGCCGACC |
| | | GAACCGAAAAAAGCGGCGTACCACCACCACCACCACCACTAATTGTTCAGAA |
| | | CGCTCGGTCTTGCACACCGGGCGTTTTTTCTTTGTGAGTCCAGTCGA |
| B16 | SEQ | MKKTAIAIAVALAGFATVAQATPQNITDLCAEYHNTQIHTLNDKIFSYTESLAGKRE |
| aa | ID | MAIITFKNGATFQVEVPGSQHIDSQKKAIERMKDTLRIAYLTEAKVEKLCVWNNK |
| | NO: | TPHAIAAISMANEAAAKAALALLLLDRLNQLEGPGPGKSAAEASKKPRQKRTAT |
| | 166 | KAYNVTQAFGRRGPEQTQGNFGDIELIRQGTDYKHWPQIAQFAPSASAFFG |
| | | MSRIGMEVTPSGTWLTYTGAIKLDDKDPNFKDQVILLNKHIDAYKTFPPTEPKK |
| | | AAYKTFPPTEPKKAAYKTFPPTEPKKAAYHHHHHH* |

Note that the end of the translated sequence is denoted by an asterisk (*).

TABLE 16
optimized CDS inclusive internal linker (underlined) and amino
acid sequences (aa) inclusive internal linker (underlined) of
viral antigen units in fusion proteins of B-site in accordance
with the invention

| Viral | | |
|---|---|---|
| antigen | | DNA-sequence: 5′-> 3 |
| unit in | SEQ ID | Amino acid-sequence: Start -> end |
| B3 | SEQ ID | GCGGCGCTGGCGCTGCTGCTGCTGGACCGTCTGAACCAGCTGGAAGGCCC |
| CDS | NO: | GGGCCCGGGCAAATCCGCGGCGGAAGCGTCCAAAAAACCGCGTCAGAAAC |
| | 167 | GTACCGCGACCAAAGCGTACAACGTTACCCAGGCGTTCGGCCGTCGTGGCC |
| | | CGGAACAGACCCAGGGCAACTTCGGCGACCAGGAACTGATCCGTCAGGGC |
| | | ACCGACTACAAACACTGGCCGCAGATCGCGCAGTTCGCGCCGTCCGCGTCC |
| | | GCGTTCTTCGGCATGTCCCGTATCGGCATGGAAGTTACCCCGTCCGGCACCT |
| | | GGCTGACCTACACCGGCGCGATCAAACTGGACGACAAAGACCCGAACTTCA |
| | | AAGACCAGGTTATCCTGCTGAACAAACACATCGACGCGTACAAAACCTTCCC |
| | | GCCGACCGAACCGAAAAAAGCGGCGTACAAAACCTTCCCGCCGACCGAACC |
| | | GAAAAAAGCGGCGTACAAAACCTTCCCGCCGACCGAACCGAAAAAA |
| B3 | SEQ ID | AALALLLLDRLNQLEGPGPGKSAAEASKKPRQKRTATKAYNVTQAFGRRGPEQ |
| aa | NO: | TQGNFGDQELIRQGTDYKHWPQIAQFAPSASAFFGMSRIGMEVTPSGTWLTY |
| | 168 | TGAIKLDDKDPNFKDQVILLNKHIDAYKTFPPTEPKKAAYKTFPPTEPKKAAY |
| | | KTFPPTEPKK |
| B5 | SEQ ID | CCGCGTCAGAAACGTACCGCGACCAAAGCGTACAACGTTACCCAGGCGTTC |
| CDS | NO: | GGCCGTCGTGGCCCGGAACAGACCCAGGGCAACTTCGGCGACCAGGAACT |
| | 169 | GATCCGTCAGGGCACCGACTACAAACACTGGCCGCAGATCGCGCAGTTCGC |
| | | GCCGTCCGCGTCCGCGTTCTTCGGCATGTCCCGTATCGGCATGGAAGTTACC |
| | | CCGTCCGGCACCTGGCTGACCTACACCGGCGCGATCAAACTGGACGACAAA |
| | | GACCCGAACTTCAAAGACCAGGTTATCCTGCTGAACAAACACATCGACGCG |
| | | TACAAA |
| B5 | SEQ ID | PRQKRTATKAYNVTQAFGRRGPEQTQGNFGDQELIRQGTDYKHWPQIAQFA |
| Aa | NO: | PSASAFFGMSRIGMEVTPSGTWLTYTGAIKLDDKDPNFKDQVILLNKHIDAYK |
| | 170 | |
| B7 | SEQ ID | CCGCGTCAGAAACGTACCGCGACCAAAGCGTACAACGTTACCCAGGCGTTC |
| CDS | NO: | GGCCGTCGTGGCCCGGAACAGACCCAGGGCAACTTCGGCGACCAGGAACT |
| | 169 | GATCCGTCAGGGCACCGACTACAAACACTGGCCGCAGATCGCGCAGTTCGC |
| | | GCCGTCCGCGTCCGCGTTCTTCGGCATGTCCCGTATCGGCATGGAAGTTACC |
| | | CCGTCCGGCACCTGGCTGACCTACACCGGCGCGATCAAACTGGACGACAAA |
| | | GACCCGAACTTCAAAGACCAGGTTATCCTGCTGAACAAACACATCGACGCG |
| | | TACAAA |
| B7 | SEQ ID | PRQKRTATKAYNVTQAFGRRGPEQTQGNFGDQELIRQGTDYKHWPQIAQFA |
| aa | NO: | PSASAFFGMSRIGMEVTPSGTWLTYTGAIKLDDKDPNFKDQVILLNKHIDAYK |
| | 170 | |
| B9 | SEQ ID | CCGCGTCAGAAACGTACCGCGACCAAAGCGTACAACGTTACCCAGGCGTTC |
| CDS | NO: | GGCCGTCGTGGCCCGGAACAGACCCAGGGCAACTTCGGCGACCAGGAACT |
| | 169 | GATCCGTCAGGGCACCGACTACAAACACTGGCCGCAGATCGCGCAGTTCGC |
| | | GCCGTCCGCGTCCGCGTTCTTCGGCATGTCCCGTATCGGCATGGAAGTTACC |
| | | CCGTCCGGCACCTGGCTGACCTACACCGGCGCGATCAAACTGGACGACAAA |
| | | GACCCGAACTTCAAAGACCAGGTTATCCTGCTGAACAAACACATCGACGCG |
| | | TACAAA |
| B9 | SEQ ID | PRQKRTATKAYNVTQAFGRRGPEQTQGNFGDQELIRQGTDYKHWPQIAQFA |
| aa | NO: | PSASAFFGMSRIGMEVTPSGTWLTYTGAIKLDDKDPNFKDQVILLNKHIDAYK |
| | 170 | |
| B10 | SEQ ID | CCGCGTCAGAAACGTACCGCGACCAAAGCGTACAACGTTACCCAGGCGTTC |
| CDS | NO: | GGCCGTCGTGGCCCGGAACAGACCCAGGGCAACTTCGGCGACCAGGAACT |
| | 169 | GATCCGTCAGGGCACCGACTACAAACACTGGCCGCAGATCGCGCAGTTCGC |
| | | GCCGTCCGCGTCCGCGTTCTTCGGCATGTCCCGTATCGGCATGGAAGTTACC |
| | | CCGTCCGGCACCTGGCTGACCTACACCGGCGCGATCAAACTGGACGACAAA |
| | | GACCCGAACTTCAAAGACCAGGTTATCCTGCTGAACAAACACATCGACGCG |
| | | TACAAA |
| B10 | SEQ ID | PRQKRTATKAYNVTQAFGRRGPEQTQGNFGDQELIRQGTDYKHWPQIAQFA |
| aa | NO: | PSASAFFGMSRIGMEVTPSGTWLTYTGAIKLDDKDPNFKDQVILLNKHIDAYK |
| | 170 | |
| B11 | SEQ ID | CCGCGTCAGAAACGTACCGCGACCAAAGCGTACAACGTTACCCAGGCGTTC |
| CDS | NO: | GGCCGTCGTGGCCCGGAACAGACCCAGGGCAACTTCGGCGACCAGGAACT |
| | SEQ ID | GATCCGTCAGGGCACCGACTACAAACACTGGCCGCAGATCGCGCAGTTCGC |
| | NO: | GCCGTCCGCGTCCGCGTTCTTCGGCATGTCCCGTATCGGCATGGAAGTTACC |
| | 169 | CCGTCCGGCACCTGGCTGACCTACACCGGCGCGATCAAACTGGACGACAAA |
| | | GACCCGAACTTCAAAGACCAGGTTATCCTGCTGAACAAACACATCGACGCG |
| | | TACAAA |
| B11 | SEQ ID | PRQKRTATKAYNVTQAFGRRGPEQTQGNFGDQELIRQGTDYKHWPQIAQFA |
| aa | NO: | PSASAFFGMSRIGMEVTPSGTWLTYTGAIKLDDKDPNFKDQVILLNKHIDAYK |
| | 170 | |
| B12 | none | none |
| CDS | | |
| B12 | none | none |
| aa | | |
| B13 | none | none |
| CDS | | |
| B13 | none | none |
| aa | | |
| B14 | SEQ ID | CCGCGTCAGAAACGTACCGCGACCAAAGCGTACAACGTTACCCAGGCGTTC |
| CDS | NO: | GGCCGTCGTGGCCCGGAACAGACCCAGGGCAACTTCGGCGACCAGGAACT |
| | 169 | GATCCGTCAGGGCACCGACTACAAACACTGGCCGCAGATCGCGCAGTTCGC |
| | | GCCGTCCGCGTCCGCGTTCTTCGGCATGTCCCGTATCGGCATGGAAGTTACC |
| | | CCGTCCGGCACCTGGCTGACCTACACCGGCGCGATCAAACTGGACGACAAA |
| | | GACCCGAACTTCAAAGACCAGGTTATCCTGCTGAACAAACACATCGACGCG |
| | | TACAAA |
| B14 | SEQ ID | PRQKRTATKAYNVTQAFGRRGPEQTQGNFGDQELIRQGTDYKHWPQIAQFA |
| aa | NO: | PSASAFFGMSRIGMEVTPSGTWLTYTGAIKLDDKDPNFKDQVILLNKHIDAYK |
| | 170 | |
| B15 | SEQ ID | GCGGCGCTGGCGCTGCTGCTGCTGGACCGTCTGAACCAGCTGGAAGGCCC |
| CDS | NO: | GGGCCCGGGCAAATCCGCGGCGGAAGCGTCCAAAAAACCGCGTCAGAAAC |
| | 167 | GTACCGCGACCAAAGCGTACAACGTTACCCAGGCGTTCGGCCGTCGTGGCC |
| | | CGGAACAGACCCAGGGCAACTTCGGCGACCAGGAACTGATCCGTCAGGGC |
| | | ACCGACTACAAACACTGGCCGCAGATCGCGCAGTTCGCGCCGTCCGCGTCC |
| | | GCGTTCTTCGGCATGTCCCGTATCGGCATGGAAGTTACCCCGTCCGGCACCT |
| | | GGCTGACCTACACCGGCGCGATCAAACTGGACGACAAAGACCCGAACTTCA |
| | | AAGACCAGGTTATCCTGCTGAACAAACACATCGACGCGTACAAAACCTTCCC |
| | | GCCGACCGAACCGAAAAAAGCGGCGTACAAAACCTTCCCGCCGACCGAACC |
| | | GAAAAAAGCGGCGTACAAAACCTTCCCGCCGACCGAACCGAAAAAA |
| B15 | SEQ ID | AALALLLLDRLNQLEGPGPGKSAAEASKKPRQKRTATKAYNVTQAFGRRGPEQ |
| aa | NO: | TQGNFGDQELIRQGTDYKHWPQIAQFAPSASAFFGMSRIGMEVTPSGTWLTY |
| | 168 | TGAIKLDDKDPNFKDQVILLNKHIDAYKTFPPTEPKKAAYKTFPPTEPKKAAY |
| | | KTFPPTEPKK |
| B16 | SEQ ID | GCGGCGCTGGCGCTGCTGCTGCTGGACCGTCTGAACCAGCTGGAAGGCCC |
| CDS | NO: | GGGCCCGGGCAAATCCGCGGCGGAAGCGTCCAAAAAACCGCGTCAGAAAC |
| | 167 | GTACCGCGACCAAAGCGTACAACGTTACCCAGGCGTTCGGCCGTCGTGGCC |
| | | CGGAACAGACCCAGGGCAACTTCGGCGACCAGGAACTGATCCGTCAGGGC |
| | | ACCGACTACAAACACTGGCCGCAGATCGCGCAGTTCGCGCCGTCCGCGTCC |
| | | GCGTTCTTCGGCATGTCCCGTATCGGCATGGAAGTTACCCCGTCCGGCACCT |
| | | GGCTGACCTACACCGGCGCGATCAAACTGGACGACAAAGACCCGAACTTCA |
| | | AAGACCAGGTTATCCTGCTGAACAAACACATCGACGCGTACAAAACCTTCCC |
| | | GCCGACCGAACCGAAAAAAGCGGCGTACAAAACCTTCCCGCCGACCGAACC |
| | | GAAAAAAGCGGCGTACAAAACCTTCCCGCCGACCGAACCGAAAAAA |
| B16 | SEQ ID | AALALLLLDRLNQLEGPGPGKSAAEASKKPRQKRTATKAYNVTQAFGRRGPEQ |
| aa | NO: | TQGNFGDQELIRQGTDYKHWPQIAQFAPSASAFFGMSRIGMEVTPSGTWLTY |
| | 168 | TGAIKLDDKDPNFKDQVILLNKHIDAYKTFPPTEPKKAAYKTFPPTEPKKAAY |
| | | KTFPPTEPKK |

TABLE 17
TyrS expression cassettes (EPC) used in accordance with the invention

| | | DNA-sequence: 5′-> 3 |
|---|---|---|
| | | Spel-Sites: ACTAGT |
| | | DNA with optimized codon usage: underlined |
| | | CDS in bold |
| Expression | SEQ | Amino acid-sequence: Start -> end |
| cassettes | ID | Amino acids (aa) with optimized codon usage: underlined |
| P_{lacl-like} tyrS | SEQ | ACTAGTGCTAGCGACACCATCGAATGGCGCAAACCTTTCGCGGTATGGCATG |
| EPC | ID | ATAGCGCCCGAAGTCGTGTACCGGCAAAGGTGAGTCGTTATATACATGGAG |
| | NO: | ATTTTGATGGCAAGCAGTAACTTGATTAAACAATTGCAAGAGCGGGGGCT |
| | 171 | GGTAGCCCAGGTGACGGACGAGGAAGCGTTAGCAGAGCGACTGGCGCAA |
| | | GGCCCGATCGCGCTCTATTGCGGCTTCGATCCTACCGCTGACAGCTTGCATT |
| | | TGGGGCATCTTGTTCCATTGTTATGCCTGAAACGCTTCCAGCAGGCGGGCC |
| | | ACAAGCCGGTTGCGCTGGTAGGCGGCGCGACGGGTCTGATTGGCGACCCG |
| | | AGCTTCAAAGCTGCCGAGCGTAAGCTGAACACCGAAGAAACTGTTCAGGA |
| | | GTGGGTGGACAAAATCCGTAAGCAGGTTGCCCCGTTCCTCGATTTCGACTG |
| | | TGGAGAAAACTCTGCTATCGCGGCGAACAACTATGACTGGTTCGGCAATAT |
| | | GAATGTGCTGACCTTCCTGCGCGATATTGGCAAACACTTCTCCGTTAACCAG |
| | | ATGATCAACAAAGAAGCGGTTAAGCAGCGTCTCAACCGTGAAGATCAGGG |
| | | GATTTCGTTCACTGAGTTTTCCTACAACCTGTTGCAGGGTTATGACTTCGCC |
| | | TGTCTGAACAAACAGTACGGTGTGGTGCTGCAAATTGGTGGTTCTGACCAG |
| | | TGGGGTAACATCACTTCTGGTATCGACCTGACCCGTCGTCTGCATCAGAATC |
| | | AGGTGTTTGGCCTGACCGTTCCGCTGATCACTAAAGCAGATGGCACCAAAT |
| | | TTGGTAAAACTGAAGGCGGCGCAGTCTGGTTGGATCCGAAGAAAACCAGC |
| | | CCGTACAAATTCTACCAGTTCTGGATCAACACTGCGGATGCCGACGTTTACC |
| | | GCTTCCTGAAGTTCTTCACCTTTATGAGCATTGAAGAGATCAACGCCCTGG |
| | | AAGAAGAAGATAAAAACAGCGGTAAAGCACCGCGCGCCCAGTATGTACT |
| | | GGCGGAGCAGGTGACTCGTCTGGTTCACGGTGAAGAAGGTTTACAGGCGG |
| | | CAAAACGTATTACCGAATGCCTGTTCAGCGGTTCTTTGAGTGCGCTGAGTG |
| | | AAGCGGACTTCGAACAGCTGGCGCAGGACGGCGTACCGATGGTTGAGATG |
| | | GAAAAGGGCGCAGACCTGATGCAGGCACTGGTCGATTCTGAACTGCAACC |
| | | TTCCCGTGGTCAGGCACGTAAAACTATCGCCTCCAATGCCATCACCATTAAC |
| | | GGTGAAAAACAGTCCGATCCTGAATACTTCTTTAAAGAAGAAGATCGTCTG |
| | | TTTGGTCGTTTTACCTTACTGCGTCGCGGTAAAAAGAATTACTGTCTGATTT |
| | | GCTGGAAACATCACCATCACCATCACTAATCCACGGCCGCCAGTTTGGGCT |
| | | GGCGGCATTTTGGTACCACTAGT |
| P_{lacl-like} | SEQ | MASSNLIKQLQERGLVAQVTDEEALAERLAQGPIALYCGFDPTADSLHLGHLVP |
| tyrS EPC | ID | LLCLKRFQQAGHKPVALVGGATGLIGDPSFKAAERKLNTEETVQEWVDKIRKQV |
| aa | NO: | APFLDFDCGENSAIAANNYDWFGNMNVLTFLRDIGKHFSVNQMINKEAVKQR |
| | 172 | LNREDQGISFTEFSYNLLQGYDFACLNKQYGVVLQIGGSDQWGNITSGIDLTRRL |
| | | HQNQVFGLTVPLITKADGTKFGKTEGGAVWLDPKKTSPYKFYQFWINTADADV |
| | | YRFLKFFTFMSIEEINALEEEDKNSGKAPRAQYVLAEQVTRLVHGEEGLQAAKRI |
| | | TECLFSGSLSALSEADFEQLAQDGVPMVEMEKGADLMQALVDSELQPSRGQA |
| | | RKTIASNAITINGEKQSDPEYFFKEEDRLFGRFTLLRRGKKNYCLICWKHHHHHH |
| | | * |
| P_{lacl-like} tyrS | SEQ | ACTAGTGCTAGCGACACCATCGAATGGCGCAAACCTTTCGCGGTATGGCATG |
| EPC | ID | ATAGCGCCCGAAGTCGTGTACCGGCAAAGGTGAGTCGTTATATACATGGAG |
| With T0 | NO: | ATTTTGATGGCAAGCAGTAACTTGATTAAACAATTGCAAGAGCGGGGGCT |
| | 173 | GGTAGCCCAGGTGACGGACGAGGAAGCGTTAGCAGAGCGACTGGCGCAA |
| | | GGCCCGATCGCGCTCTATTGCGGCTTCGATCCTACCGCTGACAGCTTGCATT |
| | | TGGGGCATCTTGTTCCATTGTTATGCCTGAAACGCTTCCAGCAGGCGGGCC |
| | | ACAAGCCGGTTGCGCTGGTAGGCGGCGCGACGGGTCTGATTGGCGACCCG |
| | | AGCTTCAAAGCTGCCGAGCGTAAGCTGAACACCGAAGAAACTGTTCAGGA |
| | | GTGGGTGGACAAAATCCGTAAGCAGGTTGCCCCGTTCCTCGATTTCGACTG |
| | | TGGAGAAAACTCTGCTATCGCGGCGAACAACTATGACTGGTTCGGCAATAT |
| | | GAATGTGCTGACCTTCCTGCGCGATATTGGCAAACACTTCTCCGTTAACCAG |
| | | ATGATCAACAAAGAAGCGGTTAAGCAGCGTCTCAACCGTGAAGATCAGGG |
| | | GATTTCGTTCACTGAGTTTTCCTACAACCTGTTGCAGGGTTATGACTTCGCC |
| | | TGTCTGAACAAACAGTACGGTGTGGTGCTGCAAATTGGTGGTTCTGACCAG |
| | | TGGGGTAACATCACTTCTGGTATCGACCTGACCCGTCGTCTGCATCAGAATC |
| | | AGGTGTTTGGCCTGACCGTTCCGCTGATCACTAAAGCAGATGGCACCAAAT |
| | | TTGGTAAAACTGAAGGCGGCGCAGTCTGGTTGGATCCGAAGAAAACCAGC |
| | | CCGTACAAATTCTACCAGTTCTGGATCAACACTGCGGATGCCGACGTTTACC |
| | | GCTTCCTGAAGTTCTTCACCTTTATGAGCATTGAAGAGATCAACGCCCTGG |
| | | AAGAAGAAGATAAAAACAGCGGTAAAGCACCGCGCGCCCAGTATGTACT |
| | | GGCGGAGCAGGTGACTCGTCTGGTTCACGGTGAAGAAGGTTTACAGGCGG |
| | | CAAAACGTATTACCGAATGCCTGTTCAGCGGTTCTTTGAGTGCGCTGAGTG |
| | | AAGCGGACTTCGAACAGCTGGCGCAGGACGGCGTACCGATGGTTGAGATG |
| | | GAAAAGGGCGCAGACCTGATGCAGGCACTGGTCGATTCTGAACTGCAACC |
| | | TTCCCGTGGTCAGGCACGTAAAACTATCGCCTCCAATGCCATCACCATTAAC |
| | | GGTGAAAAACAGTCCGATCCTGAATACTTCTTTAAAGAAGAAGATCGTCTG |
| | | TTTGGTCGTTTTACCTTACTGCGTCGCGGTAAAAAGAATTACTGTCTGATTT |
| | | GCTGGAAACATCACCATCACCATCACTAATTGTTCAGAACGCTCGGTCTTGC |
| | | ACACCGGGCGTTTTTTCTTTGTGAGTCCAACTAGT |
| P_{lacl-like} tyrS | SEQ | MASSNLIKQLQERGLVAQVTDEEALAERLAQGPIALYCGFDPTADSLHLGHLVP |
| EPC | ID | LLCLKRFQQAGHKPVALVGGATGLIGDPSFKAAERKLNTEETVQEWVDKIRKQV |
| aa | NO: | APFLDFDCGENSAIAANNYDWFGNMNVLTFLRDIGKHFSVNQMINKEAVKQR |
| With T0 | 172 | LNREDQGISFTEFSYNLLQGYDFACLNKQYGVVLQIGGSDQWGNITSGIDLTRRL |
| | | HQNQVFGLTVPLITKADGTKFGKTEGGAVWLDPKKTSPYKFYQFWINTADADV |
| | | YRFLKFFTFMSIEEINALEEEDKNSGKAPRAQYVLAEQVTRLVHGEEGLQAAKRI |
| | | TECLFSGSLSALSEADFEQLAQDGVPMVEMEKGADLMQALVDSELQPSRGQA |
| | | RKTIASNAITINGEKQSDPEYFFKEEDRLFGRFTLLRRGKKNYCLICWKHHHHHH |
| | | * |
| P_{lacl-}tyrS EPC | SEQ | ACTAGTGACACCATCGAATGGCGCAAAACCTTTCGCGGTATGGCATGATAGC |
| With T0 | ID | GCCCGGAAGTCGTGTACCGGCAAAGGTGCAGTCGTTATATACATGGAGATT |
| | NO: | TTGATGGCAAGCAGTAACTTGATTAAACAATTGCAAGAGCGGGGGCTGGT |
| | 174 | AGCCCAGGTGACGGACGAGGAAGCGTTAGCAGAGCGACTGGCGCAAGGC |
| | | CCGATCGCGCTCTATTGCGGCTTCGATCCTACCGCTGACAGCTTGCATTTGG |
| | | GGCATCTTGTTCCATTGTTATGCCTGAAACGCTTCCAGCAGGCGGGCCACA |
| | | AGCCGGTTGCGCTGGTAGGCGGCGCGACGGGTCTGATTGGCGACCCGAGC |
| | | TTCAAAGCTGCCGAGCGTAAGCTGAACACCGAAGAAACTGTTCAGGAGTG |
| | | GGTGGACAAAATCCGTAAGCAGGTTGCCCCGTTCCTCGATTTCGACTGTGG |
| | | AGAAAACTCTGCTATCGCGGCGAACAACTATGACTGGTTCGGCAATATGA |
| | | ATGTGCTGACCTTCCTGCGCGATATTGGCAAACACTTCTCCGTTAACCAGAT |
| | | GATCAACAAAGAAGCGGTTAAGCAGCGTCTCAACCGTGAAGATCAGGGG |
| | | ATTTCGTTCACTGAGTTTTCCTACAACCTGTTGCAGGGTTATGACTTCGCCT |
| | | GTCTGAACAAACAGTACGGTGTGGTGCTGCAAATTGGTGGTTCTGACCAGT |
| | | GGGGTAACATCACTTCTGGTATCGACCTGACCCGTCGTCTGCATCAGAATC |
| | | AGGTGTTTGGCCTGACCGTTCCGCTGATCACTAAAGCAGATGGCACCAAAT |
| | | TTGGTAAAACTGAAGGCGGCGCAGTCTGGTTGGATCCGAAGAAAACCAGC |
| | | CCGTACAAATTCTACCAGTTCTGGATCAACACTGCGGATGCCGACGTTTACC |
| | | GCTTCCTGAAGTTCTTCACCTTTATGAGCATTGAAGAGATCAACGCCCTGG |
| | | AAGAAGAAGATAAAAACAGCGGTAAAGCACCGCGCGCCCAGTATGTACT |
| | | GGCGGAGCAGGTGACTCGTCTGGTTCACGGTGAAGAAGGTTTACAGGCGG |
| | | CAAAACGTATTACCGAATGCCTGTTCAGCGGTTCTTTGAGTGCGCTGAGTG |
| | | AAGCGGACTTCGAACAGCTGGCGCAGGACGGCGTACCGATGGTTGAGATG |
| | | GAAAAGGGCGCAGACCTGATGCAGGCACTGGTCGATTCTGAACTGCAACC |
| | | TTCCCGTGGTCAGGCACGTAAAACTATCGCCTCCAATGCCATCACCATTAAC |
| | | GGTGAAAAACAGTCCGATCCTGAATACTTCTTTAAAGAAGAAGATCGTCTG |
| | | TTTGGTCGTTTTACCTTACTGCGTCGCGGTAAAAAGAATTACTGTCTGATTT |
| | | GCTGGAAACATCACCATCACCATCACTAATTGTTCAGAACGCTCGGTCTTGC |
| | | ACACCGGGCGTTTTTTCTTTGTGAGTCCAACTAGT |
| P_{lacl-} tyrS EPC | SEQ | MASSNLIKQLQERGLVAQVTDEEALAERLAQGPIALYCGFDPTADSLHLGHLVP |
| aa | ID | LLCLKRFQQAGHKPVALVGGATGLIGDPSFKAAERKLNTEETVQEWVDKIRKQV |
| With T0 | NO: | APFLDFDCGENSAIAANNYDWFGNMNVLTFLRDIGKHFSVNQMINKEAVKQR |
| | 172 | LNREDQGISFTEFSYNLLQGYDFACLNKQYGVVLQIGGSDQWGNITSGIDLTRRL |
| | | HQNQVFGLTVPLITKADGTKFGKTEGGAVWLDPKKTSPYKFYQFWINTADADV |
| | | YRFLKFFTFMSIEEINALEEEDKNSGKAPRAQYVLAEQVTRLVHGEEGLQAAKRI |
| | | TECLFSGSLSALSEADFEQLAQDGVPMVEMEKGADLMQALVDSELQPSRGQA |
| | | RKTIASNAITINGEKQSDPEYFFKEEDRLFGRFTLLRRGKKNYCLICWKHHHHHH |
| | | * |
| P_{lacl}tyrS EPC | SEQ | ACTAGTGACACCATCGAATGGCGCAAAACCTTTCGCGGTATGGCATGATAGC |
| With T0 | ID | GCCCGGAAGTCGTGTACCGGCAAAGGTGCAGTCGTTATATACATGGAGATT |
| And | NO: | TTGATGGCGTCCTCCAACCTGATCAAACAGCTGCAGGAACGTGGCCTGGTT |
| optimized | 175 | GCGCAGGTTACCGACGAAGAAGCGCTGGCGGAACGTCTGGCGCAGGGCC |
| | | CGATCGCGCTGTACTGCGGCTTCGACCCGACCGCGGACTCCCTGCACCTGG |
| | | GCCACCTGGTTCCGCTGCTGTGCCTGAAACGTTTCCAGCAGGCGGGCCACA |
| | | AACCGGTTGCGCTGGTTGGCGGCGCGACCGGCCTGATCGGCGACCCGTCCT |
| | | TCAAAGCGGCGGAACGTAAACTGAACACCGAAGAAACCGTTCAGGAATG |
| | | GGTTGACAAAATCCGTAAACAGGTTGCGCCGTTCCTGGACTTCGACTGCGG |
| | | CGAAAACTCCGCGATCGCGGCGAACAACTACGACTGGTTCGGCAACATGA |
| | | ACGTTCTGACCTTCCTGCGTGACATCGGCAAACACTTCTCCGTTAACCAGAT |
| | | GATCAACAAAGAAGCGGTTAAACAGCGTCTGAACCGTGAAGACCAGGGC |
| | | ATCTCCTTCACCGAATTCTCCTACAACCTGCTGCAGGGCTACGACTTCGCGT |
| | | GCCTGAACAAACAGTACGGCGTTGTTCTGCAGATCGGCGGCTCCGACCAGT |
| | | GGGGCAACATCACCTCCGGCATCGACCTGACCCGTCGTCTGCACCAAAATC |
| | | AGGTGTTCGGGCTGACCGTTCCGCTGATCACCAAAGCGGACGGCACCAAA |
| | | TTCGGCAAAACCGAAGGCGGCGCGGTTTGGCTGGACCCGAAAAAAACCTC |
| | | CCCGTACAAATTCTACCAGTTCTGGATCAACACAGCGGACGCGGACGTATA |
| | | CAGATTCCTGAAATTCTTCACCTTCATGTCCATCGAAGAAATCAACGCGCTG |
| | | GAAGAAGAAGACAAAAACTCCGGCAAAGCGCCGCGTGCGCAGTACGTTCT |
| | | GGCGGAACAGGTTACCCGTCTGGTTCACGGCGAAGAAGGCCTGCAGGCGG |
| | | CGAAACGTATCACCGAATGCCTGTTCTCCGGCTCCCTGTCCGCGCTGTCCGA |
| | | AGCGGACTTCGAACAGCTGGCGCAGGACGGCGTTCCGATGGTTGAAATGG |
| | | AAAAAGGCGCGGACCTGATGCAGGCGCTGGTTGACTCCGAACTGCAGCCG |
| | | TCCCGTGGCCAGGCGCGTAAAACCATCGCGTCCAACGCGATCACCATCAAC |
| | | GGCGAAAAACAGTCCGACCCGGAATACTTCTTCAAAGAAGAAGACCGTCT |
| | | GTTCGGCCGTTTCACCCTGCTGCGTCGTGGCAAAAAAAACTACTGCCTGAT |
| | | CTGCTGGAAACACCACCACCACCACCACTAATTGTTCAGAACGCTCGGTCTT |
| | | GCACACCGGGCGTTTTTTCTTTGTGAGTCCAACTAGT |
| P_{lacl}tyrS EPC | SEQ | MASSNLIKQLQERGLVAQVTDEEALAERLAQGPIALYCGFDPTADSLHLGHLVP |
| 1 | ID | LLCLKRFQQAGHKPVALVGGATGLIGDPSFKAAERKLNTEETVQEWVDKIRKQV |
| aa | NO: | APFLDFDCGENSAIAANNYDWFGNMNVLTFLRDIGKHFSVNQMINKEAVKQR |
| | 172 | LNREDQGISFTEFSYNLLQGYDFACLNKQYGVVLQIGGSDQWGNITSGIDLTRRL |
| | | HQNQVFGLTVPLITKADGTKFGKTEGGAVWLDPKKTSPYKFYQFWINTADADV |
| | | YRFLKFFTFMSIEEINALEEEDKNSGKAPRAQYVLAEQVTRLVHGEEGLQAAKRI |
| | | TECLFSGSLSALSEADFEQLAQDGVPMVEMEKGADLMQALVDSELQPSRGQA |
| | | RKTIASNAITINGEKQSDPEYFFKEEDRLFGRFTLLRRGKKNYCLICWKHHHHHH |
| | | * |

Note that the end of the translated sequence is denoted by an asterisk (*).

CDS of CtxB - mature protein - AAC34728.1
(SEQ ID NO: 176)
ACACCTCAAAATATTACTGATTTGTGTGCAGAATACCACAACACACAAATACATACGCTA
AATGATAAGATATTTTCGTATACAGAATCTCTAGCTGGAAAAAGAGAGATGGCTATCATT
ACTTTTAAGAATGGTGCAACTTTTCAAGTAGAAGTACCAGGTAGTCAACATATAGATTCA
CAAAAAAAAGCGATTGAAAGGATGAAGGATACCCTGAGGATTGCATATCTTACTGAAGC
TAAAGTCGAAAAGTTATGTGTATGGAATAATAAAACGCCTCATGCGATTGCCGCAATTAG
TATGGCAAAT
CDS CtxB unit in JMU-SalVac-100 System (improved DNA)
(SEQ ID NO: 177)
ACCCCGCAGAACATCACCGACCTGTGCGCGGAATACCACAACACCCAGATCCACACCCTG
AACGACAAAATCTTCTCCTACACCGAATCCCTGGCGGGCAAACGTGAAATGGCGATCATC
ACCTTCAAAAACGGCGCGACCTTCCAGGTTGAAGTTCCGGGCTCCCAGCACATCGACTCC
CAGAAAAAAGCGATCGAACGTATGAAAGACACCCTGCGTATCGCGTACCTGACCGAAGC
GAAAGTTGAAAAACTGTGCGTTTGGAACAACAAAACCCCGCACGCGATCGCGGCGATCT
CCATGGCGAAC
S-Protein Wuhan Hu-1, GeneID 43740568 - NC_045512.2, Us
converrted to Ts
(SEQ ID NO: 178)
ATGTTTGTTTTTCTTGTTTTATTGCCACTAGTCTCTAGTCAGTGTGTTAATCTTACAACCAG
AACTCAATTACCCCCTGCATACACTAATTCTTTCACACGTGGTGTTTATTACCCTGACAAA
GTTTTCAGATCCTCAGTTTTACATTCAACTCAGGACTTGTTCTTACCTTTCTTTTCCAATGT
TACTTGGTTCCATGCTATACATGTCTCTGGGACCAATGGTACTAAGAGGTTTGATAACCCT
GTCCTACCATTTAATGATGGTGTTTATTTTGCTTCCACTGAGAAGTCTAACATAATAAGAG
GCTGGATTTTTGGTACTACTTTAGATTCGAAGACCCAGTCCCTACTTATTGTTAATAACGC
TACTAATGTTGTTATTAAAGTCTGTGAATTTCAATTTTGTAATGATCCATTTTTGGGTGTTT
ATTACCACAAAAACAACAAAAGTTGGATGGAAAGTGAGTTCAGAGTTTATTCTAGTGCGA
ATAATTGCACTTTTGAATATGTCTCTCAGCCTTTTCTTATGGACCTTGAAGGAAAACAGGG
TAATTTCAAAAATCTTAGGGAATTTGTGTTTAAGAATATTGATGGTTATTTTAAAATATAT
TCTAAGCACACGCCTATTAATTTAGTGCGTGATCTCCCTCAGGGTTTTTCGGCTTTAGAAC
CATTGGTAGATTTGCCAATAGGTATTAACATCACTAGGTTTCAAACTTTACTTGCTTTACA
TAGAAGTTATTTGACTCCTGGTGATTCTTCTTCAGGTTGGACAGCTGGTGCTGCAGCTTAT
TATGTGGGTTATCTTCAACCTAGGACTTTTCTATTAAAATATAATGAAAATGGAACCATTA
CAGATGCTGTAGACTGTGCACTTGACCCTCTCTCAGAAACAAAGTGTACGTTGAAATCCT
TCACTGTAGAAAAAGGAATCTATCAAACTTCTAACTTTAGAGTCCAACCAACAGAATCTA
TTGTTAGATTTCCTAATATTACAAACTTGTGCCCTTTTGGTGAAGTTTTTAACGCCACCAG
ATTTGCATCTGTTTATGCTTGGAACAGGAAGAGAATCAGCAACTGTGTTGCTGATTATTCT
GTCCTATATAATTCCGCATCATTTTCCACTTTTAAGTGTTATGGAGTGTCTCCTACTAAATT
AAATGATCTCTGCTTTACTAATGTCTATGCAGATTCATTTGTAATTAGAGGTGATGAAGTC
AGACAAATCGCTCCAGGGCAAACTGGAAAGATTGCTGATTATAATTATAAATTACCAGAT
GATTTTACAGGCTGCGTTATAGCTTGGAATTCTAACAATCTTGATTCTAAGGTTGGTGGTA
ATTATAATTACCTGTATAGATTGTTTAGGAAGTCTAATCTCAAACCTTTTGAGAGAGATAT
TTCAACTGAAATCTATCAGGCCGGTAGCACACCTTGTAATGGTGTTGAAGGTTTTAATTGT
TACTTTCCTTTACAATCATATGGTTTCCAACCCACTAATGGTGTTGGTTACCAACCATACA
GAGTAGTAGTACTTTCTTTTGAACTTCTACATGCACCAGCAACTGTTTGTGGACCTAAAAA
GTCTACTAATTTGGTTAAAAACAAATGTGTCAATTTCAACTTCAATGGTTTAACAGGCACA
GGTGTTCTTACTGAGTCTAACAAAAAGTTTCTGCCTTTCCAACAATTTGGCAGAGACATTG
CTGACACTACTGATGCTGTCCGTGATCCACAGACACTTGAGATTCTTGACATTACACCATG
TTCTTTTGGTGGTGTCAGTGTTATAACACCAGGAACAAATACTTCTAACCAGGTTGCTGTT
CTTTATCAGGATGTTAACTGCACAGAAGTCCCTGTTGCTATTCATGCAGATCAACTTACTC
CTACTTGGCGTGTTTATTCTACAGGTTCTAATGTTTTTCAAACACGTGCAGGCTGTTTAAT
AGGGGCTGAACATGTCAACAACTCATATGAGTGTGACATACCCATTGGTGCAGGTATATG
CGCTAGTTATCAGACTCAGACTAATTCTCCTCGGCGGGCACGTAGTGTAGCTAGTCAATC
CATCATTGCCTACACTATGTCACTTGGTGCAGAAAATTCAGTTGCTTACTCTAATAACTCT
ATTGCCATACCCACAAATTTTACTATTAGTGTTACCACAGAAATTCTACCAGTGTCTATGA
CCAAGACATCAGTAGATTGTACAATGTACATTTGTGGTGATTCAACTGAATGCAGCAATC
TTTTGTTGCAATATGGCAGTTTTTGTACACAATTAAACCGTGCTTTAACTGGAATAGCTGT
TGAACAAGACAAAAACACCCAAGAAGTTTTTGCACAAGTCAAACAAATTTACAAAACAC
CACCAATTAAAGATTTTGGTGGTTTTAATTTTTCACAAATATTACCAGATCCATCAAAACC
AAGCAAGAGGTCATTTATTGAAGATCTACTTTTCAACAAAGTGACACTTGCAGATGCTGG
CTTCATCAAACAATATGGTGATTGCCTTGGTGATATTGCTGCTAGAGACCTCATTTGTGCA
CAAAAGTTTAACGGCCTTACTGTTTTGCCACCTTTGCTCACAGATGAAATGATTGCTCAAT
ACACTTCTGCACTGTTAGCGGGTACAATCACTTCTGGTTGGACCTTTGGTGCAGGTGCTGC
ATTACAAATACCATTTGCTATGCAAATGGCTTATAGGTTTAATGGTATTGGAGTTACACAG
AATGTTCTCTATGAGAACCAAAAATTGATTGCCAACCAATTTAATAGTGCTATTGGCAAA
ATTCAAGACTCACTTTCTTCCACAGCAAGTGCACTTGGAAAACTTCAAGATGTGGTCAAC
CAAAATGCACAAGCTTTAAACACGCTTGTTAAACAACTTAGCTCCAATTTTGGTGCAATTT
CAAGTGTTTTAAATGATATCCTTTCACGTCTTGACAAAGTTGAGGCTGAAGTGCAAATTG
ATAGGTTGATCACAGGCAGACTTCAAAGTTTGCAGACATATGTGACTCAACAATTAATTA
GAGCTGCAGAAATCAGAGCTTCTGCTAATCTTGCTGCTACTAAAATGTCAGAGTGTGTAC
TTGGACAATCAAAAAGAGTTGATTTTTGTGGAAAGGGCTATCATCTTATGTCCTTCCCTCA
GTCAGCACCTCATGGTGTAGTCTTCTTGCATGTGACTTATGTCCCTGCACAAGAAAAGAA
CTTCACAACTGCTCCTGCCATTTGTCATGATGGAAAAGCACACTTTCCTCGTGAAGGTGTC
TTTGTTTCAAATGGCACACACTGGTTTGTAACACAAAGGAATTTTTATGAACCACAAATC
ATTACTACAGACAACACATTTGTGTCTGGTAACTGTGATGTTGTAATAGGAATTGTCAAC
AACACAGTTTATGATCCTTTGCAACCTGAATTAGACTCATTCAAGGAGGAGTTAGATAAA
TATTTTAAGAATCATACATCACCAGATGTTGATTTAGGTGACATCTCTGGCATTAATGCTT
CAGTTGTAAACATTCAAAAAGAAATTGACCGCCTCAATGAGGTTGCCAAGAATTTAAATG
AATCTCTCATCGATCTCCAAGAACTTGGAAAGTATGAGCAGTATATAAAATGGCCATGGT
ACATTTGGCTAGGTTTTATAGCTGGCTTGATTGCCATAGTAATGGTGACAATTATGCTTTG
CTGTATGACCAGTTGCTGTAGTTGTCTCAAGGGCTGTTGTTCTTGTGGATCCTGCTGCAAA
TTTGATGAAGACGACTCTGAGCCAGTGCTCAAAGGAGTCAAATTACATTACACATAA,
CDS RBD Gene ID 43740568 - NC_045512.2
(SEQ ID NO: 179)
AGAGTCCAACCAACAGAATCTATTGTTAGATTTCCTAATATTACAAACTTGTGCCCTTTTG
GTGAAGTTTTTAACGCCACCAGATTTGCATCTGTTTATGCTTGGAACAGGAAGAGAATCA
GCAACTGTGTTGCTGATTATTCTGTCCTATATAATTCCGCATCATTTTCCACTTTTAAGTGT
TATGGAGTGTCTCCTACTAAATTAAATGATCTCTGCTTTACTAATGTCTATGCAGATTCAT
TTGTAATTAGAGGTGATGAAGTCAGACAAATCGCTCCAGGGCAAACTGGAAAGATTGCTG
ATTATAATTATAAATTACCAGATGATTTTACAGGCTGCGTTATAGCTTGGAATTCTAACAA
TCTTGATTCTAAGGTTGGTGGTAATTATAATTACCTGTATAGATTGTTTAGGAAGTCTAAT
CTCAAACCTTTTGAGAGAGATATTTCAACTGAAATCTATCAGGCCGGTAGCACACCTTGT
AATGGTGTTGAAGGTTTTAATTGTTACTTTCCTTTACAATCATATGGTTTCCAACCCACTA
ATGGTGTTGGTTACCAACCATACAGAGTAGTAGTACTTTCTTTTGAACTTCTACATGCACC
AGCAACTGTTTGTGGACCTAAAAAGTCTACTAATTTGGTTAAAAACAAATGTGTCAATTT
C
CDS S-Protein Wuhan-Hu-1 (Wuhan-Hu-1) (improved DNA)
(SEQ ID NO: 180)
ATGTTCGTTTTCCTGGTTCTGCTGCCGCTGGTTTCCTCCCAGTGCGTTAACCTGACCACCCG
TACCCAGCTGCCGCCGGCGTACACCAACTCCTTCACTCGTGGCGTATACTACCCGGACAA
AGTTTTCCGTTCCTCCGTTCTGCACTCCACCCAGGACCTGTTCCTGCCGTTCTTCTCCAACG
TTACCTGGTTCCACGCTATACACGTAAGCGGCACCAACGGCACCAAACGTTTCGACAACC
CGGTTCTGCCATTCAATGACGGCGTGTACTTCGCGAGCACCGAAAAATCCAACATCATCC
GTGGCTGGATCTTCGGCACCACCCTGGACTCCAAAACCCAGTCCCTGCTGATCGTTAACA
ACGCGACCAACGTAGTTATCAAAGTCTGCGAATTCCAGTTCTGCAACGACCCGTTTCTCG
GCGTGTACTACCACAAAAACAACAAATCCTGGATGGAGTCCGAGTTCCGGGTGTACAGCT
CCGCGAACAACTGCACCTTCGAATACGTTTCCCAGCCGTTCCTGATGGACCTGGAAGGCA
AACAGGGCAACTTCAAAAACCTGCGTGAATTCGTTTTCAAAAACATCGACGGCTACTTCA
AAATCTACTCCAAACACACCCCGATCAACCTGGTTCGTGACCTGCCGCAGGGCTTCTCCG
CGCTGGAACCGCTGGTTGACCTGCCGATCGGCATCAACATCACCCGTTTCCAGACCCTGC
TGGCGCTGCACCGTTCCTACCTGACCCCGGGCGACTCCTCCTCCGGCTGGACCGCGGGCG
CGGCGGCGTACTACGTTGGCTACCTGCAGCCGCGTACCTTCCTGCTGAAATACAACGAAA
ACGGCACCATCACCGACGCGGTTGACTGCGCGCTGGACCCGCTGTCCGAAACCAAATGCA
CCCTGAAATCCTTCACCGTTGAAAAAGGCATCTACCAGACCTCCAACTTCCGTGTTCAGCC
GACCGAATCCATAGTTAGGTTCCCGAACATCACTAACCTGTGTCCGTTTGGCGAAGTGTTC
AACGCGACCCGTTTTGCGTCCGTCTACGCCTGGAACCGTAAACGTATCTCCAACTGCGTTG
CGGACTACTCCGTTCTGTACAACTCCGCGTCCTTCTCCACCTTCAAATGCTACGGCGTTTC
CCCGACCAAACTGAACGACCTGTGCTTCACCAACGTTTACGCGGACTCCTTCGTTATCCGT
GGCGACGAAGTTCGTCAGATCGCGCCGGGCCAGACCGGCAAAATCGCGGACTACAACTA
CAAACTGCCGGACGACTTCACCGGCTGCGTTATCGCGTGGAACTCCAACAACCTGGACTC
CAAAGTTGGCGGCAACTACAACTACCTGTACCGTCTGTTCCGTAAATCCAACCTGAAACC
GTTCGAACGTGACATCTCCACCGAAATCTACCAGGCGGGCTCCACCCCGTGCAACGGCGT
TGAAGGCTTCAACTGCTACTTCCCGCTGCAGTCCTACGGCTTCCAGCCGACCAACGGCGTT
GGCTACCAGCCGTACCGTGTTGTTGTTCTGTCCTTCGAACTGCTGCACGCGCCGGCGACCG
TTTGCGGCCCGAAAAAATCCACCAACCTGGTTAAAAACAAATGCGTTAACTTCAACTTCA
ACGGCCTGACCGGCACCGGCGTTCTGACCGAATCCAACAAAAAATTCCTGCCGTTCCAGC
AGTTCGGCCGTGACATCGCGGACACCACCGACGCGGTTCGTGACCCGCAGACCCTGGAAA
TCCTGGACATCACCCCGTGCTCGTTCGGCGGCGTGAGCGTTATCACCCCGGGCACCAACA
CCTCCAACCAGGTTGCGGTTCTGTACCAGGACGTTAACTGCACCGAAGTTCCGGTTGCGA
TCCACGCGGACCAGCTGACCCCGACCTGGCGTGTTTACTCCACCGGCTCCAACGTTTTCCA
GACCCGTGCGGGCTGCCTGATCGGCGCGGAACACGTTAACAACTCCTACGAATGCGACAT
CCCGATCGGCGCGGGCATCTGCGCGTCCTACCAGACCCAGACCAACTCCCCGCGTCGTGC
GCGTTCCGTTGCGTCCCAGTCCATCATCGCGTACACCATGTCCCTGGGCGCGGAAAACTC
CGTTGCGTACTCCAACAACTCCATCGCGATCCCGACCAACTTCACCATCTCCGTTACCACC
GAAATCCTGCCGGTTTCCATGACCAAAACCTCCGTTGACTGCACCATGTACATCTGCGGC
GACTCCACCGAATGCTCCAACCTGCTGCTGCAGTACGGCTCCTTCTGCACCCAGCTGAAC
CGTGCGCTGACCGGCATCGCGGTTGAACAGGACAAAAACACCCAGGAAGTTTTCGCGCA
GGTTAAACAGATCTACAAAACCCCGCCGATCAAAGACTTCGGCGGCTTCAACTTCTCCCA
GATCCTGCCGGACCCGTCCAAACCGTCCAAACGTTCCTTCATCGAAGACCTGCTGTTCAA
CAAAGTTACCCTGGCGGACGCGGGCTTCATCAAACAGTACGGCGACTGCCTGGGCGACAT
CGCGGCGCGTGACCTGATCTGCGCGCAGAAATTCAACGGCCTGACCGTTCTGCCGCCGCT
GCTGACCGACGAAATGATCGCGCAGTACACCTCCGCGCTGCTGGCGGGCACCATCACCTC
CGGCTGGACCTTCGGCGCGGGCGCGGCGTTACAGATCCCGTTCGCGATGCAGATGGCGTA
CAGGTTCAACGGCATCGGCGTTACCCAGAACGTTCTGTACGAAAACCAGAAACTGATCGC
GAACCAGTTCAACTCCGCGATCGGCAAAATCCAGGACTCCCTGTCCTCCACCGCGTCCGC
GCTGGGCAAACTGCAGGACGTTGTTAACCAGAACGCGCAGGCGCTGAACACCCTGGTTA
AACAGCTGTCCTCCAACTTCGGCGCGATCTCCTCCGTTCTGAACGACATCCTGTCCCGTCT
GGACAAAGTTGAAGCGGAAGTTCAGATCGACCGTCTGATCACCGGCCGTCTGCAGTCCCT
GCAGACCTACGTTACCCAGCAGCTGATCCGTGCGGCGGAAATCCGTGCGTCCGCGAACCT
GGCGGCGACCAAAATGTCCGAATGCGTTCTGGGCCAGTCCAAACGTGTTGACTTCTGCGG
CAAAGGCTACCACCTGATGTCCTTCCCGCAGTCCGCTCCGCACGGCGTTGTGTTCCTGCAC
GTAACCTACGTTCCGGCGCAGGAAAAAAACTTCACCACCGCGCCGGCGATCTGCCACGAC
GGCAAAGCGCACTTCCCGCGTGAGGGCGTCTTCGTATCCAACGGCACCCACTGGTTCGTT
ACCCAGCGTAACTTCTACGAACCGCAGATCATCACCACCGACAACACCTTCGTTTCCGGC
AACTGCGACGTTGTTATCGGCATCGTAAATAACACCGTGTACGACCCCCTGCAGCCGGAA
CTGGACTCCTTCAAAGAAGAACTGGACAAATACTTCAAAAACCACACCTCCCCGGACGTT
GACCTGGGCGACATCTCCGGCATCAACGCGTCCGTTGTTAACATCCAGAAAGAAATCGAC
CGTCTGAACGAAGTTGCGAAAAACCTGAACGAATCCCTGATCGACCTGCAGGAACTGGG
CAAATACGAACAGTACATCAAATGGCCGTGGTACATCTGGCTGGGCTTCATCGCGGGCCT
GATCGCGATCGTTATGGTTACCATCATGCTGTGCTGCATGACCTCCTGCTGCTCCTGCCTG
AAAGGCTGCTGCTCCTGCGGCTCCTGCTGCAAATTCGACGAAGACGACTCCGAACCGGTT
CTGAAAGGCGTTAAACTGCACTACACC
CDS N-Protein NC_045512.2, GeneID: 43740575, Us converted to
Ts
(SEQ ID NO: 181)
ATGTCTGATAATGGACCCCAAAATCAGCGAAATGCACCCCGCATTACGTTTGGTGGACCC
TCAGATTCAACTGGCAGTAACCAGAATGGAGAACGCAGTGGGGCGCGATCAAAACAACG
TCGGCCCCAAGGTTTACCCAATAATACTGCGTCTTGGTTCACCGCTCTCACTCAACATGGC
AAGGAAGACCTTAAATTCCCTCGAGGACAAGGCGTTCCAATTAACACCAATAGCAGTCCA
GATGACCAAATTGGCTACTACCGAAGAGCTACCAGACGAATTCGTGGTGGTGACGGTAA
AATGAAAGATCTCAGTCCAAGATGGTATTTCTACTACCTAGGAACTGGGCCAGAAGCTGG
ACTTCCCTATGGTGCTAACAAAGACGGCATCATATGGGTTGCAACTGAGGGAGCCTTGAA
TACACCAAAAGATCACATTGGCACCCGCAATCCTGCTAACAATGCTGCAATCGTGCTACA
ACTTCCTCAAGGAACAACATTGCCAAAAGGCTTCTACGCAGAAGGGAGCAGAGGCGGCA
GTCAAGCCTCTTCTCGTTCCTCATCACGTAGTCGCAACAGTTCAAGAAATTCAACTCCAGG
CAGCAGTAGGGGAACTTCTCCTGCTAGAATGGCTGGCAATGGCGGTGATGCTGCTCTTGC
TTTGCTGCTGCTTGACAGATTGAACCAGCTTGAGAGCAAAATGTCTGGTAAAGGCCAACA
ACAACAAGGCCAAACTGTCACTAAGAAATCTGCTGCTGAGGCTTCTAAGAAGCCTCGGCA
AAAACGTACTGCCACTAAAGCATACAATGTAACACAAGCTTTCGGCAGACGTGGTCCAGA
ACAAACCCAAGGAAATTTTGGGGACCAGGAACTAATCAGACAAGGAACTGATTACAAAC
ATTGGCCGCAAATTGCACAATTTGCCCCCAGCGCTTCAGCGTTCTTCGGAATGTCGCGCAT
TGGCATGGAAGTCACACCTTCGGGAACGTGGTTGACCTACACAGGTGCCATCAAATTGGA
TGACAAAGATCCAAATTTCAAAGATCAAGTCATTTTGCTGAATAAGCATATTGACGCATA
CAAAACATTCCCACCAACAGAGCCTAAAAAGGACAAAAAGAAGAAGGCTGATGAAACTC
AAGCCTTACCGCAGAGACAGAAGAAACAGCAAACTGTGACTCTTCTTCCTGCTGCAGATT
TGGATGATTTCTCCAAACAATTGCAACAATCCATGAGCAGTGCTGACTCAACTCAGGCCT
AA
CDS DR (N-Protein) GeneID: 43740575 - NC_045512.2
(SEQ ID NO: 182)
CCTCGGCAAAAACGTACTGCCACTAAAGCATACAATGTAACACAAGCTTTCGGCAGACGT
GGTCCAGAACAAACCCAAGGAAATTTTGGGGACCAGGAACTAATCAGACAAGGAACTGA
TTACAAACATTGGCCGCAAATTGCACAATTTGCCCCCAGCGCTTCAGCGTTCTTCGGAATG
TCGCGCATTGGCATGGAAGTCACACCTTCGGGAACGTGGTTGACCTACACAGGTGCCATC
AAATTGGATGACAAAGATCCAAATTTCAAAGATCAAGTCATTTTGCTGAATAAGCATATT
GACGCATACAAA
CDS N-Protein, whole Protein (improved DNA)
(SEQ ID NO: 183)
ATGTCCGACAACGGCCCGCAGAACCAGCGTAACGCGCCGCGTATCACCTTCGGCGGCCCG
TCCGACTCCACCGGCTCCAACCAGAACGGCGAACGTTCCGGCGCGCGTTCCAAACAGCGT
CGTCCGCAGGGCCTGCCGAACAACACCGCGTCCTGGTTCACCGCGCTGACCCAGCACGGC
AAAGAAGACCTGAAATTCCCGCGTGGCCAGGGCGTTCCGATCAACACCAACTCCTCCCCG
GACGACCAGATCGGCTACTACCGTCGTGCGACCCGTCGTATCCGTGGCGGCGACGGCAAA
ATGAAAGACCTGTCCCCGCGTTGGTACTTCTACTACCTGGGCACCGGCCCGGAAGCGGGC
CTGCCGTACGGCGCGAACAAAGACGGCATCATCTGGGTTGCGACCGAAGGCGCGCTGAA
CACCCCGAAAGACCACATCGGCACCCGTAACCCGGCGAACAACGCGGCGATCGTTCTGC
AGCTGCCGCAGGGCACCACCCTGCCGAAAGGCTTCTACGCGGAAGGCTCCCGTGGCGGCT
CCCAGGCGTCCTCCCGTTCCTCCTCCCGTTCCCGTAACTCCTCCCGTAACTCCACCCCGGG
CTCCTCCCGTGGCACCTCCCCGGCGCGTATGGCGGGCAACGGCGGCGACGCGGCGCTGGC
GCTGCTGCTGCTGGACCGTCTGAACCAGCTGGAATCCAAAATGTCCGGCAAAGGCCAGCA
GCAGCAGGGCCAGACCGTTACCAAAAAATCCGCGGCGGAAGCGTCCAAAAAACCGCGTC
AGAAACGTACCGCGACCAAAGCGTACAACGTTACCCAGGCGTTCGGCCGTCGTGGCCCG
GAACAGACCCAGGGCAACTTCGGCGACCAGGAACTGATCCGTCAGGGCACCGACTACAA
ACACTGGCCGCAGATCGCGCAGTTCGCGCCGTCCGCGTCCGCGTTCTTCGGCATGTCCCGT
ATCGGCATGGAAGTTACCCCGTCCGGCACCTGGCTGACCTACACCGGCGCGATCAAACTG
GACGACAAAGACCCGAACTTCAAAGACCAGGTTATCCTGCTGAACAAACACATCGACGC
GTACAAAACCTTCCCGCCGACCGAACCGAAAAAAGACAAAAAAAAAAAAGCGGACGAA
ACCCAGGCGCTGCCGCAGCGTCAGAAAAAACAGCAGACCGTTACCCTGCTGCCGGCGGC
GGACCTGGACGACTTCTCCAAACAGCTGCAGCAGTCCATGTCCTCCGCGGACTCCACCCA
GGCG

### INDUSTRIAL APPLICABILITY

The bacterium, combination product and vaccine of the present invention are susceptible of industrial application. The invention can be manufactured for use in the medical and healthcare industry. In particular, the invention can be used to provide patients with an active adaptive immunity towards members of the coronavirus family.

The invention is exemplified by the following non-limiting Examples:

### EXAMPLES

### Example 1: Antigenic Plots

Antigenic plots of SEQ ID NO: 30 and SEQ ID NO: 41 were generated using the method disclosed in Kolaskar & Tongaonkar, 1990. FEBS Lett. 276(1-2):172-4. These plots are provided in FIGS. 4 and 5.

According to the antigenic plots, the herein disclosed fusion proteins have the potential to induce an immune response in a subject. Thus, they have the potential to function as a vaccine.

Further, antigenic plots were used to identify SARS-CoV-2 antigens with an antigenic propensity score of greater than 0.9. All the SARS-CoV-2 antigens disclosed herein have an antigenic propensity score of greater than 0.9.

### Example 2: Plasmid

The constructs disclosed herein can be introduced into a Ty21a Salmonella strain via the pSalVac plasmid. The pSalVac 001 A0_B0 plasmid is depicted in FIG. 1. Sequences encoding fusion proteins can be inserted at the SalI recognition site and/or at the NsiI recognition site.

The sequence of the pSalVac 001 A0_B0 KanR plasmid is provided in SEQ ID NO: 42:

GAATTCCAAGCGAAGTCCATCCCCCTCCCTCTTGATTACAAGGGTGATAATTATTATTCGC
ATTTGTGTGGTAATGGGATAGAAAGGAATGGATAGAAAAAGAACAAAATTAGTATAGCA
ATAGATATGCCCACTGCATTGAATACTTACAGGGCATTATTTTATTATGTTTAAATTGAAG
TGGTCTCTGGTTTGATTTATTTGTTATTCAAGGGGGCTGTTTGGAGATCGGAAAATTCTGT
ACGTTAAGTGTATTATTTAACCAGTTTCGATGCGTAACAGATTGATTTTGCGTCAGCGGTT
ATCGCTTTTAAGTTGTTGCTCTTGCGCTATCGCGTTTAGGTTATCCGATTAAAGTCAAATTT
CCTGAAAATGCTGTATAGCGCGGGAGTGCACCTTATAGCTGTAGGTAAGTATGTTCAAAA
AATAGTCTTGCCGTACAATAATTTTCCATATCCAAACTCACTCCTTCAAGATTCTGGTCCC
GGTTTACGGGTAGTTTCCGGAAGGGCGGTAGCATGCTGATTCAAACTGCAAGATGAAACA
TTGTCGGAGTTGGATGGAATTAAGTCATGGCTATAGCATTTGGGCGTGCATAACAAAATT
GGTCCTCATATTTTAGAGTATGATTGCATATTCACTAATATTTTTACTTTCTGATGCGTGGT
GGCATCATGCTTTATGAGATAAACAATCCTGGTAGACTAGCCCCCTGAATCTCCAGACAA
CCAATATCACTTATTTAAGTGATAGTCTTAATACTAGTGCTAGCGACACCATCGAATGGC
GCAAACCTTTCGCGGTATGGCATGATAGCGCCCGAAGTCGTGTACCGGCAAAGGTGCAGT
CGTTATATACATGGAGATTTTGATGGCAAGCAGTAACTTGATTAAACAATTGCAAGAGCG
GGGGCTGGTAGCCCAGGTGACGGACGAGGAAGCGTTAGCAGAGCGACTGGCGCAAGGCC
CGATCGCGCTCTATTGCGGCTTCGATCCTACCGCTGACAGCTTGCATTTGGGGCATCTTGT
TCCATTGTTATGCCTGAAACGCTTCCAGCAGGCGGGCCACAAGCCGGTTGCGCTGGTAGG
CGGCGCGACGGGTCTGATTGGCGACCCGAGCTTCAAAGCTGCCGAGCGTAAGCTGAACA
CCGAAGAAACTGTTCAGGAGTGGGTGGACAAAATCCGTAAGCAGGTTGCCCCGTTCCTCG
ATTTCGACTGTGGAGAAAACTCTGCTATCGCGGCGAACAACTATGACTGGTTCGGCAATA
TGAATGTGCTGACCTTCCTGCGCGATATTGGCAAACACTTCTCCGTTAACCAGATGATCAA
CAAAGAAGCGGTTAAGCAGCGTCTCAACCGTGAAGATCAGGGGATTTCGTTCACTGAGTT
TTCCTACAACCTGTTGCAGGGTTATGACTTCGCCTGTCTGAACAAACAGTACGGTGTGGTG
CTGCAAATTGGTGGTTCTGACCAGTGGGGTAACATCACTTCTGGTATCGACCTGACCCGTC
GTCTGCATCAGAATCAGGTGTTTGGCCTGACCGTTCCGCTGATCACTAAAGCAGATGGCA
CCAAATTTGGTAAAACTGAAGGCGGCGCAGTCTGGTTGGATCCGAAGAAAACCAGCCCG
TACAAATTCTACCAGTTCTGGATCAACACTGCGGATGCCGACGTTTACCGCTTCCTGAAGT
TCTTCACCTTTATGAGCATTGAAGAGATCAACGCCCTGGAAGAAGAAGATAAAAACAGC
GGTAAAGCACCGCGCGCCCAGTATGTACTGGCGGAGCAGGTGACTCGTCTGGTTCACGGT
GAAGAAGGTTTACAGGCGGCAAAACGTATTACCGAATGCCTGTTCAGCGGTTCTTTGAGT
GCGCTGAGTGAAGCGGACTTCGAACAGCTGGCGCAGGACGGCGTACCGATGGTTGAGAT
GGAAAAGGGCGCAGACCTGATGCAGGCACTGGTCGATTCTGAACTGCAACCTTCCCGTGG
TCAGGCACGTAAAACTATCGCCTCCAATGCCATCACCATTAACGGTGAAAAACAGTCCGA
TCCTGAATACTTCTTTAAAGAAGAAGATCGTCTGTTTGGTCGTTTTACCTTACTGCGTCGC
GGTAAAAAGAATTACTGTCTGATTTGCTGGAAACATCACCATCACCATCACTAATCCACG
GCCGCCAGTTTGGGCTGGCGGCATTTTGGTACCACTAGTGATAATGGTTCATGCTACCGG
GCGAATGAAACACGTCAGTTCGCCAGGATGTTGGGACTTGAACCGAAGAACACGGCAGT
GCGGAGTCCGGAGAGTAACGGAATAACAGAGAGCTTCGTGAAAACGATAAAGCGTGATT
ACATAAGTATCATGCCCAAACCAGACGGGTTAACGGCAGCAAAGAACCTTGCAGAGGCG
TTCGAGCATTATAACGAATGGCATCCGCATAGTGCGCTGGGTTATCGCTCGCCACGGGAA
TATCTGCGGCAGCGGGCCAGTAATGGGTTAAGTGATAACAGGTATCTGGAAATATAGGG
GCAAATCCACCTGGTCATTATCTGGAATTTGACGAAGTGTGATAACTGGTATAGCCAGAT
TAATCTAAACCTTTGTCTGACAAAATCAGATAAAGAAGAGTAGTTCAAAAGACAACTCGT
GGACTCTCATTCAGAGAGATAGGCGTTACCAAAATTTGTTTGGAACTGAACAAGAAAATT
GTATTTGTGTAACTATAATCTTAATGTAAAATAAAAGACACCAGTTCTGTAGAATATGCTT
ATTGAAGAGAGTGTAATAATAATTTTATATAGATGTTGTACAAAGAACAGGAATGAGTAA
TTATTTATGCTTGATGTTTTTTGACTCTTGCTTTTTATAGTTATTATTTTTAAGTTAGTCAGC
GCAATAAAAACTTGCTTTTAATATTAATGCGAGTTATGACATTAAACGGAAGAAACATAA
AGGCATATTTTTGCCACAATATTTAATCATATAATTTAAGTTGTAGTGAGTTTATTATGAA
TATAAACAAACCATTAGAGATTCTTGGGCATGTATCCTGGCTATGGGCCAGTTCTCCACTA
CACAGAAACTGGCCAGTATCTTTGTTTGCAATAAATGTATTACCCGCAATACAGGCTAAC
CAATATGTTTTATTAACCCGGGATGATTACCCTGTCGCGTATTGTAGTTGGGCTAATTTAA
GTTTAGAAAATGAAATTAAATATCTTAATGATGTTACCTCATTAGTTGCAGAAGACTGGA
CTTCAGGTGATCGTAAATGGTTCATTGACTGGATTGCTCCTTTCGGGGATAACGGTGCCCT
GTACAAATATATGCGAAAAAAATTCCCTGATGAACTATTCAGAGCCATCAGGGTGGATCC
CAAAACTCATGTTGGTAAAGTATCAGAATTTCATGGAGGTAAAATTGATAAACAGTTAGC
GAATAAAATTTTTAAACAATATCACCACGAGTTAATAACTGAAGTAAAAAGAAAGTCAG
ATTTTAATTTTTCATTAACTGGTTAAGAGGTAATTAAATGCCAACAATAACCACTGCACAA
ATTAAAAGCACACTGCAGTCTGCAAAGCAATCCGCTGCAAATAAATTGCACTCAGCAGGA
CAAAGCACGAAAGATGCATTAGCCTATGGAAGTCAGGGTGATCTTAATCCATTAATTAAT
GAAATCAGCAAAATCATTTCAGCTGCAGGTAGCTTCGATGTTAAAGAGGAAAGAACTGC
AGCTTCTTTATTGCAGTTGTCCGGTAATGCCAGTGATTTTTCATATGGACGGAACTCAATA
ACCCTGACCACATCAGCATAATATATTAATTTAAATGATAGCAATCTTACTGGGCTGTGCC
ACATAAGATTGCTATTTTTTTTGGAGTCATAATGGATTCTTGTCATAAAATTGATTATGGG
TTATACGCCCTGGAGATTTTAGCCCAATACCATAACGTCTCTGTTAACCCGGAAGAAATT
AAACATAGATTTGATACAGACGGGACAGGTCTGGGATTAACGTCATGGTTGCTTGCTGCG
AAATCTTTAGAACTAAAGGTAAAACAGGTAAAAAAAACAATTGATCGATTAAACTTTATT
TTTCTGCCCGCATTAGTCTGGAGAGAGGATGGACGTCATTTTATTCTGACTAAAATCAGCA
AAGAAGTAAACAGATATCTTATTTTTGATTTGGAGCAGCGAAATCCCCGTGTTCTCGAAC
AGTCTGAGTTTGAGGCGTTATATCAGGGGCATATTATTCTTATTACTTCCCGTTCTTCTGTT
ACCGGGAAACTGGCAAAATTTGACTTTACCTGGTTTATTCCTGCCATTATAAAATACAGG
AGAATATTTATTGAAACCCTTGTTGTATCTGTTTTTTTACAATTATTTGCATTAATAACCCC
CCTTTTTTTCCAGGTGGTTATGGACAAAGTATTAGTGCACAGGGGGTTTTCAACCCTTAAT
GTTATTACTGTTGCATTATCTGTTGTAGTGGTGTTTGAGATTATACTCAGCGGTTTAAGAA
CTTACATTTTTGCACATAGTACAAGTCGGATTGATGTTGAGTTGGGTGCCAAACTCTTCCG
GCATTTACTGGCGCTACCGATCTCTTATTTTGAGAGTCGTCGTGTTGGTGATACTGTTGCG
AGGGTAAGAGAATTAGACCAGATCCGTAATTTTCTGACAGGACAGGCATTAACATCTGTT
TTGGACTTATTATTTTCACTCATATTTTTTGCGGTAATGTGGTATTACAGCCCAAAGCTTAC
TCTGGTGATCTTATTTTCGCTGCCTTGTTATGCTGCATGGTCTGTTTTTATTAGCCCCATTT
TGCGACGTCGCCTTGATGATAAGTTTTCACGGAATGCGGATAATCAATCTTTCCTGGTGGA
ATCAGTAACGGCGATTAACACTATAAAAGCTATGGCAGTCTCACCTCAGATGACGAACAT
ATGGGACAAACAATTGGCAGGATATGTTGCTGCAGGCTTTAAAGTGACAGTATTAGCAAC
CATTGGTCAACAAGGAATACAGTTAATACAAAAGACTGTTATGATCATCAACCTATGGTT
GGGAGCACACCTGGTTATTTCCGGGGATTTAAGTATTGGTCAGTTAATTGCTTTTAATATG
CTTGCTGGTCAGATTGTTGCACCGGTTATTCGCCTTGCACAAATCTGGCAGGATTTCCAGC
AGGTTGGTATATCAGTTACCCGCCTTGGTGATGTGCTTAACTCTCCAACTGAAAGTTATCA
TGGGAAACTGACATTGCCGGAAATTAATGGTGATATCACTTTTCGTAATATCCGGTTTCGC
TATAAACCTGATTCTCCGGTTATTTTGGACAATATCAATCTTAGTATTAAGCAGGGGGAG
GTTATTGGTATTGTCGGACGTTCTGGTTCAGGAAAAAGCACATTAACTAAATTAATTCAA
CGTTTTTATATTCCTGAAAATGGCCAGGTATTAATTGATGGACATGATCTTGCGTTGGCTG
ATCCTAACTGGTTACGTCGTCAGGTGGGGGTTGTGTTGCAGGACAATGTGCTGCTTAATC
GCAGTATTATTGATAATATTTCACTGGCTAATCCTGGCATGTCCGTCGAAAAAGTTATTTA
TGCAGCGAAATTAGCAGGCGCTCATGATTTTATTTCTGATTTGCGTGAGGGGTATAACAC
CATTGTCGGGGAACAGGGGGCAGGATTATCCGGAGGTCAACGTCAACGCATCGCAATTG
CAAGGGCGCTGGTGAACAACCCTAAAATACTCATTTTTGATGAAGCAACCAGTGCTCTGG
ATTATGAGTCGGAGCATGTCATCATGCGCAATATGCACAAAATATGTAAGGGCAGAACG
GTTATAATCATTGCTCATCGTCTGTCTACAGTAAAAAATGCAGACCGCATTATTGTCATGG
AAAAAGGGAAAATTGTTGAACAGGGTAAACATAAGGAGCTGCTTTCTGAACCGGAAAGT
TTATACAGTTACTTATATCAGTTACAGTCAGACTAACAGAAAGAACAGAAGAATATGAAA
ACATGGTTAATGGGGTTCAGCGAGTTCCTGTTGCGCTATAAACTTGTCTGGAGTGAAACA
TGGAAAATCCGGAAGCAATTAGATACTCCGGTACGTGAAAAGGACGAAAATGAATTCTT
ACCCGCTCATCTGGAATTAATTGAAACGCCAGTATCCAGACGGCCGCGTCTGGTTGCTTA
TTTTATTATGGGGTTTCTGGTTATTGCTTTTATTTTATCTGTTTTAGGCCAAGTGGAAATTG
TTGCCACTGCAAATGGGAAATTAACACACAGTGGGCGTAGTAAAGAAATTAAACCTATTG
AAAACTCAATAGTTAAAGAAATTATCGTAAAAGAAGGAGAGTCAGTCCGGAAAGGGGAT
GTGTTATTAAAGCTTACAGCACTGGGAGCTGAAGCTGATACGTTAAAAACACAGTCATCA
CTGTTACAGGCCAGGCTGGAACAAACTCGGTATCAAATTCTGAGCAGGTCAATTGAATTA
AATAAACTACCTGAACTAAAGCTTCCTGATGAGCCTTATTTTCAGAATGTATCTGAAGAG
GAAGTACTGCGTTTAACTTCTTTGATAAAAGAACAGTTTTCCACATGGCAAAATCAGAAG
TATCAAAAAGAACTGAATTTGGATAAGAAAAGAGCAGAGCGATTAACAGTACTTGCCCG
TATAAACCGTTATGAAAATTTATCAAGGGTTGAAAAAAGCCGTCTGGATGATTTCAGTAG
TTTATTGCATAAACAGGCAATTGCAAAACATGCTGTACTTGAGCAGGAGAATAAATATGT
CGAAGCAGTAAATGAATTACGAGTTTATAAATCACAACTGGAGCAAATTGAGAGTGAGA
TATTGTCTGCAAAAGAAGAATATCAGCTTGTTACGCAGCTTTTTAAAAATGAAATTTTAG
ATAAGCTAAGACAAACAACAGACAACATTGGGTTATTAACTCTGGAATTAGCGAAAAAT
GAAGAGCGTCAACAGGCTTCAGTAATCAGGGCCCCAGTTTCGGGAAAAGTTCAGCAACT
GAAGGTTCATACTGAAGGTGGGGTTGTTACAACAGCGGAAACACTGATGGTCATCGTTCC
GGAAGATGACACGCTGGAGGTTACTGCTCTGGTACAAAATAAAGATATTGGTTTTATTAA
CGTCGGGCAGAATGCCATCATTAAAGTGGAGGCATTTCCTTATACACGATATGGTTATCT
GGTGGGTAAGGTGAAAAATATAAATTTAGATGCAATAGAAGACCAGAGACTGGGACTTG
TTTTTAATGTTATTATTTCTATTGAAGAGAATTGTTTGTCAACCGGGAATAAAAACATTCC
ATTAAGCTCGGGTATGGCAGTCACTGCAGAAATAAAGACAGGTATGCGAAGTGTAATCA
GTTATCTTCTTAGTCCTTTAGAAGAGTCAGTAACAGAAAGTTTACGTGAGCGTTAAGTTTC
AGAAGTCCAGTATTTGCTGCTATACGTGCTGCGTGGCACTTGCCGTCTGAACGGCATTGAT
CCGGAAGCCAAGTCAAACAACAGCGTGATGAGCGTCAGGGCAAAACACCAAGGCTCTCT
CGATGACACCAGAACAAATTGAAATACGTGAGCTGAGGAAAAAGCTACCGAGTTCTTGA
TGTTGGACTCCCTGAACAGTTCTCTGTAATCGGGAAACTCAGGACGCGTTATCCTGTGGTC
ACACTCTGCCATGTGTTTAGGGTTCATCACAGCAGCTACAGATACTGGTAAAACCGTCCT
GAAAAACCAGACGGCAGACGGGCTGTATTACGTAGTCAGGTACTTGAGCTACATGGCATC
AGTCACGGTTTGGCCGGAGCAAGACGTATCACCACAATGGCAACCCGGAGAGGTGTCAG
CGCCAGTGATATAAGACGGTTAACGGTTAAAAATCGTGGCGTTGACAACATCCCAGTGGA
CTGAGGTCACACAGGCCTGGCAGCATTCCTCTTCCGGCCGGATGACCCGGATTTCACGGG
GAAAGTACGCCGATAACAGTTTACGGGCTGAAGATTGGCGTAGGGAGGATAGCAGACGT
TTTGCCGCCCCCATTGTCTGGAGTTGGGTGAGAAGGCATCATTTCACCAACACCAACATTT
CACAGTTACACCCCACAGCTACATGAAGCGCTTCCATGAATTATCGCTTTGATTTATCATG
TTAAAATAGCTCTACACGGTTGGTTCAGGATTGCGCACCGAAACCCTCTAAAATCCACTG
ACGCGCCTGCGAATTATCCAGCACCGCGCCTTTCGAGATCCTCTACGCCGGACGCATCGT
GGCCGGCATCACCGGCGCCACAGGTGCGGTTGCTGGCGCCTATATCGCCGACATCACCGA
TGGGGAAGATCGGGCTCGCCACTTCGGGCTCATGAGCGCTTGTTTCGGCGTGGGTATGGT
GGCAGGCCCCGTGGCCGGGGGACTGTTGGGCGCCATCTCCTTGCATGCACCATTCCTTGC
GGCGGCGGTGCTCAACGGCCTCAACCTACTACTGGGCTGCTTCCTAATGCAGGAGTCGCA
TAAGGGAGAGCGTCGACCGATGCCCTTGAGAGCCTTCAACCCAGTCAGCTCCTTCCGGTG
GGCGCGGGGCATGACTATCGTCGCCGCACTTATGACTGTCTTCTTTATCATGCAACTCGTA
GGACAGGTGCCGGCAGCGCTCTGGGTCATTTTCGGCGAGGACCGCTTTCGCTGGAGCGCG
ACGATGATCGGCCTGTCGCTTGCGGTATTCGGAATCTTGCACGCCCTCGCTCAAGCCTTCG
TCACTGGTCCCGCCACCAAACGTTTCGGCGAGAAGCAGGCCATTATCGCCGGCATGGCGG
CCGACGCGCTGGGCTACGTCTTGCTGGCGTTCGCGACGCGAGGCTGGATGGCCTTCCCCA
TTATGATTCTTCTCGCTTCCGGCGGCATCGGGATGCCCGCGTTGCAGGCCATGCTGTCCAG
GCAGGTAGATGACGACCATCAGGGACAGCTTCAAGGATCGCTCGCGGCTCTTACCAGCCT
AACTTCGATCATTGGACCGCTGATCGTCACGGCGATTTATGCCGCCTCGGCGAGCACATG
GAACGGGTTGGCATGGATTGTAGGCGCCGCCCTATACCTTGTCTGCCTCCCCGCGTTGCGT
CGCGGTGCATGGAGCCGGGCCACCTCGACCTGAATGGAAGCCGGCGGCACCTCGCTAAC
GGATTCACCACTCCAAGAATTGGAGCCAATCAATTCTTGCGGAGAACTGTGAATGCGCAA
ACCAACCCTTGGCAGAACATATCCATCGCGTCCGCCATCTCCAGCAGCCGCACGCGGCGC
ATCTCGGGCAGCGTTGGGTCCTGGCCACGGGTGCGCATGATCGTGCTCCTGTCGTTGAGG
ACCCGGCTAGGCTGGCGGGGTTGCCTTACTGGTTAGCAGAATGAATCACCGATACGCGAG
CGAACGTGAAGCGACTGCTGCTGCAAAACGTCTGCGACCTGAGCAACAACATGAATGGT
CTTCGGTTTCCGTGTTTCGTAAAGTCTGGAAACGCGGAAGTCAGCGCCCTGCACCATTATG
TTCCGGATCTGCATCGCAGGATGCTGCTGGCTACCCTGTGGAACACCTACATCTGTATTAA
CGAAGCGCTGGCATTGACCCTGAGTGATTTTTCTCTGGTCCCGCCGCATCCATACCGCCAG
TTGTTTACCCTCACAACGTTCCAGTAACCGGGCATGTTCATCATCAGTAACCCGTATCGTG
AGCATCCTCTCTCGTTTCATCGGTATCATTACCCCCATGAACAGAAATCCCCCTTACACGG
AGGCATCAGTGACCAAACAGGAAAAAACCGCCCTTAACATGGCCCGCTTTATCAGAAGC
CAGACATTAACGCTTCTGGAGAAACTCAACGAGCTGGACGCGGATGAACAGGCAGACAT
CTGTGAATCGCTTCACGACCACGCTGATGAGCTTTACCGCAGCTGCCTCGCGCGTTTCGGT
GATGACGGTGAAAACCTCTGACACATGCAGCTCCCGGAGACGGTCACAGCTTGTCTGTAA
GCGGATGCCGGGAGCAGACAAGCCCGTCAGGGCGCGTCAGCGGGTGTTGGCGGGTGTCG
GGGCGCAGCCATGACCCAGTCACGTAGCGATAGCGGAGTGTATACTGGCTTAACTATGCG
GCATCAGAGCAGATTGTACTGAGAGTGCACCATATGCGGTGTGAAATACCGCACAGATGC
GTAAGGAGAAAATACCGCATCAGGCGCTCTTCCGCTTCCTCGCTCACTGACTCGCTGCGC
TCGGTCGTTCGGCTGCGGCGAGCGGTATCAGCTCACTCAAAGGCGGTAATACGGTTATCC
ACAGAATCAGGGGATAACGCAGGAAAGAACATGTGAGCAAAAGGCCAGCAAAAGGCCA
GGAACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAG
GTATCTCAGTTCGGTGTAGGTCGTTCGCTCCAAGCTGGGCTGTGTGCACGAACCCCCCGTT
CAGCCCGACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGGACAGTATT
TGGTATCTGCGCTCTGCTGAAGCCAGTTACCTTCGGAAAAAGAGTTGGTAGCTCTTGATCC
GGCAAACAAACCACCGCTGGTAGCGGTGGTTTTTTTGTTTGCAAGCAGCAGATTACGCGC
AGAAAAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACCTAG
ATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTC
ATCCATAGTTGCCTGACTCCCCATATGAATATCCTCCTTAGTTCCTATTCCGAAGTTCCTAT
TCTCTAGAAAGTATAGGAACTTCAGAGCGCTTTTGAAGCTGGGGTGGGCGAAGAACTCCA
GCATGAGATCCCCGCGCTGGAGGATCATCCAGCCGGCGTCCCGGAAAACGATTCCGAAG
CCCAACCTTTCATAGAAGGCGGCGGTGGAATCGAAATCTCGTGATGGCAGGTTGGGCGTC
GCTTGGTCGGTCATTTCGAACCCCAGAGTCCCGCTCAGAAGAACTCGTCAAGAAGGCGAT
AGAAGGCGATGCGCTGCGAATCGGGAGCGGCGATACCGTAAAGCACGAGGAAGCGGTCA
GCCCATTCGCCGCCAAGCTCTTCAGCAATATCACGGGTAGCCAACGCTATGTCCTGATAG
CGGTCCGCCACACCCAGCCGGCCACAGTCGATGAATCCAGAAAAGCGGCCATTTTCCACC
ATGATATTCGGCAAGCAGGCATCGCCATGGGTCACGACGAGATCCTCGCCGTCGGGCATG
CGCGCCTTGAGCCTGGCGAACAGTTCGGCTGGCGCGAGCCCCTGATGCTCTTCGTCCAGA
TCATCCTGATCGACAAGACCGGCTTCCATCCGAGTACGTGCTCGCTCGATGCGATGTTTCG
CTTGGTGGTCGAATGGGCAGGTAGCCGGATCAAGCGTATGCAGCCGCCGCATTGCATCAG
CCATGATGGATACTTTCTCGGCAGGAGCAAGGTGAGATGACAGGAGATCCTGCCCCGGCA
CTTCGCCCAATAGCAGCCAGTCCCTTCCCGCTTCAGTGACAACGTCGAGCACAGCTGCGC
AAGGAACGCCCGTCGTGGCCAGCCACGATAGCCGCGCTGCCTCGTCCTGCAGTTCATTCA
GGGCACCGGACAGGTCGGTCTTGACAAAAAGAACCGGGCGCCCCTGCGCTGACAGCCGG
AACACGGCGGCATCAGAGCAGCCGATTGTCTGTTGTGCCCAGTCATAGCCGAATAGCCTC
TCCACCCAAGCGGCCGGAGAACCTGCGTGCAATCCATCTTGTTCAATCATGCGAAACGAT
CCTCATCCTGTCTCTTGATCAGATCTTGATCCCCTGCGCCATCAGATCCTTGGCGGCAAGA
AAGCCATCCAGTTTACTTTGCAGGGCTTCCCAACCTTACCAGAGGGCGCCCCAGCTGGCA
ATTCCGGTTCGCTTGCTGTCCATAAAACCGCCCAGTCTAGCTATCGCCATGTAAGCCCACT
GCAAGCTACCTGCTTTCTCTTTGCGCTTGCGTTTTCCCTTGTCCAGATAGCCCAGTAGCTG
ACATTCATCCGGGGTCAGCACCGTTTCTGCGGACTGGCTTTCTACGTGTTCCGCTTCCTTT
AGCAGCCCTTGCGCCCTGAGTGCTTGCGGCAGCGTGGGGGATCTTGAAGTTCCTATTCCG
AAGTTCCTATTCTCTAGAAAGTATAGGAACTTCGAAGCAGCTCCAGCCTACACCAAAAAA
GGGAATAAGGGCGACACGGAAATGTTGAATACTCATACTCTTCCTTTTTCAATATTATTGA
AGCATTTATCAGGGTTATTGTCTCATGAGCGGATACATATTTGAATGTATTTAGAAAAATA
AACAAATAGGGGTTCCGCGCACATTTCCCCGAAAAGTGCCACCTGACGTCTAAGAAACCA
TTATTATCATGACATTAACCTATAAAAATAGGCGTATCACGAGGCCCTTTCGTCTTCAAGA
ATTCTCATGTTTGACAGCTTATCATCGATGGACATTATTTTTGTGGAGCCGGAGGAAACAG
ACCAGACGGTTCAGATGAGGCGCTTACCACCAGAACCGCTGTTGTCCCACCATTCTGGCG
ATTCCCAAACGCTATTTGGATAAAAAGTAGCCTTAACGTGGTTTATTTTCC

Methods for inserting plasmids into S. typhi strains are known in the art (see Callaghan & Charbit, 1990. Mol Gen Genet. 223(1):156-8).

### Example 3: Preparation and Testing of Vaccines According to the Invention

1. Materials

1.1 Bacterial Strains

Bacterial strains are depicted in table 1 (E. coli, Salmonella initial strains), table 10 (Salmonella intermediate and recipient strains) and table 11 (BLS vaccine strains).

1.2 Plasmids

Plasmids are listed in table 6 (codon optimized synthetic antigen fragments in delivery plasmids by manufacturer), table 7A, and table 9 (plasmids for the construction of BLS strains and the JMU SalVac-100 series).

1.3 Primers

Primes are listed in table 7B (construction of BLS strains), table 8 (sequencing and PCR) and table 12 (qPCR).

1.4 Media

For strain construction purposes:
LB-Broth
20 g Luria Bertani (LB) broth (Lennox) vegetal, animal-free (Roth)
ad 1000 ml Roti-Cell water, CELLPURE sterile
LB-Agar
35 g LB-Agar (Lennox) vegetal, animal-free (Roth)
ad 1000 ml Roti-Cell water, CELLPURE sterile

For quality control and characterization purposes:
TS-Broth (TSM)
30 g Tryptic Soy Broth (Sigma-Aldrich)
ad 1000 ml dest. Water
TS-Agar (TSA)
30 g Tryptic Soy Broth (Sigma-Aldrich)
15 g Agar (BD)
ad 1000 ml dest. Water

Media for bacterial culture were autoclaved for 20 min at 121° C. Antibiotics and other temperature sensitive supplements were added after autoclaving and cooling of the media.

1.5 Chemicals

Unless otherwise stated, all chemicals were obtained from Sigma-Aldrich, Difco, Roth and Applichem.

1.6 Buffers and Solutions

### 50×TAE Buffer:

242 g Tris

100 ml 0.5 M EDTA pH 8.0

57.1 ml acetic acid

Ad 1000 ml ddH₂O

### 1×TBE (Tris-Borat-EDTA):

100 ml 10×TBE-Puffer (ThermoFisher)

Ad to 1000 ml ddH₂O

### 2× Laemmli:

10 ml 1.5 M Tris/HCl pH 6.8

40 ml 10% SDS

30 ml Glycerol

5 mg Bromophenol blue

1.5 ml β-mercaptoethanol

Ad to 100 ml ddH₂O

### Lower Buffer:

90.85 g Tris

20 mil 10% SDS

Ad 500 ml ddH₂O

Set pH to 8.8

### Upper Buffer:

30.3 g Tris

20 mil 10% SDS

Ad 500 ml ddH₂O

Set pH to 6.8

### 10% Separating Gel:

4.15 ml millipore H₂O

2.5 ml lower buffer

3.35 ml Rotiphorese Gel 30 (37.5:1)

75 μl 10% APS

7.5 μl TEMED

### 3.75% Stacking Gel:

6.25 ml millipore H₂O

2.5 ml upper buffer

1.25 ml Rotiphorese Gel 30 (37.5:1)

100 μl 10% APS

20 μl TEMED

### 10×SDS Running Buffer:

10 g SDS

30.3 g Tris

144.1 g Glycine

Ad 1 1 ddH₂O

### 10× Semi-Dry Transfer Buffer:

77.5 g Glycine

100 ml 10% SDS

250 ml 1 M Tris pH 7.5-8.0

Ad 1 1 ddH₂O

Set pH to 8.3

### 10×Tbs-T Buffer:

60.5 g Tris

87.6 g NaCl

Ad 1 1 ddH₂O

Set pH to 7.5

5 ml Tween-20

### ECL-Solution 1:

5 ml 1 M Tris/HCl pH 8.5

500 μl 250 mM Luminol in DMSO

220 μl 90 mM cumeric acid in DMSO

Add to 50 ml ddH₂O

### ECL-Solution 2:

5 ml 1 M Tris/HCl pH 8.5

32 μl 35% H₂O₂

Add to 50 ml ddH₂O

2. Methods

2.1 Bacterial Strains and Media

E. coli DH5α (Invitrogen) were utilized for subcloning, plasmid amplification and maintenance. S. enterica serovar Typhi strain Ty21a and its ΔtyrS derivative were used as the basis for the generation of human vaccine strains. S. enterica serovar Typhimurium ΔaroA strain SL7207 was utilized for oral immunization studies in mice (Table 1). Unless otherwise stated, bacterial strains were grown aerobically in LB broth (Lennox) vegetal (Roth) at 37° C. with rigorous shaking (180-200 rpm), or on LB-Agar (Lennox) vegetal (Roth). Unless otherwise stated, antibiotic selection, as if necessary, was carried out using ampicillin (Sigma-Aldrich), kanamycin (Sigma-Aldrich) and chloramphenicol (Sigma-Aldrich) at final concentrations of 100, 25 and 20 μg/ml, respectively. For characterization experiments Salmonella spp. were grown in tryptic soy (TS) broth (Sigma-Aldrich) supplemented with appropriate antibiotics, if necessary. All strains were stored as glycerol (Roth) stock cultures (25-40%) at −80° C. For preparation of immunization aliquots, S. enterica serovar Typhi Ty21a ΔtyrS vaccine strains were grown in tryptic soy broth supplemented with 0.001% galactose (Merck).

2.2 in Silico Design of Antigen Selection

For vaccine construction, we have selected the structural proteins of SARS-CoV-2. The protein sequences of SARS-CoV-2 and the protein sequences of the adjuvant proteins for vaccine development were retrieved from UniProt database (https://www.uniprot.org/). Each of these protein sequences was screened for their average antigenic propensity using the antigenic peptides prediction tool (http://imed.med.ucm.es/Tools/antigenic.pl) (Kolaskar et al., 1990).

In silico cloning was performed using the SnapGene Viewer 5.3 and SnapGene 5.3.1. The optimized sequences of the NsiI- and SalI-fragments were synthesized by Invitrogen GeneArt Gene Synthesis (ThermoFisher scientific) and then cloned into one of their Standard GeneArt delivery vectors with ampicillin or kanamycin resistance markers (pMA respectively pMK)(Table 6). The DNA was delivered as 5 μg lyophilized plasmid DNA in microcentrifuge tube. After resolving in 50 μg Roti-CELL water (Roth) plasmid DNA was stored at −20° C.

2.3 Molecular Cloning

2.3.1. Standard Techniques.

All standard molecular methods were performed following published protocols (Sambroock and Russell, 2001). PCR-products and digests were purified either with QIAquick PCR Purification Kit (Qiagen) or the QIAquick Gel Extraction Kit (Qiagen) following the manufacturer's recommendations.

Restriction enzymes (FastDigest Mph1103I, FastDigest SalI) and T4 DNA ligase were purchased from Thermo Fisher Scientific. Oligonucleotides were synthesized by Sigma-Aldrich Chemie GmbH. PCR was performed with Biometra T3 Thermocycler Triple Block Laboratory PCR Thermal Cycler.

2.3.2 DNA Isolation.

Plasmids were purified with QIAprep Spin Miniprep Kit (Qiagen) and QIAGEN Plasmid Midi Kit (Qiagen) following the manufacturer's instructions. Chromosomal DNA was isolated using QIAamp DNA Mini Ki (Qiagen) following the manufacturer's instructions. The amount of DNA was measured using NanoDrop (Peglab, ND-1000).

2.3.3 Electroporation.

E. coli and Salmonella spp. strains were electroporated with recombinant plasmids using standard techniques. In brief, electrocompetent cultures were generated by harvesting them at an OD₆₀₀ of 0.6-1.2 by centrifugation. Pellets were washed three times with ice-cold 10% glycerol (Roth), concentrated 100× in 10% glycerol and stored at −80° C. For electroporation, cells were thawed on ice. Subsequently, 0.1-1 μg of DNA was mixed with 40 to 100 μl cell suspension and incubated on ice for approximately 1 min. DNA was introduced into the bacteria by using a Bio-Rad MicroPulser following the manufacturer's recommendations. For electroporation, 0.1 cm or 0.2 cm cuvettes (VWR) were used. After pulsing, the bacteria were incubated in SOB-broth (Roth) supplemented with 20 mM Glucose (Roth) for 1 h at 37° C., respectively at 30° C. when the cells were harboring the temperature-sensitive plasmid pCP20. After 1 h the bacteria were plated out on LB-Agar plates with the appropriate antibiotic selection.

2.3.4 PCR.

DNA templates were prepared by different methods.

For screening purposes, DNA was obtained from the supernatant after heat-inactivation of bacteria at 100° C. for 5 min and a following centrifugation step for 2 min at ≥10.000 rpm, 4° C. in a microcentrifuge. After the centrifugation step the lysate was cooled on ice and 1 to 2 μl were used as template for the PCR reactions using MyTaq HS Red Mix (Bioline, cat. BIO-25048, lot. PM348-BO82870).

For sequencing, chromosomal DNA of selected strains was isolated using QIAamp DNA Mini Ki (Quiagen) following the manufacturer's instructions and used as template in PCR-Reactions using primers flanking the tyrS-region in the chromosome (primer pair No 17 and 18, see table 8) using Phusion Plus DNA polymerase (ThermoFisher Scientific) following the manufacturer's instructions.

PCR cycle program:
12.5 μl Polymerase Mix
0.25 μl Primer forward (10 μM)
0.25 μl Primer reverse (10 μM)
2 μl DNA
10 μl H₂O ultrapure

Program:
Denaturation: 94° C. for 3 minutes
Cycling Stage (35 cycles): 94° C. for 45 seconds
   50-70° C. for 30 seconds
   72° C. for 2 minutes
Final Elongation: 72° C. for 5 minutes
Holding Stage: 4° C.

2.3.5 Agarose Gel Electrophoresis.

DNA fragments, if necessary and PCR products were mixed with 5× GelPilot DNA Loading Dye (Qiagen) and loaded on 1% agarose gels for subsequent control of PCR reactions and purification of desired DNA fragments. DNA bands of interest were excised from agarose gels and purified by GeneJET Gel Extraction Kit (ThermoFisher Scientific) or QIAquick Gel Extraction Kit (Quiagen) according to manufacturer's instructions.

Electrophoresis was performed with 1% agarose gels with 10 μl of the samples, 1×TAE buffer and at 110 V for around 30 minutes.

2.4 Construction of the Balanced-Lethal-System (BLS) for Plasmid Stabilization

Antibiotics are commonly used and are effective in providing plasmid stability under selective conditions. However, their use to stabilize plasmids in live vaccines is usually not applicable. Thus, without the selective pressure of antibiotics, plasmids might become unstable leading to their segregational loss. This in consequence leads to a sub-optimal efficacy of any bacterial live vector vaccine due to insufficient expression and presentation of the vaccine antigen to the human immune system (Spreng et al., 2005). The plasmid maintenance system the inventors previously designed to stabilize plasmids without any antibiotic selection pressure is made up of the chromosomal knockout of the gene tyrS encoding for the tyrosyl-tRNA-synthetase and the in trans complementation of this gene on the respective antigen-delivery-plasmid (Diessner, 2009).

2.4.1 Construction of the Chromosomal tyrS-Knockout-Strains

For the construction of the chromosomal tyrS knockout the inventors modified the method of “one-step inactivation of chromosomal genes using PCR products” which was described by Datsenko and Wanner, (Datsenko et al., 2000). As tyrS is an essential gene, this approach had to be adapted to avoid the lethal knockout of a gene without genetic complementation. A functionally active TyrS-expression cassette was therefore inserted into the PCR-template-plasmid pKD3. The TyrS expression cassette is located upstream of the promoter of the chloramphenicol resistance gene (cat) within the two FRT-sites. Hence the chromosomal tyrS was replaced by a fragment encoding for the antibiotic resistance and the gene encoding E. coli tyrS.

In brief, the FRT-flanked knock in fragment was amplified by PCR. The purified PCR-fragment was electroporated into S. typhi Ty21a, harbouring the temperature-sensitive easily curable Red helper plasmid pKD46 which carries the Red recombination system with the phage a Red recombinase under the control of an arabinose-inducible promoter. The chromosomal tyrS sequence was then replaced by the knock-in fragment by Red-mediated recombination in the flanking homologies (H1 and H2-region) resulting in strain S. enterica serovar Typhi Ty21a ΔtyrS (tyrS Cm)⁺ (Diessner, 2009).

This strain (clone 120) was transformed with the helper plasmid pCP20. The resulting strain is designated Ty21a-BLS-R (recipient) strain. The respective tyrS-complementing antigen delivery plasmids of the pSalVac Ax_By series was then electroporation. As a last step, all regions flanked by FRT-sites are eliminated by thermal induction of the pCP20 encoded flippase (Flp). The heat-induction simultaneously cured the strains from plasmid pCP20 due to its temperature-sensitive replication (Cherepanov et al., 1995). This generated the final antibiotic resistance gene free vaccine strain of the JMU-SalVac-100 series (S. enterica serovar Typhi Ty21a ΔtyrS pSalVac Ax_By ΔKan^{R}.

2.4.2 Construction of Template Plasmid pKD3-SpeI-tyrS-HisTag-s (Diessner, 2009)

The E. coli strain used for pKD3-derivate constructions was the pir-positive E. coli strain CC118 λpir (Herrero et al., 1990). In brief, first a SpeI-(BcuI)-restriction site was introduced into plasmid pKD3 by PCR using QuickChange Site-directed Mutagenesis Kit (Stratagene) according to manufacturers' instructions.

The oligonucleotides used for mutagenesis were Mut-pKD3-SpeI-forward and Mut-pKD3-SpeI-reverse (see table 7B)

The DNA was then transformed into electrocompetent cells of pir-positive E. coli strain CC118 λpir. After 1 h incubation at 37° C., the entire transformation reaction was plated on LB agar plates containing the appropriate antibiotics. The plates were incubated at 37° C. for >16 h. Plasmid DNA of several colonies was isolated and screened for positive clones by SpeI restriction analysis. One positive clone of putative pKD3-SpeI was selected and further confirmed by sequencing.

For construction of template plasmid pKD3-SpeI-tyrS-HisTag-s, E. coli DH5α chromosomal DNA was used as template to create the tyrS×6His expression cassette (tyrS EPC). The tyrS EPC in which the tyrS gene is under control of its native 5′-flanking DNA region (P_{WT}) was constructed as follows: first, a 1638 bp fragment was amplified with Pfu-Polymerase (Stratagene) by PCR using the forward primer tyrS-EPK-SpeI-reverse which binds 313-288 bp upstream from start codon of tyrS introducing a SpeI site and the reverse primer Ter-HisTag-1-forward 5′ which introduce a 6×His-tag upstream of the stop codon of the tyrS gene. The amplified DNA-fragment was then used as template in a second PCR using the same forward primer but a different reverse primer, namely SpeI-Ter-HisTag-2-forward which prolongs the template at the 3′-end to overall 1688 bp. Furthermore, the primer contains a SpeI recognition site. The resulting SpeI-P_{WT}tyrS6×his-fragment included 313 bp flanking the open reading frame (ORF) of the tyrS gene at its 5′ end, as well as 58 bp following the stop codon of this gene. After digestion with the SpeI restriction enzyme the DNA fragment was inserted into the single SpeI site of the template vector pKD3-SpeI resulting in plasmid pKD3-SpeI-tyrS-HisTag-s which bears the tyrS gene in the same orientation as the cat gene. The correct clone was confirmed by sequencing.

2.4.3 Chromosomal Integration of the (FRT-tyrS CmR-FRT)-PCR-Fragment into S. typhi Ty21a

Disruption of chromosomal tyrS by integration of a FRT-tyrS CmR-FRT-knock-in PCR fragment was performed following the method of Datsenko and Wanner (2000) but with modifications.

Briefly, S. typhi Ty21a was transformed with the temperature-sensitive Red recombinase helper plasmid pKD46. Transformants were grown in LB at 30° C. supplemented with ampicillin and 0.2% L-(+)-arabinose and then made electrocompetent as described by Datsenko and Wanner (2000). The plasmid pKD46 express the Red system under control of an arabinose-inducible promoter conferring the ability for homologous recombination with linear PCR under inducing conditions (Datsenko and Wanner, 2000).

The knock-in PCR fragment to disrupt chromosomal tyrS in S. typhi Ty21a was generated by amplifying the FRT site flanked tyrS-CmR cassette on plasmid pKD3-SpeI tyrS HisTag-s using BioTherm™ Taq polymerase (Genecraft). To minimize possible polar effects on downstream gene expression, primer were designed to yield in the final step of the procedure a tyrS in-frame deletion to begin 6 bp downstream of the translation start site and end 168 bp upstream of the stop codon. Design of primers were based on the published sequences S. enterica subsp. enterica serovar Typhi Ty2 (GenBank accession no. NC_004631). The primer knockout-forward 5′ has a 49 nt extension that is homologous to the 5′-region adjacent to tyrS (H1), including the start codon and the first codon of the gene as well as 20 nt homologous priming site 1 (P1) of template plasmid pKD3-SpeI tyrS HisTag-s. The primer knockout-reverse (Table 7B) binds to priming site 2 (P2) of the template plasmid and has a 51 nt extension that is homologous to region 1108-1158 bp downstream the start codon of tyrS (H2). The knock-in-PCR-product has an overall length of 2803 bp. The PCR products were gel-purified, digested with DpnI, repurified, and suspended in elution buffer (10 mM Tris, pH 8.0). Subsequently, the PCR products were transformed into S. typhi Ty21a harbouring pKD46. After one hour incubation at 30° C. in TS medium clones were selected on TS agar plates containing 5 μg/ml chloramphenicol and 0.2% arabinose. Following primary selection at 30° C., mutants were maintained on TS medium without selection. Single colonies were then grown on TS agar without antibiotics at 37° C. and then tested for ampicillin sensitivity to confirm the loss of the helper plasmid pKD46 (Datsenko and Wanner, 2000). Correct insertion of the knock-in PCR-product into the chromosomal tyrS gene of S. typhi Ty21 was investigated by PCR analysis. Subsequently clone 120 of S. enterica serovar Typhi Ty21a ΔtyrS (tyrS Cm)⁺ (clone 120) was selected and confirmed by sequencing (Diessner, 2009).

2.4.4 Cloning of P_{lacI-like} tyrS expression cassette in pMKhlyAIS2-CtxB-PSA (Gesser, 2010)

The plasmid pKD3 P_{WT} tyrS EPC was digested with the SpeI restriction enzyme. Subsequently the DNA-Fragment carrying the SpeI-P_{WT}tyrS EPC-fragment was inserted into the single SpeI site of pMKhlyAIS2 CtxB-PSA resulting in the plasmid pMKhlyAIS2 P_{WT}tyrS CtxB-PSA which bears the tyrS gene in the same orientation as the recombinant Hly gene cluster. The correct clone was confirmed by sequencing.

In E. coli, the LacI repressor which regulates expression of the lactose metabolic genes by binding to the lacO operator sequence (Lewis, 2005) is synthesized constitutively at a very low level, approximately 5 to 10 copies per cell (Gilbert et al., 1966, Muller-Hill et al., 1968). Thus, to reduce the expression on each single plasmid and therefore to favour the regulation of expression towards a higher plasmid copy number the tyrS×6his-coding sequence was cloned under the control of a lacI-derived promoter and integrated into the single SpeI-site of pMKhlyAIS2-CtxB-PSA. First, a PCR was performed using pMKhly CtxB-PSA P_{WT} tyrS EPC as template. The forward primer LacI-Prom.for binds to the region 48 nt to 21 nt upstream the start codon of the tyrS coding sequence. The Primer has an extension of 70 nt containing a lacI derived promoter sequence (P_{lacI-like}) and moreover a SalI plus a SpeI-restriction-site at the 5′-end. The reverse primer LacI-Ter-rev spans the terminal 29 nucleotides including the stop codon of the tyrS6×His coding sequence. The 55 nt-extension of the primer contains a transcription terminator sequence and a SalI plus a SpeI-restriction-site at the 5′-end. The PCR-product was cleaved with SpeI and cloned into the SpeI-site of pMKhlyAIS2 CtxB-PSA. In the resulting plasmid the orientation of the putative tyrS EPC is likewise the same as that of the recombinant hly gene cluster of the vector resulting in plasmid pMKhlyAIS2 P_{lac-like}tyrS CtxB-PSA (Gesser, 2010).

2.5 SDS-PAGE of Cell-Associated and Secreted Proteins.

Bacterial lysates were prepared from mid-log cultures grown in trypticase soy broth or LB medium containing appropriate antibiotics (if applicable). 0.5-2 ml of this culture were harvested by centrifugation and the supernatant was removed. The cell pellets were stored at −20° C. For SDS-PAGE, the pellets were resuspended in 100 to 200 μl of 1× Laemmli buffer with β-mercaptoethanol (Laemmli, 1970), boiled for 5 min and stored at −20° C. for SDS polyacrylamide gel electrophoresis (PAGE) analysis (->cell-associated proteins).

Periplasmic proteins were isolated by osmotic shock as previously described (Ludwig et al., 1999) with only slight modifications. In brief, the bacteria from a defined culture volume were centrifuged (Hereaus Megafuge, 30 min, 4° C., 6,000 rpm), washed with 10 mM Tris-HCl (pH 8.0) and resuspended in 0.25 volume (compared to the starting culture volume) of a solution containing 20% sucrose, 30 mM Tris-HCl (pH 8.0) and 1 mM Na-EDTA (shock buffer). After the addition of 2 μl 500 mM Na-EDTA, pH 8.0 per ml shock buffer, the mixture was incubated for 10 min at room temperature under gentle shaking. Subsequently, the bacteria were pelleted (Hereaus Megafuge, 30 min, 4° C., 6,000 rpm) and resuspended in 1 vol. of ice-cold H₂O. After incubation on ice for 10 min, bacteria were pelleted (Hereaus Megafuge, 30 min, 4° C., 6,000 rpm). The supernatant was used as periplasmic protein extract. For the analysis by SDS-PAGE, periplasmic proteins were precipitated by addition of ice-cold trichloroacetic acid (final concentration: 10%) and carefully resuspended in appropriate volume of 1× Laemmli buffer with β-mercaptoethanol by rinsing the walls of the centrifugation tube. Finally, the pH was neutralized by adding 10 μl of saturated Tris solution.

Supernatant proteins were obtained by precipitating proteins from the culture medium of bacteria grown as described above. Bacteria were pelleted from 12 to 50 ml of culture medium by centrifugation (Hereaus Megafuge, 30 min, 4° C., 6,000 rpm). 10 to 45 ml of the supernatant was transferred to a fresh tube and proteins were precipitated with ice-cold 10% trichloric acid (Applichem) overnight at 4° C. The next day, the precipitates were collected by centrifugation (Hereaus Megafuge, 30 min, 4° C., 6,000 rpm), washed with 1 ml ice-cold acetone p.a. (Applichem), air-dried and carefully resuspended in 250 to 450 μl 1× Laemmli buffer with β-mercaptoethanol (Laemmli, 1970) by rinsing the walls of the centrifugation tube. Finally, the pH was neutralized by adding 10 μl of saturated Tris solution. Alternatively, the pellets were resuspended in 250 to 450 μl native sample buffer (BioRad) following manufacturer's instructions.

Unless otherwise stated, SDS-PAGE was performed using the PerfectBlue Vertical Double Gel System from Peqlab. For one gel, 4 ml of 10% separating gel and 2.5 ml of 3.75% stacking gel was used. After gel polymerization and addition of 1×SDS running buffer to the chamber, the gel was loaded with the samples and 5 μl PageRuler Prestained Protein Ladder 10-180 kDa (ThermoFisher, cat. 26617). SDS-PAGE was performed at 90V for 20 min and then increased to 135V for another 2 h depending on the desired separation. The gel was then used for Coomassie staining using Bio-Safe™ Coomassie Stain (BioRAD, cat. 1610786) according to the manufacturer's protocol or by Western blotting.

2.6 Western Blot Analysis.

Unless otherwise stated, Western blotting was performed using the PerfectBlue Semi-Dry Blotter from Peqlab. For the transfer, 3 Whatman paper (Hartenstein, cat. GB33, 580×600, 330 g/m³) were cut to the size of 6×9 cm and, unless otherwise stated, 1 PVDF membrane (Roche, cat. 03010040001, lot. 46099200) were used. The membrane was activated in MeOH for 1 min and the Whatman papers were soaked in 1× Semi-Dry transfer buffer and finally assembled in the following order in the Blotter: 1 Whatman paper, membrane, gel, 2 Whatman paper. The transfer was achieved by applying 1 mA/cm² gel for 2 h. Transfer was controlled by staining the membranes with Ponceau-S solution (BioMol, cat. MB-072-0500) according to the manufacturer's instructions. Then the membrane was blocked in 5% milk for 1 h at RT and then rinsed 3 times with 1×TBS-T.

The primary antibody was then added overnight at 4° C. in TBS-T. The following day, the membrane was washed 3× for 5 min in 1×TBS-T. Afterwards, the membrane was incubated in the according secondary antibody in 5% milk for 1 h at RT and then washed again 3× for 5 min in 1×TBS-T. For detection, ECL solution 1 and 2 were mixed 1:1 and added to the membrane. If appropriate, Pierce™ ECL Plus Western Blotting Substrate (ThermoFisher scientific) was used according to manufacturer's instructions. Detection was performed using an Intas Chemiluminescence Imager.

Primary antibodies used for Western blotting: α-SARS-CoV-II Spike (Invitrogen, RBD, cat. PA5-114551, lot. WA3165784B, polyclonal rabbit), α-Flag (Sigma Aldrich, cat. F7425, polyclonal rabbit), α-CtxB (CytoMed Systems, cat. 203-1542, lot. 13031207, polyclonal rabbit), α-His (Novagen, cat. 70796_4, lot. 3290351, monoclonal mouse).

Secondary antibodies used: Mouse IgG HRP (Santa Cruz, cat. sc-2005), rabbit IgG HRP (Santa Cruz, cat. sc-2004).

2.7 Sequence Analysis.

Relevant regions of chromosomal or plasmid DNA were analyzed by PCR using appropriate primers (table 8) and/or sequenced. Sequencing was performed by Microsynth following manufacturer's recommendations. (Primer sequences for PCR analysis and for sequencing see table 8).

E coli NightSeq (Only for Screening Purposes)

In brief, clearly visible colonies were picked into E coli NightSeq® tubes (Microsynth) and also streaked out on LB-Agar plates containing appropriate antibiotic, if necessary, for preserving. Tubes were then sent to Microsynth and probes were sequenced by Sanger Sequencing.

### Microsynth Single-Tube Sequencing, Economy Run (Sequence Validation)

Purified or gel-extracted PCR-Products and Plasmid DNA of selected positive clones were isolated (QIAprep Spin Miniprep Kit, Quiagen and QIAGEN Plasmid Midi Kit, Quiagen) and relevant regions were sequenced by Microsynth Single-Tube Sequencing, economy run, following manufacturer's recommendations.

PCR products were loaded on 1% agarose gels and purified by GeneJET Gel Extraction Kit (ThermoFisher Scientific). Finally, concentration of gel extracted products were measured via NanoDrop and prepared for Microsynth Single-Tube Sequencing, economy run. See also methods 2.3.5.

### Next Generation Sequencing (Plasmid and Genome Sequencing)

Furthermore, selected plasmids as well as the genome of BLS-R-strain, clone 1 was sequenced (Microsynth).

In brief, BLS-R-strain harboring pCP20, clone 1 was cultured overnight in liquid LB broth without any antibiotic pressure at 37° C. with shaking. This strain was then grown on LB-Agar plates to obtain single colonies. Depletion of pCP20 was confirmed by picking colonies on TS-Agar with and without 100 μg/ml ampicillin and incubation at 30° C. for two days. No growth was detected on TS-Agar containing ampicillin. In parallel, colonies were picked on TS-Agar plates containing 20 μg/ml chloramphenicol to confirm chromosomal chloramphenicol resistance. A colony that fulfilled all requirements (chloramphenicol resistant, ampicillin sensitive) was taken from the LB-Agar plate and preserved (BLS-R, clone 1, ΔpCP20).

For sequencing chromosomal DNA was isolated using QIAamp DNA Mini Ki (Quiagen) following the manufacturer's instructions and then prepared according to Microsynths recommendations.

2.8 Confirmation of Strain Identity by Multiplex PCR.

JMU-SalVac-100 strain identity was confirmed by Multiplex PCR of genomic DNA according to a protocol published by Kumar et al. (2006)(Kumar et al., 2006) with slight modifications.

In brief, Multiplex PCR was performed using MyTaq HS Red Mix (Bioline, cat. BIO-25048, lot. PM348-BO82870). PCR primer see table 8.
12.5 μl MyTaq Mix
0.25 μl Primer #7 (10 μM)
0.25 μl Primer #8 (10 μM)
0.25 μl Primer #9 (10 μM)
0.25 μl Primer #10 (10 μM)
0.25 μl Primer #11 (10 μM)
0.25 μl Primer #12 (10 μM)
0.25 μl Primer #13 (10 μM)
0.25 μl Primer #14 (10 μM)
2 μl DNA
8.5 μl H₂O

Program:
Denaturation Stage: 94° C. for 3 minutes
Cycling Stage (35 cycles): 94° C. for 45 seconds
   50-70° C. for 30 seconds
   72° C. for 2 minutes
Final Elongation: 72° C. for 5 minutes
Holding Stage: 4° C.

Strain identification:

Salmonella Typhy Ty21a: 4 bands

Salmonella Typhimurium: 1 band

2.9 Bacterial Growth

Bacterial strains were plated on LB agar plates with appropriate antibiotics if required from glycerol stocks. Plates were incubated over night at 37° C. for at least 24 h. The bacteria were then transferred to TSA plates containing appropriate antibiotics and grown for another 24 h at 37° C. At the day of growth measurements, bacteria were suspended in 1 ml of TS medium and vortexed several times until the bacterial suspension was homogenous. Bacteria were then diluted 1:10 with TS medium in semi-micro cuvettes to determine the optical density (OD) at 600 nm wavelength. Subsequently bacterial solutions were diluted to yield an OD₆₀₀ of 0.1/ml. Finally, 300 μl of the diluted solutions were transferred to a 48-well cell culture dish in triplicates and growth was eventually measured by the TECAN MPlex software iControl 2.0.

2.10 Detection of mRNA Expression by qPCR.

Unless otherwise stated, bacterial pellets of 1 ml mid-log culture were used for RNA isolation with the miRNeasy micro Kit (50) (Qiagen, cat. 1071023, lot 166024980) following the manufacture's protocol. Amount of RNA was measured using NanoDrop (Peglab, ND-1000).

For cDNA synthesis, the RevertAid First Strand cDNA Synthesis Kit (ThermoFisher, cat. K1622) was used. One pg RNA was added to 1 μl Random Hexamer Primer and add RNase-free water to a total volume of 12 μl. After an incubation for 5 min at 65° C., 8 μl of the following master mix was added:
4 μl 5× reaction buffer
1 μl Ribolock RI (20 U/μl)
2 μl dNTP-Mix (10 mM)
1 μl RevertAid Reverse Transcriptase (200 U/μl)

The cDNA synthesis was performed by incubation for 5 min at 25° C., 60 min at 42° C. and 5 min at 70° C., and finally diluted 1:5 with RNase-free water.

5 μl of the diluted cDNA was added to 21 μl of the following master mix:
0.5 μl Primer forward (10 μM)
0.5 μl Primer reverse (10 μM)
10 μl 10×SyBrGreen
10 μl H₂O

qPCR was then performed in a One step Thermo Fisher and the following program was used:
Holding Stage: 95° C. for 10 minutes
Cycling Stage (40 cycles): 95° C. for 15 seconds
   60° C. for 1 minute
Melt Curve Stage: 95° C. for 15 seconds
   60° C. for 1 minute
   +0.3° C. up to 95° C. for 15 seconds

Primers used for qPCR are listed in table 12.

2.11. Method to Determine Plasmid Stability and Copy Number.

Plasmid maintenance in vitro was determined by serial passage of bacteria without any selective pressure. A “Generation 0” was generated from several strains and these bacteria were grown over 5 consecutive days in the absence of antibiotics. Each day and from each strain, at least 100 individual colonies were tested for the presence of the plasmid.

2.11.1 Production of “Generation 0”, the Starting Cultures for Plasmid Stability Testing.

Bacteria with plasmids stabilized by the BLS or antibiotic selection were plated from frozen stocks on TS-Agar or on TS-Agar supplemented with 25 μg/ml kanamycin and incubated at 37° C. overnight. The next day bacteria from each strain were transferred into 25 ml TS medium. After mixing by vortexing, the optical density OD₆₀₀ (Eppendorf Biophotometer) was adjusted in TS-Medium to about 0.05 to 0.1 in a final volume of about 120 ml TS medium with or without 25 μg/ml kanamycin. The cultures were incubated aerobically in 500 ml culture media flasks DURAN®, baffled, at 37° C. under rigorous shaking (180 rpm). After reaching an OD₆₀₀ of about 1.5 (mid-logarithmic phase), each culture was cooled at least for 15 min on ice to stop bacterial growth. These cultures were the starting point (Generation 0) to determine the kinetics of plasmid loss or maintenance.

2.11.2 Serial Passage and Plasmid Stability Testing and Copy Number Determination

The bacteria were transferred at 1:1000 to 1:2500 dilutions into fresh liquid medium (TS-Medium) and cultured to stationary phase (25% filling in flasks DURAN®, baffled at 37° C., 180 rpm). In the same way, bacterial cultures were passaged up to 5 times. Each day, serial dilutions of the strains harboring plasmids with kanamycin resistance gene were plated on TS agar plates without antibiotic selection and incubated at 37° C. for 18-24 h to obtain single colonies. At least 100 colonies per day and strain harboring plasmids with kanamycin resistance gene were selected randomly and grown on a fresh TS-agar plates containing 25 μg/ml kanamycin and on TS Agar without antibiotics for growth control, preserving and further testing. In case of the investigated BLS-stabilized vaccine strains cultures of day 5 were serial diluted and plated on TS agar plates. After incubation overnight at 37° C. at least 100 colonies of each strain were picked on TS agar for preserving and further testing. The presence of the BLS-stabilized plasmid (ΔKanR) in the investigated strains was monitored by PCR amplification assays using plasmid specific primers. In brief, bacterial material of each colony were transferred in 50 μl sterile water, lysed by boiling at 100° C. for 5 min, and cooled on ice. After centrifugation at 13,000 rpm for 2 min, 2 μl of the lysates were used as a template in PCR reactions using primer pairs 4/6, 6/23 and/or 68/69. Additionally, some PCR reactions were performed with primer pair 17/18 to confirm chromosomal deletion of tyrS.

For copy number determination, qPCR was performed (2.10) with the primers 62 and 63 (hlyB) for the quantification of the plasmid and primers 73 and 75 (slyB) for normalization against a single copy genomic gene.

2.11.3 Stability of Antigen Expression and Secretion

5×2 ml and 4×1 ml culture were transferred into Eppendorf tubes. After a centrifugation step of at least 1 min, 4° C., 20,817 rcf, (Eppendorf centrifuge 5174R), the supernatants were removed quantitatively and the cell pellets were stored at −20° C. until further analysis were performed (see Western blotting, qPCR, plasmid copy number determination). Unless otherwise stated, from each culture 2×47 ml were collected for preparation of extracellular proteins by TCA-precipitation of proteins from culture supernatant) (see 3.7.1 SDS-PAGE of bacterial lysates and secreted proteins).

2.12. Methods to Measure the Immune Response Elicited by JMU-SalVac-100 Strains

2.12.1 Preparation of Immunization Aliquots

Immunization aliquots of S. typhi Ty21a ΔtyrS-strains harboring one of the pSalVac Ax_By ΔKan vaccine plasmids were prepared as follows: Bacteria were cultivated in 500 ml TS-Medium (2 liter flask Duran, baffled) supplemented with 0.001% Galactose (Merck) at 37° C. with shaking until they reach mid-log phase (OD₆₀₀: about 1.5, Eppendorf BioPhotometer). Subsequently, strains were cooled down on ice for 30 min and then harvested by centrifugation in a Beckmann-Coulter centrifuge, JA 10 Rotor, 4° C., 30 min, 10,000 rpm. The pellets were resuspended and washed with approximately 40 ml 1× in ice-cold 1×DPBS (Gibco): 100% Glycerin (Roth) (4:1). The bacterial suspensions were then transferred into 50 ml Greiner tubes and centrifuged for 30 min, 4° C. (Hereaus, Megafuge 1.0). Subsequently, the cell pellets were resuspended in 5 ml 1×DPBS (Gibco): 100% Glycerin (Roth) (4:1) (concentration factor: about 100-fold) and aliquoted in 500-1000 ml portions for storage at −80° C.

Immunization aliquots of S. typhimurium SL7207 strains harboring one of our pSalVac Ax_By KanR vaccine plasmids were prepared as follows: Bacteria were cultivated in 500 ml TS-Medium (2 liter flask Duran, baffled) containing appropriate antibiotics for at least 12 h at 37° C. with shaking until they reach late-log phase (OD₆₀₀: about 5, Eppendorf BioPhotometer). Subsequently, strains were cooled down on ice for 30 min and then harvested by centrifugation in a Beckmann-Coulter centrifuge, JA 10 Rotor, 4° C., 30 min, 10,000 rpm. The Pellets were resuspended and washed with approximately 40 ml 1× in ice-cold 1×DPBS (Gibco): 100% Glycerin (Roth) (4:1). The bacterial suspensions were then transferred into 50 ml Greiner tubes and centrifuged for 30 min, 4° (Hereaus, Megafuge 1.0). Subsequently, the cell pellets were resuspended in 5 ml 1×DPBS (Gibco): 100% Glycerin (Roth) (4:1) (concentration factor: about 100-fold) and aliquoted in 500-1000 ml portions for storage at −80° C.

Aliquots were stored at −80° C. for at least 24 h before the CFU was determined by plating serial dilutions on BHI agar plates. The number of live colonies was determined by plating 100 μl of serial dilutions (10⁻⁶ to 10⁻⁸, each in duplicate) on TS agar plates without any antibiotic selection. Plating was performed using a sterile Drigalski-spatule. After incubation o/n at 37° C. colonies were counted. For counting, at least two agar-plates per serial dilution were counted, where the colony number is between 20 and 500 colonies. The CFU per ml per dilution series were calculated using the formula: CFU=(counts*dilution factor)×10.

2.12.2 Tolerability Study in Mice

Adult female BALB/c mice were randomly allocated to experimental groups and allowed to acclimatise for one week. The vaccine strains of Salmonella typhi and Salmonella typhimurium were prepared directly from the glycerol stocks as described (2.12.1). The adequate number of cryotubes of respective strains were thawed on ice, with each tube vortexed for 5 seconds at full speed every 30 seconds. Once fully thawed, the samples were vortexed again for 5 seconds. Immediately afterwards the adequate volumes of bacterial stocks were pipetted into a new, sterile 1.5 ml Eppendorf Safe-Lock Tube which were subsequently centrifuged at 14,000 rpm, 2 min, 4° C. Supernatants were discarded quantitatively by pipetting and pellets resuspended in an initial volume of 1×PBS by pipetting up and down at least 10 times. The exact volume of bacterial suspension was determined with the pipette and, if required, additional 1×PBS was added to achieve the desired bacterial concentration. Bacterial suspension was vortexed again at full speed for 5 seconds before being administered. For Salmonella typhi strains 30 μl of the suspension was applied intranasally per mouse (15 μl per nare). For Salmonella typhimurium, 200 μl were applied per oral per mouse. The remaining bacterial suspension was used to determine the actual dose by carrying out back plating. Serial dilutions were set up in duplicates for each of the bacterial strains.

All animals were observed for signs of ill health throughout the study. From Day 0 until the end of the experiment, animals were weighed three times each week. Animals with a bodyweight loss greater than fifteen percent (15%) of their initial (Day 0) bodyweight were culled.

2.12.3 Immunization of Mice

Intranasal Immunization with S. typhi Ty21a ΔtyrS Vaccine Strains.

The frozen immunization aliquots of S. typhi Ty21a ΔtyrS vaccine strains were thawed on ice, centrifuged, resuspended in PBS and adjusted to 1×10⁷ CFU per 30 μl. For intranasal immunization, adult BALB/c mice were anesthetized with isoflurane. Under the magnifying lamp, 10 μl of inoculant solution containing 1×10⁷ CFU of the S. typhi Ty21a ΔtyrS vaccine strain were applied to the nostrils of the mouse using a 20 μl pipette. To avoid aspiration of the infectious solution, the mouse was not returned to the cage until it has awakened.

Oral Immunization with S. typhimurium aroA SL7207 Vaccine Strains.

The frozen immunization aliquots of S. typhimurium aroA SL7207 vaccine strains were thawed on ice, centrifuged, resuspended in PBS and adjusted to 5×10¹⁰ CFU per 200 μl. This solution was first placed on ice and taken up into a 1 ml syringe and administered by gavage (22 G feeding needle).

At termination, bronchoalveolar lavage (BAL) and terminal blood samples were taken. Blood was processed to serum, and serum and BAL were analyzed by ELISA with antigens: Salmonella LPS (positive control), SARS-CoV-2: S-protein, N-protein.

2.12.4 ELISA

ELISA was used to detect IgM and IgG antibodies directed against the SARS-CoV 2 Spike 1 receptor binding domain (RBD) and the Nucleocapsid N Protein by ELISA kits (Alpha Diagnostic International). Samples were thawed on ice diluted with working sample solution. Immunoassays were performed according to the manufacturer's instructions and plates were analyzed on a microplate reader (TECAN MPlex) at wavelength 405 nm.

2.13.5 ELISpot

The ELISpot assay was used to determine the number of interferon-gamma (IFN-γ) secreting T cells from a given number of splenic leukocytes. The spleen cells of immunized and sham-immunized mice were restimulated with appropriate vaccine protein in vitro and thus used to demonstrate the formation of IFN-γ. This was demonstrated by a specific color reaction of the IFN-γ producing cells (spots) on a support membrane. PHA-M or PMA/Ionomycin was used as positive control for ELISpot readout, SARS-CoV-2 S-protein and N-protein as specific stimuli. Cell were left unstimulated as negative control for ELISpot readout.

3. Results

3.1 in Silico Design of Vaccine Antigens

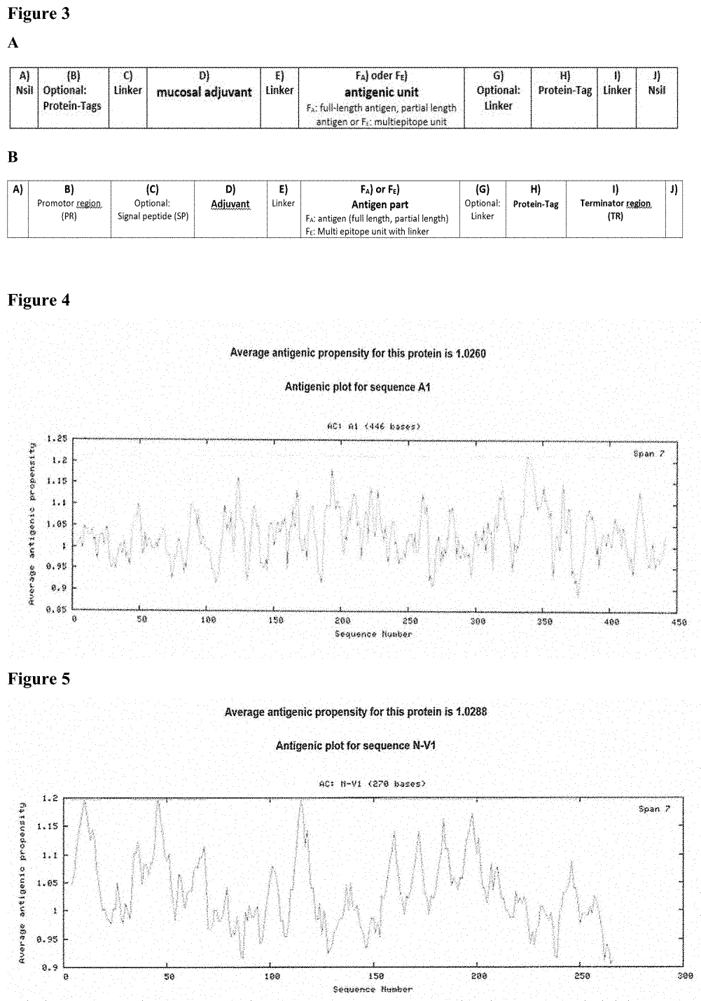

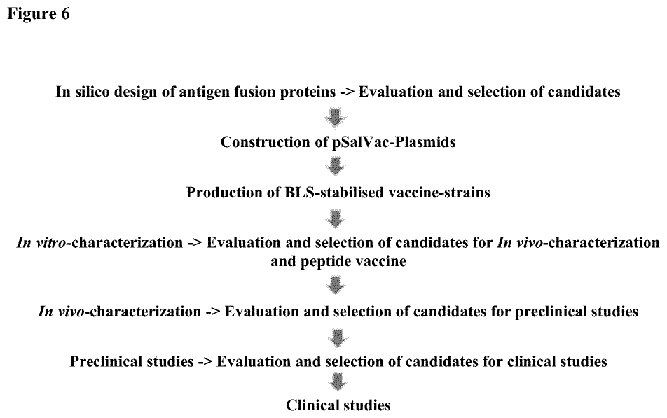

Predictions for SARS-CoV-2 antigens and adjuvants were performed as described (2.2) and the results are shown in table 2 and table 3, respectively. Proteins (full length, partial) with an average antigenic propensity score of greater than 0.9 were considered for vaccine construction. The various fusion protein subunits were designed by adding an adjuvant and an antigenic unit connected by specific linkers to provide adequate separation. EAAAK linker (Srivastava et al., 2020) was used to join the adjuvant and the adjacent sequence to facilitates domain formation and improve the adjuvant effect. If applicable, intra HTL, CTL, and B-cell epitopes were joined using GPGPG, AAY, and KK (Kalita et al., 2020), respectively to provide adequate separation of epitopes in vivo. (FIG. 3A, Table 4, A site; FIG. 3B, Table 5, B site). The average antigenic propensity of the antigens expressed in the A- and B-site is shown in FIGS. 4 and 5, respectively.

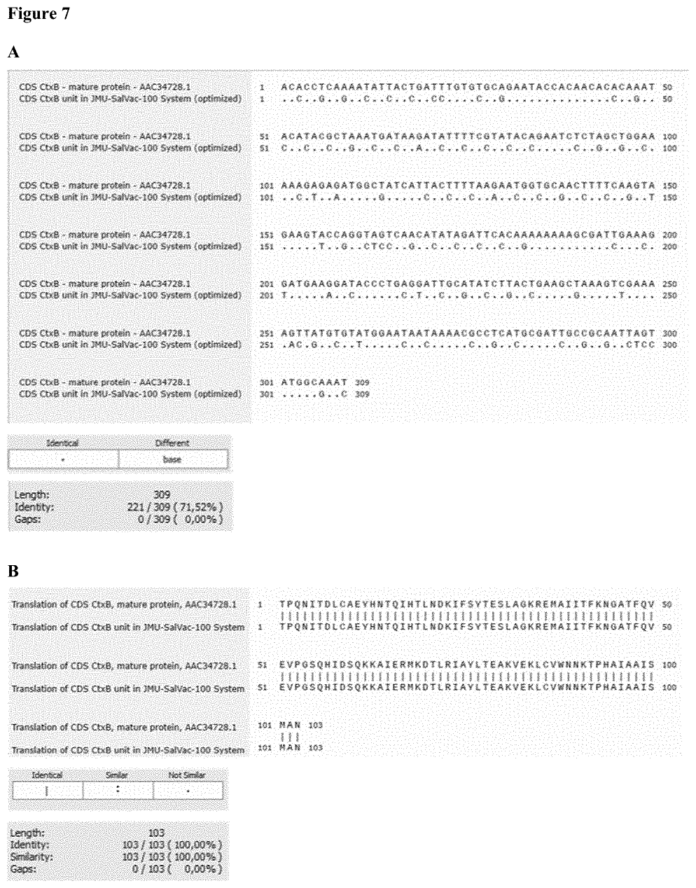

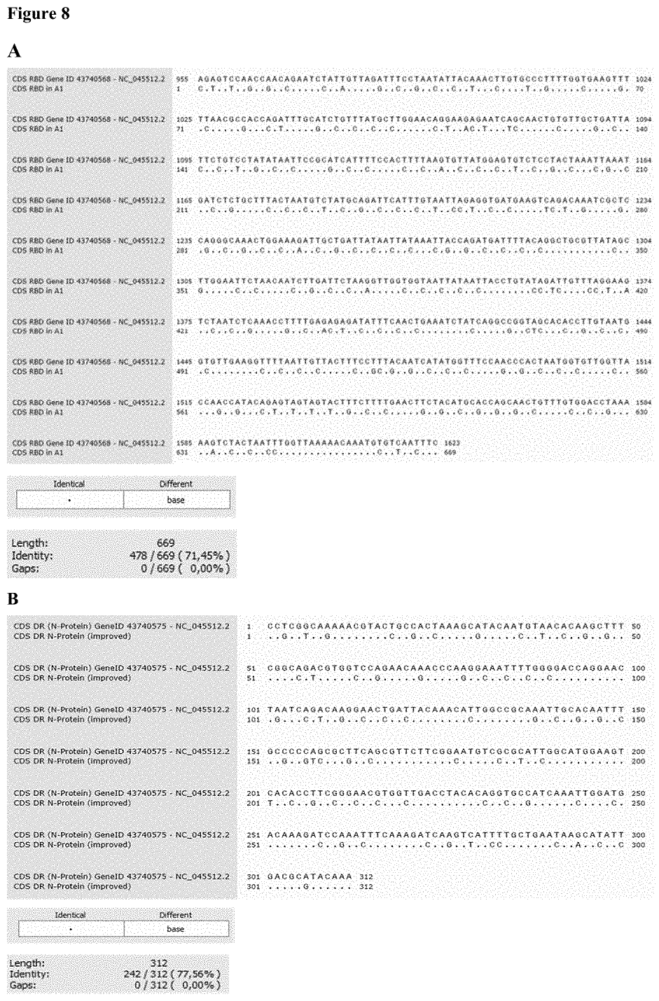

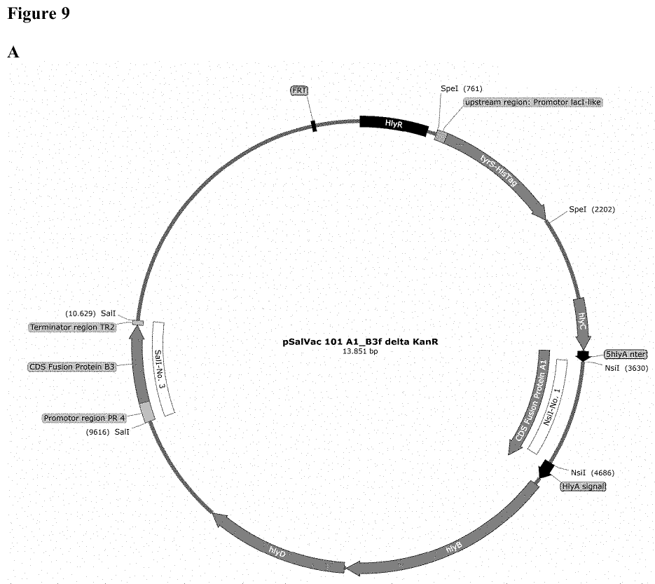

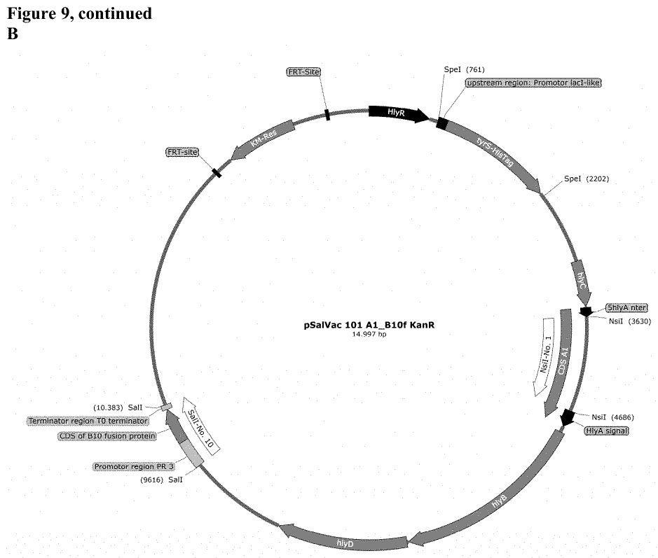

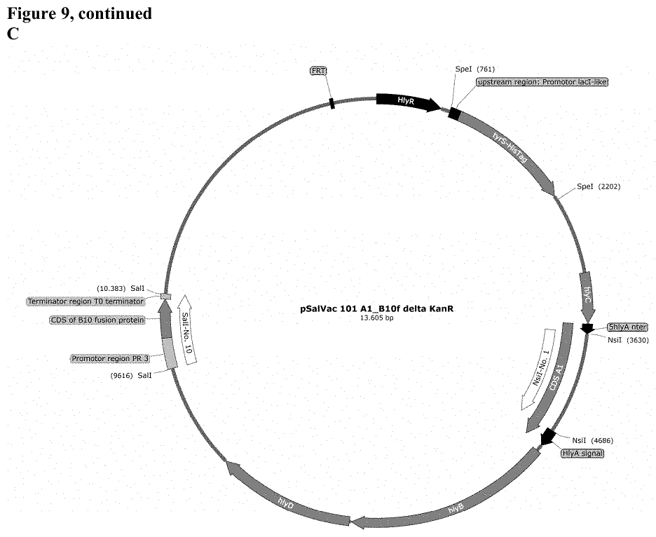

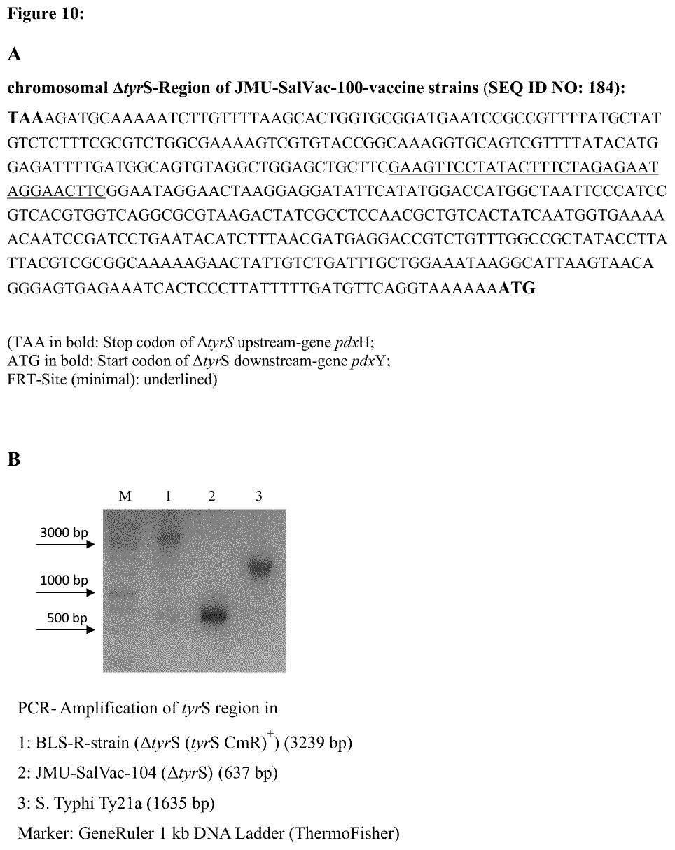

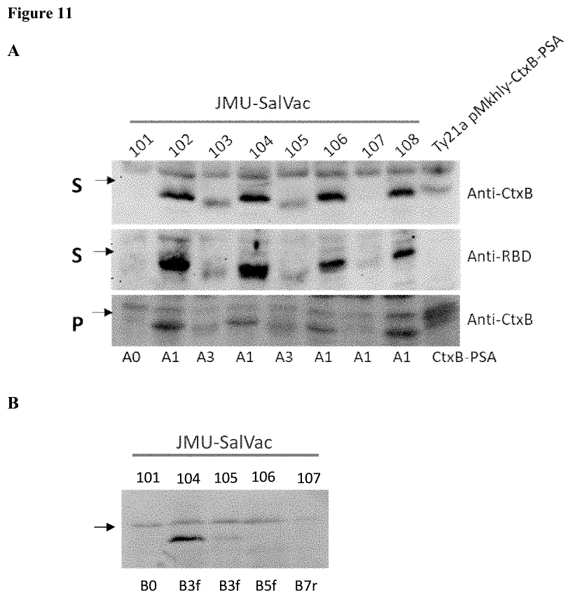

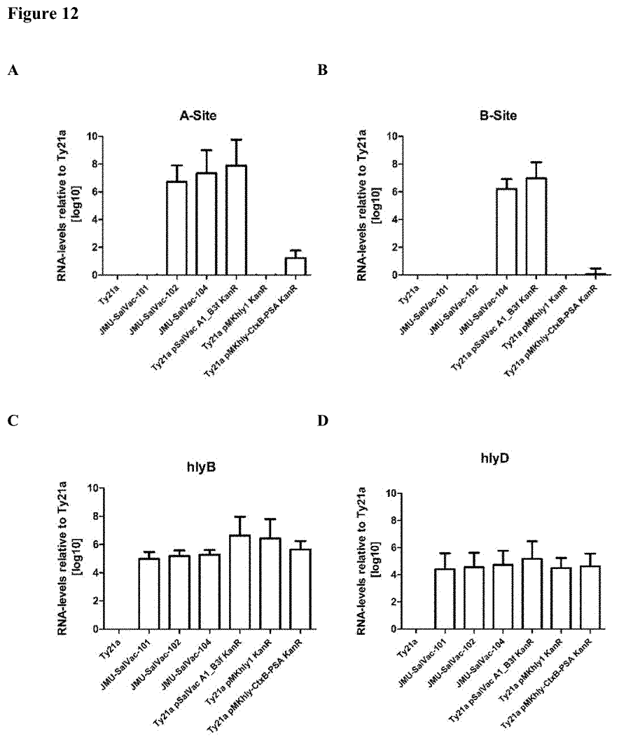

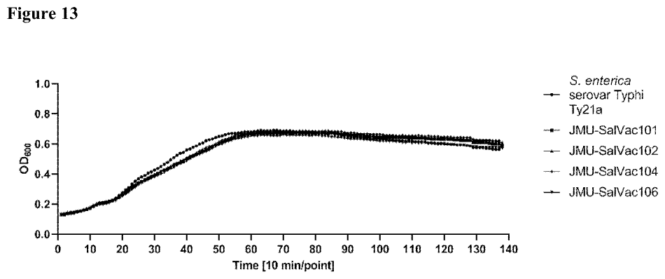

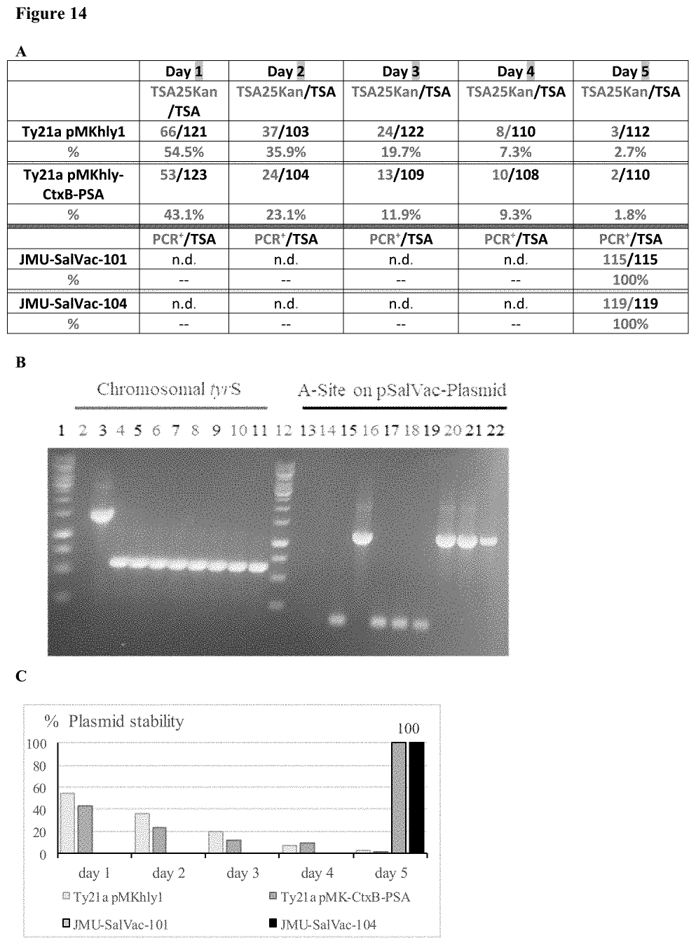

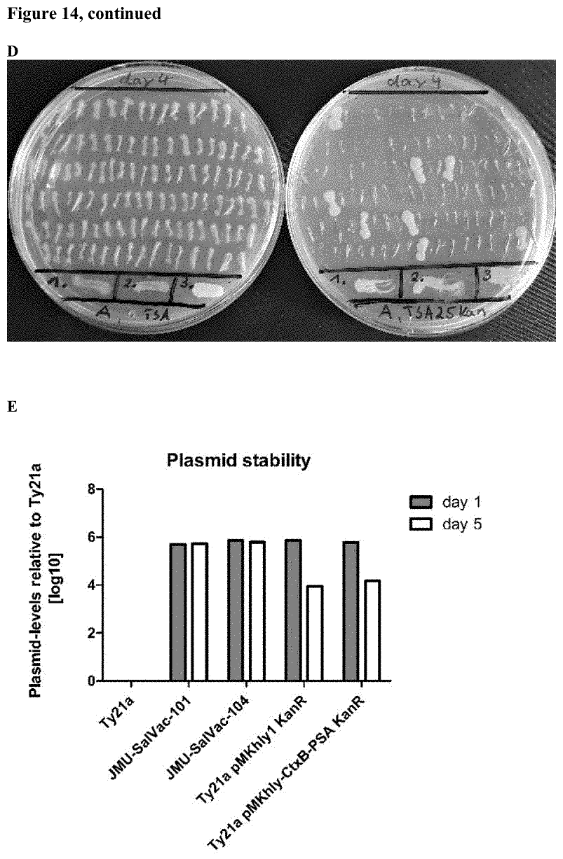

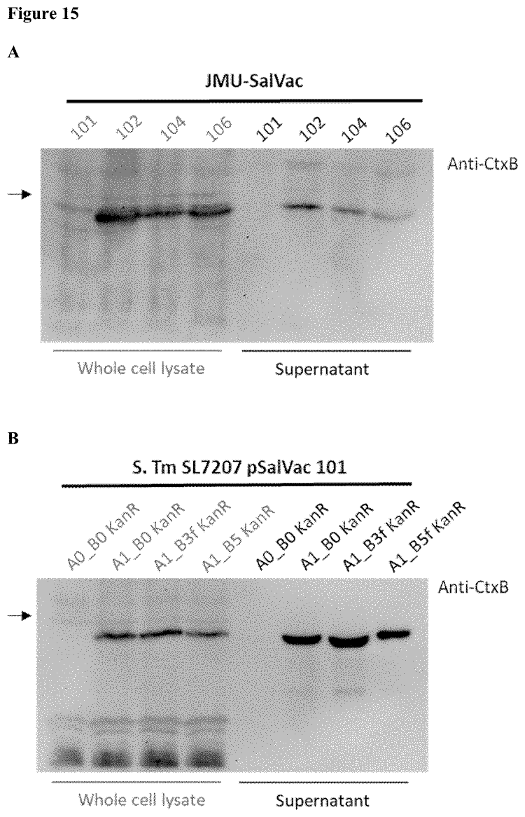

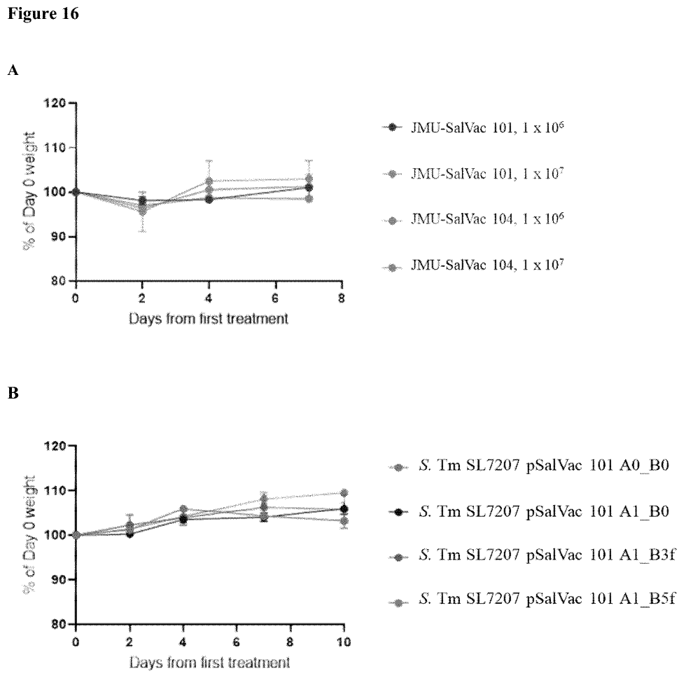

Java Codon Adaptation Tool (JCAT) (http://www.jcat.de/) (Grote et al., 2005) was used for codon optimization of the NsiI- and SalI-fragments to S. enterica Typhi (strain ATCC 700931/Ty2). The codon-optimized sequence for the CtxB adjuvant and the S-protein RBD are shown in FIGS. 7 and 8, respectively.

3.2 Generation of the Basic Vector pSalVac 001 A0_B0 KanR

For the generation of pSalVac 001 A0_B0 KanR, the plasmid pMKhly1ΔIS2 P_{lac-like}tyrS CtxB-PSA (Gesser, 2010) was digested with NsiI (FastDigest Mph1103I, Thermo Fisher Scientific). The 1017 bp-CtxB-PSA-NsiI-Fragment was cut out and the remaining plasmid backbone pMKhly1ΔIS2 P_{lac-like}tyrS was religated resulting in pSalVac 001 A0_B0 KanR (Table 9).

pSalVac 001 A0_B0 KanR, clone 2 was isolated from E. coli DH5 α and the correct sequence was confirmed by PCR using primer pair Nr. 4 and 6 (Table 8). DNA sequence of the entire plasmid was further analysed by sequencing (Microsynth). The map of the plasmid is shown in FIG. 1.

3.3 Generation of Plasmids of the pSalVac Ax_By-100 Series

pSalVac 001 A0_B0 KanR provides the basis of our various antigen delivery plasmids of the pSalVac Ax_By-100 series. It is derived from pBR322 and has a pMB1 origin of replication. For selection in vitro it has a kanamycin resistance expression cassette (KanR) that is flanked by two sites of flippase recognition targets (FRT-Sites).

Functional features of the pSalVac Ax_By plasmid 100 series are two independent expression cassettes for the expression of different combinations of adjuvant-antigen-fusion proteins.

The first expression cassette, named A-Site consists of the transcription enhancer sequence hlyR, the structural genes hlyC, hlyB and hlyD and two short residual sequences of the hlyA gene separated by an NsiI-restriction site (FIG. 2, FIG. 9).

The second expression cassette for Adjuvant-Antigen-fusion proteins, named B-site, is integrated into the unique SalI site of pSalVac 001 A0_B0 KanR.

For the generation of the different plasmids of the pSalVac Ax_By-100 series the NsiI-fragments were cloned into the A-(NsiI)-expression site, whereas the SalI-fragments were cloned into the B-(SalI)-expression site of the pSalVac 001 A0_B0 KanR vector.

In brief, the pSalVac 001 A0_B0 KanR vector or its derivates were digested with either NsiI (FastDigest Mph1103I, ThermoFisher Scientific) or with SalI (FastDigest SalI, ThermoFisher Scientific). Successful linearization of the plasmid was confirmed by agarose gel electrophoresis. Subsequently, Thermo Scientific™ FastAP™ Thermosensitive Alkaline Phosphatase (Thermo Fisher Scientific) was added for dephosphorylation of the vector DNA to prevent recircularization during ligation.

The respective pMK or pMA-Vector carrying the synthetic NsiI-fragments, respectively SalI-fragments (Table 6) (GeneArt Gene Synthesis, ThermoFisher scientific) were also digested with NsiI (FastDigest Mph1103I, ThermoFisher Scientific), respective with SalI (FastDigest SalI, ThermoFisher Scientific). After separation by agarose (Agarose NEEO ultra-quality, Roth) gel electrophoresis the fragments were cut out and purified with QIAquick Gel Extraction Kit (Qiagen) following the manufacturer's recommendations. The purified NsiI-, respective SalI-fragments were then ligated into the NsiI-, respectively SalI-digested, AP-treated vector plasmid. For ligation, T4 DNA-Ligase from ThermoFisher Scientific was used following manufacturer's instructions.

Clones were screened by PCR using priming pairs 4/6, 4/45, 68/69 and/or 6/23 for integration and orientation of NsiI-fragments into the A-site (FIG. 2). For integration and determination of orientation in the B-site, following primer pairs were used 21/22, 59/22, 21/34 and/or 39/40. Positive clones were further confirmed by sequencing (Microsynth) relevant regions (primer sequences for PCR analysis and for sequencing see Table 8). The plasmid pSalVac 101_A1_B3f ΔKanR is shown as an example in FIG. 9A, a list of generated pSalVac plasmids is shown in table 9.

3.4 Generation of the Balanced-Lethal Stabilized Vaccine Strains

In pSalVac 001/101 Ax_By KanR-plasmids, the kanamycin resistance gene is flanked by two Flippase (FLP) recognition target sites (FRT)-sites. This feature allows the excision by the site-specific enzyme FLP recombinase, which acts on the direct repeats of the FRT-sites. The FLP recombinase is encoded on the temperature-sensitive helper plasmid pCP20 and its temporal synthesis is induced by temperature. The vector that is inherited stably at temperatures of 30° C. and lower contains furthermore an ampicillin and chloramphenicol resistance gene for selection (Cherepanov et al., 1995, Datsenko et al., 2000).

For generation of the balanced-lethal stabilized vaccine strains, the flp-encoding helper plasmid pCP20 was electroporated into electrocompetent cells of S. typhi Ty21a (ΔtyrS (tyrS Cm)+, clone 120 and incubated for 2 days at 30° C. Subsequently a single clone (clone 1) was selected and used to make electrocompetent cells. This clone represents our BLS-(R)-recipient strain (Table 10).

Electrocompetent cells of BLS-R were then transformed with one of our tyrS-complementing antigen expressing plasmids of the pSalVac Ax_By KanR-100 series. After 1 h incubation at 30° C. in LB broth without antibiotic pressure, kanamycin/ampicillin/chloramphenicol triple resistant transformants were selected at 30° C. on LB agar plates containing 25 μg/ml kanamycin and 100 μg/ml ampicillin.

In contrast to the method described by Datsenko and Wanner (Datsenko et al., 2000) not only the FRT-flanking fragment in the chromosome but also the FRT-flanking kanamycin resistance gene fragment in the plasmid had to be eliminated. To assure elimination of all FRT flanked sequences we established a modified protocol for the elimination step.

In brief, BLS-intermediate strains (e.g. S. enterica serovar Typhi Ty21a ΔtyrS (tyrS Cm)+ harbouring pCP20 and one of our pSalVac 001/101 Ax_By KanR plasmids) were cultivated at 30° C. with rigorous shaking (180-200 rpm) in LB-broth containing 25 μg/ml kanamycin and 100 μg/ml ampicillin. The next day, the cultures were diluted 1:1000 into fresh LB-broth containing 100 μg/ml ampicillin to ensure selective pressure on the maintenance of the FLP helper plasmid pCP20. The diluted cultures were then subjected to temperature shifts starting with 1 h at 37° C. (flippase expression and induction), 1 min on ice and then 1 h at 30° C. (to allow replication of FLP helper plasmid pCP20). These temperature shifts were repeated 4 times resulting in an overall incubation time of about 8 h. After the last incubation step at 30° C., the cultures were grown on LB-agar plates supplemented with 100 μg/ml ampicillin to obtain single colonies. The plates were incubated at 30° C. until colonies were clearly visible. Then 4 to 10 single colonies were individually transferred to fresh LB-agar plates supplemented with 100 μg/ml ampicillin and incubated at 30° C. The same colonies were tested in parallel for the loss of the kanamycin resistance gene by growing them on TS-Agar supplemented with 25 μg/ml kanamycin and on TS-Agar-plates without any antibiotic as growth control. The TS-Agar plates were incubated over night at 37° C. Kanamycin sensitive (loss of resistance on pSalVac 001/101 Ax_By plasmid; FIG. 9A,C), ampicillin resistant (positive for helper plasmid) colonies were then grown in LB-broth without any antibiotics and incubated under rigorous shaking at 37° C. overnight to get deplete the temperature-sensitive helper plasmid pCP20. The next day cultures were grown on LB-agar plates without any antibiotic pressure to receive single colonies. About 5 colonies of each strain were then tested for sensitivity towards kanamycin, chloramphenicol and ampicillin: Chloramphenicol to test for loss of chromosomal integrated tyrS/CmR knock-in fragment, kanamycin to test for loss of resistance encoded on antigen delivery plasmid and furthermore ampicillin to test for loss of antibiotic resistance encoded on helper plasmid pCP20 and therefore for loss of pCP20 itself. All tested clones were also grown on LB-Agar plates without any antibiotic pressure for preserving and further characterization of each clone. Antibiotic sensitive clones were selected and the correct deletions of the FRT-intervening regions were further confirmed by PCR using primers flanking the deleted tyrS-Cm knock-in fragment on the chromosome (primer pair No 17 and 18, see Table 8) and also with primers flanking the kanamycin resistance gene on the plasmid (primer pair No 37 and 38, Table 8). Positive clones were further confirmed by complete or partial sequencing (Microsynth). The final strains without antibiotics resistance genes were designated JMU-SalVac-100 and numbered consecutively (-101, -102 etc.)(see Table 11).

3.5 Characterization of the Vaccine Strains

3.5.1. Expression of Antigens

The expression of antigens was tested by SDS-PAGE and Western blotting of bacterial lysates and supernatants (see 2.5 and 2.6). All strains of the JMU-SalVac-102 to 108 expressed the adjuvant-antigen fusions of the A site (FIG. 11A). However, strains with the designed A1 cassette secreted the fusion protein with high, those with the A3 cassette with low efficiency (FIG. 11A), since only the A1 antigen was detected in high amounts in the supernatant. From the vaccine adjuvant-antigen fusion proteins expressed in the B site only the B3f cassette was detectable (FIG. 11B). The inventors therefore selected JMU-SalVac-104 as initial candidate for further testing.

Expression of the antigens in the A- and B-sites was also determined by qRT-PCR (method 2.10; FIG. 12).

These results show that the bacteria of the invention can be used to achieve high antigen expression, which is expected to be advantageous for effective immunization in humans.

3.5.2. Growth Behavior of JMU-SalVac 100 Strains

Since the JMU-SalVac 100 strains produced large amounts of antigen the growth behavior was tested as described (2.9). There was no significant difference in growth behavior of the strains that produced the different antigens indicating that antigen production has no adverse effect on the Salmonella vaccine stains (FIG. 13).

3.5.3. Stability of the JMU-SalVac 100 plasmids

The stability of JMU-SalVac 100 plasmids was tested in the absence of antibiotics selection as described (2.11). There was a clear difference between the strains harboring plasmids with antibiotic resistance genes but without BLS and those with only the BLS and without antibiotics genes (FIG. 14A-C). Without stabilization by the BLS, the respective plasmid was retained in the experimental time frame of 5 days in less than 3% of the bacteria. But 100% of the strains JMU-SalVac-101 and JMU-SalVac-104 replicated the plasmids stabilized by BLS. As a result, the BLS-stabilized vaccine plasmids have a high degree of stability without antibiotics selection (FIG. 14A,B). A similar result was obtained when the copy number of the plasmid was determined on day 1 and day 5 in strains with and without BLS (FIG. 14E). The high stability of the plasmids was surprising and is expected to contribute to effective immunization by using the vaccines of the invention, while retaining an advantageous safety profile.

3.5.4. Characterization of the Selected Vaccine Strains

Based on the antigen expression (3.5.1.), bacterial growth (3.5.2.), and plasmid stability studies (3.5.3.), the S. typhi Ty21a vaccine strains JMU-SalVac-101 (control), JMU-SalVac-102 and JMU-SalVac-104 as well as S. typhimurium SL7207 with the respective plasmids pSalVac 001 A0_B0 (STM-pSalVac 001 A0_B0 KanR), pSalVac 101 A1_B0 KanR (STM-pSalVac 101 A1_B0) and pSalVac 101 A1_B3 KanR (STM-pSalVac 101 A1_B3) were selected for efficacy testing in mouse models. Immunization aliquots were prepared (2.12.1) and tested for expression and secretion of antigens. All strains expressed and secreted antigens as expected (FIG. 15).

3.6 Tolerability Study with the Vaccine Strains in Mouse Models

Following acclimatization, the animals were treated according to the schedule found below.

| | | Treatments | | |
|---|---|---|---|---|
| Groups | | Dose (ul or CFU) | Route | Regimen |
| 1 | Salmonella typhimurium SL7207 | 5 × 10¹⁰ CFU | PO | D0, D7 |
| | pSalVac 001 A0_B0 KanR | | | |
| | (vector control) | | | |
| 2 | Salmonella typhimurium SL7207 | 5 × 10¹⁰ CFU | PO | |
| | pSalVac 101 A1_B0 KanR | | | |
| 3 | Salmonella typhimurium SL7207 | 5 × 10¹⁰ CFU | PO | |
| | pSalVac 101 A1_B3f KanR | | | |
| 4 | Salmonella typhimurium SL7207 | 5 × 10¹⁰ CFU | PO | |
| | pSalVac 101 A1_B5f KanR | | | |
| 5 | JMU-SalVac-101 (control) | 10⁶ CFU | IN | D0, D7 |
| 6 | JMU-SalVac-101 (control) | 10⁷ CFU | IN | |
| 7 | JMU-SalVac-104 | 10⁶ CFU | IN | |
| 8 | JMU-SalVac-104 | 10⁷ CFU | IN | |

Following administrations of bacterial strains, animals were monitored for any signs of adverse effects for 10 days. Oral treatments with Salmonella typhimurium showed no adverse effects, with the proposed dose of 5×10¹⁰ well tolerated (FIG. 14A). Based on initial testing results, the intranasal application of S. typhi was performed with two different doses. The protocol identified doses of 1×10⁶ and 1×10⁷ of S. typhi were equally well tolerated (FIG. 14B).

The tolerated doses reported in the present Example indicate that the vaccines of the present invention are safe in mice. Furthermore, combined oral and intranasal testing of attenuated Salmonella-based vaccines in mice is an accepted tolerability test with predictive value for the safety of such vaccines in humans (see, for instance, Reddy et al., 2021). The tolerated doses which are reported in the present application indicate that the vaccines of the invention are also safe in humans, at doses which are expected to be efficacious in humans.

3.7 Humoral and Cellular Immune Response to JMU-SalVac 100 Strains

S. Tm SL7207 pSalVac 101 A0_B0 (vector control), S. Tm SL7207 pSalVac 101 A1_B0, S. Tm SL7207 pSalVac 101 A1_B3f, and S. Tm SL7207 pSalVac 101 A1_B5f were used for peroral immunization as described in chapter 2.12.3 In addition, JMU-SalVac 101 (A0_B0), -102 (A1_B0), -104 (A1_B3f) and -106 (A1_B5f) were applied intranasally as described in 2.12.3 All the strains expressing the RBD of the S-protein elicited a significant IgG response as measured by ELISA (2.12.4). The response against the N-protein was higher against the B3f antigen compared to the B5f antigen (e.g. strains S. Tm SL7207 pSalVac 101 A1_B3f: JMU-SalVac 104).

ELISpot assays revealed increased IFN-7 responses in S- and N-protein stimulated splenocytes in mice immunized with antigen-expressing S. typhimurium and S. typhi strains, indicative of a T cell response.

In view of these results, it is expected that the vaccines of the invention will provide effective protection against the respective corona viruses in humans.

### REFERENCES

Arakawa, T., Yu, J., Chong, D. K., Hough, J., Engen, P. C. and Langridge, W. H. (1998). A plant-based cholera toxin B subunit-insulin fusion protein protects against the development of autoimmune diabetes. Nat Biotechnol 16, 934-938.
Cheminay, C. and Hensel, M. (2008). Rational design of Salmonella recombinant vaccines. Int J Med Microbiol 298, 87-98.
Chen, Y., Liu, Q. and Guo, D. (2020). Emerging coronaviruses: Genome structure, replication, and pathogenesis. J Med Virol 92, 418-423.
Cherepanov, P. P. and Wackernagel, W. (1995). Gene disruption in Escherichia coli: TcR and KmR cassettes with the option of Flp-catalyzed excision of the antibiotic-resistance determinant. Gene 158, 9-14.
Condor Capcha, J. M., Lambert, G., Dykxhoorn, D. M., Salerno, A. G., Hare, J. M., Whitt, M. A., et al. (2020). Generation of SARS-CoV-2 Spike Pseudotyped Virus for Viral Entry and Neutralization Assays: A 1-Week Protocol. Front Cardiovasc Med 7, 618651.
Datsenko, K. A. and Wanner, B. L. (2000). One-step inactivation of chromosomal genes in Escherichia coli K-12 using PCR products. Proc Natl Acad Sci USA 97, 6640-6645.
Diessner, E. J. (2009) Construction of a balanced lethal systems for Salmonella typhi Ty21a. In Dissertation
Fensterle, J., Bergmann, B., Yone, C. L., Hotz, C., Meyer, S. R., Spreng, S., et al. (2008). Cancer immunotherapy based on recombinant Salmonella enterica serovar Typhimurium aroA strains secreting prostate-specific antigen and cholera toxin subunit B. Cancer Gene Ther 15, 85-93.
Galan, J. E., Nakayama, K. and Curtiss, R., 3rd (1990). Cloning and characterization of the asd gene of Salmonella typhimurium: use in stable maintenance of recombinant plasmids in Salmonella vaccine strains. Gene 94, 29-35.
Gao, W., Tamin, A., Soloff, A., D'Aiuto, L., Nwanegbo, E., Robbins, P. D., et al. (2003). Effects of a SARS-associated coronavirus vaccine in monkeys. Lancet 362, 1895-1896.
Gentschev, I., Mollenkopf, H., Sokolovic, Z., Hess, J., Kaufmann, S. H. and Goebel, W. (1996). Development of antigen-delivery systems, based on the Escherichia coli hemolysin secretion pathway. Gene 179, 133-140.
Germanier, R. and Fuer, E. (1975). Isolation and characterization of Gal E mutant Ty 21a of Salmonella typhi: a candidate strain for a live, oral typhoid vaccine. J Infect Dis 131, 553-558.
Gesser, M. B. A. (2010). Optimierung eines Balanced Lethal Systems für Salmonella typhi Ty21a. Dissertation
Gilbert, W. and Muller-Hill, B. (1966). Isolation of the lac repressor. Proc Natl Acad Sci USA 56, 1891-1898.
Gomez-Duarte, O. G., Pasetti, M. F., Santiago, A., Sztein, M. B., Hoffman, S. L. and Levine, M. M. (2001). Expression, extracellular secretion, and immunogenicity of the Plasmodium falciparum sporozoite surface protein 2 in Salmonella vaccine strains. Infect Immun 69, 1192-1198.
Grote, A., Hiller, K., Scheer, M., Munch, R., Nortemann, B., Hempel, D. C. and Jahn, D. (2005). JCat: a novel tool to adapt codon usage of a target gene to its potential expression host. Nucleic Acids Res 33, W526-531.
Herrero, M., de Lorenzo, V. and Timmis, K. N. (1990). Transposon vectors containing non-antibiotic resistance selection markers for cloning and stable chromosomal insertion of foreign genes in gram-negative bacteria. J Bacteriol 172, 6557-6567.
Hess, J., Gentschev, I., Miko, D., Welzel, M., Ladel, C., Goebel, W. and Kaufmann, S. H. (1996). Superior efficacy of secreted over somatic antigen display in recombinant Salmonella vaccine induced protection against listeriosis. Proc Natl Acad Sci USA 93, 1458-1463.
Hoffmann, M., Kleine-Weber, H., Schroeder, S., Kruger, N., Herrler, T., Erichsen, S., et al. (2020). SARS-CoV-2 Cell Entry Depends on ACE2 and TMPRSS2 and Is Blocked by a Clinically Proven Protease Inhibitor. Cell 181, 271-280 e278.
Hoiseth, S. K. and Stocker, B. A. (1981). Aromatic-dependent Salmonella typhimurium are non-virulent and effective as live vaccines. Nature 291, 238-239.
Holmgren, J., Adamsson, J., Anjuere, F., Clemens, J., Czerkinsky, C., Eriksson, K., et al. (2005). Mucosal adjuvants and anti-infection and anti-immunopathology vaccines based on cholera toxin, cholera toxin B subunit and CpG DNA. Immunol Lett 97, 181-188.
Kalita, P., Padhi, A. K., Zhang, K. Y. J. and Tripathi, T. (2020). Design of a peptide-based subunit vaccine against novel coronavirus SARS-CoV-2. Microb Pathog 145, 104236.
Kim, T. W., Lee, J. H., Hung, C. F., Peng, S., Roden, R., Wang, M. C., et al. (2004). Generation and characterization of DNA vaccines targeting the nucleocapsid protein of severe acute respiratory syndrome coronavirus. J Virol 78, 4638-4645.
Kolaskar, A. S. and Tongaonkar, P. C. (1990). A semi-empirical method for prediction of antigenic determinants on protein antigens. FEBS Lett 276, 172-174.
Kopecko, D. J., Sieber, H., Ures, J. A., Furer, A., Schlup, J., Knof, U., et al. (2009). Genetic stability of vaccine strain Salmonella typhi Ty21a over 25 years. Int J Med Microbiol 299, 233-246.
Kumar, S., Balakrishna, K. and Batra, H. V. (2006). Detection of Salmonella enterica serovar Typhi (S. Typhi) by selective amplification of invA, viaB, fliC-d and prt genes by polymerase chain reaction in multiplex format. Lett Appl Microbiol 42, 149-154.
Laemmli, U. K. (1970). Cleavage of structural proteins during the assembly of the head of bacteriophage T4. Nature 227, 680-685.
Letko, M., Marzi, A. and Munster, V. (2020). Functional assessment of cell entry and receptor usage for SARS-CoV-2 and other lineage B betacoronaviruses. Nat Microbiol 5, 562-569.
Lewis, M. (2005). The lac repressor. C R Biol 328, 521-548.
Ludwig, A., Bauer, S., Benz, R., Bergmann, B. and Goebel, W. (1999). Analysis of the SlyA-controlled expression, subcellular localization and pore-forming activity of a 34 kDa haemolysin (ClyA) from Escherichia coli K-12. Mol Microbiol 31, 557-567.
Lycke, N. (2005). Targeted vaccine adjuvants based on modified cholera toxin. Curr Mol Med 5, 591-597.
Muller-Hill, B., Crapo, L. and Gilbert, W. (1968). Mutants that make more lac repressor. Proc Natl Acad Sci USA 59, 1259-1264.
Ou, X., Liu, Y., Lei, X., Li, P., Mi, D., Ren, L., et al. (2020). Characterization of spike glycoprotein of SARS-CoV-2 on virus entry and its immune cross-reactivity with SARS-CoV. Nat Commun 11, 1620.
Sadeghi, H., Bregenholt, S., Wegmann, D., Petersen, J. S., Holmgren, J. and Lebens, M. (2002). Genetic fusion of human insulin B-chain to the B-subunit of cholera toxin enhances in vitro antigen presentation and induction of bystander suppression in vivo. Immunology 106, 237-245.
Spreng, S. and Viret, J. F. (2005). Plasmid maintenance systems suitable for GMO-based bacterial vaccines. Vaccine 23, 2060-2065.
Srivastava, S., Verma, S., Kamthania, M., Kaur, R., Badyal, R. K., Saxena, A. K., et al. (2020). Structural Basis for Designing Multiepitope Vaccines Against COVID-19 Infection: In Silico Vaccine Design and Validation. JMIR Bioinform Biotech 1, e19371.
Suthar, M. S., Zimmerman, M. G., Kauffman, R. C., Mantus, G., Linderman, S. L., Hudson, W. H., et al. (2020). Rapid Generation of Neutralizing Antibody Responses in COVID-19 Patients. Cell Rep Med 1, 100040.
To, K. K., Tsang, O. T., Leung, W. S., Tam, A. R., Wu, T. C., Lung, D. C., et al. (2020). Temporal profiles of viral load in posterior oropharyngeal saliva samples and serum antibody responses during infection by SARS-CoV-2: an observational cohort study. Lancet Infect Dis 20, 565-574.
Wan, Y., Shang, J., Graham, R., Baric, R. S. and Li, F. (2020). Receptor Recognition by the Novel Coronavirus from Wuhan: an Analysis Based on Decade-Long Structural Studies of SARS Coronavirus. J Virol 94.
Xu, D., Cisar, J. O., Poly, F., Yang, J., Albanese, J., Dharmasena, M., et al. (2013). Genome Sequence of Salmonella enterica Serovar Typhi Oral Vaccine Strain Ty21a. Genome Announc 1.
Yuki, Y., Byun, Y., Fujita, M., Izutani, W., Suzuki, T., Udaka, S., et al. (2001). Production of a recombinant hybrid molecule of cholera toxin-B-subunit and proteolipid-protein-peptide for the treatment of experimental encephalomyelitis. Biotechnol Bioeng 74, 62-69.
Zhou, P., Yang, X. L., Wang, X. G., Hu, B., Zhang, L., Zhang, W., et al. (2020). A pneumonia outbreak associated with a new coronavirus of probable bat origin. Nature 579, 270-273.

1. A live-attenuated bacterium of the genus Salmonella comprising a recombinant plasmid encoding a fusion protein, wherein the fusion protein comprises:
(i) a coronavirus antigen; and
(ii) an adjuvant peptide.

2. The bacterium of claim 1, wherein the bacterium is of the species Salmonella enterica.

3. The bacterium of claim 1, wherein the bacterium is a Salmonella enterica serovar Typhi strain.

4. The bacterium of claim 3, wherein the bacterium is the Ty21 a strain.

5. The bacterium of claim 1, wherein the adjuvant is a (i) mucosal adjuvant, or (ii) a toll-like receptor agonist or β-defensin.

6. The bacterium of claim 1, wherein the plasmid encodes a first fusion protein and a second fusion protein, wherein each fusion protein comprises:
(i) a coronavirus antigen; and
(ii) an adjuvant peptide.

7. The bacterium of claim 6, wherein the first fusion protein comprises:
(i) a coronavirus antigen; and
(ii) a mucosal adjuvant peptide.

8. The bacterium of claim 7, wherein the second fusion protein comprises:
(i) a coronavirus antigen; and
(ii) a toll-like receptor agonist or β-defensin.

9. The bacterium of claim 5, wherein the mucosal adjuvant is an interleukin-2 or a cholera toxin B subunit.

10. The bacterium of claim 5, wherein the toll-like receptor agonist is a Neisseria PorB or 50 s ribosomal protein L7/L12.

11. The bacterium of claim 5, wherein the β-defensin is human β-defensin 1, human β-defensin 2, human β-defensin 3 or human β-defensin 4.

12. The bacterium of claim 1, wherein the coronavirus antigen is a SARS-CoV-2 antigen.

13. The bacterium of claim 1, wherein the coronavirus antigen is selected from any one of SEQ ID NOs: 11-18, 120, 122, 124, 126, 128, 130, 132, 134, 136, 138, 140, 142, 144, 168, or 170 or is an antigenic fragment of any one of SEQ ID NOs: 11-18, 120, 122, 124, 126, 128, 130, 132, 134, 136, 138, 140, 142, 144, 168, or 170.

14. The bacterium of claim 1, wherein the coronavirus antigen is SEQ ID NO: 11 or an antigenic fragment thereof.

15. The bacterium of claim 1, wherein the coronavirus antigen is SEQ ID NO: 12 or an antigenic fragment thereof.

16. The bacterium of claim 1, wherein the coronavirus antigen is SEQ ID NO: 13 or an antigenic fragment thereof.

17. The bacterium of claim 1, wherein the coronavirus antigen is SEQ ID NO: 14 or an antigenic fragment thereof.

18. The bacterium of claim 1, wherein the coronavirus antigen is SEQ ID NO: 15 or an antigenic fragment thereof.

19. The bacterium of claim 1, wherein the coronavirus antigen is SEQ ID NO: 16 or an antigenic fragment thereof.

20. The bacterium of claim 1, wherein the coronavirus antigen is SEQ ID NO: 17 or an antigenic fragment thereof.

21. The bacterium of claim 1, wherein the coronavirus antigen is SEQ ID NO: 18 or an antigenic fragment thereof.

22. The bacterium of claim 1, wherein the fusion protein further comprises a secretion signal peptide.

23. The bacterium of claim 22, wherein the secretion signal peptide is the hemolysin A secretion signal peptide, and the plasmid further encodes HlyB and HlyD.

24. The bacterium of claim 23, wherein the plasmid further encodes HlyC and/or HlyR.

25. The bacterium of claim 1, wherein the bacterium and/or plasmid does not comprise an antibiotic marker.

26. The bacterium of claim 1, wherein the bacterium is a ΔtyrS strain and the plasmid further encodes tyrS.

27. The bacterium of claim 1, wherein the plasmid is integrated into the chromosome of the bacterium or replicates independently of the chromosome of the bacterium.

28. A combination product comprising:
(a) the bacterium of claim 1; and
(b) at least one of the one or more fusion proteins encoded by the plasmid of said bacterium.

29. A vaccine comprising the bacterium of claim 1.

30. (canceled)

31. A method of treating a disease or disorder caused by a member of the coronavirus family, the method comprising administering to a subject in need thereof the bacterium of claim 1.

32. The method of claim 31, wherein the disease or disorder is COVID-19.

33. A kit comprising:
(a) a live-attenuated bacterium of the genus Salmonella; and
(b) a recombinant plasmid encoding a fusion protein, wherein the fusion protein comprises:
(i) a coronavirus antigen; and
(ii) an adjuvant peptide.

34. The kit of claim 33, wherein the live-attenuated bacterium and the recombinant plasmid are according to claim 1.
